(19) United States
(12) Patent Application Publication
(10) Pub. No.: US 2023/0203044 A1
(43) Pub. Date: Jun. 29, 2023

(54) PYRAZOLO[1,5-D][1,2,4]TRIAZINE-5(4H)-ACETAMIDES AS INHIBITORS OF THE NLRP3 INFLAMMASOME

(71) Applicant: JANSSEN PHARMACEUTICA NV, Beerse (BE)

(72) Inventors: Daniel OEHLRICH, Beerse (BE); Michiel Luc Maria VAN GOOL, Madrid (ES); Nina VAN OPDENBOSCH, Beerse (BE); Mohamed LAMKANFI, Beerse (BE); Josep LLAVERIA CROS, Madrid (ES); Carlos Manuel MARTINEZ VITURRO, Madrid (ES); Dries VAN ROMPAEY, Beerse (BE)

(21) Appl. No.: 17/996,090

(22) PCT Filed: Apr. 15, 2021
(86) PCT No.: PCT/EP2021/059789

§ 371 Date: Oct. 13, 2022

(30) Foreign Application Priority Data
Apr. 15, 2020 (EP) 20382297.8

(51) Int. Cl. C07D 487/04; C07D 519/00; C07F 7/08; A61K 45/06

(52) U.S. Cl. CPC C07D 487/04; C07D 519/00; C07F 7/0812; A61K 45/06

(57) ABSTRACT

The invention relates to novel compounds for use as in-hibitors of NLRP3 inflammasone production, wherein such compounds are as defined by compounds of formula (I) and wherein the integers

R¹, R² and R³ are defined in the description, and where the compounds may be useful as medicaments, for instance for use in the treatment of a disease or disorder that is associated with NLRP3 inflammasome activity.

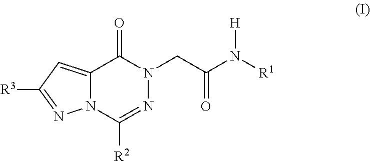

### FIELD OF THE INVENTION

The present invention relates to novel pyrrolotriazinones that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND OF THE INVENTION

Inflammasomes, considered as central signalling hubs of the innate immune system, are multi-protein complexes that are assembled upon activation of a specific set of intracellular pattern recognition receptors (PRRs) by a wide variety of pathogen- or danger-associated molecular patterns (PAMPs or DAMPs). To date, it was shown that inflammasomes can be formed by nucleotide-binding oligomerization domain (NOD)-like receptors (NLRs) and Pyrin- and HIN200-domain-containing proteins (Van Opdenbosch N and Lamkanfi M. Immunity, 2019 Jun. 18; 50(6):1352-1364). The NLRP3 inflammasome is assembled upon detection of environmental crystals, pollutants, host-derived DAMPs and protein aggregates (Tartey S and Kanneganti TD. Immunology, 2019 April; 156(4):329-338). Clinically relevant DAMPs that engage NLRP3 include uric acid and cholesterol crystals that cause gout and atherosclerosis, amyloid-β fibrils that are neurotoxic in Alzheimer's disease and asbestos particles that cause mesothelioma (Kelley et al., Int J Mol Sci, 2019 Jul. 6; 20(13)). Additionally, NLRP3 is activated by infectious agents such as Vibrio cholerae; fungal pathogens such as Aspergillus fumigatus and Candida albicans; adenoviruses, influenza A virus and SARS-CoV-2 (Tartey and Kanneganti, 2019 (see above); Fung et al. Emerg Microbes Infect, 2020 Mar. 14; 9(1):558-570).

Although the precise NLRP3 activation mechanism remains unclear, for human monocytes, it has been suggested that a one-step activation is sufficient while in mice a two-step mechanism is in place. Given the multitude in triggers, the NLRP3 inflammasome requires add-on regulation at both transcriptional and post-transcriptional level (Yang Yet al., Cell Death Dis, 2019 Feb 12;10(2):128). The NLRP3 protein consists of an N-terminal pyrin domain, followed by a nucleotide-binding site domain (NBD) and a leucine-rich repeat (LRR) motif on C-terminal end (Sharif et al., Nature, 2019 June; 570(7761):338-343). Upon recognition of PAMP or DAMP, NLRP3 aggregates with the adaptor protein, apoptosis-associated speck-like protein (ASC), and with the protease caspase-1 to form a functional inflammasome. Upon activation, procaspase-1 undergoes autoproteolysis and consequently cleaves gasdermin D (Gsdmd) to produce the N-terminal Gsdmd molecule that will ultimately lead to pore-formation in the plasma membrane and a lytic form of cell death called pyroptosis. Alternatively, caspase-1 cleaves the pro-inflammatory cytokines pro-IL-1β and pro-IL-18 to allow release of its biological active form by pyroptosis (Kelley et al., 2019—see above).

Dysregulation of the NLRP3 inflammasome or its downstream mediators are associated with numerous pathologies ranging from immune/inflammatory diseases, auto-immune/auto-inflammatory diseases (Cryopyrin-associated Periodic Syndrome (Miyamae T. Paediatr Drugs, 2012 Apr. 1; 14(2):109-17); sickle cell disease; systemic lupus erythematosus (SLE)) to hepatic disorders (eg. non-alcoholic steatohepatitis (NASH), chronic liver disease, viral hepatitis, alcoholic steatohepatitis, and alcoholic liver disease) (Szabo G and Petrasek J. Nat Rev Gastroenterol Hepatol, 2015 Jul.; 12(7):387-400) and inflammatory bowel diseases (eg. Crohn's disease, ulcerative colitis) (Zhen Y and Zhang H. Front Immunol, 2019 Feb, 28; 10:276). Also, inflammatory joint disorders (eg. gout, pseudogout (chondrocalcinosis), arthropathy, osteoarthritis, and rheumatoid arthritis (Vande Walle L et al., Nature, 2014 Aug. 7; 512(7512):69-73) were linked to NLRP3. Additionally, kidney related diseases (hyperoxaluria (Knauf et al., Kidney Int, 2013 November; 84(5):895-901), lupus nephritis, hypertensive nephropathy (Krishnan et al., Br J Pharmacol, 2016 Febuary; 173 (4):752-65), hemodialysis related inflammation and diabetic nephropathy which is a kidney-related complication of diabetes (Type 1, Type 2 and mellitus diabetes), also called diabetic kidney disease (Shahzad et al., Kidney Int, 2015 Jan;87(1):74-84) are associated to NLRP3 inflammasome activation. Reports link onset and progression of neuroinflammation-related disorders (eg. brain infection, acute injury, multiple sclerosis, Alzheimer's disease) and neurodegenerative diseases (Parkinsons disease) to NLRP3 inflammasome activation (Sarkar et al., NPJ Parkinsons Dis, 2017 Oct. 17; 3:30). In addition, cardiovascular or metabolic disorders (eg. cardiovascular risk reduction (CvRR), atherosclerosis, type I and type II diabetes and related complications (e.g. nephropathy, retinopathy), peripheral artery disease (PAD), acute heart failure and hypertension (Ridker et al., CANTOS Trial Group. N Engl J Med, 2017 Sep. 21; 377(12):1119-1131; and Toldo S and Abbate A. Nat Rev Cardiol, 2018 Apr.; 15(4):203-214) have recently been associated to NLRP3. Also, skin associated diseases were described (eg. wound healing and scar formation; inflammatory skin diseases, eg. acne, hidradenitis suppurativa (Kelly et al., Br J Dermatol, 2015 December; 173(6)). In addition, respiratory conditions have been associated with NLRP3 inflammasome activity (eg. asthma, sarcoidosis, Severe Acute Respiratory Syndrome (SARS) (Nieto-Torres et al., Virology, 2015 November; 485:330-9)) but also age-related macular degeneration (Doyle et al., Nat Med, 2012 May; 18(5):791-8). Several cancer related diseases/disorders were described linked to NLRP3 (eg. myeloproliferative neoplasms, leukemias, myelodysplastic syndromes (MOS), myelofibrosis, lung cancer, colon cancer (Ridker et al., Lancet, 2017 Oct. 21; 390(10105):1833-1842; Derangere et al., Cell Death Differ. 2014 Dec.; 21(12):1914-24; Basiorka et al., Lancet Haematol, 2018 Sep.; 5(9): e393-e402, Zhang et al., Hum Immunol, 2018 January; 79(1):57-62).

Several patent applications describe NLRP3 inhibitors, with recent ones including for instance international patent application WO 2020/018975, WO 2020/037116, WO 2020/021447, WO 2020/010143, WO 2019/079119, WO 2019/0166621 and WO 2019/121691, which disclose a range of specific compounds. Various specific compounds can be identified through the Chemical Abstracts Service, for instance compounds that may be available via a commercial source as that compound may be part of a library, but which has no ascribed use.

There is a need for inhibitors of the NLRP3 inflammasome pathway to provide new and/or alternative treatments for the diseases/disorders mentioned herein.

### SUMMARY OF THE INVENTION

The invention provides compounds which inhibit the NLRP3 inflammasome pathway.

Thus, in an aspect of the invention, there is now provided a compound of formula (I),

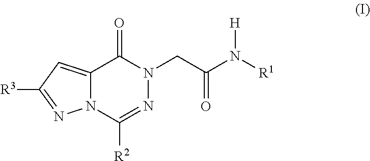

or a pharmaceutically acceptable salt thereof, wherein:
R¹ represents:
   (i) C₃₋₆ cycloalkyl optionally substituted with one or more substituents independently selected from —OH and —C₁₋₃ alkyl;
   (ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
   (iii) heterocyclyl, optionally substituted with 1 to 3 substituents independently selected from C₁₋₃ alkyl and C₃₋₆ cycloalkyl;
R² represents:
   (i) C₁₋₃ alkyl optionally substituted with one or more substituents independently selected from halo, —OH, —OC₁₋₃alkyl and oxo;
   (ii) C₃₋₆cycloalkyl;
   (iii) C₂₋₄alkenyl optionally substituted with —O'C₁₋₃alkyl;
   (iv) —O—C₁₋₃alkyl;
   (v) —N(H)alkyl or —N—(C₁₋₃alkyl)₂, where each alkyl may be optionally substituted with —OC₁₋₃ alkyl; or
   (vi) heterocyclyl;
R³ represents:
   (i) hydrogen;
   (ii) halo;
   (iii) C₁₋₄ alkyl optionally substituted with one or more substituents independently selected from halo, —OH, oxo, —O—C₁₋₃alkyl, —C(O)OH, —C(O)N(H)heteroaryl, —C(O)N(H)aryl, —C(O)N(H)C₁₋₃ alkyl and —C(O)N(C₁₋₃alkyl)₂;
   (iv) C₂₋₄ alkenyl;
   (v) C₃₋₆ cycloalkyl;
   (vi) —OC₁₋₄ alkyl;
   (vii) —N(H)C₁₋₃alkyl or —N(C₁₋₃alkyl)₂;
   (viii) —C(O)N(H)C₁₋₃ alkyl or —C(O)N(C₁₋₃alkyl)₂;
   (ix) aryl or heteroaryl; or
   (x) heterocyclyl,
which compounds may referred to herein as “compounds of the invention”.

In an embodiment, compounds are of the invention that may be mentioned include those in which:
(i) when R² represents methyl, and R³ represents 4-methoxyphenyl, then R¹ does not represent 3-ethylphenyl, 2,4-dimethylphenyl,3-fluorophenyl, 2,4-dimethoxyphenyl, 2-methoxy-5-methylphenyl, 3,5-dimethylphenyl, 3-chloro-4-methylphenyl, 2-ethylphenyl, 4-fluorophenyl, 3,5-dimethoxyphenyl, cyclopropyl, 2-methylphenyl, 2,3-dimethylphenyl, 3-(methylthio)phenyl, 4-methoxyphenyl, cyclohexyl, 3-chlorophenyl, cyclopentyl, cycloheptyl, 4-chlorophenyl, 1,2,3,4-tetrahydro-1-naphthalenyl or 1,3-benzodioxol-5-yl;
(ii) when R² represents methyl, and R³ represents 4-ethylphenyl, then R¹ does not represent 2,3-dimethylcyclohexyl, 4-chlorophenyl, 1,2,3,4-tetrahydro-1-naphthalenyl, 3-chlorophenyl, cyclohexyl, 2-methylcyclohexyl, 3,5-dimethoxyphenyl, 2,4-difluorophenyl, cyclopentyl, cycloheptyl or 4-acetylphenyl;
(iii) when R² represents methyl, R³ represents phenyl, then R¹ does not represent cycloheptyl, 4-chlorophenyl, 3-chlorophenyl, 4-methoxyphenyl, cyclohexyl, cyclopentyl or 4-acetylphenyl,
which we may refer to herein as “the provisos”.

Hence, there is provided a compound of formula (I) as hereinbefore defined, or a pharmaceutically acceptable salt thereof, provided that it is not a compound of the provisos.

In another embodiment of the invention, there is provided compounds of the invention in which there is provided a compound of formula (I) as hereinbefore defined, or a pharmaceutically acceptable salt thereof, but in which: R¹ represents —R^{x}-R¹, in which R^{x} is —CH₂— or a direct bond and R¹ represents:
(i) C₃₋₆ cycloalkyl optionally substituted with one or more substituents independently selected from —OH, —C₁₋₃ alkyl (itself optionally substituted by one or more substituents selected from fluoro, —OH, —OC₁₋₃ alkyl, heteocyclyl, aryl and heteroaryl), —OC₁₋₃alkyl, —C(O)—C₁₋₄ alkyl, —C(O)OC₁₋₄alkyl, heterocyclyl, aryl and heteoaryl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy, C₃₋₆ cycloalkyl; or
(iii) heterocyclyl, optionally substituted with 1 to 3 substituents independently selected from C₁₋₃ alkyl and C₃₋₆ cycloalkyl;
R² represents:
   (i) C₁₋₆ alkyl optionally substituted with one or more substituents independently selected from halo, —OH, —OC₁₋₃alkyl, —OC₃₋₆ cycloalkyl, —N(H)C(O)C₁₋₃ alkyl and oxo;
   (ii) C₃₋₆ cycloalkyl;

(iii) C₂₋₄alkenyl optionally substituted with —O—C₁₋₃alkyl;
(iv) —O—C₁₋₃alkyl;
(v) —C(O)C₁₋₃alkyl;
(vi) —N(H)C₁₋₃alkyl or —N—(C₁₋₃alkyl)₂, where each alkyl may be optionally substituted with —OC₁₋₃ alkyl or C₃₋₆ cycloalkyl; or
(vii) heterocyclyl (optionally substituted by one or more substituents selected from halo, C₁₋₃ alkyl, —C(O)C₁₋₃alkyl and —C(O)OC₁₋₄alkyl);
R³ represents:
   (i) hydrogen;
   (ii) halo or —CN;
   (iii) C₁₋₆ alkyl optionally substituted with one or more substituents independently selected from halo, —OH, oxo, —O—C₁₋₃alkyl, —C(O)OH, —C(O)N(H)heteroaryl, —C(O)N(H)aryl, —C(O)N(H)C₁₋₃ alkyl, —C(O)N(C₁₋₃-alkyl)2, —N(C₁₋₃ alkyl)-C(O)-C₁₋₃alkyl, —N(H)C₁₋₃ alkyl, heterocyclyl, aryl and heteroaryl (which latter three groups may themselves be optionally substituted by one or more substituents selected from halo, C₁₋₃ alkyl and, where applicable, ═O);
   (iv) C₂₋₄ alkenyl;
   (v) C₃₋₆ cycloalkyl optionally subttiuted by one or more substituents selected from fluoro, C₁₋₃ alkyl and —OC₁₋₃alkyl;
   (vi) —OC₁₋₄ alkyl;
   (vii) —C(O)H or —C(O)C₁₋₃ alkyl;
   (viii) —N(H)C₁₋₃alkyl or —N(C₁₋₃alkyl)₂, wherein all the alkyl moieties are optionally substituted by one or more substituents selected from fluoro, —OC₁₋₃alkyl, aryl and heteroaryl;
   (ix) —C(O)N(H)C₁₋₃alkyl or —C(O)N(C₁₋₃alkyl)₂;
   (x) aryl or heteroaryl (which groups may themselves be optionally substituted by one or more substituents selected from halo, C₁₋₃ alkyl, —C(O)OC₁₋₄alkyl and, where applicable, ═O); or
   (xi) heterocyclyl (optionally substituted by one or more substituents selected from halo, —OH, C₁₋₃ alkyl, —C(O)OC₁₋₄alkyl and, where applicable, ═O), provided that it is not a compound of the provisos, which compounds may also be referred to herein as “compounds of the invention”.

In a further embodiment, compounds are of the invention that may be mentioned include those in which:
(i) when R² represents methyl, R³ represents phenyl, then R¹ does not represent 4-piperidinyl substituted on the nitogen with a —CH₂-phenyl group or a —C(O)O—CH₂CH₃ group.
(ii) when R² represents methyl, R³ represents 4-methoxyphenyl, then R¹ does not represent 4-piperidinyl substituted on the nitogen with a —CH₂-phenyl group or a —C(O)O-CH₂CH₃ group;
(iii) when R² represents methyl, R³ represents 4-ethylphenyl, then R¹ does not represent 4-piperidinyl substituted on the nitogen with a —CH₂-phenyl group or a —C(O)O-CH₂CH₃ group.

In another aspect, there is provided compounds of the inventon (without the provisos, where applicable) for use as a medicament. In another aspect, there is provided a pharmaceutical composition comprising a therapeutically effective amount of a compound of the invention (without the provisos, again where applicable).

In a further aspect, there is provided compounds of the invention (and/or pharmaceutical compositions comprising such compounds) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor. Specific diseases or disorders may be mentioned herein, and may for instance be selected from inflammasome-related diseases or disorders, immune diseases, inflammatory diseases, auto-immune diseases, or auto-inflmmatory diseases.

In another aspect, there is provided a use of compounds of the invention (without the provisos) (and/or pharmaceutical compositions comprising such compounds): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In another aspect, there is provided use of compounds of the invention (without the provisos) (and/or pharmaceutical compositions comprising such compounds) in the manufacture of a medicament for: the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In another aspect, there is provided a method of treating a disease or disorder in which the NLRP3 signalling contributes to the pathology, and/or symptoms, and/or progression, of said disease/disorder, comprising administering a therapeutically effective amount of a compound of the invention (without the provisos), for instance to a subject (in need thereof). In a further aspect there is provided a method of inhibiting the NLRP3 inflammasome activity in a subject (in need thereof), the method comprising administering to the subject in need thereof a therapeutically effective amount of a compound of the invention (without the provisos).

In further aspect, there is a provided a compound of the invention (without the provisos) in combination (including a pharmaceutical combination) with one or more therapeutic agents (for instance as described herein). Such combination may also be provided for use as described herein in respect of compounds of the invention (without the provisos), or, a use of such combination as described herein in respect of compounds of the invention (without the provisos). There may also be provided methods as described herein in repsect of compounds of the invention (without the provisos), but wherein the method comprises administering a therapeutically effective amount of such combination.

### DETAILED DESCRIPTION OF THE INVENTION

The invention provides a compound of formula (I),

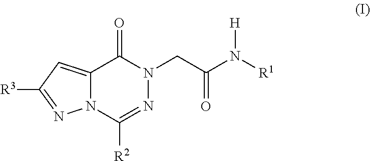

or a pharmaceutically acceptable salt thereof, wherein:
R¹ represents:
   (i) C₃₋₆ cycloalkyl optionally substituted with one or more substituents independently selected from —OH and —C₁₋₃ alkyl;
   (ii) aryl or heteroaryl, each of which is optionally substituted with 1 to 3 substituents independently selected from halo, —OH, —O—C1.3 alkyl, —C₁₋₃ alkyl, haloC₁₋₃alkyl, hydroxyC₁₋₃ alkyl, C₁₋₃ alkoxy, haloC₁₋₃alkoxy; or
   (iii) heterocyclyl, optionally substituted with 1 to 3 substituents independently selected from C₁₋₃ alkyl and C₃₋₆ cycloalkyl;
R² represents:
   (i) C₁₋₃ alkyl optionally substituted with one or more substituents independently selected from halo, —OH, —OC₁₋₃alkyl and oxo;
   (ii) C₃₋₆cycloalkyl;
   (iii) C₂₋₄alkenyl optionally substituted with —O—C₁₋₃alkyl;
   (iv) —O—C₁₋₃alkyl;
   (v) —N(H)alkyl or —N-(C₁₋₃alkyl)₂, where each alkyl may be optionally substituted with —OC₁₋₃ alkyl; or
   (vi) heterocyclyl;
R³ represents:
   (i) hydrogen;
   (ii) halo;
   (iii) C₁₋₄ alkyl optionally substituted with one or more substituents independently selected from halo, —OH, oxo, —O—C₁₋₃alkyl, —C(O)OH, —C(O)N(H)heteroaryl, —C(O)N(H)aryl, —C(O)N(H)C₁₋₃ alkyl and —C(O)N(C₁₋₃alkyl)₂;
   (iv) C₂₋₄ alkenyl;
   (v) C₃₋₆ cycloalkyl;
   (vi) —OC₁₋₄ alkyl;
   (vii) —N(H)C₁₋₃alkyl or —N(C₁₋₃alkyl)₂;
   (viii) —C(O)N(H)C₁₋₃alkyl or —C(O)N(C₁₋₃alkyl)₂;
   (ix) aryl or heteroaryl; or
   (x) heterocyclyl.

As indicated above, such compounds may be referred to herein as “compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns Ito XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985). Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

When an absolute configuration is specified, it is according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C , ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain. Such a group is attached to the rest of the molecule by a single bond.

C_{2-q} alkenyl when used herein (again where q is the upper limit of the range) refers to an alkyl group that contains unsaturation, i.e. at least one double bond.

C_{3-q} cycloalkyl (where q is the upper limit of the range) refers to an alkyl group that is cyclic, for instance cycloalkyl groups may be monocyclic or, if there are sufficient atoms, bicyclic. In an embodiment, such cycloalkyl groups are monocyclic. Such cycloalkyl groups are unsaturated. Substituents may be attached at any point on the cycloalkyl group.

The term “halo”, when used herein, preferably includes fluoro, chloro, bromo and iodo.

C_{1-q} alkoxy groups (where q is the upper limit of the range) refers to the radical of formula —OR^{a}, where R^{a} is a C_{1-q} alkyl group as defined herein.

HaloC_{1-q} alkyl (where q is the upper limit of the range) goups refer to C_{1-q} alkyl groups, as defined herein, where such group is substituted by one or more halo. HydroxyC_{1-q} alkyl (where q is the upper limit of the range) refers to C_{1-q} alkyl groups, as defined herein, where such group is substituted by one or more (e.g. one) hydroxy (—OH) groups (or one or more, e.g. one, of the hydrogen atoms is replaced with —OH). Similarly, haloC_{1-q} alkoxy and hydroxyC_{1-q} alkoxy represent corresponding —OC_{1-q} alkyl groups that are substituted by one or more halo, or, substituted by one or more (e.g. one) hydroxy, respectively.

Heterocyclyl groups that may be mentioned include non-aromatic monocyclic and bicyclic heterocyclyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocyclyl groups may also be bridged. Such heterocyclyl groups are saturated. C_{2-q} heterocyclyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like.

Substituents on heterocyclyl groups may, where appropriate, be located on any atom in the ring system including a heteroatom. The point of attachment of heterocyclyl groups may be via any atom in the ring system including (where appropriate) a heteroatom (such as a nitrogen atom), or an atom on any fused carbocyclic ring that may be present as part of the ring system. Heterocyclyl groups may also be in the N- or S-oxidised form. In an embodiment, heterocyclyl groups mentioned herein are monocyclic.

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. When aryl groups are polycyclic, in an embodiment, each ring is aromatic. In an embodiment, aryl groups mentioned herein are monocyclic or bicyclic. In a further embodiment, aryl groups mentioned herein are monocyclic.

“Heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. In an embodiment, when heteroaryl groups are polycyclic, then each ring is aromatic. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. When heteroaryl groups are polycyclic in which there is a non-aromatic ring present, then that non-aromatic ring may be substituted by one or more ═O group. In an embodiment, heteroaryl groups mentioned herein may be monocyclic or bicyclic. In a further embodiment, heteroaryl groups mentioned herein are monocyclic.

Heteroatoms that may be mentioned include phosphorus, silicon, boron and, preferably, oxygen, nitrogen and sulfur.

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them). The skilled person will appreciate that compounds of the invention that are the subject of this invention include those that are stable. That is, compounds of the invention include those that are sufficiently robust to survive isolation from e.g. a reaction mixture to a useful degree of purity.

Various embodiments of the invention will now be described, including embodiments of the compounds of the invention.

In an embodiment, R³ does not represent optionally substituted phenyl. In another embodiment, R³ does not represents optionally substituted aryl or heteroaryl). Alternatively or in addition, in a further embodment, R² does not represent unsubstituted methyl or does not represent unsubstituted C₁₋₂ alkyl.

In an embodiment, compounds of the invention include those in which le represents: (i) C₃₋₆ cycloalkyl; (ii) aryl or heteroaryl; or (iii) or heterocyclyl, all of which are optionally substituted as herein defined.

In an embodiment when R¹ represents optionally substituted C₃₋₆ cycloalkyl, then it represents C₃₋₄ cycloalkyl optionally substituted by one or two substituents selected from C₁₋₃ alkyl (e.g. methyl) and —OH. In a further embodiment, R¹ represents cyclopropyl (e.g. unsubstituted) or cyclobutyl. In yet a further embodiment, le represents unsubstituted cyclopropyl or cyclobutyl substituted by —OH and methyl (e.g. at the same carbon atom). In an embodiment therefore, R¹ represents:

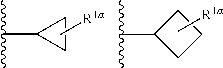

where each R^{1a} represents one or two optional substituents selected from —OH and C₁₋₃ alkyl (e.g. methyl). In a particular embodiment of this aspect, R¹ represents C₃₋₆ cyclolkyl, such as substituted cyclobutyl or unsubstituted cyclopropyl, for instance:

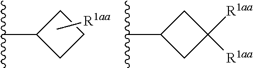

where each R^{1aa} represents one or two optional substituents selected from those defined by R^{1a}, and in an embodiment represents two substituents, methyl and —OH; or

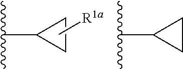

where R^{1a} is as defined above, but where, in a particular embodiment, it is not present.

In an embodiment where R¹ represents aryl or heteroaryl, optionally substituted as defined herein, then it may represent: (i) phenyl; (ii) a 5- or 6-membered mono-cyclic heteroaryl group; or (iii) a 9- or 10-membered bicyclic heteroaryl group, all of which are optionally substituted by one to three substituents as defined herein. In an embodiment, the aforementioned aryl and heteroaryl groups are optionally substituted with one or two (e.g. one) substituent(s) selected from halo (e.g. fluoro), —OH and —OC₁₋₃ alkyl. In a further embodiment, such optional substituents are selected from fluoro and methoxy. In one embodiment, R¹ represents phenyl or a mono-cyclic 6-membered heteroaryl group and in another embodiment it may represent a 9- or 10-membered (e.g. 9-membered) bicyclic heteroaryl group. Hence, in an embodiment, le may represent:

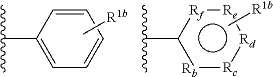

wherein R^{1b} represents one or two optional substituents selected from halo, —OH and —OCH₃ (and in a further embodiment, such optional substituents are selected from fluoro and methoxy), and at least one of R_{b}, R_{c}, R_{d}, Rₑ and R_{f} represents a nitrogen heteroatom (and the others represent CH). In an embodiment, either one or two of R_{b}, R_{c}, R_{d}, Rₑ and Rf represent(s) a nitrogen heteroatom, for instance, R_{d} represents nitrogen and, optionally, R_{b} represents nitogen, or, R_{c} represents nitogen. In an aspect: (i) Rb and R_{d} represent nitrogen; (ii) R_{d} represents nitrogen; or (iii) R_{c} represents nitrogen. Hence, R¹ may represent 3-pyridyl, 4-pyridyl or 4-pyrimidinyl, all of which are optionally substituted as herein defined, for instance with one substituent selected from fluoro and methoxy (and in a further embodiment in this aspect, R¹ represents unsubstituted 4-pyrimidinyl, unsubstituted 4-pyridyl, unsubstituted 3-pyridyl, 3-fluoro-4-pyridyl or 3-methoxy-4-pyridyl). In another embodiment, R¹ may represent:

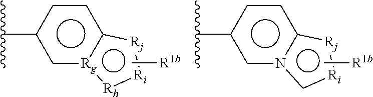

wherein R^{1b} is as defined above (i.e. represents one or two optional substituent as defined above), each ring of the bicyclic system is aromatic, R_{g} represents a N or C atom and any one or two of Rh, R₁ and (for instance, one or two of Rᵢ and Rⱼ) represents N and the other(s) represent(s) C (provided that, as the skilled person would understand, the rules of valency are adhered to).

In an embodiment R¹ represents:

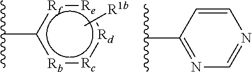

in which R_{b} and R_{d} represent a nitrogen atom, and, in an embodiment, there is no R^{1b} substituent present.

In another embodiment, R¹ represents:

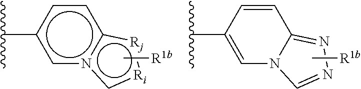

in which one of Rᵢ and Rⱼ and represents N and the other represents C, or, both Rᵢ and Rⱼ represent N, and, in an embodiment, there is no R^{1b} substituent present.

In an embodiment where R¹ represents heterocyclyl, optionally substituted as defined herein, such goup is in a further aspect a 5- or 6-membered heterocyclyl group, for instance containing at least one nitrogen heteroatom; for instance, in a particular embodiment, in this instance R¹ may represent a 6-membered nitrogen-containing heterocyclyl group optionally substituted by one substituent selected from C₁₋₃ alkyl and C₃₋₆ cycloalkyl. In an aspect of this embodiment, the 6-membered heterocyclyl group may be piperidinyl (e.g. 3-piperidinyl) optionally substituted by C₃₋₄ cycloalkyl (e.g. cyclobutyl).

In an embodiment where R¹ represents aryl, specific groups that may be mentioned include phenyl and methoxy-phenyl (such as 2-methoxy-phenyl). In an embodiment where R¹ represents heteroaryl, it is preferably a mono-cyclic 6-membered ring, for instance containing at least one nitrogen heteroatom and thereby forming a pyridyl or pyrimidinyl group. Specific groups that R¹ may represent include 4-pyridyl, 3-pyridyl and 4-pyrimidinyl (all of which are optionally substituted as defined herein). In view of the optionaly substitution mentioned herein, such groups may represent an unsubstituted 4-pyrimidinyl, unsubstituted 3-pyridyl, 3-fluoro-4-pyridyl and 3-methoxy-pyridyl.

In a particular embodiment, R¹ represents cyclopropyl or a mono-cyclic heteraryl group optionally substituted as defined herein. In an aspect, R¹ represents a mono-cyclic heteroaryl group, for instance a 6-membered mono-cyclic heteroaryl group containing one or two nitrogen heteroatoms, and which groups is optionally substituted by one or more substituents selected from fluoro and methoxy.

In an embodiment, R¹ represents phenyl or a 6-membered heteroaryl group (containing between one and three heteroatoms) and which is optionally substituted as defined herein. In an embodiment, R¹ represents a 6,5-fused bicyclic ring containing one of five heteroatoms (wherein at least two are nitrogen) and which group is optionally substituted as herein defined.

In an embodiment R² represents: (i) C₁₋₃ alkyl optionally substituted as defined herein (e.g. with one or more substituents independently selected from —OH, —OC₁₋₃ alkyl) and oxo); (ii) C₂₋₄alkenyl optionally substituted with —O—C₁₋₃alkyl; (iii) —N—(C₁₋₃alkyl)_{2;} (iv) heterocyclyl. In a further embodiment, R² represents (i) C₁₋₃ alkyl optionally substituted by one or two (e.g. one) sub stituent(s) selected from —OH, methoxy, ethoxy and oxo ; (ii) C₂₋₄alkenyl optionally substituted with methoxy or ethoxy; (iii) —N—(C₁₋₃alkyl)₂, where the alkyl moieties are unsubstituted; (iv) a 5- or 6-membered heterocyclyl group, for example a 6-membered heterocyclyl group (e.g. in which there is at least one nitrogen heteroatom and optionally an oxygen heteroatom, so forming for example a morpholinyl group).

In an embodiment when R² represents C₁₋₃ alkyl, then it represents isopropyl, ethyl, —(CH₂)₂—OCH₃, —C(H)(CH₃)-OCH₃, —C(H)(CH₃)-OCH₂CH₃, —C(O)—CH₃, —C(H)(OH)—CH₃ or —C(OH)(CH₃)—CH₃.

In an embodiment when R² represents C₂₋₄ alkenyl, then it represents —C(═CH₂)—OCH₃ or —C(═CH₂)—OCH₂CH₃.

In an embodiment when R² represent —N—(C₁₋₃alkyl)₂, then it represents —N(CH₃)(CH₂CH₃).

In an embodiment when R² represents heterocyclyl, then it represents morpholinyl, for instance 4-morpholinyl. In an embodiment, R² represents C₁₋₃ alkyl optionally substituted as defined herein (for instance, it may be unsubsituted or substituted by one subsituent selected from —OH and —O—C₁₋₂ alkyl). In an embodiment, R² represents unsubstituted C₁₋₃ alkyl.

In an embodiment, R³ represents (i) hydrogen; (ii) halo (bromo, chloro, iodo, fluoro); (iii) C₁₋₄ alkyl optionally substituted by one or more substituent selected from fluoro, —OH and —O—C₁₋₂ alkyl; (iv) C₂₋₄ alkenyl (which is, in an embodiment, unsubstituted); or (v) C₃₋₆ cycloalkyl (which is, in an embodiment, C₃₋₄ cycloalkyl). In a further embodiment, R³ may, additionally or alternatively, represent (vi) —N(H)C₁₋₂ alkyl or —N(C₁₋₂ alkyl)_{2;} or (vii) heterocyclyl (e.g. in a nitogen containing 3- to 6-membered heterocyclyl group, which, in an embodiment, is attached via the nitrogen atom).

In a particular embodiment, R³ represents hydrogen, halo (bromo, iodo, fluoro, chloro), methyl, ethyl, isopropyl, isobutyl (—CH₂C(H)(CH₃)₂), —CHF2, —CF3, —CH₂OH, —CH₂OCH₃, —C(F)(H)CH₃, —(CH₂)₂CF3, —C(CH₃)═CH₂, —C(H)═CH₂, cyclopropyl, cycylobutyl. In a further embodiment, additional or alternative R³ groups that may be mentioned include —N(H)CH₃, —N(CH₃)₂ and azetidinyl (attached via the nitrogen atom).

The names of the compounds of the present invention were generated according to the nomenclature rules agreed upon by the Chemical Abstracts Service (CAS) using Advanced Chemical Development, Inc., software (ACD/Name product version 10.01; Build 15494, 1 Dec 2006) or according to the nomenclature rules agreed upon by the

International Union of Pure and Applied Chemistry (IUPAC) using Advanced Chemical Development, Inc., software (ACD/Name product version 10.01.0.14105, October 2006). In case of tautomeric forms, the name of the depicted tautomeric form of the structure was generated. The other non-depicted tautomeric form is also included within the scope of the present invention.

### Preparation of the Compounds

In an aspect of the invention, there is provided a process for the preparation of compounds of the invention, where reference here is made to compounds of formula (I) as defined herein.

Compounds of formula (I) may be prepared by:

(i) reaction of a compound of formula (II),

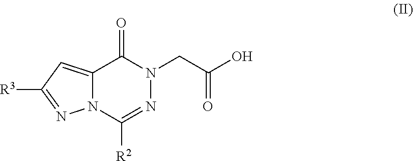

or a derivative thereof (e.g. a salt), wherein R² and R³ are as hereinbefore defined, with a compound of formula (III),

H₂N—R¹ (III)

or a derivative thereof, wherein R¹ is as hereinbefore defined, under amide-forming reaction conditions (also referred to as amidation), for example in the presence of a suitable coupling reagent (e.g. [4bis(dimethylamino)methylene]-1H-1,2,3-triazolo[4,5-b]pyridinium 3-oxide hexafluorophosphate (O-(7-azabenzotriazol-1-yl)-N,N,N′,N′-tetramethyluronium hexafluorophosphate), 1,1′-carbonyldiimidazole, N′,N′-dicyclohexylcarbodiimide, 1-(3-dimethylaminopropyl)-3-ethylcarbodiimide (or hydrochloride thereof), N,N′-disuccinimidyl carbonate, benzotriazol-1-yl-oxytris(dimethylamino)phosphonium hexafluoro-phosphate, 2-(1H-benzotriazol-1-yl)-1,1,3,3-tetramethyluronium hexa-fluorophosphate (i.e. O-(1H-b enzotriazol-1-yl)-N,N,N′,N′-tetramethyluronium hexafluorophosphate), benzotriazol-1-yloxytris-pyrrolidinophosphonium hexa-fluorophosphate, bromo-tris-pyrrolidinophosponium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. Alternatively, a carboxylic acid group may be converted under standard conditions to the corresponding acyl chloride (e.g. in the presence of SOC₂ or oxalyl chloride), which acyl chloride is then reacted with a compound of formula (II), for example under similar conditions to those mentioned above;

(ii) reaction of a compound of formula (IV),

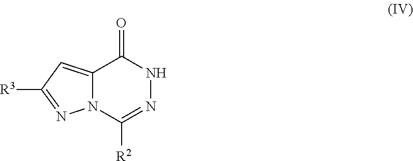

wherein R² and R³ are as hereinbefore defined, with a compound of formula (V),

LG^{a}-CH₂—C(O)—N(H)R¹ (V)

wherein LG^{a} represents a suitable leaving group (e.g. halo, such as chloro) and R¹ is as defined herein, under suitable reaction conditions, e.g. in the presence of an appropriate base, e.g. Cs₂CO₃ or LiHMDS, or the like, or alternative alkylation reaction conditions;
(iii) by transformation (such transformation steps may also take place on intermediates) of a certain compound of formula (I) into another, for example:
   for compounds of formula (I) in which R³ represents —N(H)C₁₋₆ alkyl or —N(C₁₋₆ alkyl)₂, reaction of a corresponding compound of formula (I) in which R³ represents halo, with an appropriate amine H₂NC₁₋₆ alkyl or HN(C₁₋₆ alkyl)₂, in an amination reaction under appropriate conditions, e.g. using under standard coupling conditions, in the presence of a catalyst, e.g. CuI, a ligand, e.g. D/L-proline and a base, e.g. K₂CO₃; similar transformations may be performed on compounds in which R² represents halo, and an amine (or heterocyclyl group attached via a nitrogen atom) is desired at R²;
   for compounds of formula (I) containing an alkene, reduction to a corresponding compound of formula (I) containing an alkane, under reduction conditions, e.g. with hydrogen in the presence of a suitable catalyst such as, for example, palladium on carbon, in a suitable reaction-inert solvent, such as, for example, ethanol or methanol;
   coupling to convert a halo group to e.g. an alkyl, alkenyl or aryl/heteroaryl group, for example in the presence of a suitable coupling reagent, e.g. where the reagent comprises the appropriate alkyl, alkenyl or aryl/heteroaryl group attached to a suitable group such as —B(OH)₂, —B(OR^{wx})₂, zincates (e.g. including —Zn(R^{wx})₂,
   —ZnBrR^{wx}) or —Sn(R^{wx})₃, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), thereby forming e.g. a pinacolato boronate ester group. The reaction may be performed in the presence of a suitable catalyst system, e.g. a metal (or a salt or complex thereof) such as Pd, CuI, Pd/C, PdC1₂, Pd(OAc)₂, Pd(Ph₃P)₂Cl₂, Pd(Ph₃P)₄ (i.e. palladium tetrakistriphenylphosphine), Pd₂(dba)3 and/or NiCl₂ (preferred cataysts include RuPhos Pd G3, XPhos Pd and bis(tri-tert-butylphosphine)palladium(O)) and optionally a ligand such as PdCl(dppf).DCM, t-Bu₃P, (C₆H₁₁)₃P, Ph₃P, AsPh₃, P(o-To1)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenyl-phosphino-ferrocene), 1,3-bis(diphenylphosphino)propane, xantphos, or a mixture thereof, together with a suitable base, such as Na₂CO₃, K₃PO₄, Cs₂CO₃, NaOH, KOH, K₂CO₃, CsF, Et3N, (i-Pr)₂NEt, t-BuONa or t-BuOK (or mixtures thereof; preferred bases include Na₂CO₃ and K₂CO₃) in a suitable solvent such as dioxane, toluene, ethanol, dimethylformamide, dimethoxyethane, ethylene glycol dimethyl ether, water, dimethylsulfoxide, acetonitrile, dimethylacetamide, N-methylpyrrolidinone, tetrahydrofuran or mixtures thereof (preferred solvents include dimethylformamide and dimethoxyethane);
   reduction of a ketone to an alcohol, in the presence of suitable reducing conditions, e.g. NaBH₄ or the like;
   conversion of —C(CH₂)-OCH₂CH₃ to —C(O)CH₃, by reaction in the presence of HCl, e.g. also in a suitable solvent such as THF;
   conversion of a —C(O)alkyl moiety to a —C(OH)(alkyl)(alkyl) moiety by reaction of an appropriate Grignard reagent, e.g. alkylMgBr;
   transformation of a alkene ═CH₂ moiety to a carbonyl ═O moiety, for instance, in the presence of AD-mix-Alpha and methane-sulfonamide;
   transformation of a ketone to an alcohol —OH moiety;
   alkylation of a —OH moiety (to —O-alkyl), under appropriate reaction conditions.

The compound of formula (II) may be prepared by hydrolysis of the corresponding carboxylic acid ester (for example under standard hydrolysis conditions, e.g. base hydrolysis in the presence of an alkai metal hydroxide (such as lithium hydroxide)), which in turn is prepared by reaction of a compound of formula (IV),

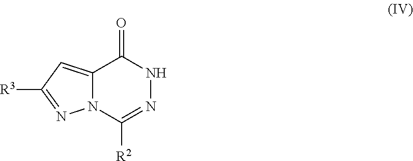

wherein R² and R³ are as hereinbefore defined, with a compound of formula (VI),

LG—CH₂—C(O)O—R^{aa} (VI)

wherein R^{aa} represents C₁₋₆ alkyl (e.g ethyl) and LG represents a suitable leaving group, such as halo (e.g. chloro), for instance under reaction conditions and using reagent such as those described herein.

In general the compounds of the invention can therefore be made with reference to the procedures above. However, in the interests of versatility, further schemes are provided below in order to provide intermediate and final compounds of the invention. Further details are provided in the schemes below (as well as in the specific details of the experimental described hereinafter).

In this respect, Scheme 1 outlines a typical synthesis:

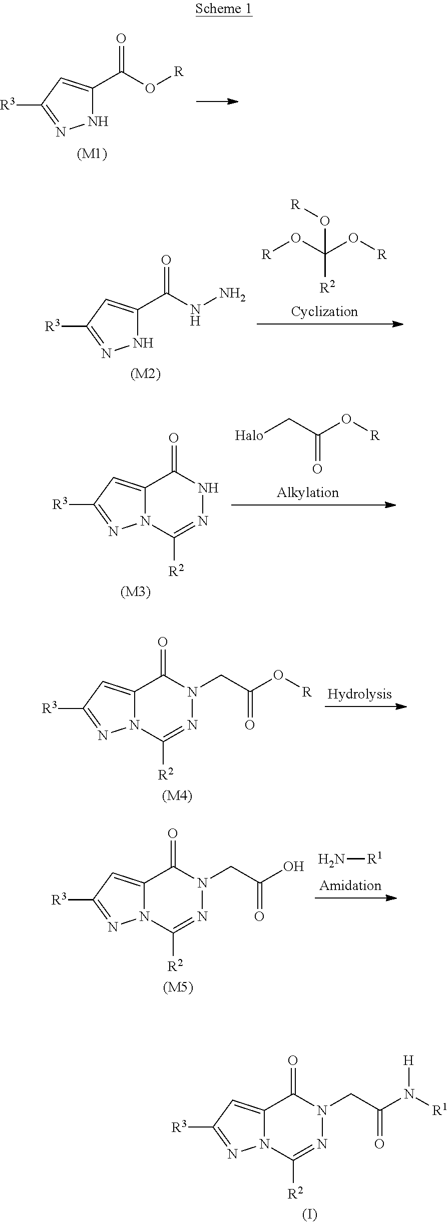

Compounds of the invention, as described herein, can be prepared by a reaction sequence shown in Scheme 1 (above), whereby an appropriately substituted pyrrole (M1), wherein R is C₁₋₄ alkyl (and R³ is as defined herein), is reacted with hydrazine to give hydrazide (M2), which is then cyclized by reaction with an appropriate orthoester, wherein R is C₁₋₄ alkyl (and R² is as defined herein), in the presence of a Lewis acid, e.g. aluminum isopropoxide, to the triazinone (M3) (also referred to herein as compound of formula (IV)) which is then alkylated with an appropriate alkyl haloacetate, wherein R is C₁₋₄ alkyl, in the presence of a base, e.g. K₂CO₃, a nucleophilic catalyst, e.g. KI and a crown ether, e.g. 18-crown-6, to provide ester (M4) which is typically cleaved e. g. under basic conditions, e.g. aqueous LiOH in THF or NaOH in MeOH to yield the acid intermediate (M5) (also referred to herein as compound of formula (II)), followed by amidation with R¹-NH₂ (wherein if R¹ has a functional group such as OH, NH₂, CO₂H, such group is optionally protected) using standard coupling conditions, e.g. HATU and a base, e.g. Hünig's base, optionally followed by an additional deprotection step to provide a compound of Formula (I), or a pharmaceutically acceptable salt thereof

Modifications and transformations may also be done on intermediates and, in this respect, the processes described above may also be applied to intermediates, as depicted for instance in the following Scheme 2:

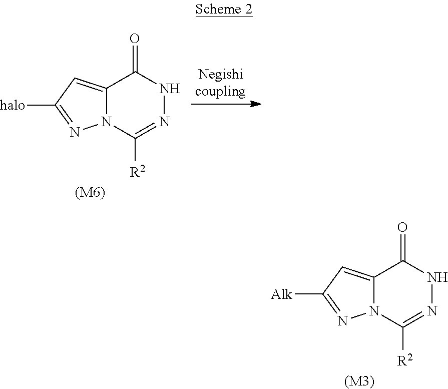

For instance, as per Scheme 2 above, the triazinone (M3) wherein R³ is C₁₋₄ alkyl, as defined herein, may be prepared by a reaction of a halo-triazinone (M6) (i.e. where R³ is halo) with a zincate, e.g. diethylzinc, using standard Negishi cross-coupling conditions, in the presence of a catalyst, e.g. XPhos Pd G3 and a base, e.g. K₂CO₃, to provide the alkyl-triazinone (M3).

Further the following transformations, depicted in Scheme 3 below, show versatility in allowing introduction of other substituents at the R² position of such intermediates too (as well as for final compounds):

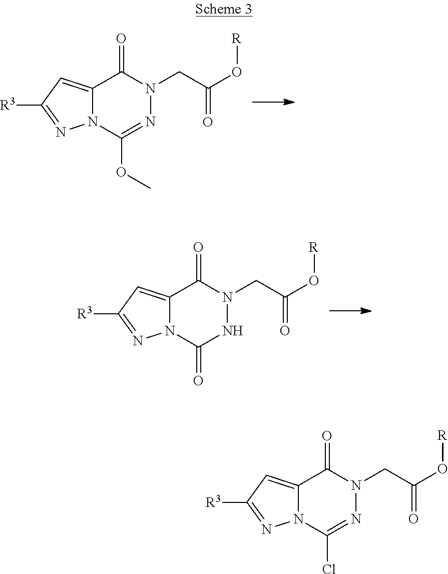

In Scheme 3 (where R represents C₁₋₆ alkyl, and R³ is as defined herein, e.g. an alkyl group, such as isopropyl), the methoxy moiety is coverted to a carbonyl group by reaction with sodium iodide and trimethylsilyl chloride, then the carbonyl group is converted to a chloro group by reaction with a chlorinating reagent such as POCl_{3;} such intermediate is then very versatile as the chloro moiety may be replaced in a variety of coupling reactions, for instance as described above in respect of the final compound transformations, e.g. coupling with an amine, or with an alkyl/alkenyl group, etc.

Certain intermediate compounds may be commercially available, may be known in the literature, or may be obtained either by analogy with the processes described herein, or by conventional synthetic procedures, in accordance with standard techniques, from available starting materials using appropriate reagents and reaction conditions.

Certain substituents on/in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations, nitrations or couplings.

Compounds of the invention may be isolated from their reaction mixtures using conventional techniques (e.g. recrystallisations, where possible under standard conditions).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenyl-methyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art. For example the a —C(O)O-tent-butyl ester moiety may serve as a protecting group for a —C(O)OH moiety, and hence the former may be converted to the latter for instance by reaction in the presence of a mild acid (e.g. TFA, or the like).

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P.G.M. Wutz, Wiley-Interscience (1999).

The compounds of the invention as prepared in the hereinabove described processes may be synthesized in the form of racemic mixtures of enantiomers which can be separated from one another following art-known resolution procedures. Those compounds of the invention that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of the invention involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

### Pharmacology

There is evidence for a role of NLRP3-induced IL-1 and IL-18 in the inflammatory responses occurring in connection with, or as a result of, a multitude of different disorders (Menu et al., Clinical and Experimental Immunology, 2011, 166, 1-15; Strowig et al., Nature, 2012, 481, 278-286). NLRP3 mutations have been found to be responsible for a set of rare autoinflammatory diseases known as CAPS (Ozaki et al., J. Inflammation Research, 2015, 8,15-27; Schroder et al., Cell, 2010, 140: 821-832; Menu et al., Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1 beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al., Eur. J. lmmunol., 2010, 40, 595-653).

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Braddock et al., Nat. Rev. Drug Disc. 2004, 3, 1-10; Inoue et at, Immunology, 2013, 139, 11-18; Coll et a.\, Nat. Med. 2015, 21(3), 248-55; Scott et al., Clin. Exp. Rheumatol. 2016, 34(1), 88-93), systemic lupus erythematosus and its complications such as lupus nephritis (Lu et al., J. lmmunol. , 2017, 198(3), 1119-29), and systemic sclerosis (Artlett et al., Arthritis Rheum. 2011, 63(11), 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. I Pathol., 2014, 184: 42-54; Kim et al., Am. J. Respir. Crit. Care Med, 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al., Nature Reviews, 2014, 15, 84-97; and Dempsey et al., Brain. Behay. lmmun. 2017, 61, 306-16), intracranial aneurysms (Zhang et al., J. Stroke and Cerebrovascular Dis., 2015, 24, 5, 972-9), and traumatic brain injury (Ismael et al., J. Neurotrauma., 2018, 35(11), 1294-1303). NLRP3 activity has also been shown to be involved in various metabolic diseases including type 2 diabetes (T2D) and its organ-specific complications, atherosclerosis, obesity, gout, pseudo-gout, metabolic syndrome (Wen et al., Nature Immunology, 2012, 13, 352-357; Duewell et al., Nature, 2010, 464, 1357-1361; Strowig et al., Nature, 2014, 481, 278-286), and non-alcoholic steatohepatitis (Mridha et al., J. Hepatol. 2017, 66(5), 1037-46). A role for NLRP3 via IL-1 beta has also been suggested in atherosclerosis, myocardial infarction (van Hout et al., Eur. Heart J. 2017, 38(11), 828-36), heart failure (Sano et al., J. Am. Coll. Cardiol. 2018, 71(8), 875-66), aortic aneurysm and dissection (Wu et al., Arteriosc/er. Thromb. Vase. Biol., 2017,37(4), 694-706), and other cardiovascular events (Ridker et al., N. Engl. J. Med., 2017, 377(12), 1119-31).

Other diseases in which NLRP3 has been shown to be involved include: ocular diseases such as both wet and dry age-related macular degeneration (Doyle et al., Nature Medicine, 2012, 18, 791-798; Tarallo et al., Cell 2012, 149(4), 847-59), diabetic retinopathy (Loukovaara et al., Acta Ophthalmol., 2017, 95(8), 803-8), non-infectious uveitis and optic nerve damage (Puyang et al., Sci. Rep. 2016, 6, 20998); liver diseases including non-alcoholic steatohepatitis (NASH) and acute alcoholic hepatitis (Henao-Meija et al., Nature, 2012, 482, 179-185); inflammatory reactions in the lung and skin (Primiano et al., J. lmmunol. 2016, 197(6), 2421-33) including contact hypersensitivity (such as bullous pemphigoid (Fang et al., J Dermatol Sci. 2016, 83(2), 116-23)), atopic dermatitis (Niebuhr et al., Allergy, 2014, 69(8), 1058-67), Hidradenitis suppurativa (Alikhan et al., J. Am. Acad. Dermatol. , 2009 ,60(4), 539-61), and sarcoidosis (Jager et al., Am. J. Respir. Crit. Care Med., 2015, 191, A5816); inflammatory reactions in the joints (Braddock et al., Nat. Rev. Drug Disc, 2004, 3, 1-10); amyotrophic lateral sclerosis (Gugliandolo et al., Int. J. Mo/. Sci., 2018, 19(7), E1992); cystic fibrosis (lannitti et al., Nat. Commun., 2016, 7, 10791); stroke (Walsh et al., Nature Reviews, 2014, 15, 84-97); chronic kidney disease (Granata et al., PLoS One 2015, 10(3), eoi22272); and inflammatory bowel diseases including ulcerative colitis and Crohn's disease (Braddock et al., Nat. Rev. Drug Disc, 2004, 3, 1-10; Neudecker et a/., J. Exp. Med. 2017, 214(6), 1737-52; Lazaridis et al., Dig. Dis. Sci. 2017, 62(9), 2348-56). The NLRP3 inflammasome has been found to be activated in response to oxidative stress. NLRP3 has also been shown to be involved in inflammatory hyperalgesia (Dolunay et al., Inflammation, 2017, 40, 366-86).

Activation of the NLRP3 inflammasome has been shown to potentiate some pathogenic infections such as influenza and Leishmaniasis (Tate et al., Sci Rep., 2016, 10(6), 27912-20; Novias et al., PLOS Pathogens 2017, 13(2), e1006196).

NLRP3 has also been implicated in the pathogenesis of many cancers (Menu et al., Clinical and Experimental Immunology, 2011, 166, 1-15). For example, several previous studies have suggested a role for IL-1 beta in cancer invasiveness, growth and metastasis, and inhibition of IL-1 beta with canakinumab has been shown to reduce the incidence of lung cancer and total cancer mortality in a randomised, double-blind, placebo-controlled trial (Ridker et al., Lancet. , 2017, 390(10105), 1833-42). Inhibition of the NLRP3 inflammasome or IL-1 beta has also been shown to inhibit the proliferation and migration of lung cancer cells in vitro (Wang et al., Onco/Rep., 2016, 35(4), 2053-64). A role for the NLRP3 inflammasome has been suggested in myelodysplastic syndromes, myelofibrosis and other myeloproliferative neoplasms, and acute myeloid leukemia (AML) (Basiorka et al., Blood, 2016, 128(25), 2960-75.) and also in the carcinogenesis of various other cancers including glioma (Li et al., Am. J. Cancer Res. 2015, 5(1), 442-9), inflammation-induced tumors (Allen et al., J. Exp. Med. 2010, 207(5), 1045-56; Hu et al., PNAS., 2010, 107(50), 21635-40), multiple myeloma (Li et al., Hematology, 2016 21(3), 144-51), and squamous cell carcinoma of the head and neck (Huang et al., J. Exp. Clin. Cancer Res., 2017, 36(1), 116). Activation of the NLRP3 inflammasome has also been shown to mediate chemoresistance of tumor cells to 5-Fluorouracil (Feng et al., J. Exp. Clin. Cancer Res., 2017, 36(1), 81), and activation of NLRP3 inflammasome in peripheral nerve contributes to chemotherapy-induced neuropathic pain (Jia et al., Mol. Pain., 2017, 13, 1-11). NLRP3 has also been shown to be required for the efficient control of viruses, bacteria, and fungi.

The activation of NLRP3 leads to cell pyroptosis and this feature plays an important part in the manifestation of clinical disease (Yan-gang et al., Cell Death and Disease, 2017, 8(2), 2579; Alexander et al., Hepatology, 2014, 59(3), 898-910; Baldwin et al., J. Med. Chem., 2016, 59(5), 1691-1710; Ozaki et a/., J. Inflammation Research, 2015, 8, 15-27; Zhen et a/., Neuroimmunology Neuroinflammation, 2014, 1(2), 60-65; Mattia et a/., J. Med. Chem., 2014, 57(24), 10366-82; Satoh et al., Cell Death and Disease, 2013, 4, 644). Therefore, it is anticipated that inhibitors of NLRP3 will block pyroptosis, as well as the release of pro-inflammatory cytokines (e.g. IL-1 beta) from the cell.

Hence, the compounds of the invention (without the provisos), as described herein (e.g. in any of the embodiments described herein, including by the examples, and/or in any of the forms described herein, e.g. in a salt form or free form, etc) exhibit valuable pharmacological properties, e.g. NLRP3 inhibiting properties on the NLRP3 inflammasome pathway e.g. as indicated in vitro tests as provided herein, and are therefore indicated for therapy or for use as research chemicals, e.g. as tool compounds. Compounds of the invention (without the provisos) may be useful in the treatment of an indication selected from: inflammasome-related diseases/disorders, immune diseases, inflammatory diseases, auto-immune diseases, or auto-inflammatory diseases, for example, of diseases, disorders or conditions in which NLRP3 signaling contributes to the pathology, and/or symptoms, and/or progression, and which may be responsive to NLRP3 inhibition and which may be treated or prevented, according to any of the methods/uses described herein, e.g. by use or administration of a compound of the invention, and, hence, in an embodiment, such indications may include:
I. Inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of:
   a. a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
   b. a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
   c. a muscular condition such as polymyositis or myasthenia gravis;
   d. a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
   e. a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
   f. a vascular condition such as atherosclerosis, Behcet's disease, vasculitides, or Wegener's granulomatosis;
   g. an immune condition, e.g. autoimmune condition, such as systemic lupus erythematosus (SLE), Sjogren's syndrome, systemic sclerosis, Hashimoto's thyroiditis, type I diabetes, idiopathic thrombocytopenia purpura, or Graves disease;
   h. an ocular condition such as uveitis, allergic conjunctivitis, or vernal conjunctivitis;
   i. a nervous condition such as multiple sclerosis or encephalomyelitis;
   j. an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, mycobacterium tuberculosis, mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
   k. a renal condition such as mesangial proliferative glomerulonephritis, nephrotic syndrome, nephritis, glomerular nephritis, acute renal failure, uremia, or nephritic syndrome;
   l. a lymphatic condition such as Castleman's disease;
   m. a condition of, or involving, the immune system, such as hyper lgE syndrome, lepromatous leprosy, familial hemophagocytic lymphohistiocytosis, or graft versus host disease;
   n. a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
   o. a cancer, including those cancers listed herein below;
   p. a burn, wound, trauma, haemorrhage or stroke;
   q. radiation exposure;
   r. obesity; and/or
   s. pain such as inflammatory hyperalgesia;
II. Inflammatory disease, including inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2—associated autoinflammatory, antibody deficiency and immune dysregulation (APLAID), or sideroblastic anaemia with B-cell immunodeficiency, periodic fevers and developmental delay (SIFD);
III. Immune diseases, e.g. auto-immune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia;
IV. Cancer including lung cancer, renal cell carcinoma, non-small cell lung carcinoma (NSCLC), Langerhans cell histiocytosis (LCH), myeloproliferative neoplams (MPN), pancreatic cancer, gastric cancer, myelodysplastic syndrome (MOS), leukaemia including acute lymphocytic leukaemia (ALL) and acute myeloid leukaemia (AML), promyelocytic leukemia (APML, or APL), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
V. Infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), papillomavirus, or SARS-CoV-2) bacterial infections (e.g. from Staphylococcus aureus, Helicobacter pylori, Bacillus anthracia, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), and prion infections;
VI. Central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, multiple sclerosis, and amyotrophic lateral sclerosis;
VII. Metabolic diseases such as type 2 diabetes (T2D), atherosclerosis, obesity, gout, and pseudo-gout;
VIII. Cardiovascular diseases such as hypertension, ischaemia, reperfusion injury including post-Ml ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome;
IX. Respiratory diseases including chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis;
X. Liver diseases including non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH);
XI. Renal diseases including acute kidney disease, hyperoxaluria, chronic kidney disease, oxalate nephropathy, nephrocalcinosis, glomerulonephritis, and diabetic nephropathy;
XII. Ocular diseases including those of the ocular epithelium, age-related macular degeneration (AMO) (dry and wet), uveitis, corneal infection, diabetic retinopathy, optic nerve damage, dry eye, and glaucoma;
XIII Skin diseases including dermatitis such as contact dermatitis and atopic dermatitis, contact hypersensitivity, sunburn, skin lesions, hidradenitis suppurativa (HS), other cyst-causing skin diseases, and acne conglobata;
XIV. Lymphatic conditions such as lymphangitis, and Castleman's disease;
XV. Psychological disorders such as depression, and psychological stress;
XVI. Graft versus host disease;
XVII. Bone diseases including osteoporosis, osteopetrosis;
XVIII. Blood disease including sickle cell disease;
XIX. Allodynia including mechanical allodynia; and
XX. Any disease where an individual has been determined to carry a germline or somatic non-silent mutation in NLRP3.

More specifically the compounds of the invention (without the provisos) may be useful in the treatment of an indication selected from: inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), sickle cell disease, systemic lupus erythematosus (SLE), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g.

hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MOS), myelofibrosis). In particular, autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis).

In particular, compounds of the invention (without the provisos), may be useful in the treatment of a disease or disorder selected from autoinflammatory fever syndromes (e.g. CAPS), sickle cell disease, Type I Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). Thus, as a further aspect, the present invention provides the use of a compound of the invention (without the provisos) (hence, including a compound as defined by any of the embodiments/forms/examples herein) in therapy. In a further embodiment, the therapy is selected from a disease, which may be treated by inhibition of NLRP3 inflammasome. In another embodiment, the disease is as defined in any of the lists herein. Hence, there is provided any one of the compounds of the invention (without the provisos) described herein (including any of the embodiments/forms/examples) for use in the treatment of any of the diseases or disorders described herein (e.g. as described in the aforementioned lists).

### Pharmaceutical Compositions and Combinations

In an embodiment, the invention also relates to a composition comprising a pharmaceutically acceptable carrier and, as active ingredient, a therapeutically effective amount of a compound of the invention (without the provisos). The compounds of the invention (without the provisos) may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

In an embodiment, and depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

In an embodiment, there is provided a combination comprising a therapeutically effective amount of a compound of the invention (without the provisos), according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH₄) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention (without the provisos), e.g. for use in the treatment of a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in repsect of compounds of the invention (without the provisos), but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein), for instance, in an embodiment, the combination may be a kit of parts. Such combinations may be referred to as “pharmaceutical combinations”. The route of administration for a compound of the invention (without the provisos) as a component of a combination may be the same or different to the one or more other therapeutic agent(s) with which it is combined. The other therapeutic agent is, for example, a chemical compound, peptide, antibody, antibody fragment or nucleic acid, which is therapeutically active or enhances the therapeutic activity when administered to a patient in combination with a compound of the invention (without the provisos).

The weight ratio of (a) the compound according to the invention and (b) the other therapeutic agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1. The pharmaceutical composition or combination of the present invention can be in unit dosage of about 1-1000 mg of active ingredient(s) for a subject of about 50-70 kg, or about 1-500 mg, or about 1-250 mg, or about 1-150 mg, or about 1-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-³ molar and 10-⁹ molar concentrations. A therapeutically effective amount in vivo may range depending on the route of administration, between about 0.1-500 mg/kg, or between about 1-100 mg/kg.

As used herein, term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “subject” as used herein, refers to an animal, preferably a mammal, most preferably a human, for example who is or has been the object of treatment, observation or experiment.

The term “therapeutically effective amount” as used herein, means that amount of compound of the invention (including, where applicable, form, composition, combination comprising such compound of the invention) elicits the biological or medicinal response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by NLRP3, or (ii) associated with NLRP3 activity, or (iii) characterised by activity (normal or abnormal) of NLRP3; or (2) reduce or inhibit the activity of NLRP3; or (3) reduce or inhibit the expression of NLRP3. In another non-limiting embodiment, the term “a therapeutically effective amount” refers to the amount of the compound of the present invention that, when administered to a cell, or a tissue, or a non-cellular biological material, or a medium, is effective to at least partially reduce or inhibit the activity of NLRP3; or at least partially reduce or inhibit the expression of NLRP3.

As used herein, the term “inhibit”, “inhibition” or “inhibiting” refers to the reduction or suppression of a given condition, symptom, or disorder, or disease, or a significant decrease in the baseline activity of a biological activity or process. Specifically, inhibiting NLRP3 or inhibiting NLRP3 inflammasome pathway comprises reducing the ability of NLRP3 or NLRP3 inflammasome pathway to induce the production of IL-1 and/or IL-18. This can be achieved by mechanisms including, but not limited to, inactivating, destabilizing, and/or altering distribution of NLRP3.

As used herein, the term “NLRP3” is meant to include, without limitation, nucleic acids, polynucleotides, oligonucleotides, sense and anti-sense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder;

or delaying the onset or progression of the disease or disorder.

As used herein, a subject is “in need of” a treatment if such subject would benefit biologically, medically or in quality of life from such treatment.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “pharmaceutical combination” as used herein refers to either a fixed combination in one dosage unit form, or non-fixed combination or a kit of parts for the combined administration where two or more therapeutic agents may be administered independently at the same time or separately within time intervals. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Summary of Pharmacology, uses, Compositions and Combinations

In an embodiment, there is provided a pharmaceutical composition comprising a therapeutically effective amount of a compound of the invention (without the provisos), according to any one of the embodiments described herein, and a pharmaceutically acceptable carrier (including one or more pharmaceutically acceptale carriers).

In an embodiment, there is provided a compound of the invention (without the provisos), according to any one of the embodiments described herein, for use as a medicament.

In an embodiment, there is provided a compound of the invention (without the provisos), according to any one of the embodiments described herein (and/or pharmaceutical compositions comprising such compound of the invention (without the provisos), according to any one of the embodiment described herein) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided a use of compounds of the invention (without the provisos), according to any one of the embodiments described herein (and/or pharmaceutical compositions comprising such compound of the invention (without the provisos), according to any one of the embodiment described herein): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided use of compounds of the invention (without the provisos), according to any one of the embodiments described herein (and/or pharmaceutical compositions comprising such compound of the invention (without the provisos), according to any one of the embodiment described herein), in the manufacture of a medicament for: the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In an embodiment, there is provided a method of treating a disease or disorder in which the NLRP3 signalling contributes to the pathology, and/or symptoms, and/or progression, of said disease/disorder, comprising administering a therapeutically effective amount of a compound of the invention (without the provisos), according to any one of the embodiments described herein (and/or pharmaceutical compositions comprising such compound of the invention (without the provisos), according to any one of the embodiment described herein), for instance to a subject (in need thereof). In a further embodiment, there is provided a method of inhibiting the NLRP3 inflammasome activity in a subject (in need thereof), the method comprising administering to the subject in need thereof a therapeutically effective amount of a compound of the invention (without the provisos), according to any one of the embodiments described herein (and/or pharmaceutical compositions comprising such compound of the invention (without the provisos), according to any one of the embodiment described herein).

In all relevant embodiment of the invention, where a disease or disorder is mentioned (e.g. hereinabove), for instance a disease or disorder in which the NLRP3 signalling contributes to the pathology, and/or symptoms, and/or progression, of said disease/disorder, or, a disease or disorder associated with NLRP3 activity (including

NLRP3 inflammasome activity), including inhibiting NLRP3 inflammasome activity, then such disease may include inflammasome-related diseases or disorders, immune diseases, inflammatory diseases, auto-immune diseases, or auto-inflammatory diseases. In a further embodiment, such disease or disorder may include autoinflammatory fever syndromes (e.g cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukaemia, myelodysplastic syndromes (MOS), myelofibrosis). In a particular aspect, such disease or disorder is selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). In a particular embodiment, the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

In an embodiment, there is provided a combination comprising a therapeutically effective amount of a compound of the invention (without the provisos), according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH₄) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention (without the provisos), e.g. for use in the treatment of a disease or disorder in which the

NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in repsect of compounds of the invention (without the provisos), but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

For instance, compounds of the invention may have the advantage that they have a good or an improved thermodynamic solubility (e.g. compared to compounds known in the prior art; and for instance as determined by a known method and/or a method described herein). Compounds of the invention may have the advantage that they will block pyroptosis, as well as the release of pro-inflammatory cytokines (e.g. IL-113) from the cell. Compounds of the invention may also have the advantage that they avoid side-effects, for instance as compared to compounds of the prior art, which may be due to selectivity of NLRP3 inhibition. Compounds of the invention may also have the advantage that they have good or improved in vivo pharmacokinetics and oral bioavailabilty. They may also have the advantage that they have good or improved in vivo efficacy. Specifically, compounds of the invention may also have advantages over prior art compounds when compared in the tests outlined hereinafter (e.g. in Examples C and D).

### General Preparation and Analytical Processes

The compounds according to the invention can generally be prepared by a succession of steps, each of which may be known to the skilled person or described herein.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SFC).

The starting materials and the intermediates are compounds that are either commercially available or may be prepared according to conventional reaction procedures generally known in the art.

### Analytical Part

LC-MS (LIQUID CHROMATOGRAPHY/MASS SPECTROMETRY)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified differently in the table of data, the reported molecular ion corresponds to the [M+H]⁺ (protonated molecule) and/or [M-H]⁻(deprotonated molecule). In case the compound was not directly ionizable the type of adduct is specified (i.e. [M+NH₄]⁺, [M+HCOO]⁻, etc . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

TABLE
LCMS Method codes (Flow expressed in mL/min; column
temperature (T) in ° C.; Run time in minutes).

| | | | | | | Run |
|---|---|---|---|---|---|---|
| Method code | Instrument | column | mobile phase | gradient | Flow/Col T | time |
| Method A | Waters: | Waters: | A: 95% | From | 1/50 | 5 |
| | Acquity ® | BEH C18 | CH₃COONH₄ | 95% A | | |
| | IClass | (1.7 μm, | 6.5 mM + 5% | to 5% A | | |
| | UPLC ® - | 2.1 × 50 mm) | CH₃CN, B: | in | | |
| | DAD and | | CH₃CN | 4.6 min, | | |
| | Xevo G2-S | | | held for | | |
| | QTOF | | | 0.4 min | | |
| Method B | Waters: | Agilent: | A: 95% | From | 0.8/50 | 5 |
| | Acquity ® | RRHD | CH₃COONH₄ | 95% A | | |
| | IClass | (1.8 μm, | 6.5 mM + 5% | to 5% A | | |
| | UPLC ® - | 2.1 × 50 mm) | CH₃CN, B: | in | | |
| | DAD and | | CH₃CN | 4.5 min, | | |
| | SQD | | | held for | | |
| | | | | 0.5 min | | |
| Method C | Agilent: | Waters: | A: HCO₃NH₄ | From | 0.8/25 | 5 |
| | 1290 | XBridge C18 | 2.5 g/L (32 | 90% A | | |
| | Infinity II - | (2.5 μm, | mM) | to 0% A | | |
| | DAD and | 2.1 × 50 mm) | B: CH₃CN | in | | |
| | MSD/XT | | | 3.0 min, | | |
| | | | | held for | | |
| | | | | 0.5 min, | | |
| | | | | to 90% | | |
| | | | | A in 0.7 | | |
| | | | | min, | | |
| | | | | held for | | |
| | | | | 0.8 min | | |
| Method D | Waters: | Waters: | A: 95% | From | 0.8/50 | 2.5 |
| | Acquity ® | BEH C18 | CH3COONH4 | 95% A | | |
| | UPLC ® - | (1.7 μm, | 6.5 mM + 5% | to 5% A | | |
| | DAD and | 2.1 × 50 mm) | CH3CN, B: | in 2.0 | | |
| | SQD | | CH3CN | min, | | |
| | | | | held for | | |
| | | | | 0.5 min | | |
| Method E | Waters: | Waters: | A: 95% | From | 0.8/50 | 5 |
| | Acquity ® | BEH C18 | CH3COONH4 | 95% A | | |
| | UPLC ® - | (1.7 μm, | 6.5 mM + 5% | to 5% A | | |
| | DAD and | 2.1 × 50 mm) | CH3CN, B: | in | | |
| | SQD | | CH3CN | 4.5 min, | | |
| | | | | held for | | |
| | | | | 0.5 min | | |
| Method F | Waters: | Waters: | A: HCO3NH4 | From | 0.8/25 | 3.0 |
| | Acquity ® | XBridgeC18 | 2.5 g/L (32 | 90% A | | |
| | HClass | (2.5 μm, | mM) | to 0% A | | |
| | UPLC ® - | 2.1 × 50 mm) | B: CH3CN | in | | |
| | DAD and | | | 2.0 min, | | |
| | QDa | | | held for | | |
| | | | | 0.5 min, | | |
| | | | | to 90% | | |
| | | | | A, held | | |
| | | | | for 0.5 | | |
| | | | | min | | |
| Method G | Waters: | Waters: | A: 0.1% | From | 0.6/55 | 3.5 |
| | Acquity ® | BEH | NH4HCO3 | 100% A | | |
| | UPLC ® - | (1.8 μm, | in 95% H2O + | to | | |
| | DAD and | 2.1*100 mm) | 5% CH3CN | 5% A in | | |
| | SQD | | B: CH3CN | 2.10 min, | | |
| | | | | to 0% A | | |
| | | | | in | | |
| | | | | 0.9 min, | | |
| | | | | to 5% A | | |
| | | | | in | | |
| | | | | 0.5 min | | |
| Method H | Waters: | Waters: | A: 0.1% | From | 0.6/55 | 3.5 |
| | Acquity ® | BEH | NH4HCO3 | 100% A | | |
| | UPLC ® - | (1.8 μm, | in 95% H2O + | to | | |
| | DAD and | 2.1*100 mm) | 5% CH3CN | 5% A in | | |
| | SQD | | B: MeOH | 2.10 min, | | |
| | | | | to 0% A | | |
| | | | | in | | |
| | | | | 0.9 min, | | |
| | | | | to 5% A | | |
| | | | | in | | |
| | | | | 0.5 min | | |
| Method I | Waters: | Waters: | A: 0.1% | From | 0.8/55 | 2.0 |
| | Acquity ® | BEH | NH4HCO3 | 100% A | | |
| | UPLC ® - | (1.8 μm, | in 95% H2O + | to | | |
| | DAD and | 2.1*50 mm) | 5% CH3CN | 5% A in | | |
| | SQD | | B: CH3CN | 1.3 min, | | |
| | | | | hold | | |
| | | | | 0.7 min | | |
| Method J | Waters: | Waters: | A: 0.1% | From | 0.6/55 | 3.5 |
| | Acquity ® | BEH | NH4HCO3 | 100% A | | |
| | UPLC ® - | (1.8 μm, | in 95% H2O + | to | | |
| | DAD and | 2.1*100 mm) | 5% CH3CN | 5% A in | | |
| | SQD2 | | B: CH3CN | 2.10 min, | | |
| | | | | to 0% A | | |
| | | | | in | | |
| | | | | 1.4 min | | |
| Method K | Waters: | Waters: | A: 10 mM | From | 0.6/55 | 3.5 |
| | Acquity ® | BEH | CH3COONH4 | 100% A | | |
| | UPLC ® - | (1.8 μm, | in 95% H2O + | to | | |
| | DAD and | 2.1*100 mm) | 5% CH3CN | 5% A in | | |
| | SQD | | B: CH3CN | 2.00 min, | | |
| | | | | to 0% A | | |
| | | | | in | | |
| | | | | 0.9 min, | | |
| | | | | to 5% A | | |
| | | | | in | | |
| | | | | 0.5 min | | |
| Method L | Waters: | Waters: | A: 0.1% | From | 0.6/55 | 3.5 |
| | Acquity ® | BEH | NH4HCO3 | 100% A | | |
| | UPLC ® - | (1.8 μm, | in 95% H2O + | to | | |
| | DAD and | 2.1*100 mm) | 5% CH3CN | 5% A in | | |
| | SQD | | B: CH3CN | 2.10 min, | | |
| | | | | to 0% A | | |
| | | | | in | | |
| | | | | 0.9 min, | | |
| | | | | to 5% A | | |
| | | | | in | | |
| | | | | 0.4 min | | |
| Method M | Agilent | YMC-pack | A: 0.1% | From | 2.6/35 | 6.2 |
| | 1100 | ODS-AQ | HCOOH in | 95% A | | |
| | HPLC | C18 (50 × | H2O | to 5% A | | |
| | DAD | 4.6 mm, 3 | B: CH3CN | in 4.8 | | |
| | LC/MS | μm) | | min, | | |
| | G1956A | | | held for | | |
| | | | | 1.0 min, | | |
| | | | | to 95% | | |
| | | | | A in 0.2 | | |
| | | | | min | | |
| Method N | Waters: | Waters: | A: 10 mM | From | 0.6/55 | 3.5 |
| | Acquity ® | BEH | CH3COONH4 | 100% A | | |
| | UPLC ® - | (1.8 μm, | in 95% H2O + | to | | |
| | DAD and | 2.1*100 mm) | 5% CH3CN | 5% A in | | |
| | SQD | | B: CH3CN | 2.10 min, | | |
| | | | | to 0% A | | |
| | | | | in | | |
| | | | | 0.9 min, | | |
| | | | | to 5% A | | |
| | | | | in | | |
| | | | | 0.5 min | | |
| Method O | Waters: | Waters: BEH | A: 95% | 84.2% A | 0.343/40 | 6.2 |
| | Acquity | C18 (1.7 μm, | CH₃COONH₄ | for 0.49 | | |
| | UPLC ® - | 2.1 × 100 mm) | 7 mM/5% | min, to | | |
| | DAD and | | CH₃CN | 10.5% A | | |
| | Quattro | | B: CH₃CN | in 2.18 | | |
| | Micro ™ | | | min, held | | |
| | | | | for 1.94 | | |
| | | | | min, back | | |
| | | | | to 84.2% | | |
| | | | | A in 0.73 | | |
| | | | | min, held | | |
| | | | | for 0.73 | | |
| | | | | min. | | |
| Method P | Waters: | Waters: BEH | A: 95% | From | 1/50 | 2 |
| | Acquity ® | C18 (1.7 μm, | CH3COONH4 | 95% A to | | |
| | IClass | 2.1 × 50 mm) | 6.5 mM + 5% | 40% A | | |
| | UPLC ® - | | CH3CN, B: | in | | |
| | DAD and | | CH3CN | 1.2 min, | | |
| | Xevo G2-S | | | to 5% A | | |
| | QTOF | | | in | | |
| | | | | 0.6 min, | | |
| | | | | held for | | |
| | | | | 0.2 min | | |
| Method Q | Waters: | Waters: | A: 95% | From | 1/50 | 5 |
| | Acquity ® | XBridge C18 | CH3COONH4 | 95% A to | | |
| | IClass | (2.5 μm, | 6.5 mM + 5% | 5% A in | | |
| | UPLC ® - | 2.1 × 50 mm) | CH3CN, B: | 4.6 min, | | |
| | DAD and | | CH3CN | held for | | |
| | SQD | | | 0.5 min | | |
| Method R | Waters: | BEH C18 | A:10 mM | 95% A | 0.8/55 | 2 |
| | Acquity ® | column (1.7 | ammonium | and 5% | | |
| | UPLC ® - | μm. 2.1 × 50 | acetate in | B to 5% | | |
| | DAD and | mm; Waters | H2O/acetonitrile | A and 95 | | |
| | SQD | Acquity) | 95/5; | % B in | | |
| | | | B: acetonitrile | 1.3 | | |
| | | | | minutes | | |
| | | | | and hold | | |
| | | | | for 0.7 | | |
| | | | | minutes | | |

### NMR

For a number of compounds, ¹H NMR spectra were recorded on a Bruker Avance III spectrometer operating at 400 MHz, on a Bruker Avance III-HD operating at 400 MHz, on a Bruker Avance NEO spectrometer operating at 400 MHz, on a

Bruker Avance Neo spectrometer operating at 500 MHz, or on a Bruker Avance 600 spectrometer operating at 600 MHz, using CHLOROFORM-d (deuterated chloroform, CDCl₃), DMSO-d₆ (deuterated DMSO, dimethyl-d₆ sulfoxide), METHANOL-d₄ (deuterated methanol), BENZENE-d₆ (deuterated benzene, C₆D₆) or ACETONE-d₆ (deuterated acetone, (CD₃)₂CO) as solvents. Chemical shifts (6) are reported in parts per million (ppm) relative to tetramethylsilane (TMS), which was used as internal standard.

### Melting Points

Values are either peak values or melt ranges, and are obtained with experimental uncertainties that are commonly associated with this analytical method. For a number of compounds, melting points were determined with a DSC823e

(Mettler-Toledo) apparatus. Melting points were measured with a temperature gradient of 10° C/minute. Standard maximum temperature was 300° C.

### Experimental Part

Hereinafter, the term “m.p.” means melting point, “aq.” means aqueous, “r.m.” means reaction mixture, “rt” means room temperature, ‘DIPEA’ means N,N-diiso-propylethylamine, “DIPE” means diisopropylether, ‘THF’ means tetrahydrofuran, ‘DMF’ means dimethylformamide, ‘DCM’ means dichloromethane, “EtOH” means ethanol ‘ETOAc’ means ethyl acetate, “AcOH” means acetic acid, “iPrOH” means isopropanol, “iPrNH₂” means isopropylamine, “MeCN” or “ACN” means acetonitrile, “MeOH” means methanol, “Pd(OAc)₂” means palladium(II)diacetate, “rac” means racemic, ‘sat.’ means saturated, ‘SFC’ means supercritical fluid chromatography, ‘SFC-MS’ means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rₜ” (or “RT”) means retention time (in minutes), “[M+H]⁺” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate (1-[bis(dimethylamino)methylene]-1H-1,2,3-triazolo[4,5-b]pyridinium 3-oxide hexafluorophosphate), “Xantphos” means (9,9-dimethyl-9H-xanthene-4,5-diyl)bis[diphenylphosphine], “TBAT” means tetrabutyl ammonium triphenyldifluorosilicate, “TFA” means trifuoroacetic acid, “Et2O” means diethylether, “DMSO” means dimethylsulfoxide, “SiO₂” means silica, “XPhos Pd G3” means (2-dicyclohexylphosphino-2′,4′,6′-triisopropyl-1,1′-biphenyl)[2-(2′-amino-1,1′-biphenyl)]palladium(II) ethanesulfonate.

For key intermediates, as well as some final compounds, the absolute configuration of chiral centers (indicated as R and/or 5) were established via comparison with samples of known configuration, or the use of analytical techniques suitable for the determination of absolute configuration, such as VCD (vibrational cicular dichroism) or X-ray crystallography. When the absolute configuration at a chiral center is unknown, it is arbitrarily designated R*.

### EXAMPLES

### Synthesis of ethyl 5-nitro-1H-pyrazole-3-carboxylate

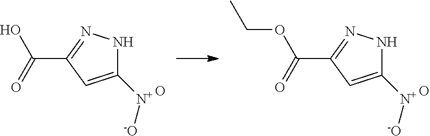

5-nitro-1H-pyrazole-3-carboxylic acid [198348-89-9] (42 g, 267.37 mmol) was dissolved in EtOH (500 mL) and acetyl chloride (37.8 mL) was added at 0° C. The mixture was warmed to rt and stirred for 2h .The mixture was extracted with EtOAc.

The combined organic layers were concentrated to give ethyl 5-nitro-1H-pyrazole-3-carboxylate (45 g, yield 90%).

Synthesis of ethyl 5-amino-1H-pyrazole-3-carboxylate

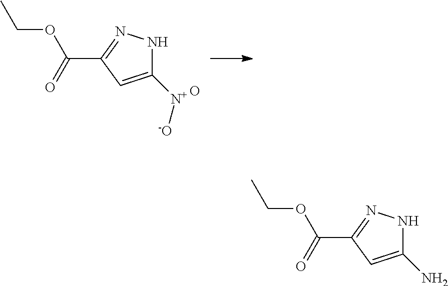

Ethyl 5-nitro-1H-pyrazole-3-carboxylate (47 g, 253.87 mmol) was dissolved in THF (300 mL) and Pd/C (2 g) was added. The mixture was stirred 12h at rt under a H₂ atmosphere. The mixture was evaporated. H₂O was added, the mixture was extracted with EtOAc, the combined organic layers were dried over Na₂SO₄, filtered and evaporated to yield ethyl 5-amino-1H-pyrazole-3-carboxylate (37 g, yield 92%).

Synthesis of 3-iodo-1H-pyrazole-3-carboxylate

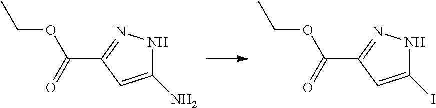

Ethyl 5-amino-1H-pyrazole-3-carboxylate (40 g, 257.81 mmol) was dissolved in HCl (1800 mL), NaNO₂ (40 g, 579.75 mmol) was added, the mixtured is stirred for 5 minutes then KI (110g, 662.64 mmol) was added .The mixture was stirred at 0° C. for 40min, then the mixture was evaporated. H₂O was added, the mixture was extracted with EtOAc, the combined organic layers were dried over Na₂SO₄, filtered and evaporated to give 3-iodo-1H-pyrazole-3-carboxylate (21328.3 mg, yield 31%).

¹H NMR (400 MHz, DMSO-d₆) d ppm 1.27 (t, J=7.06 Hz, 3 H) 4.27 (q, J=6.98 Hz, 2 H) 6.84-7.08 (m, 1 H) 14.20 (br. s., 1 H)

Synthesis of 1-(2,2-difluorocyclopropyl)ethan-1-one

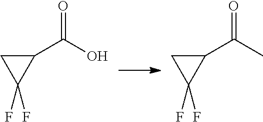

Methyllithium 1.6 M in diethylether [917-54-4] (45 mL, 72 mmol) was added dropwise to a mixture of 2,2-difluorocyclopropanecarboxylic acid [107873-03-0] (5 g, 41.0 mmol) in Et2O (100 mL) at ˜78° C. (during the addition, the internal temperature was kept below −50° C.). The resulting mixture was stirred at −78° C. for 1 h and warmed slowly (with the cooling bath) to rt. When the resulting mixture was at 10° C., it was poured into crushed ice. The aqueous layers were extracted with Et2O (once). The combined organic layers were evaporated under reduced pressure (800 mbars) at 40° C. to give 1-(2,2-difluorocyclopropyl)ethan-1-one (3.5 g, yield 14%, 80% w/w Et2O) as a yellow oil.
Synthesis of ethyl 3-(2,2-difluorocyclopropyl)-1H-pyrazole-5-carboxylate

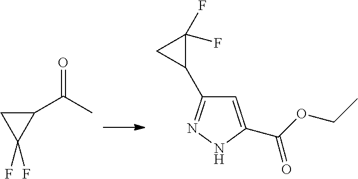

t-BuOK [865-47-4] (915 mg, 8.15 mmol) in THF (4 mL) was added dropwise to a stirred solution of 1-(2,2-difluorocyclopropyl)ethan-1-one (3.5 g, 5.83 mmol, 20% purity) and diethyl oxalate [95-92-1] (793 5.83 mmol) in THF (4 mL) under nitrogen at 0° C. The mixture was stirred at rt for 20h. Then, HOAc [64-19-7] (667 11.7 mmol) and hydrazine hydrate [7803-57-8] (402 5.39 mmol) were added to the reaction mixture at rt and the mixture was stirred at 80° C. for 4 h. Then the solution was evaporated to dryness, the residue was diluted with H₂O and extracted with DCM. The organic layer was separated, dried over MgSO₄, filtered and evaporated to dryness to give a residue which was purified by flash chromatography (irregular SiOH 15-40 p.m, 24 g Grace, liquid injection (DCM), mobile phase gradient: from Heptane/EtOAc 90/10 to 40/60) to give ethyl 3-(2,2-difluorocyclopropyl)-1H-pyrazole-5-carboxylate (430 mg, global yield=5% over 2 steps) as a yellow solid.
Synthesis of ethyl 3-(2,2,2-trifluoroethyl)-1H-pyrazole-5-carboxylate

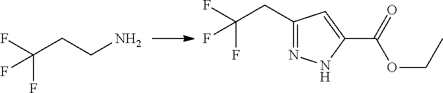

NaNO_{2 [7632}-00-0] (7.69 g, 111.45 mmol) in water (21 mL) was added to a solution of 3,3,3-trifluoropropylamine hydrochloride [2968-33-4] (10 g, 66.87 mmol) in DCM (437 mL) at 0° C. Then, ethyl propiolate [623-47-2] (2.26 mL, 22.29 mmol) was added to the reaction mixture and stirred at rt for 90 h. After 30 min bubbling N2, the mixture was diluted with water and extracted with DCM (3x). The organic layer was separated, dried (MgSO₄), filtered and the solvents evaporated in vacuo. The crude product was purified by flash column chromatography (silica, 80 g; AcOEt in heptane 0/100 to 30/70). The desired fractions were collected and concentrated in vacuo to yield 3-(2,2,2-trifluoroethyl)-1H-pyrazole-5-carboxylate (1.05 g, yield 19%) as a pink pale solid.

1H NMR (300 MHz, DMSO) d 6.76 (s, 1H), 4.29 (q, J=7.1 Hz, 2H), 3.74 (q, J=11.2 Hz, 2H), 1.29 (t, J=7.1 Hz, 3H).

Synthesis of 5-iodo-1H-pyrazole-3-carbohydrazide (I-1)

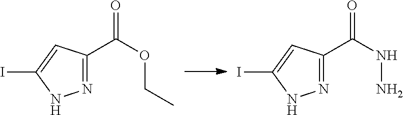

Hydrazine hydrate [7803-57-8] (6 mL, 1.032 g/mL, 123.69 mmol) was added dropwise to a stirred solution of ethyl 3-iodo-1H-pyrazole-3-carboxylate [141998-77-8] (5.3 g, 19.90 mmol) in EtOH (60 mL) in a sealed tube and under N2. The mixture was stirred at 80° C. for 2.5 days. The solvent was evaporated in vacuo and co-evaporated with toluene. The crude was treated with a 1/1 mixture of water/MeOH, filtered and washed with more MeOH. The solid was dried under vacuum to yield 5-iodo-1H-pyrazole-3-carbohydrazide (I-1) (3.63 g, 72%) as a white solid. The filtrate was extracted with a 9/1 mixture of DCM/iPrOH. The organic layer was separated, dried (MgSO4), filtered and the solvent evaporated in vacuo to yield additional 5-iodo-1H-pyrazole-3-carbohydrazide (I-1) (0.91 g, 18%) as a white solid.

1H NMR (400 MHz, DMSO-d₆) δ ppm 4.48 (br s, 2 H) 6.91 (s, 1 H) 9.66 (br s, 1 H) 13.10-14.00 (m, 1 H)

Structure analogs were synthesized using the same procedure.

| Starting material | Intermediate |
|---|---|
| 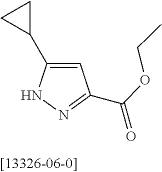 | 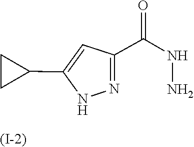 [SMILES: NNC(=O)c1cc(C2CC2)[nH]n1 ; "(I-2)"] |
| 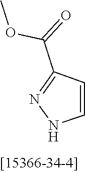 | 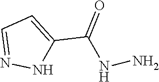 |
| 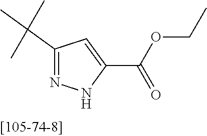 | 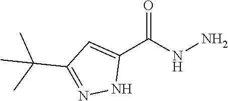 |
| 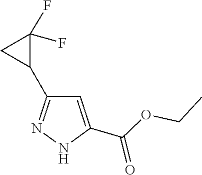 | 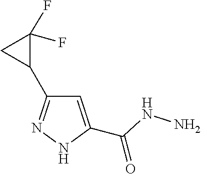 [SMILES: NNC(=O)c1cc(C2CC2(F)F)n[nH]1] |
| 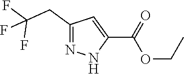 | 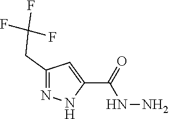 [SMILES: NNC(=O)c1cc(CC(F)(F)F)n[nH]1] |

Synthesis of 2-iodo-7-isopropyl-5H-pyrazolo[1,5-d][1,2,4]triazin-4-one (I-3)

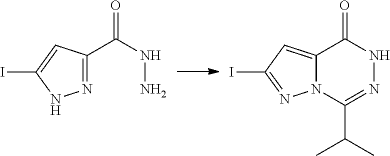

Aluminum isopropoxide [555-31-7] (160 mg, 0.78 mmol) followed by 1,1,1-trimethoxy-2-methylpropane [52698-46-1] (1.55 mL, 0.93 g/mL, 9.73 mmol) were added to a stirred suspension of 5-iodo-1H-pyrazole-3-carbohydrazide (I-1) (2 g, 7.94 mmol) in butyronitrile [109-74-0] (40 mL) in a sealed tube and under N2. The mixture was stirred at 115° C. for 24 h. The mixture was basified with 1 N aqueous solution of NaOH until pH=10, diluted with water and extracted with EtOAc. The organic layer was separated, dried (MgSO₄), filtered and the solvents evaporated in vacuo. The crude product was purified by flash column chromatography (SiO2, EtOAc in heptane 10/90 to 100/0). The desired fractions were collected and concentrated in vacuo to yield 2-iodo-7-isopropyl-5H-pyrazolo[1,5-d][1,2,4]triazin-4-one (I-3) (1.979 g, 82%) as a white solid.

1H NMR (500 MHz, DMSO-d₆) δ ppm 1.29 (d, J=6.87 Hz, 6 H) 3.53 (quin, J=6.87

Hz, 1 H) 7.40 (s, 1 H) 12.38 (s, 1 H)

Structure analogs were synthesized using the same procedure.

| Reagent | Starting material | Intermediate |
|---|---|---|
| 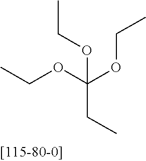 | 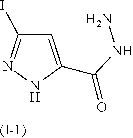 | 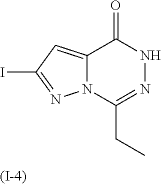 |
| 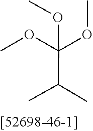 | 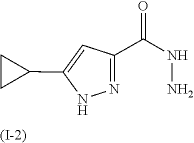 | 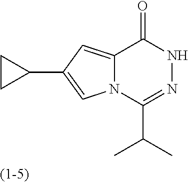 [SMILES: CC(C)c1n[nH]c(=O)c2cc(C3CC3)cn12] |
| 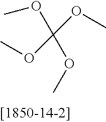 | 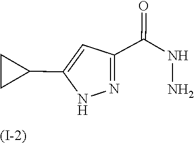 [SMILES: NNC(=O)c1cc(C2CC2)[nH]n1 ; "(I-2)"] | 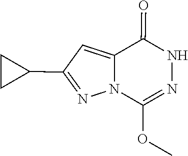 |
| 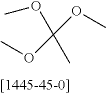 | 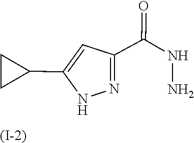 [SMILES: NNC(=O)c1cc(C2CC2)[nH]n1 ; "(I-2)"] | 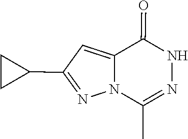 |
| 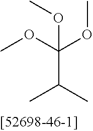 | 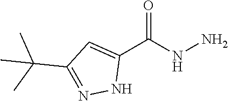 | 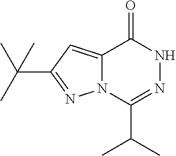 [SMILES: CC(C)c1n[nH]c(=O)c2cc(C(C)(C)C)nn12] |
| 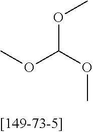 | 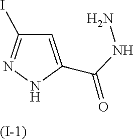 | 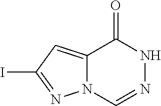 [SMILES: O=c1[nH]ncn2nc(I)cc12] |
| 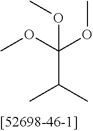 | 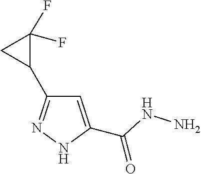 [SMILES: NNC(=O)c1cc(C2CC2(F)F)n[nH]1] | 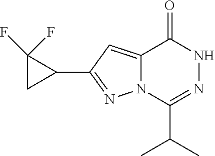 [SMILES: CC(C)c1n[nH]c(=O)c2cc(C3CC3(F)F)nn12] |
| 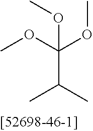 | 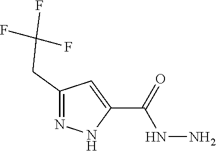 [SMILES: NNC(=O)c1cc(CC(F)(F)F)n[nH]1] | 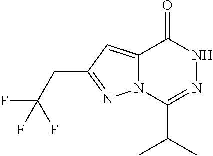 [SMILES: CC(C)c1n[nH]c(=O)c2cc(CC(F)(F)F)nn12] |

Synthesis of 7-ethylpyrazolo[1,5-d][1,2,4]triazin-4(5H)-one

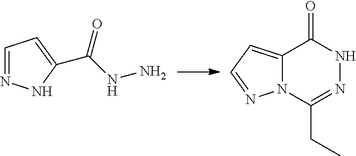

A mixture of 1H-pyrazole-5-carbohydrazide (2 g, 15.86 mmol) and trethyl orthopropionate [115-80-0] (3.07 g, 17.44 mmol) in DMF [68-12-2] (14.8 mL) was heated at 165° C. in a pressure tube overnight. The RM was cooled, the solids were filtered and washed with EtOH to yield 7-ethylpyrazolo[1,5-d][1,2,4]triazin-4(5H)-one (1.27 g, yield 49%) as a white solid.

Structure analogs were synthesized using the same procedure.

| Reagent | Starting material | Intermediate |
|---|---|---|
| 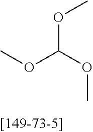 | 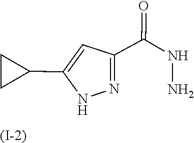 [SMILES: NNC(=O)c1cc(C2CC2)[nH]n1 ; "(I-2)"] | 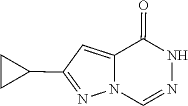 [SMILES: O=c1[nH]ncn2nc(C3CC3)cc12] |

Synthesis of 2-ethyl-7-isopropyl-5H-pyrazolo[1,5-d][1,2,4]triazin-4-one (I-6)

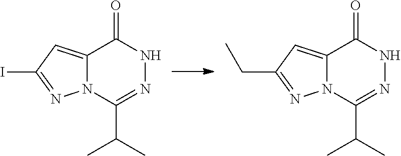

A 1M solution of diethylzinc solution in hexanes [557-20-0] (4.8 mL, 1 M, 4.8 mmol) was added dropwise to a stirred solution of 2-iodo-7-isopropyl-5H-pyrazolo[1,5-d][1,2,4]triazin-4-one (I-3) (0.48 g, 1.57 mmol) and XPhos Pd G3 [1445085-55-1] (111 mg, 0.13 mmol) in THF (8 mL) at 0° C. in a tube and under N2. The mixture was stirred at 0° C. for 5 min and at rt for 2 h. The mixture was cooled down to 0° C. and treated carefully and dropwise with 20% aqueous solution of NH₄C1. The mixture was extracted with DCM. The organic layer was separated, dried (MgSO₄), filtered and the solvents evaporated in vacuo. The crude product was purified by flash column chromatography (SiO2, EtOAc in heptane 10/90 to 100/0). The desired fractions were collected and concentrated in vacuo to yield 2-ethyl-7-isopropyl-5H-pyrazolo[1,5-d][1,2,4]triazin-4-one (I-6) (140 mg, 43%) as a white solid.

¹H NMR (400 MHz, DMSO-d₆) δ ppm 1.16-1.44 (m, 9 H) 2.78 (q, J=7.55 Hz, 2 H) 3.57 (quin, J=6.88 Hz, 1 H) 7.03 (s, 1 H) 12.21 (br s, 1 H)

Synthesis of 2-isopropenyl-7-isopropyl-5H-pyrazolo[1,5-d][1,2,4]triazin-4-one (I-7)

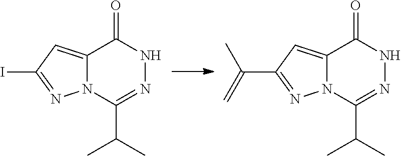 [SMILES: CC(C)c1n[nH]c(=O)c2cc(I)nn12 ; C=C(C)c1cc2c(=O)[nH]nc(C(C)C)n2n1]

To a stirred solution of 2-iodo-7-isopropyl-5H-pyrazolo[1,5-d][1,2,4]triazin-4-one (I-3) (0.4 g, 1.32 mmol) in dioxane (6.5 mL) was added 4,4,5,5-tetramethyl-2-(prop-1-en-2-yl)-1,3,2-dioxaborolane [126726-62-3] (0.41 mL, 2.15 mmol), potassium phosphate tribasic (4mL, 1M in water, 4 mmol) and Ruphos Pd G3 [1445085-77-7] (97 mg, 0.116 mmol). The reaction mixture was then heated at 100° C. for 3 h. The cooled mixture was extracted with DCM. The organic layer was separated, dried (MgSO₄), filtered and the solvents evaporated in vacuo. The crude product was purified by flash column chromatography (SiO₂, EtOAc in heptane 10/90 to 100/0). The desired fractions were collected and concentrated in vacuo to yield 2-isopropenyl-7-isopropyl-5H-pyrazolo[1,5-d][1,2,4]triazin-4-one (I-7) (268 mg, 92.4%) as a white solid.

Synthesis of 2-(prop-1-en-2-yl)pyrazolo[1,5-d][1,2,4]triazin-4(5H)-one

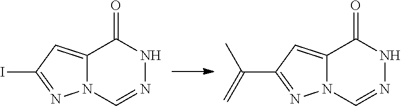

Cesium carbonate [534-17-8] (0.98 g, 3.02 mmol) was added to a stirred solution of [1,1′-bis(diphenylphosphino)ferrocene]dichloropalladium(II) dichloromethane [95464-05-4] (62 mg, 0.075 mmol) in water (1.2 mL) and 1,4-dioxane (9.2 mL) (previously bubbled with nitrogen for 5 min). The mixture was stirred at rt for 5 min, then 2-iodopyrazolo[1,5-d][1,2,4]triazin-4(5H)-one (366 mg, 1.26 mmol) and potassium trifluoro(prop-1-en-2-yl)borate [395083-14-4] (242 mg, 1.63 mmol) were sequentially added. The reaction mixture was stirred at 90° C. for 16h. The mixture was diluted with sat. NaHCO₃ and extracted with AcOEt. The organic layer was separated, dried (MgSO₄), filtered and the solvents evaporated in vacuo. The crude product was purified by flash column chromatography (silica; AcOEt in heptane 0/100 to 10/90). The desired fractions were collected and concentrated in vacuo to yield 2-(prop-1-en-2-yl)pyrazolo[1,5-d][1,2,4]triazin-4(5H)-one (162.1 mg, yield 72%) as a cream solid.

Structure analogs were synthesized using the same procedure.

| Reagent | Starting material | Intermediate |
|---|---|---|
| 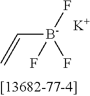 | 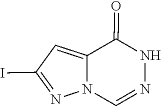 [SMILES: O=c1[nH]ncn2nc(I)cc12] | 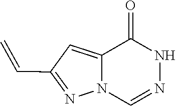 |

Synthesis of 2-isopropylpyrazolo[1,5-d][1,2,4]triazin-4(5H)-one

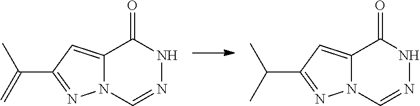

Palladium on carbon 10% [7440-05-3] (40 mg, 0.037 mmol) was added to a stirred solution of 2-(prop-1-en-2-yl)pyrazolo[1,5-d][1,2,4]triazin-4(5H)-one (160 mg, 0.91 mmol) in MeOH (20 mL) at rt under nitrogen atomosphere. Then, nitrogen atmosphere was replaced by hydrogen (balloon) and the reaction mixture was stirred at rt for 8 h.

The mixture was filtered over a pad of celite and was washed with MeOH/DCM mixture, then solvents were removed in vacuo to yield 2-isopropylpyrazolo[1,5-d][1,2,4]triazin-4(5H)-one (153.2 mg, yield 94%) as a yellow solid.

Structure analogs were synthesized using the same procedure.

| Starting material | Intermediate |
|---|---|
| 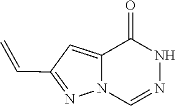 | 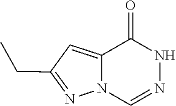 |

Synthesis of 7-bromo-2-isopropylpyrazolo[1,5-d][1,2,4]triazin-4(5H)-one and 3,7-dibromo-2-isopropylpyrazolo[1,5-d][1,2,4]triazin-4(5H)-one

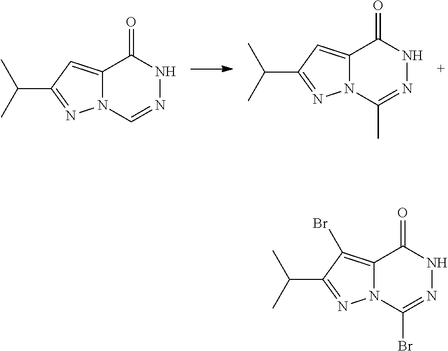

BTMATB [111865-47-5] (495 mg, 1.27 mmol) was added portionwise to a stirred solution of 2-isopropylpyrazolo[1,5-d][1,2,4]triazin-4(5H)-one (226 mg, 1.27 mmol) and K₂CO_{3 [584}-08-7] (193 mg, 1.4 mmol) in DMF dry (7.5 mL) under nitrogen. The mixture was stirred at rt for 3 hr. BTMATB [111865-47-5] (247 mg, 0.63 mmol) was added and the reaction was stirred at rt for another 16 hr. The mixture was diluted with sat NaHCO₃ and extracted with AcOEt. The organic layer was separated, dried (Na₂SO₄), filtered and the solvents evaporated in vacuo. The crude product was purified by flash column chromatography (silica 25g; AcOEt in heptane 0/100 to 15/85). The desired fractions were collected and concentrated in vacuo to yield 7-bromo-2-isopropylpyrazolo[1,5-d][1,2,4]triazin-4(5H)-one (173 mg, yield 49%) and 3,7-dibromo-2-isopropylpyrazolo[1,5-d][1,2,4]triazin-4(5H)-one (83 mg, yield 19%) as a white solid.

Monobromo Compound

1H NMR (300 MHz, CDCl₃) d 9.55 (s, 1H), 7.15 (s, 1H), 3.31 — 3.14 (m, 1H), 1.36 (d, J=7.0 Hz, 6H).

Dibromo-Compound

1H NMR (300 MHz, CDCl3) d 9.62 (s, 1H), 3.25 (hept, J=7.0 Hz, 1H), 1.39 (d, J=7.0 Hz, 6H).

Structure analogs were synthesized using the same procedure.

| Starting material | Intermediate |
|---|---|
| 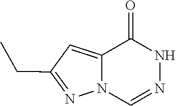 | 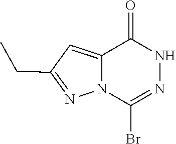 |

Synthesis of 7-bromo-2-cyclopropylpyrazolo[1,5-d][1,2,4]triazin-4(5H)-one

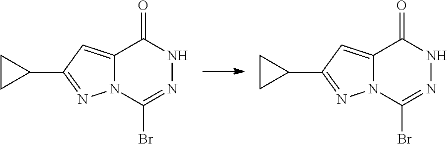 [SMILES: O=c1[nH]nc(Br)n2nc(C3CC3)cc12 ; O=c1[nH]nc(Br)n2nc(C3CC3)cc12]

Benzyltrimethylammonium tribromide [111865-47-5] (7 g, 17.95 mmol) was added portionwise to a stirred solution of 2-cyclopropylpyrazolo[1,5-d][1,2,4]triazin-4(5H)-one (2.1 g, 11.92 mmol) and tBu-TMG (Barton's base) [29166-72-1] (4.8 mL, 0.85 g/mL, 23.82 mmol) in 1,4-dioxane (70 mL) in a 100 mL vessel under N2 at 10° C. (Tj). Once the addition is completed the mixture was warmed to 20° C. (Tj) and stirred at this temperature for 18 h.

Then the mixture was treated with a mixture of a sat. sol. Na₂S2O₃ and sat.sol. NaHCO₃ and then extracted with EtOAc. The organic layer was separated and the aqueous phase was further extracted with EtOAc (x2). The combined organic layers were dired (Na₂SO₄), filtered and the solvents evaporated to yield 7-bromo-2-cyclopropylpyrazolo[1,5-d][1,2,4]triazin-4(5H)-one (3.05 g, yield quant.).

Synthesis of 2-cyclopropyl-3-fluoro-7-isopropylpyrazolo[1,5-d][1,2,4]triazin-4(5H)-one

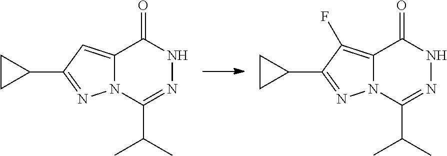 [SMILES: CC(C)c1n[nH]c(=O)c2cc(C3CC3)nn12 ; CC(C)c1n[nH]c(=O)c2c(F)c(C3CC3)nn12]

Selectfluor(R) [140681-55-6] (1 g, 2.82 mmol) was added to a stirred solution of 2-cyclopropyl-7-isopropylpyrazolo[1,5-d][1,2,4]triazin-4(5H)-one (15) (200 mg, 0.92 mmol) in ACN (7 mL). The mixture was stirred at 75° C. for 20h. The mixture was poured into sat. Na₂CO₃ and extracted with DCM. The organic layer was separated, dried (Na2SO4), filtered and the solvent evaporated to yield 2-cyclopropyl-3-fluoro isopropylpyrazolo[1,5-d][1,2,4]triazin-4(5H)-one (408 mg, yield 85%, —45% purity).

Synthesis of 7-(1-ethoxyvinyl)-2-isopropylpyrazolo[1,5-d][1,2,4]triazin-4(5H)-one

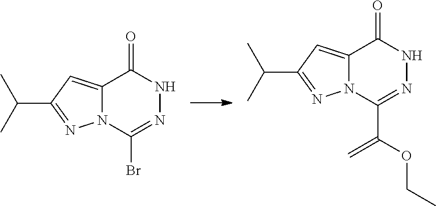 [SMILES: CC(C)c1cc2c(=O)[nH]nc(Br)n2n1 ; C=C(OCC)c1n[nH]c(=O)c2cc(C(C)C)nn12]

Bis(triphenylphosphine)palladium(II) dichloride [13965-03-2] (131 mg, 0.18 mmol) and tributyl(1-ethoxyvinyl)tin [97674-02-7] (762 2.19 mmol) were added to a stirred solution of 7-bromo-2-isopropylpyrazolo[1,5-d][1,2,4]triazin-4(5H)-one in dioxane anhydrous (9 mL) under nitrogen atmosphere in a sealed tube. The mixture was stirred at 100° C. for 16 h. The mixture was diluted with sat. aqueous NaHCO₃ and extracted with AcOEt. The organic layer was separated, dried (MgSO₄), filtered and the solvents evaporated in vacuo. The crude product was purified by flash column chromatography (silica 25g; AcOEt in heptane 0/100 to 15/85). The desired fractions were collected and concentrated in vacuo to yield 7-(1-ethoxyvinyl)-2-isopropylpyrazolo[1,5-d][1,2,4]triazin-4(5H)-one (311 mg, yield 68%) as a yellow solid.

¹H NMR (300 MHz, CDCl₃) d 9.69 (s, 1H), 7.05 (s, 1H), 5.56 (d, J=3.0 Hz, 1H), 4.87 (d, J=3.0 Hz, 1H), 4.04 (q, J=7.0 Hz, 2H), 3.19 (dt, J=13.8, 6.9 Hz, 1H), 1.44 (t, J=7.0 Hz, 3H), 1.35 (d, J=6.9 Hz, 6H).

Synthesis of 7-acetyl-2-isopropylpyrazolo[1,5-d][1,2,4]triazin-4(5H)-one

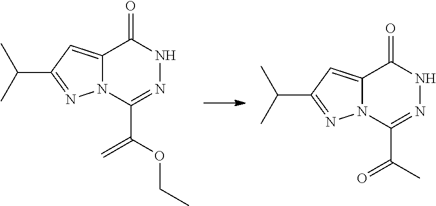 [SMILES: C=C(OCC)c1n[nH]c(=O)c2cc(C(C)C)nn12 ; CC(=O)c1n[nH]c(=O)c2cc(C(C)C)nn12]

HCl (6M in water) [7647-01-0] (1 mL, 6 M, 6.26 mmol) was added dropwise at 10° C. to a solution of 7-(1-ethoxyvinyl)-2-isopropylpyrazolo[1,5-d][1,2,4]triazin-4(5H)-one (311 mg, 1.25 mmol) in dioxane (12 mL) and the reaction mixture was stirred at rt for 1 h. The mixture was extracted with AcOEt. The organic layer was separated, dried (MgSO₄), filtered and the solvents evaporated in vacuo to yield 7-acetyl-2-isopropylpyrazolo[1,5-d][1,2,4]triazin-4(5H)-one (226 mg, yield 81%) as a white solid.

1H NMR (300 MHz, CDCl₃) d 9.79 (s, 1H), 7.09 (s, 1H), 3.28 (dt, J=13.9, 6.9 Hz, 1H), 2.71 (s, 3H), 1.35 (d, J=7.0 Hz, 6H).

Synthesis of 7-(1-hydroxyethyl)-2-isopropylpyrazolo[1,5-d][1,2,4]triazin-4(5H)-one

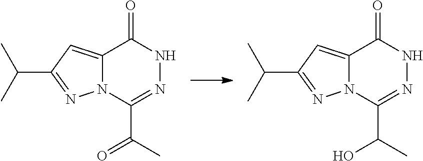 [SMILES: CC(=O)c1n[nH]c(=O)c2cc(C(C)C)nn12 ; CC(C)c1cc2c(=O)[nH]nc(C(C)O)n2n1]

Sodium borohydride [16940-66-2] (39 mg, 1.03 mmol) was added portionwise to a stirrerd solution of 7-acetyl-2-isopropylpyrazolo[1,5-d][1,2,4]triazin-4(5H)-one in THF (14 mL) and water (3 mL) at 0° C. The mixture was stirred at 0° C. for 16h. The mixture was diluted with saturated aq NaHCO₃ and extracted with AcOEt (x3). The organic layer was separated, dried (MgSO₄), filtered and the solvents evaporated in vacuo. The crude product was purified by flash column chromatography (silica 25 g; dry load in silica; EtOAc in heptane 0/100 to 100/0). The desired fractions were collected and concentrated in vacuo to yield 7-(1-hydroxyethyl)-2-isopropylpyrazolo[1,5-d][1,2,4]triazin-4(5H)-one (206 mg, yield 89%) as a yellow oil.

1H NMR (300 MHz, CDCl₃) d 9.55 (s, 1H), 7.01 (s, 1H), 5.27-5.19 (m, 1H), 3.18 (dt, J=13.8, 7.0 Hz, 1H), 1.68 (d, J=6.6 Hz, 3H), 1.35 (d, J=6.9 Hz, 6H).

Synthesis of methyl 2-(2-ethyl-7-isopropyl-4-oxo-pyrazolo[1,5-d][1,2,4]triazin yl)acetate (I-8)

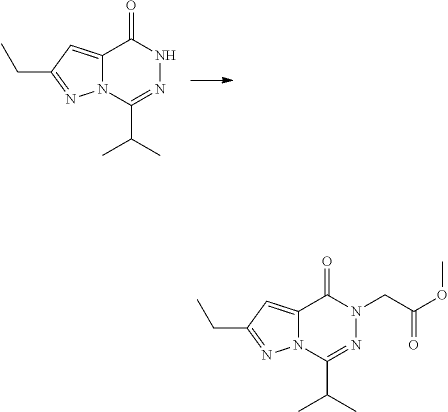 [SMILES: CCc1cc2c(=O)[nH]nc(C(C)C)n2n1 ; CCc1cc2c(=O)n(CC(=O)OC)nc(C(C)C)n2n1]

18—Crown-6 [17455-13-9] (12 mg, 0.045 mmol), followed by potassium iodide [7681-11-0] (8 mg, 0.048 mmol), K₂CO₃ [584-08-7] (111 mg, 0.80 mmol) and methyl chloroacetate [96-34-4] (50 μL, 1.42 g/mL, 0.65 mmol) were added to a stirred suspension of 2-ethyl-7-isopropyl-5H-pyrazolo[1, 5-d] [1,2,4]triazin-4-one (1-6) (108 mg, 0.52 mmol) in ACN [75-05-8] (3 mL) in a sealed tube and under N2. The mixture was stirred at 80° C. for 16 h. The mixture was treated with water and extracted with DCM. The organic layer was separated, dried (MgSO₄), filtered and the solvents evaporated in vacuo. The crude product was purified by flash column chromatography (SiO₂, EtOAc in heptane 10/90 to 100/0). The desired fractions were collected and concentrated in vacuo to yield methyl 2-(2-ethyl-7-isopropyl-4-oxo-pyrazolo[1,5-d][1,2,4]triazin-5-yl)acetate (I-8) (125 mg, 86%) as a colorless oil.

1H NMR (400 MHz, DMSO-d₆) δ ppm 1.22-1.36 (m, 9 H) 2.80 (q, J=7.63 Hz, 2 H) 3.53-3.63 (m, 1 H) 3.65-3.77 (m, 3 H) 4.81 (s, 2 H) 7.13 (s, 1 H)

Structure analogs were synthesized using the same procedure.

| Reagent | Starting material | Intermediate |
|---|---|---|
| 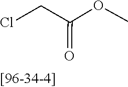 | 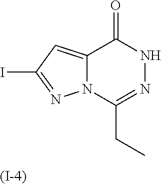 | 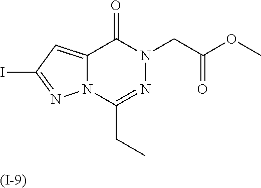 [SMILES: CCc1nn(CC(=O)OC)c(=O)c2cc(I)nn12 ; "(I-9)"] |
| 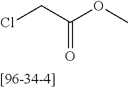 | 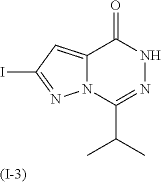 | 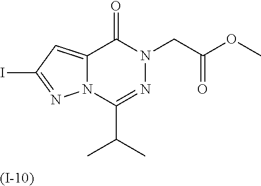 [SMILES: COC(=O)Cn1nc(C(C)C)n2nc(I)cc2c1=O ; "(I-10)"] |
| 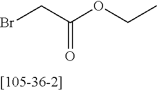 | 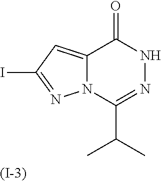 | 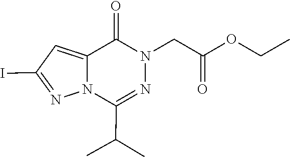 [SMILES: CCOC(=O)Cn1nc(C(C)C)n2nc(I)cc2c1=O] |
| 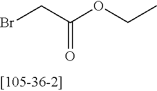 | 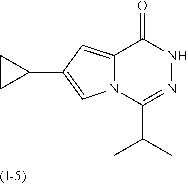 [SMILES: CC(C)c1n[nH]c(=O)c2cc(C3CC3)cn12 ; "(I-5)"] | 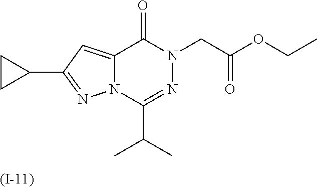 [SMILES: CCOC(=O)Cn1nc(C(C)C)n2nc(C3CC3)cc2c1=O ; "(I-11)"] |
| 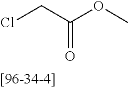 | 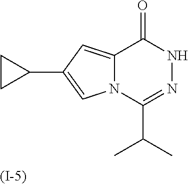 [SMILES: CC(C)c1n[nH]c(=O)c2cc(C3CC3)cn12 ; "(I-5)"] | 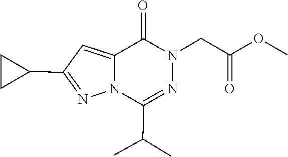 [SMILES: COC(=O)Cn1nc(C(C)C)n2nc(C3CC3)cc2c1=O] |
| 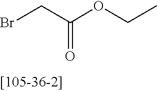 | 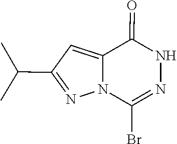 | 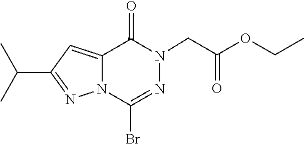 [SMILES: CCOC(=O)Cn1nc(Br)n2nc(C(C)C)cc2c1=O] |
| 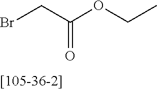 | 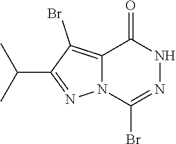 | 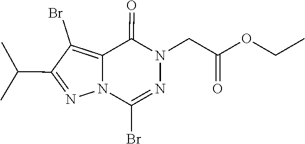 [SMILES: CCOC(=O)Cn1nc(Br)n2nc(C(C)C)c(Br)c2c1=O] |
| 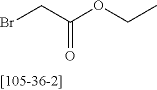 | 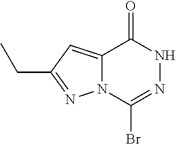 | 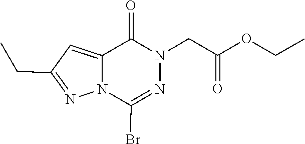 [SMILES: CCOC(=O)Cn1nc(Br)n2nc(CC)cc2c1=O] |
| 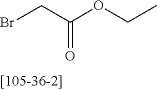 | 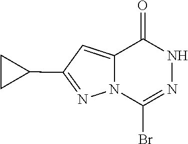 [SMILES: O=c1[nH]nc(Br)n2nc(C3CC3)cc12] | 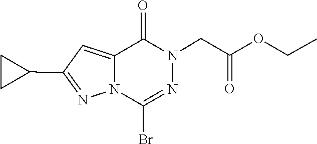 [SMILES: CCOC(=O)Cn1nc(Br)n2nc(C3CC3)cc2c1=O] |
| 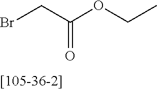 | 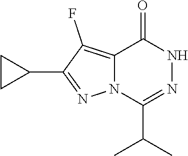 [SMILES: CC(C)c1n[nH]c(=O)c2c(F)c(C3CC3)nn12] | 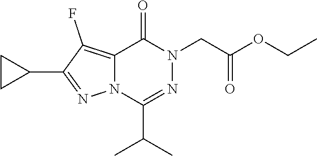 [SMILES: CCOC(=O)Cn1nc(C(C)C)n2nc(C3CC3)c(F)c2c1=O] |
| 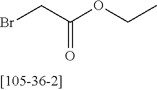 | 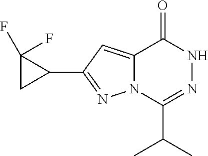 [SMILES: CC(C)c1n[nH]c(=O)c2cc(C3CC3(F)F)nn12] | 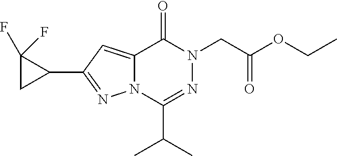 [SMILES: CCOC(=O)Cn1nc(C(C)C)n2nc(C3CC3(F)F)cc2c1=O] |
| 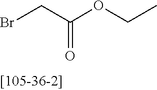 | 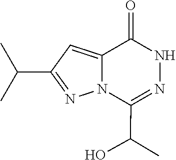 [SMILES: CC(C)c1cc2c(=O)[nH]nc(C(C)O)n2n1] | 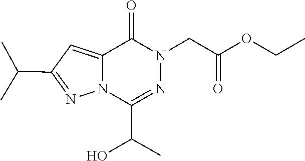 [SMILES: CCOC(=O)Cn1nc(C(C)O)n2nc(C(C)C)cc2c1=O] |
| 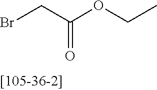 | 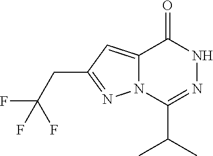 [SMILES: CC(C)c1n[nH]c(=O)c2cc(CC(F)(F)F)nn12] | 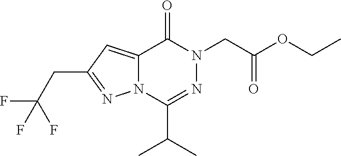 [SMILES: CCOC(=O)Cn1nc(C(C)C)n2nc(CC(F)(F)F)cc2c1=O] |
| 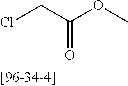 | 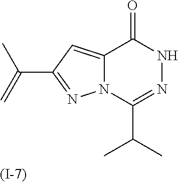 [SMILES: C=C(C)c1cc2c(=O)[nH]nc(C(C)C)n2n1 ; "(I-7)"] | 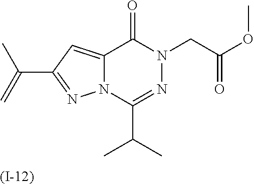 [SMILES: C=C(C)c1cc2c(=O)n(CC(=O)OC)nc(C(C)C)n2n1 ; "(I-12)"] |
| 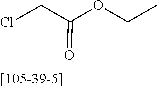 | 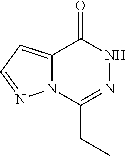 | 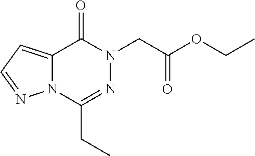 [SMILES: CCOC(=O)Cn1nc(CC)n2nccc2c1=O] |
| 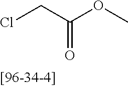 | 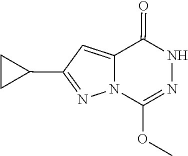 | 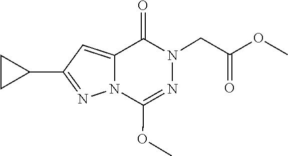 [SMILES: COC(=O)Cn1nc(OC)n2nc(C3CC3)cc2c1=O] |
| 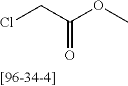 | 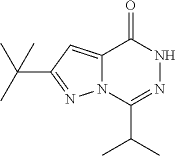 [SMILES: CC(C)c1n[nH]c(=O)c2cc(C(C)(C)C)nn12] | 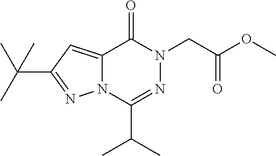 [SMILES: COC(=O)Cn1nc(C(C)C)n2nc(C(C)(C)C)cc2c1=O] |
| 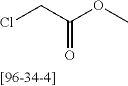 | 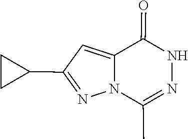 | 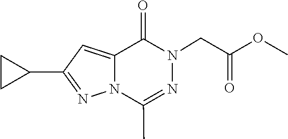 [SMILES: COC(=O)Cn1nc(C)n2nc(C3CC3)cc2c1=O] |

Synthesis of methyl 2-(2,7-diisopropyl-4-oxo-pyrazolo[1,5-d][1,2,4]triazin-5-yl)acetate (I-13)

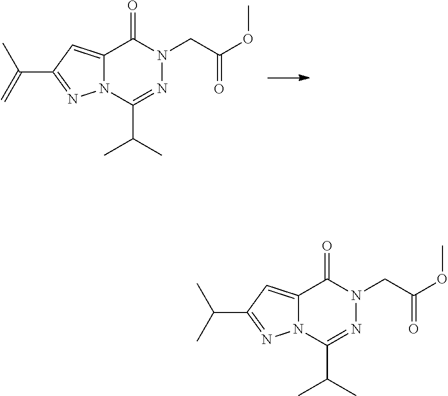 [SMILES: C=C(C)c1cc2c(=O)n(CC(=O)OC)nc(C(C)C)n2n1 ; COC(=O)Cn1nc(C(C)C)n2nc(C(C)C)cc2c1=O]

A solution of 2-isopropenyl-7-isopropyl-5H-pyrazolo[1,5-d][1,2,4]triazin-4-one (I-16) (259 mg, 0.89 mmol) in EtOH (20 mL) was hydrogenated using an H-Cube reactor (1mL/min, 30 mins, Pd/C 10% cartridge, full H₂ mode, 25° C., 1 cycle). The solvent was evaporated to afford methyl 2-(2,7-diisopropyl-4-oxo-pyrazolo[1,5-d][1,2,4]triazin yl)acetate (I-13) (209 mg, 80%) as a colorless oil.

Sythesis of ethyl 2-(2,7-diisopropyl-4-oxopyrazolo[1,5-d][1,2,4]triazin-5(4H)-yl)acetate and ethyl 2-(7-isopropyl-4-oxo-2-propylpyrazolo[1,5-d][1,2,4]triazin-5(4H)-yl)acetate

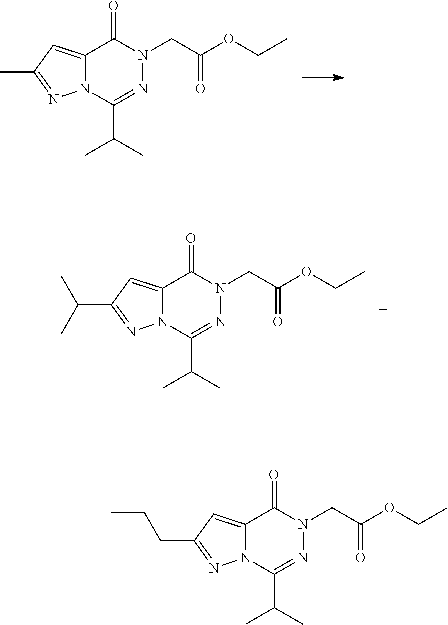

2-Propylzinc bromide solution (0.5 M in THF) [77047-87-1] (7.7 mL, 0.5 M, 3.84 mmol) was added dropwise to a stirred solution of ethyl 2-(2-iodo-7-isopropyl-4-oxopyrazolo[1,5-d][1,2,4]triazin-5(4H)-yl)acetate (500 mg, 1.28 mmol) and bis(tri-tert-butylphosphine)palladium(O) [53199-31-8] (65 mg, 0.13 mmol) in THF anhydrous (15 mL) (previously degassed bubbling nitrogen for 5 min) at rt. The resulting mixture was stirred at 40° C. for 16 h. The mixture was diluted with sat. aqueous NaHCO₃ and extracted with AcOEt. The organic layer was separated, dried (MgSO₄), filtered and the solvents evaporated in vacuo. The crude product was purified by flash column chromatography (silica 25g; AcOEt in heptane 0/100 to 20/80). The desired fractions were collected and concentrated in vacuo to yield ethyl 2-(2,7-diisopropyl-4-oxopyrazolo[1,5-d][1,2,4]triazin-5(4H)-yl)acetate and ethyl 2-(7-isopropyl-4-oxo-2-propylpyrazolo[1,5-d][1,2,4]triazin-5(4H)-yl)acetate as a 1:1 mixture as a yellow sticky solid.

Di-Isopropyl Compound

1H NMR (300 MHz, DMSO) d 7.17 (s, 1H), 4.78 (s, 2H), 4.16 (q, J=7.1 Hz, 2H), 3.59 (hept, J=6.9 Hz, 1H), 3.13 (hept, J=6.9 Hz, 1H), 1.31 (d, J=6.9 Hz, 6H), 1.30 (d, J=6.9 Hz, 6H), 1.20 (t, J=7.1 Hz, 3H).

### Mono-Isopropyl Compound

1H NMR (300 MHz, DMSO) d 7.12 (s, 1H), 4.78 (s, 2H), 4.16 (q, J=7.1 Hz, 2H), 3.59 (hept, J=6.9 Hz, 1H), 2.75 (t, J=7.4 Hz, 2H), 1.71 (h, J=7.4 Hz, 2H), 1.30 (d, J=6.9 Hz, 6H) 1.20 (t, J=7.1 Hz, 3H), 0.94 (t, J=7.4 Hz, 3H).

Structure analogs were synthesized using the same procedure.

| Reagent | Starting material | Intermediate |
|---|---|---|
| 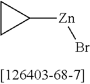 | 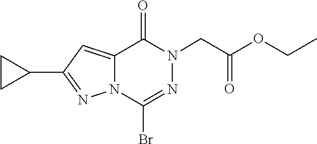 [SMILES: CCOC(=O)Cn1nc(Br)n2nc(C3CC3)cc2c1=O] | 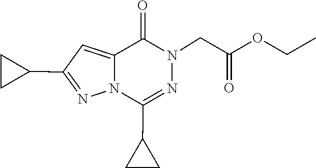 [SMILES: CCOC(=O)Cn1nc(C2CC2)n2nc(C3CC3)cc2c1=O] |
| 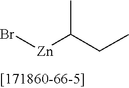 | 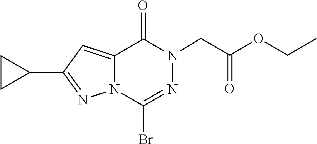 [SMILES: CCOC(=O)Cn1nc(Br)n2nc(C3CC3)cc2c1=O] | 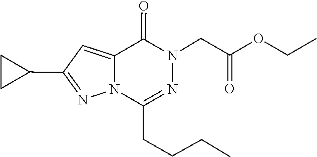 [SMILES: CCCCc1nn(CC(=O)OCC)c(=O)c2cc(C3CC3)nn12] |
| | | 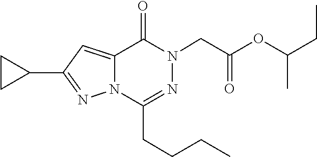 [SMILES: CCCCc1nn(CC(=O)OC(C)CC)c(=O)c2cc(C3CC3)nn12] |
| 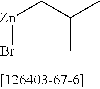 | 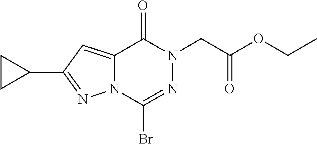 [SMILES: CCOC(=O)Cn1nc(Br)n2nc(C3CC3)cc2c1=O] | 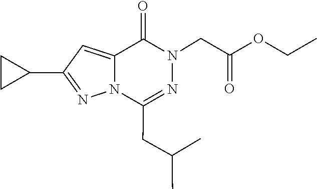 [SMILES: CCOC(=O)Cn1nc(CC(C)C)n2nc(C3CC3)cc2c1=O] |
| | | 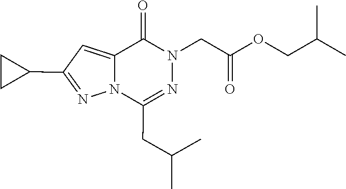 [SMILES: CC(C)COC(=O)Cn1nc(CC(C)C)n2nc(C3CC3)cc2c1=O] |

Sythesis of ethyl 2-(2-(ethylamino)-7-isopropyl-4-oxopyrazolo[1,5-d][1,2,4]triazin-5(4H)-yl)acetate

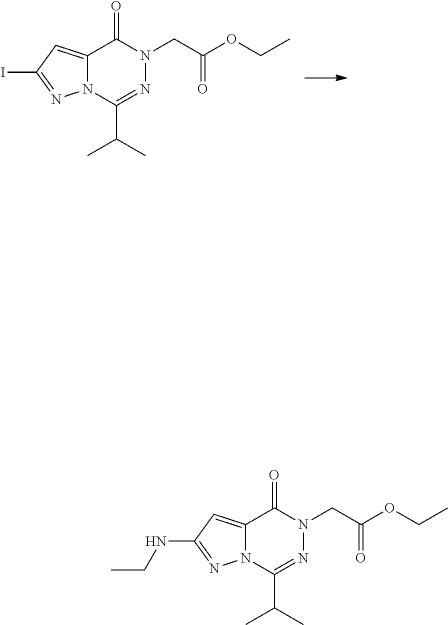

A vial containing ethyl 2-(2-iodo-7-isopropyl-4-oxopyrazolo[1,5-d][1,2,4]triazin-5(4H)-yl)acetate (850 mg, 2.18 mmol), DL-proline [609-36-9] (50 mg, 0.43 mmol), K₂CO_{3 [584}-08-7] (1.2 g, 8.71 mmol) in DMSO (15 mL) was degassed. Then, ethylamine hydrochloride [557-66-4] (532 mg, 6.54 mmol) and CuI [7681-65-4] (85 mg, 0.45 mmol) were added. The reaction mixture was stirred at 90° C. for 48 hours. H₂O and EtOAc were added to the reaction mixture and the layers were separated. The aqueous phase was extracted with EtOAc (x2). The combined organic layers were dried over MgSO₄ and the concentrated in vacuo. The residuewas purified by flash column chromatography (SiO₂, EtOAc in heptane 0/100 to 75/25). The desired fractions were collected and concentrated in vacuo to yield 2-(2-(ethylamino)-7-isopropyl-4-oxopyrazolo[1,5-d][1,2,4]triazin-5(4H)-yl)acetate (550 mg, yield 82%) as a pale solid.

1H NMR (500 MHz, DMSO-d₆) d ppm 1.16 (t, J=7.2 Hz, 3 H) 1.20 (t, J=7.1 Hz, 3 H) 1.28 (d, J=6.9 Hz, 6 H) 3.20 (qd, J=7.1, 5.8 Hz, 2 H) 3.46 (spt, J=6.9 Hz, 1 H) 4.15 (q, J=7.2 Hz, 2 H) 4.72 (s, 2 H) 6.36 (s, 1 H) 6.42 (t, J=5.6 Hz, 1 H)

Structure analogs were synthesized using the same procedure.

| Reagent | Starting material | Intermediate |
|---|---|---|
| 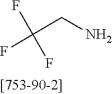 | 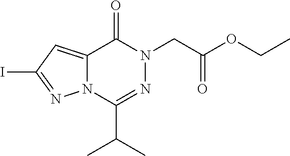 [SMILES: CCOC(=O)Cn1nc(C(C)C)n2nc(I)cc2c1=O] | 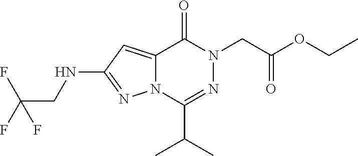 [SMILES: CCOC(=O)Cn1nc(C(C)C)n2nc(NCC(F)(F)F)cc2c1=O] |

Synthesys of ethyl 2-(7-(dimethylamino)-2-isopropyl-4-oxopyrazolo[1,5-d][1,2,4]triazin-5(4H)-yl)acetate

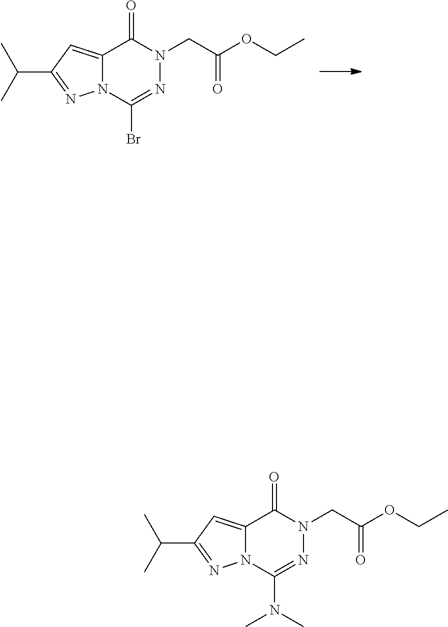

Dimethylamine (2 M in THF) [124-40-3] (0.31 mL, 2 M, 0.62 mmol) was added to a stirred solution of ethyl 2-(7-bromo-2-isopropyl-4-oxopyrazolo[1,5-d][1,2,4]triazin-5(4H)-yl)acetate (107 mg, 0.31 mmol) and N,N-diisopropylethylamine [7087-68-5] (165 0.74 g/mL, 0.94 mmol) in 1,4-dioxane (1.5 mL) in a sealed tube. The mixture was stirred at 80° C. for 16 h. The mixture was diluted with sat. aqueous NaHCO₃ and extracted with AcOEt. The organic layer was separated, dried (MgSO₄), filtered and the solvents evaporated in vacuo. The crude product was purified by flash column chromatography (silica 25g; AcOEt in heptane 0/100 to 50/50). The desired fractions were collected and concentrated in vacuo to yield ethyl 2-(7-(dimethylamino) isopropyl-4-oxopyrazolo[1,5-d][1,2,4]triazin-5(4H)-yl)acetate (93 mg, yield 96%) as a colourles oil.

1H NMR (300 MHz, CDCl₃) d 6.96 (s, 1H), 4.73 (s, 2H), 4.24 (q, J=7.1 Hz, 2H), 3.16 (dt, J=13.2, 6.6 Hz, 1H), 3.08 (s, 6H), 1.35 (s, 3H), 1.32 (d, J=5.5 Hz, 3H), 1.30-1.22 (m, 3H).

Structure analogs were synthesized using the same procedure.

| Reagent | Starting material | Intermediate |
|---|---|---|
| [124-40-3] | 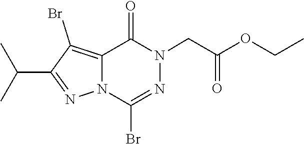 [SMILES: CCOC(=O)Cn1nc(Br)n2nc(C(C)C)c(Br)c2c1=O] | 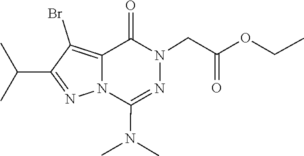 [SMILES: CCOC(=O)Cn1nc(N(C)C)n2nc(C(C)C)c(Br)c2c1=O] |
| [124-40-3] | 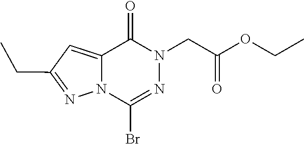 [SMILES: CCOC(=O)Cn1nc(Br)n2nc(CC)cc2c1=O] | 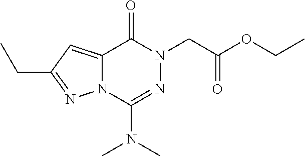 [SMILES: CCOC(=O)Cn1nc(N(C)C)n2nc(CC)cc2c1=O] |
| [124-40-3] | 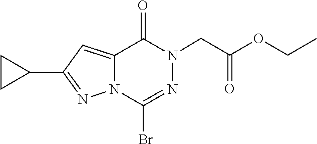 [SMILES: CCOC(=O)Cn1nc(Br)n2nc(C3CC3)cc2c1=O] | 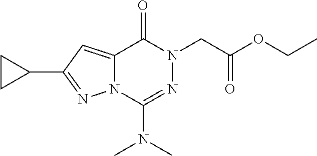 [SMILES: CCOC(=O)Cn1nc(N(C)C)n2nc(C3CC3)cc2c1=O] |
| [38256-93-8] | 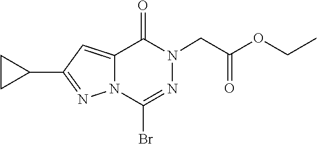 [SMILES: CCOC(=O)Cn1nc(Br)n2nc(C3CC3)cc2c1=O] | 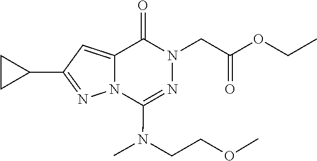 [SMILES: CCOC(=O)Cn1nc(N(C)CCOC)n2nc(C3CC3)cc2c1=O] |
| [109-85-3] | 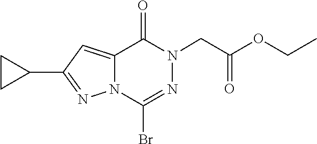 [SMILES: CCOC(=O)Cn1nc(Br)n2nc(C3CC3)cc2c1=O] | 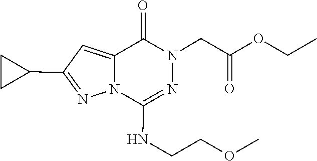 [SMILES: CCOC(=O)Cn1nc(NCCOC)n2nc(C3CC3)cc2c1=O] |

Synthesis of ethyl 2-(2-cyclopropyl-7-isopropyl-3-nitro-4-oxopyrazolo[1,5-d][1,2,4]triazin-5(4H)-yl)acetate

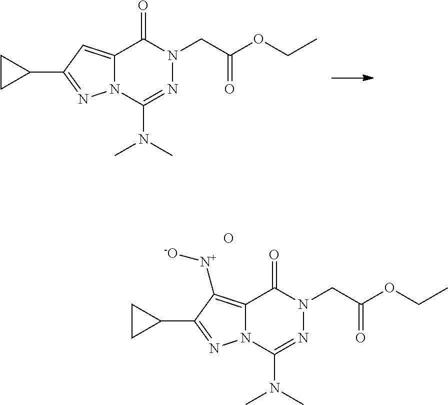

Nitric acid [7697-37-2] (0.26 mL, 3.42 mmol) was added to a stirred solution of ethyl 2-(2-cyclopropyl-7-isopropyl-4-oxopyrazolo[1,5-d][1,2,4]triazin-5(4H)-yl)acetate (I 11) (130 mg, 0.43 mmol) in acetic anhydride [108-24-7] (1.3 mL). The mixture was stirred at RT for 24 h. Then, the mixture was diluted with Na₂CO₃ (20% in water) and NaOH (1N in water) to reach pH 5-6 and extracted with EtOAc. The organic layer was separated, dried (Na₂SO₄), filtered and the solvent evaporated to yield 2-(2-cyclopropyl-7-isopropyl-3-nitro-4-oxopyrazolo[1,5-d][1,2,4]triazin-5(4H)-yl)acetate (180 mg, yield quantitative) as a yellow oil, that crystallized upon standing to a yellow solid.

1H NMR (500 MHz, DMSO-d₆) d ppm 1.03-1.08 (m, 2 H) 1.15-1.19 (m, 2 H) 1.20-1.24 (m, 3 H) 1.27 (d, J=6.87 Hz, 6 H) 2.41-2.47 (m, 1 H) 2.50 (s, 60 H) 3.49-3.59 (m, 1 H) 4.15-4.21 (m, 2 H) 4.83 (s, 2 H).

Synthesis of ethyl 2-(7-isopropyl-4-oxo-2-vinylpyrazolo[1,5-d][1,2,4]triazin-5(4H)-yl)acetate

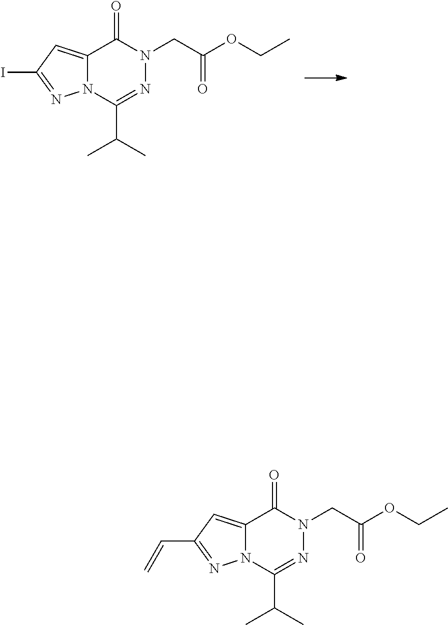

A soluiton of cesium carbonate [534-17-8] (2447 mg, 7.51 mmol) in water (degassed) (4 mL) was added to a stirred solution of ethyl 2-(2-iodo-7-isopropyl-4-oxopyrazolo[1,5-d][1,2,4]triazin-5(4H)-yl)acetate (1221 mg, 3.13 mmol) and potassium vinyltrifluoroborate [13682-77-4] (545 mg, 4.07 mmol) in 1,4-dioxane (degassed) (16 mL) at rt while nitrogen was bubbling. [1,1′-Bis(diphenylphosphino)ferrocene]dichloropalladium(II) dichloromethane [95464-05-4] (154 mg, 0.19 mmol) was added and the mixture was stirred in a sealed tube at 90° C. for 16 h. The mixture was allowed to cool to rt. The mixture was diluted with water and extracted with EtOAc. The organic layer was separated, dried (MgSO₄), filtered and the solvents evaporated in vacuo. The crude was purified by flash column chromatography (silica 80 g; EtOAc in heptane 0/100 to 20/80). The desired fractions were collected and concentrated in vacuo to yield 2-(7-isopropyl-4-oxo vinylpyrazolo[1,5-d][1,2,4]triazin-5(4H)-yl)acetate (682 mg, yield 74%) as a whitish solid.

1H NMR (300 MHz, CDCl₃) d 7.21 (s, 1H), 6.84 (dd, J=17.8, 11.1 Hz, 1H), 6.01 (dd, J=17.8, 0.8 Hz, 1H), 5.57 (dd, J=11.1, 0.8 Hz, 1H), 4.80 (s, 2H), 4.25 (q, J=7.1 Hz, 2H), 3.67 (hept, J=6.9 Hz, 1H), 1.38 (d, J=6.9 Hz, 6H), 1.29 (t, J=7.1 Hz, 3H).

Synthesis of ethyl 2-(2-formyl-7-isopropyl-4-oxopyrazolo[1,5-d][1,2,4]triazin-5(4H)-yl)acetate

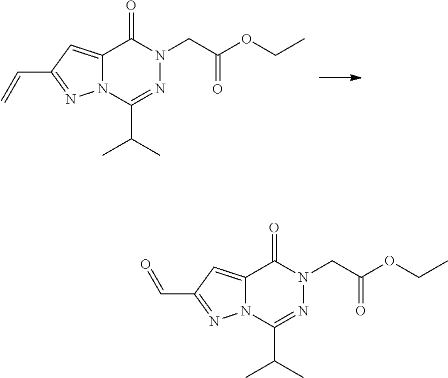 [SMILES: C=Cc1cc2c(=O)n(CC(=O)OCC)nc(C(C)C)n2n1 ; CCOC(=O)Cn1nc(C(C)C)n2nc(C=O)cc2c1=O]

Osmium tetroxide 2.5% in tert-butanol [20816-12-0] (950 1 g/mL, 0.093 mmol) was added to a stirred solution of ethyl 2-(7-isopropyl-4-oxo-2-vinylpyrazolo[1,5-d][1,2,4]triazin-5(4H)-yl)acetate (678 mg, 2.34 mmol) and sodium periodate [7790-28-5] (999 mg, 4.67 mmol) in THF:Water 1:1 (12 mL) and the mixture was stirred at rt for 16 hours. The mixture was diluted with water and sat. aq. NaHCO₃ solution and extracted with AcOEt. The organic layer was dried (MgSO₄), filtered and the solvents evaporated in vacuo. The crude product was purified by flash column chromatography (silica, 12 g; AcOEt in Heptane 0/100 to 50/50). The desired fractions were collected and concentrated in vacuo to yield ethyl 2-(2-formyl-7-isopropyl-4-oxopyrazolo[1,5-d][1,2,4]triazin-5(4H)-yl)acetate (489 mg, yield 69%) as a colorless oil.

1H NMR (300 MHz, CDCl₃) d 10.19 (s, 1H), 7.57 (s, 1H), 4.82 (s, 2H), 4.26 (q, J =7.1 Hz, 2H), 3.73 (dt, J=13.7, 6.9 Hz, 1H), 1.42 (d, J=6.9 Hz, 6H), 1.30 (t, J=7.1 Hz, 3H).

Synthesis of ethyl 2-(7-isopropyl-4-oxo-2-(2,2,2-trifluoro-1-hydroxyethyl)pyrazolo[1,5-d][1,2,4]triazin-5(4H)-yl)acetate

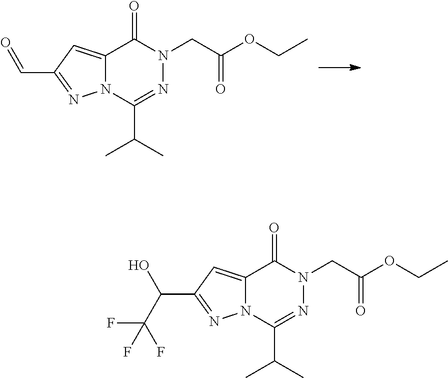 [SMILES: CCOC(=O)Cn1nc(C(C)C)n2nc(C=O)cc2c1=O ; CCOC(=O)Cn1nc(C(C)C)n2nc(C(O)C(F)(F)F)cc2c1=O]

TBAF (1M in THF) [429-41-4] (304 0.3 mmol) was added dropwise to a stirred solution of ethyl 2-(2-formyl-7-isopropyl-4-oxopyrazolo[1,5-d][1,2,4]triazin-5(4H)-yl)acetate (445 mg, 1.52 mmol) and 2-trifluoromethyltrimethylsilane [81290-20-2] (460 μL , 3.04 mmol) in THF anhydrous (12 mL) at 0° C. under nitrogen atmosphere. The reaction was stirred at 0° C. for 15 min, then at rt for 12 h. The mixture was cooled down to 0° C. and TBAF (1M in THF) [429-41-4] (3 mL, 3.04 mmol) was added and the mixture was stirred at rt for 20 min. The mixture was diluted with sat. aqueous NaHCO₃, then was extracted with AcOEt. The organic layer was separated, dried (MgSO₄), filtered and the solvents evaporated in vacuo. The crude product was purified by flash column chromatography (silica 25g; AcOEt in heptane 0/100 to 30/70). The desired fractions were collected and concentrated in vacuo to yield -(7-isopropyl-4-oxo-2-(2,2,2-trifluoro-1-hydroxyethyl)pyrazolo[1,5-d] [1,2,4]triazin-5 (4H)-yl)acetate (420 mg, yield 74%) as a yellow solid.

1H NMR (300 MHz, CDCl₃) d 7.21 (s, 1H), 5.36-5.23 (m, 1H), 4.85 (d, J=17.0 Hz, 1H), 4.79 (d, J=17.0 Hz, 1H), 4.26 (q, J=7.1 Hz, 2H), 3.66 (hept, J=6.9 Hz, 1H), 3.44-3.34 (m, 1H), 1.39 (d, J=6.9 Hz, 6H), 1.30 (t, J=7.1 Hz, 3H).

Synthesis of ethyl 2-(2-(1-ethoxyvinyl)-7-isopropyl-4-oxopyrazolo[1,5-d][1,2,4]triazin-5(4H)-yl)acetate

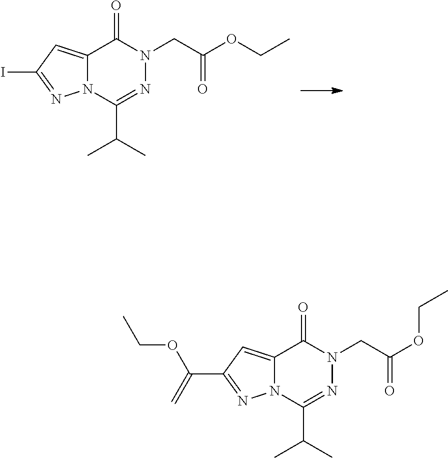

To a mixture of ethyl 2-(2-iodo-7-isopropyl-4-oxopyrazolo[1,5-d][1,2,4]triazin-5(4H)-yl)acetate (0.52 g, 1.3327 mmol) in dioxane (10 ml), bis(triphenylphosphine)palladium(II) dichloride [13965-03-2] (95 mg, 0.133 mmol) and tributyl(1-ethoxyvinyl)tin [97674-02-7] (0.51 mL, 1.069 g/mL, 1.466 mmol) were added and the mixture was heated at 100° C. for 6 h. The crude was evaporated in vacuo and purified by flash column chromatography (silica; MeOH in DCM 0/100 to 5/95). The desired fractions were collected and the solvent evaporated in vacuo to afford 2-(2-(1-ethoxyvinyl)-7-isopropyl-4-oxopyrazolo[1,5-d][1,2,4]triazin-5(4H)-yl)acetate (400 mg, yield 90%) as an oil.

Synthesis of ethyl 2-(2-acetyl-7-isopropyl-4-oxopyrazolo[1,5-d][1,2,4]triazin-5(4H)-yl)acetate

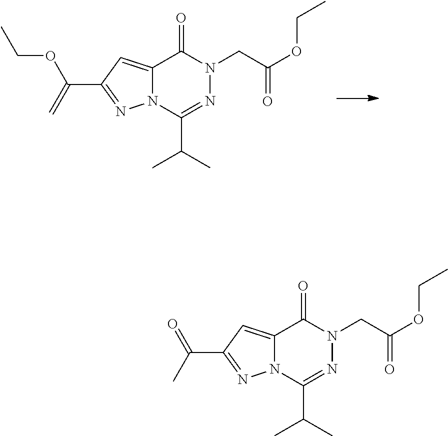

To a solution of ethyl 2-(2-(1-ethoxyvinyl)-7-isopropyl-4-oxopyrazolo[1,5-d][1,2,4]triazin-5(4H)-yl)acetate (0.4 g, 1.196 mmol) in THF (5 ml), HCl (2M in H₂O) [7647-01-0] (2 mL, 2 M, 4 mmol) was added. The mixture was stirred for 16 h at rt. The crude was extracted with AcOEt (2×10 ml). The organic layer was evaporated in vacuo to afford an oil wich was purified by flash column chromatography (silica; MeOH in DCM 0/100 to 5/95). The desired fractions were collected and the solvent evaporated in vacuo to afford ethyl 2-(2-acetyl-7-isopropyl-4-oxopyrazolo[1,5-d][1,2,4]triazin-5(4H)-yl)acetate (300 mg, yield 82%) as an oil.

1H NMR (400 MHz, CHLOROFORM-d) d ppm 1.31 (t, J=7.1 Hz, 4 H) 1.43 (d, J=6.9 Hz, 7 H) 2.71 (s, 3 H) 3.74 (quin, J=6.9 Hz, 1 H) 4.20-4.34 (m, 2 H) 4.82 (s, 2 H) 7.53-7.60 (m, 1 H).

Synthesis of ethyl 2-(2-(1,1-difluoroethyl)-7-isopropyl-4-oxopyrazolo[1,5-d][1,2,4]triazin-5(4H)-yl)acetate

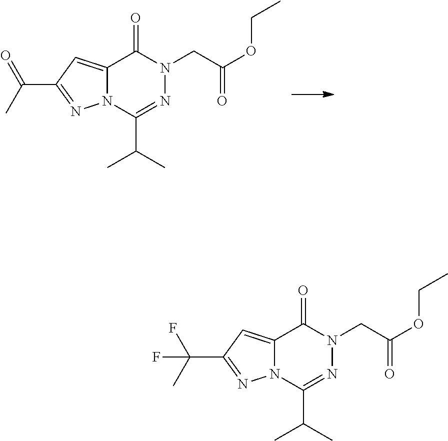 [SMILES: CCOC(=O)Cn1nc(C(C)C)n2nc(C(C)=O)cc2c1=O ; CCOC(=O)Cn1nc(C(C)C)n2nc(C(C)(F)F)cc2c1=O]

To a mixture of ethyl 2-(2-acetyl-7-isopropyl-4-oxopyrazolo[1,5-d][1,2,4]triazin-5(4H)-yl)acetate (0.3 g, 0.981 mmol) in dry DCM (8 ml), diethylaminosulfur trifluoride [38078-09-0] (0.65 mL, 1.22 g/mL, 4.89 mmol) and triethylamine trihydrofluoride [73602-61-6] (0.48 mL, 0.989 g/mL, 2.93 mmol) were added at rt. The mixture was stirred for 48 hours at 50° C. The crude was cooled with ice bath and quenched with a saturated solution of NaHCO₃ (dropwise), the organic layer was separated, dried

(Na₂SO₄) and evaporated in vacuo; the crude product was purified by flash column chromatography (silica: MeOH in DCM 0/100 to 2/98). The desired fractions were collected and concentrated in vacuo to yield 2-(2-(1,1-difluoroethyl)-7-isopropyl-4-oxopyrazolo[1,5-d][1,2,4]triazin-5(4H)-yl)acetate (190 mg, yield 59%) as a solid.

1H NMR (400 MHz, CHLOROFORM-d) d ppm 1.31 (s, 4 H) 1.40 (d, J=6.9 Hz, 7 H) 2.10 (t, J=18.5 Hz, 3 H) 3.69 (dt, J=13.7, 6.9 Hz, 1 H) 4.26 (q, J=7.2 Hz, 2 H) 4.82 (s, 2 H) 7.30 (s, 1 H).

Synthesis of 2-(2-ethyl-7-isopropyl-4-oxo-pyrazolo[1,5-d][1,2,4]triazin-5-yl)acetic acid (I-14)

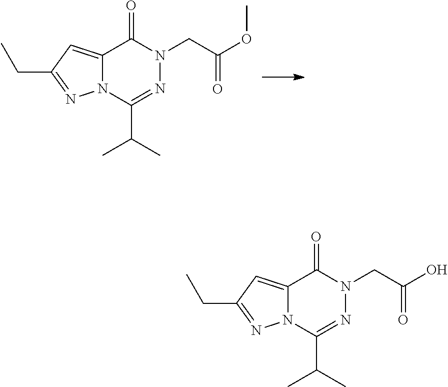 [SMILES: CCc1cc2c(=O)n(CC(=O)OC)nc(C(C)C)n2n1 ; CCc1cc2c(=O)n(CC(=O)O)nc(C(C)C)n2n1]

1M aqueous solution of lithium hydroxide [1310-65-2] (0.9 mL, 1 M, 0.9 mmol) was added to a stirred solution of methyl 2-(2-ethyl-7-isopropyl-4-oxo-pyrazolo[1,5-d][1,2,4]triazin-5-yl)acetate (I-7) (125 mg, 0.45 mmol) in THF (1.4 mL) under N2. The mixture was stirred at rt for 16 h. The mixture was acidified with 1N aqueous solution of HCl until pH=2 and extracted with DCM/iPrOH (9/1). The organic layer was separated, dried (MgSO₄), filtered and the solvents evaporated in vacuo to yield 2-(2-ethyl-7-isopropyl-4-oxo-pyrazolo[1,5-d][1,2,4]triazin-5-yl)acetic acid (I-14) (117 mg, 99%) as white solid.

Structure analogs were synthesized using the same nrocedure.

| Starting material | Intermediate |
|---|---|
| 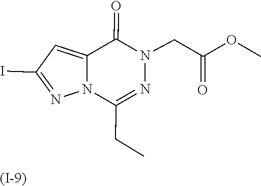 [SMILES: CCc1nn(CC(=O)OC)c(=O)c2cc(I)nn12 ; "(I-9)"] | 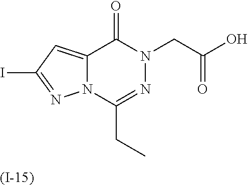 [SMILES: CCc1nn(CC(=O)O)c(=O)c2cc(I)nn12 ; "(I-15)"] |
| 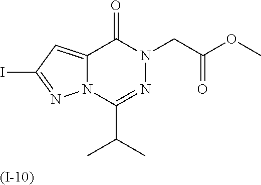 [SMILES: COC(=O)Cn1nc(C(C)C)n2nc(I)cc2c1=O ; "(I-10)"] | 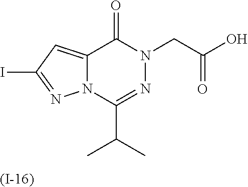 [SMILES: CC(C)c1nn(CC(=O)O)c(=O)c2cc(I)nn12 ; "(I-16)"] |
| 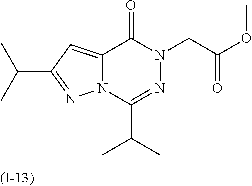 [SMILES: COC(=O)Cn1nc(C(C)C)n2nc(C(C)C)cc2c1=O ; "(I-13)"] | 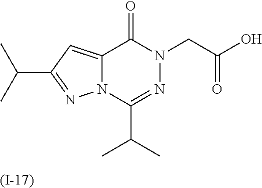 [SMILES: CC(C)c1cc2c(=O)n(CC(=O)O)nc(C(C)C)n2n1 ; "(I-17)"] |
| 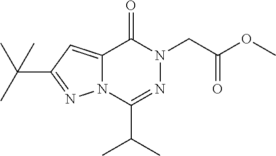 [SMILES: COC(=O)Cn1nc(C(C)C)n2nc(C(C)(C)C)cc2c1=O] | 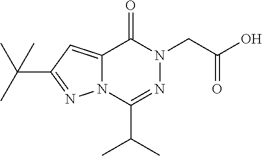 [SMILES: CC(C)c1nn(CC(=O)O)c(=O)c2cc(C(C)(C)C)nn12] |
| 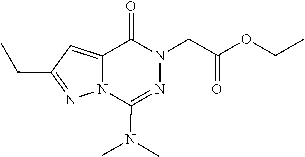 [SMILES: CCOC(=O)Cn1nc(N(C)C)n2nc(CC)cc2c1=O] | 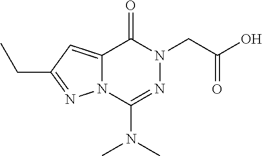 [SMILES: CCc1cc2c(=O)n(CC(=O)O)nc(N(C)C)n2n1] |
| 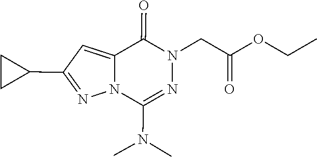 [SMILES: CCOC(=O)Cn1nc(N(C)C)n2nc(C3CC3)cc2c1=O] | 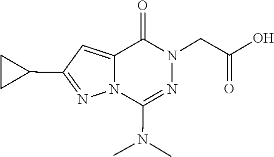 [SMILES: CN(C)c1nn(CC(=O)O)c(=O)c2cc(C3CC3)nn12] |
| 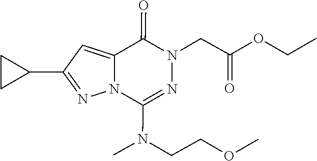 [SMILES: CCOC(=O)Cn1nc(N(C)CCOC)n2nc(C3CC3)cc2c1=O] | 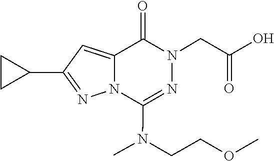 [SMILES: COCCN(C)c1nn(CC(=O)O)c(=O)c2cc(C3CC3)nn12] |
| 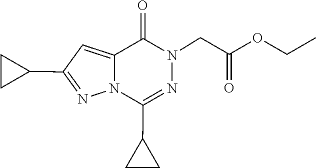 [SMILES: CCOC(=O)Cn1nc(C2CC2)n2nc(C3CC3)cc2c1=O] | 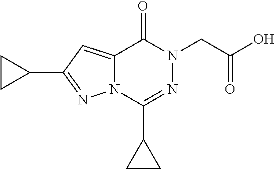 [SMILES: O=C(O)Cn1nc(C2CC2)n2nc(C3CC3)cc2c1=O] |
| 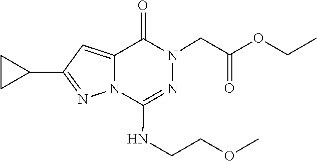 [SMILES: CCOC(=O)Cn1nc(NCCOC)n2nc(C3CC3)cc2c1=O] | 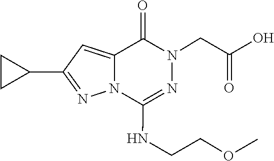 [SMILES: COCCNc1nn(CC(=O)O)c(=O)c2cc(C3CC3)nn12] |
| 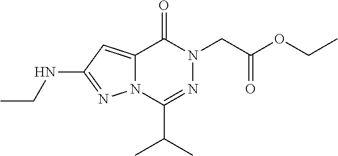 [SMILES: CCNc1cc2c(=O)n(CC(=O)OCC)nc(C(C)C)n2n1] | 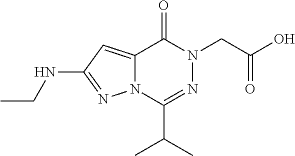 [SMILES: CCNc1cc2c(=O)n(CC(=O)O)nc(C(C)C)n2n1] |
| 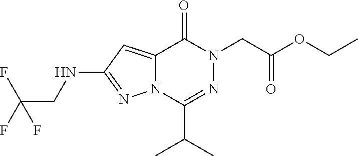 [SMILES: CCOC(=O)Cn1nc(C(C)C)n2nc(NCC(F)(F)F)cc2c1=O] | 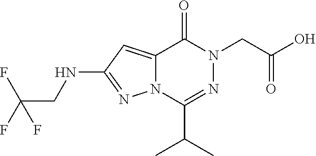 [SMILES: CC(C)c1nn(CC(=O)O)c(=O)c2cc(NCC(F)(F)F)nn12] |
| 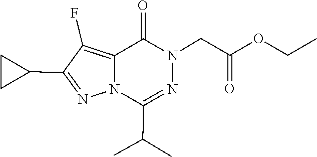 [SMILES: CCOC(=O)Cn1nc(C(C)C)n2nc(C3CC3)c(F)c2c1=O] | 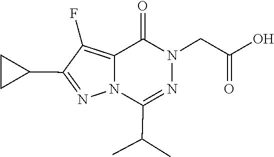 [SMILES: CC(C)c1nn(CC(=O)O)c(=O)c2c(F)c(C3CC3)nn12] |
| 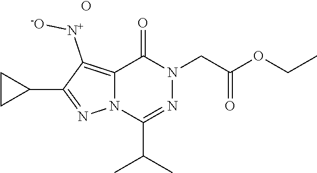 | 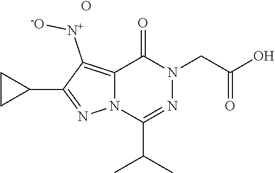 |
| 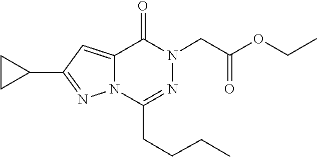 [SMILES: CCCCc1nn(CC(=O)OCC)c(=O)c2cc(C3CC3)nn12] | 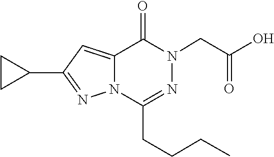 [SMILES: CCCCc1nn(CC(=O)O)c(=O)c2cc(C3CC3)nn12] |
| 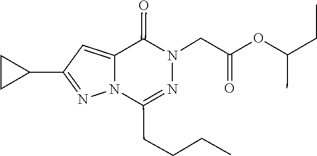 [SMILES: CCCCc1nn(CC(=O)OC(C)CC)c(=O)c2cc(C3CC3)nn12] | |
| 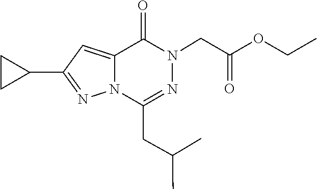 [SMILES: CCOC(=O)Cn1nc(CC(C)C)n2nc(C3CC3)cc2c1=O] | 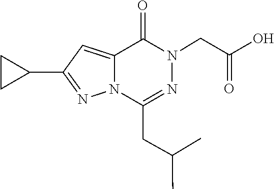 [SMILES: CC(C)Cc1nn(CC(=O)O)c(=O)c2cc(C3CC3)nn12] |
| 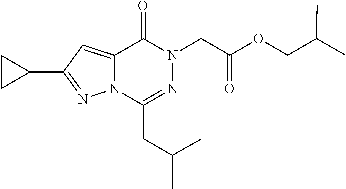 [SMILES: CC(C)COC(=O)Cn1nc(CC(C)C)n2nc(C3CC3)cc2c1=O] | |
| 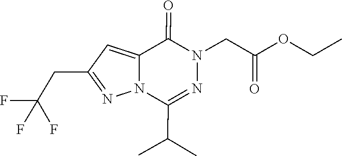 [SMILES: CCOC(=O)Cn1nc(C(C)C)n2nc(CC(F)(F)F)cc2c1=O] | 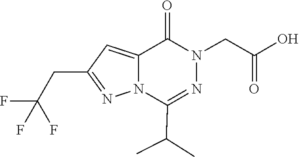 [SMILES: CC(C)c1nn(CC(=O)O)c(=O)c2cc(CC(F)(F)F)nn12] |
| 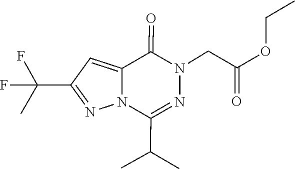 [SMILES: CCOC(=O)Cn1nc(C(C)C)n2nc(C(C)(F)F)cc2c1=O] | 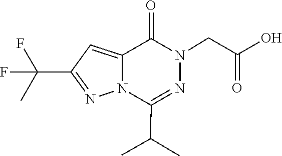 [SMILES: CC(C)c1nn(CC(=O)O)c(=O)c2cc(C(C)(F)F)nn12] |

Synthesis of 2-(2-cyclopropyl-7-methyl-4-oxopyrazolo[1,5-d][1,2,4]triazin-5(4H)-yl)acetic acid

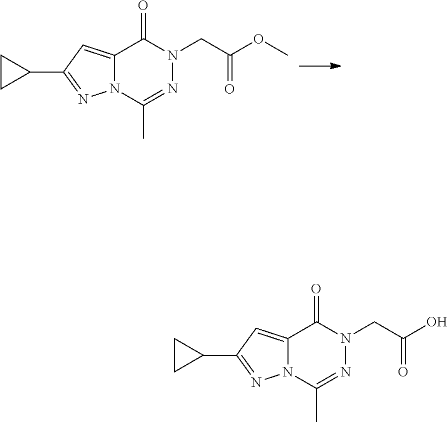 [SMILES: COC(=O)Cn1nc(C)n2nc(C3CC3)cc2c1=O ; Cc1nn(CC(=O)O)c(=O)c2cc(C3CC3)nn12]

Methyl 2-(2-cyclopropyl-7-methyl-4-oxopyrazolo[1,5-d][1,2,4]triazin-5(4H)-yl)acetate (1.21 g, 4.52 mmol) and LiOH [1310-65-2] (245 mg, 10.23 mmol) were suspended in water (20 mL). The reaction was stirred at 50° C. for 1 hour. Then, HCl (7 mL, 2 M in H₂O, 14 mmol) was added and stirred at room temperature for 5 minutes. Solids were filtered, washed with water (5 mL x 2) and dried at 50° C. under high vacuum to afford 2-(2-cyclopropyl-7-methyl-4-oxopyrazolo[1,5-d][1,2,4]triazin-5(4H)-yl)acetic acid (850 mg, yield 76%) as a white solid.

Synthesis of 2-(2-(2,2-difluorocyclopropyl)-74 sopropyl-4-oxopyrazolo[1,5-d][1,2,4]triazin-5(4H)-yl)acetic acid

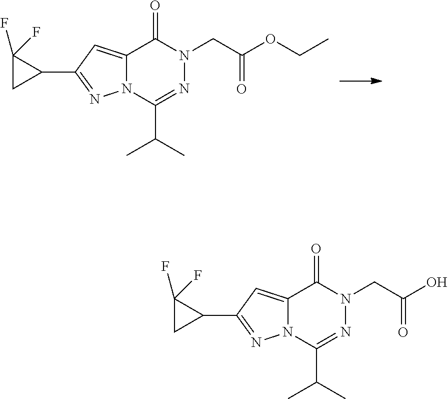 [SMILES: CCOC(=O)Cn1nc(C(C)C)n2nc(C3CC3(F)F)cc2c1=O ; CC(C)c1nn(CC(=O)O)c(=O)c2cc(C3CC3(F)F)nn12]

A mixture of ethyl 2-(2-(2,2-difluorocyclopropyl)-74 sopropyl-4-oxopyrazolo[1,5-d][1,2,4]triazin-5(4H)-yl)acetate (365 mg, 1.07 mmol) and NaOH [1310-73-2] (104 mg, 2.60 mmol) in THF (5 mL) and water (5 mL) was stirred at rt for 2 h. An aqueous solution of HCl ¹N was added then the aqueous layer was extracted with EtOAc twice. The combined organic layers were washed with brine, dried over MgSO₄, filtered and evaporated to dryness to give 2-(2-(2,2-difluorocyclopropyl)-74 sopropyl-4-oxopyrazolo[1,5-d][1,2,4]triazin-5(4H)-yl)acetic acid (309 mg, yield 92%) as a white solid.

1H NMR (400 MHz, DMSO): d ppm 13.20 (s, 1H), 7.23 (s, 1H), 4.68 (s, 2H), 3.62-3.55 (m, 1H), 3.28-3.21 (m, 1H), 2.22-2.14 (m, 2H), 1.31 (d, J=6.8 Hz, 6H)

Synthesis of 2-(7-isopropyl-4-oxo-2-vinyl-pyrazolo[1,5-d][1,2,4]triazin-5-yl)acetic acid (I-18)

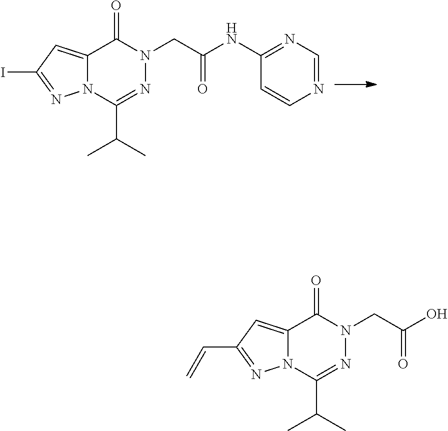 [SMILES: CC(C)c1nn(CC(=O)Nc2ccncn2)c(=O)c2cc(I)nn12 ; C=Cc1cc2c(=O)n(CC(=O)O)nc(C(C)C)n2n1]

To a stirred solution of 2-(2-iodo-7-isopropyl-4-oxo-pyrazolo[1,5-d][1,2,4]triazin yl)-N-pyrimidin-4-yl-acetamide (Final compound 2) (0.4 g, 1.32 mmol) in dioxane (1.5 mL) was added vinylboronic acid pinacol ester [75927-49-0] (0.075 mL, 0.44 mmol), potassium phosphate tribasic (0.9mL, 1M in water, 0.9 mmol) and Ruphos Pd G3 [1445085-77-7] (14 mg, 0.016 mmol). The reaction mixture was then heated at 100° C. for 3 h. The cooled mixture was extracted with DCM. The organic layer was separated, dried (MgSO₄), filtered and the solvents evaporated in vacuo. The crude product was purified by flash column chromatography (SiO₂, EtOAc in heptane 10/90 to 100/0). The desired fractions were collected and concentrated in vacuo to yield 2-(7-isopropyl-4-oxo-2-vinyl-pyrazolo[1,5-d][1,2,4]triazin-5-yl)acetic acid (I-18) (77 mg, 98%) as a yellow solid.

Synthesis of 2-(2-cyclopropyl-7-isopropyl-4-oxo-pyrazolo[1,5-d][1,2,4]triazin-5-yl)acetic acid (I-19)

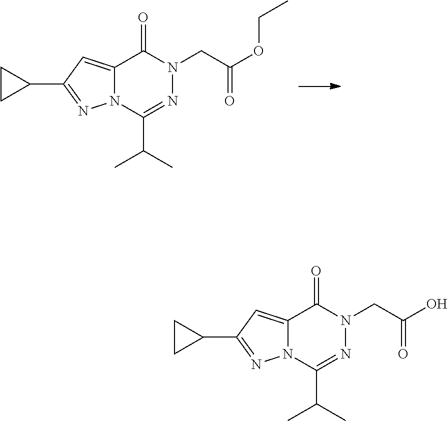 [SMILES: CCOC(=O)Cn1nc(C(C)C)n2nc(C3CC3)cc2c1=O ; CC(C)c1nn(CC(=O)O)c(=O)c2cc(C3CC3)nn12]

2M aqueous solution of NaOH [1310-73-2] (1.3 mL, 2 M, 2.6 mmol) was added to a stirred solution of ethyl 2-(2-cyclopropyl-7-isopropyl-4-oxo-pyrazolo[1,5-d][1,2,4]triazin-5-yl)acetate (I-11) (170 mg, 0.56 mmol) in MeOH (4 mL). The mixture was stirred at 70° C. for 1 h. The mixture was acidified with 1N aqueous solution of HCl until pH=1 and then extracted with DCM (several times). The organic layer was separated, dried (Na₂SO₄), filtered and the solvents evaporated in vacuo to yield 2-(2-cyclopropyl-7-isopropyl-4-oxo-pyrazolo[1,5-d][1,2,4]triazin-5-yl)acetic acid (I-19) (153 mg, 99%) as a white foam, that was used in the next step reaction without further purification.

1H NMR (500 MHz, DMSO-d₆) δ ppm 0.84-0.90 (m, 2 H) 1.01-1.09 (m, 2 H) 1.30 (d, J=6.87 Hz, 6 H) 2.09-2.19 (m, 1 H) 3.49-3.62 (m, 1 H) 4.66 (s, 2 H) 6.99 (s, 1 H) 12.65-13.59 (m, 1 H)

Structure analogs were synthesized using the same procedure.

| Starting material | Intermediate |
|---|---|
| 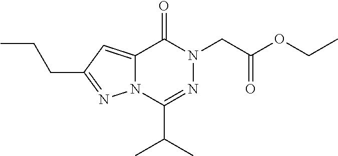 [SMILES: CCCc1cc2c(=O)n(CC(=O)OCC)nc(C(C)C)n2n1] | 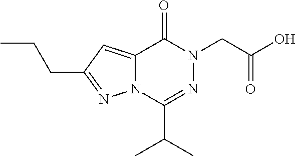 [SMILES: CCCc1cc2c(=O)n(CC(=O)O)nc(C(C)C)n2n1] |
| 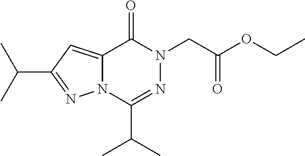 [SMILES: CCOC(=O)Cn1nc(C(C)C)n2nc(C(C)C)cc2c1=O] | 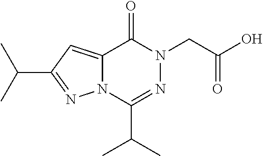 [SMILES: CC(C)c1cc2c(=O)n(CC(=O)O)nc(C(C)C)n2n1] |
| 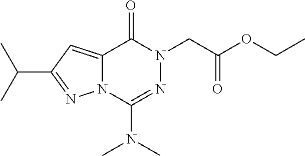 [SMILES: CCOC(=O)Cn1nc(N(C)C)n2nc(C(C)C)cc2c1=O] | 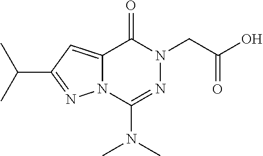 [SMILES: CC(C)c1cc2c(=O)n(CC(=O)O)nc(N(C)C)n2n1] |
| 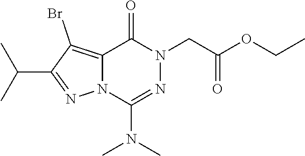 [SMILES: CCOC(=O)Cn1nc(N(C)C)n2nc(C(C)C)c(Br)c2c1=O] | 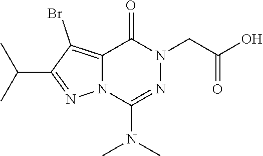 [SMILES: CC(C)c1nn2c(N(C)C)nn(CC(=O)O)c(=O)c2c1Br] |
| 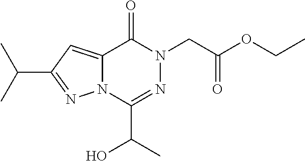 [SMILES: CCOC(=O)Cn1nc(C(C)O)n2nc(C(C)C)cc2c1=O] | 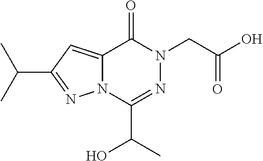 [SMILES: CC(C)c1cc2c(=O)n(CC(=O)O)nc(C(C)O)n2n1] |
| 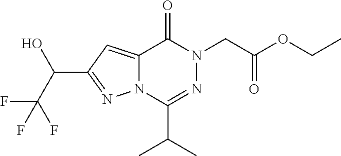 [SMILES: CCOC(=O)Cn1nc(C(C)C)n2nc(C(O)C(F)(F)F)cc2c1=O] | 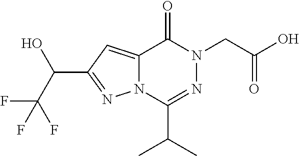 [SMILES: CC(C)c1nn(CC(=O)O)c(=O)c2cc(C(O)C(F)(F)F)nn12] |

Synthesis of 2-(7-ethyl-4-oxopyrazolo[1,5-d][1,2,4]triazin-5(4H)-yl)acetic acid (I-20)

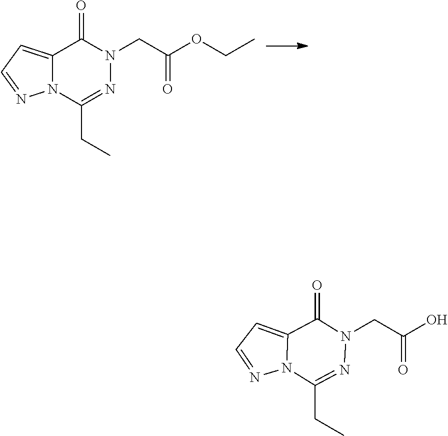 [SMILES: CCOC(=O)Cn1nc(CC)n2nccc2c1=O ; CCc1nn(CC(=O)O)c(=O)c2ccnn12]

A mixture of ethyl 2-(7-ethyl-4-oxopyrazolo[1,5-d][1,2,4]triazin-5(4H)-yl)acetate (1000 mg, 4 mmol) in NaOH (1M in H₂O) [1310-73-2] (6.1 mL, 1 M, 6.1 mmol) was heated at 80° C. in a pressure tube for 1 h.

The mixture was cooled and 6.1 ml HCL 1 N was added. The mixture was stirred 30 min at rt and then the solids were filtered, yielding 2-(7-ethyl-4-oxopyrazolo[1,5-d][1,2,4]triazin-5(4H)-yl)acetic acid (I-20) (712 mg, yield 80%) as a white solid.

Structure analogs were synthesized using the same procedure.

| Starting material | Intermediate |
|---|---|
| 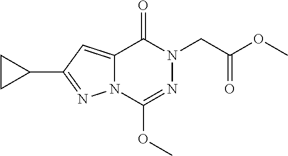 [SMILES: COC(=O)Cn1nc(OC)n2nc(C3CC3)cc2c1=O] | 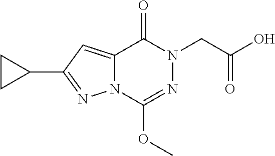 [SMILES: COc1nn(CC(=O)O)c(=O)c2cc(C3CC3)nn12] |

Synthesis of 1-(cyclopropylmethyl)-5-nitropyridin-2(1H)-one

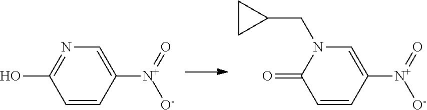 [SMILES: O=[N+]([O-])c1ccc(O)nc1 ; O=c1ccc([N+](=O)[O-])cn1CC1CC1]

In a vessel, NaH (60% dispersion in mineral oil) [7646-69-7] (560 mg, 14 mmol) was added portionwise to a solution of 2-hydroxy-5-nitropyridine [5418-51-9] (1.5 g, 10.7 mmol) in DMF [68-12-2] (40 mL) at 0° C. The reaction was stirred 10 minutes at that temperature and then warmed to ambient temperature and stirred for an additional 20 minutes. (Bromomethyl)cyclopropane [7051-34-5] (2.1 g, 16 mmol) was added dropwise to the solution at 0° C. and stirred at room temperature for 24 hours. The mixture was quenched with NH₄Cl aq. 20% solution and EtOAc was added. The layers were separated and the aqueous phase was extracted with EtOAc. Combined organic layers were dried over MgSO₄ and then concentrated in vacuo. The residue was purified by RP Flash (Stationary phase: Gotec 130g, 25-40 μm, Mobile Phase: ACN in NH₄HCO_{3 0.25}% solution in Water 0/100 to 80/20 over 20V). The desired fractions were collected and the solvent evaporated in vacuo to yield 1-(cyclopropylmethyl)-5-nitropyridin-2(1H)-one (1.4 g, yield 67%).

1H NMR (400 MHz, DMSO-d₆) d ppm 0.35-0.46 (m, 2 H) 0.46-0.55 (m, 2 H) 1.27 (quint, J=7.60, 7.60, 7.60, 7.60, 4.91, 4.91 Hz, 1 H) 3.88 (d, J=7.17 Hz, 2 H) 6.50 (d, J=9.94 Hz, 1 H) 8.14 (dd, J=9.94, 3.24 Hz, 1 H) 9.19 (d, J=3.24 Hz, 1 H)

Structure analogs were synthesized using the same procedure.

| Starting material | Intermediate |
|---|---|
| 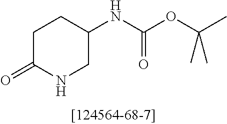 | 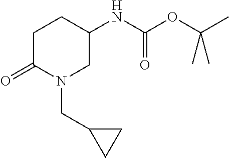 [SMILES: CC(C)(C)OC(=O)NC1CCC(=O)N(CC2CC2)C1] |

Synthesis of 5-amino-1-(cyclopropylmethyl)piperidin-2-one

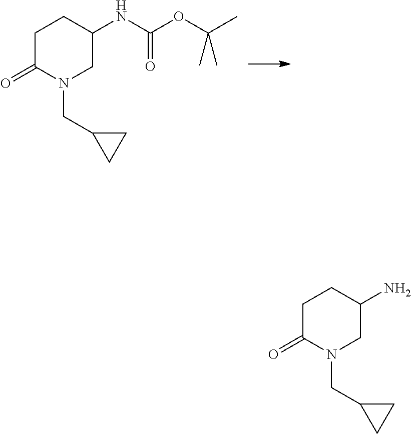 [SMILES: CC(C)(C)OC(=O)NC1CCC(=O)N(CC2CC2)C1 ; NC1CCC(=O)N(CC2CC2)C1]

TFA [76-05-1] (4.88 mL, 1.49 g/mL, 63.72 mmol) was added to a solution of tert-butyl (1-(cyclopropylmethyl)-6-oxopiperidin-3-yl)carbamate (380 mg, 1.27 mmol) in DCM (0.49 mL). The reaction was stirred at room temperature for 18 hours. The crude was concentrated in vacuo and then dissolved in DCM/MeOH and filtered over a SCX2 column, washed with DCM and MeOH. Then, the product was rinsed with NH_{3/}MeOH 3.5 M to elute the product, concentrated in vauo to yield 5-amino-1-(cyclopropylmethyl)piperidin-2-one (185 mg, yield 86%) as a colourless syrup.

Synthesis of [1,2,4]triazolo[4,3-b]pyridazin-6-amine

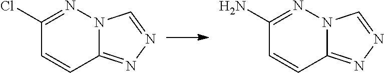 [SMILES: Clc1ccc2nncn2n1 ; Nc1ccc2nncn2n1]

Aqueous ammonia 25% [1336-21-6] (35 mL, 1 g/mL, 249.67 mmol) was added to a stirred solution of 6-chloro-[1,2,4]triazolo[4,3-b]pyridazine [28593-24-0] (3.5 g, 22.65 mmol) in 1,4-dioxane (20 mL) in a sealed tube and the mixture was heated at 90° C. for 16h. The solvents were removed in vacuo. The crude product was purified by flash column chromatography (silica 25 g; dry load in silica; DCM:MeOH (9:1) in DCM 0/100 to 100/0). The desired fractions were collected and concentrated in vacuo to yield [1,2,4]triazolo[4,3-b]pyridazin-6-amine (1.4 g, yield 42%) as a brown solid.

Structure analogs were synthesized using the same procedure.

| Starting material | Intermediate |
|---|---|
| 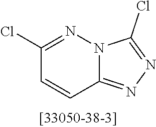 [SMILES: Clc1ccc2nnc(Cl)n2n1] | 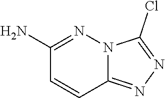 [SMILES: Nc1ccc2nnc(Cl)n2n1] |

Synthesis of 3-methyl-[1,2,4]triazolo[4,3-b]pyridazin-6-amine

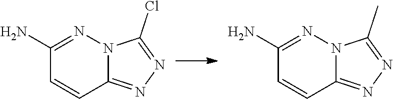

A mixture of 3-chloro-[1,2,4]triazolo[4,3-b]pyridazin-6-amine (1.25 g, 7.37 mmol) in THF, dry (60 mL) was stirred at room temp and degased with nitrogen for 5 min. Bis(tri-tert-butylphosphine)palladium(O) [53199-31-8] (565.09 mg, 1.11 mmol) was added and the reaction mixture was degassed again for 5 min. Methylzinc chloride [5158-46-3] (7.37 mL, 2 M, 14.74 mmol) was added dropwise and the rm was stirred in a pressure tube under nitrogen at 90° C. for 8 h.The RM was cooled, decomposed with 10 ml sat. NH₄Cl solution, stirred for 10 min and then neutralized with NaHCO₃ solution.The whole was evaporated and then stirred in 50 ml MeOH overnight.The solids were filtered and the filtarte was purified on Prep HPLC (Stationary phase: RP) (Bridge Prep C18 OBD-10 μm,30×150mm, Mobile phase: 0.25% NH₄HCO₃ solution in water, CH₃CN), yielding 3-methyl-[1,2,4]triazolo[4,3-b]pyridazin-6-amine (402 mg, yield 37%) as a white solid.

Synthesis of 2-(difluoromethyl)pyridin-4-amine

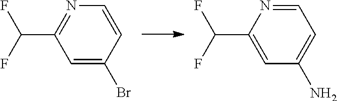 [SMILES: FC(F)c1cc(Br)ccn1 ; Nc1ccnc(C(F)F)c1]

A pressure reactor was charged with 4-bromo-2-(difluoromethyl)pyridine [1211580 9] (15 g, 72.11 mmol) and copper(I) oxide [1308-76-5] (1595.7 mg, 10.82 mmol) in NH₃ (28% in H₂O) [7664-41-7] (90 mL, 0.9 g/mL, 1331.73 mmol) and NMP [872 4] (40 mL). The reaction mixture was heated at 110° C. overnight. The reaction mixture was partitioned between water (100 mL) and diethylether (100 mL). The organic layer was isolated and the aqueous layer was extracted with diethylether (10 X 100 mL). The combined organic layers were evaporated to dryness and the residue was purified on silica using a gradient from heptane to EtOAc. The desired fractions were concentrated under reduced pressure and yielding a clearless oil. This oil was dissolved in iPrOH (100 mL) and this was cooled in an ice bath. To this solution was added slowly HCl (6M in iPrOH) [7647-01-0] (48.08 mL, 6 M, 288.45 mmol). A white precipitation was formed. The mixture was allowed to reach RT and then the white solid was collected on a filter, rinsed with 20 mL of i-PrOH and dried in a vacuum oven at 55° for 18 hours yielding 2-(difluoromethyl)pyridin-4-amine (12 g, yield 92%).

Synthesis of 3-oxocyclobutane-1-carboxylate

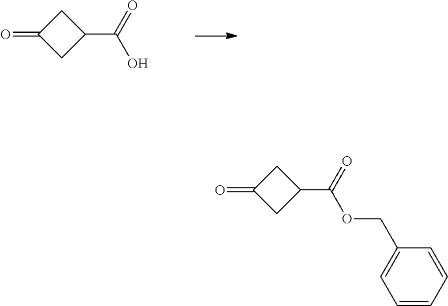 [SMILES: O=C1CC(C(=O)O)C1 ; O=C1CC(C(=O)OCc2ccccc2)C1]

To a mixture of 3-oxocyclobutane-1-carboxylic acid [23761-23-1] (10 g, 87.64 mmol) in DCM (400 ml), Et3N [121-44-8] (18.3 mL, 0.728 g/mL, 131.46 mmol) and DMAP [1122-58-3] (1.07 g, 8.764 mmol) were added at rt. Then the mixture was cooled at 0° C. and benzyl chloroformate [501-53-1] (13.76 mL, 1.195 g/mL, 96.4 mmol) was added dropwise. The mixture was stirred for 24 h at rt. Water was added and the mixture was extracted with DCM, the organic layer was separated . The combined organic layers were dried (Na₂SO₄), filtered and the solvents evaporated in vacuo. The crude product was purified by flash column chromatography (MeOH in DCM 0/100 to 3/97). The desired fractions were collected, the solvent evaporated in vacuo to yield 3-oxocyclobutane-1-carboxylate (9 g, yield 50%).

1H NMR (400 MHz, CHLOROFORM-d) d 7.32-7.45 (m, 1H), 5.20 (s, 2H), 3.22-3.53 (m, 5H)

Synthesis of benzyl 3-ethyl-3-hydroxycyclobutane-1-carboxylate

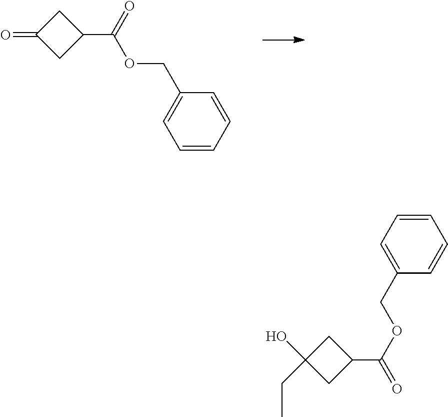 [SMILES: O=C1CC(C(=O)OCc2ccccc2)C1 ; CCC1(O)CC(C(=O)OCc2ccccc2)C1]

To a mixture of benzyl 3-oxocyclobutane-1-carboxylate (1500 mg, 7.345 mmol) in THF (20 mL), ethylmagnesium bromide [925-90-6] (3 mL, 3 M, 9 mmol) was added at -60° C. The mixture was stirred at -50° C. for 2 hours. A saturated solution of NH₄Cl was added and the crude was extracted with AcOEt (2×10 ml), the combined organic layers were dried and evaporated in vacuo to afford P1. The crude was purified by flash chromatography (silica AcOEt in Hept 0/100 to 20/80), the correspoinding layers were evaporated in vacuo to yield benzyl 3-ethyl-3-hydroxycyclobutane-1-carboxylate (750 mg, yield 44%) as oil.

1H NMR (400 MHz, CHLOROFORM-d) d 7.31-7.40 (m, 1H), 5.14 (s, 1H), 2.73 (quin, J=8.38 Hz, 1H), 2.20-2.45 (m, 5H), 1.62 (q, J=7.40 Hz, 2H), 1.28 (br s, 1H), 0.95 (t, J=7.40 Hz, 3H)

Synthesis of benzyl 3-((tert-butyldimethylsilyl)oxy)-3-ethylcyclobutane-1-carboxylate

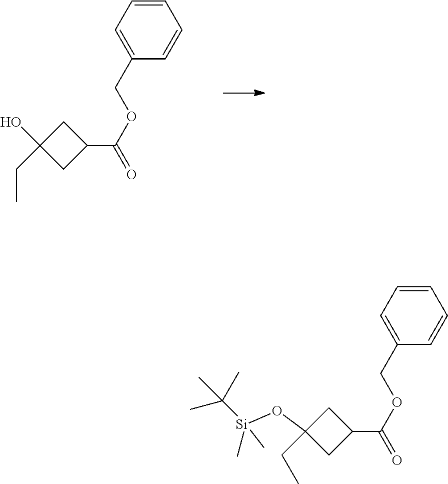 [SMILES: CCC1(O)CC(C(=O)OCc2ccccc2)C1 ; CCC1(O[Si](C)(C)C(C)(C)C)CC(C(=O)OCc2ccccc2)C1]

To a mixture of benzyl 3-ethyl-3-hydroxycyclobutane-1-carboxylate (750 mg, 3.2011 mmol) in DCM (25 ml), tert-butyldimethylsilyl trifluoromethanesulphonate [69739 0] (1015 mg, 3.84 mmol), DIPEA [7087-68-5], (0.82 mL, 0.75 g/mL, 4.8 mmol) and 4-dimethylaminopyridine [1122-58-3 ] (40 mg, 0.32 mmol) were added. The mixture was stirred for 24 h at rt. Water was added at rt, and the crude was extracted with DCM (2×10 mL), the combined organic layers were dried and evaporated in vacuo. The crude was purified by flash chromatography (silica AcOEt in Hept 0/100 to 10/90), the correspoinding layers were evaporated in vacuo to yield 3-((tert-butyldimethylsilyl)oxy)-3-ethylcyclobutane-1-carboxylate (750 mg, yield 67%) as oil.

Synthesis of benzyl ((1r,3s)-3-((tert-butyldimethylsilyl)oxy)-3-ethylcyclobutyl)carbamate

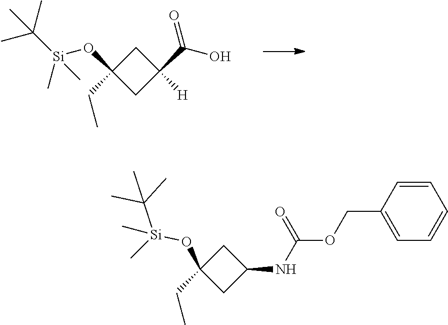 [SMILES: CC[C@]1(O[Si](C)(C)C(C)(C)C)C[C@@H](C(=O)O)C1 ; CC[C@]1(O[Si](C)(C)C(C)(C)C)C[C@@H](NC(=O)OCc2ccccc2)C1]

To a mixture of (1r,3s)-3-((tert-butyldimethylsilyl)oxy)-3-ethylcyclobutane-1-carboxylic acid (500 mg, 1.935 mmol) in toluene (20 mL) was added triethylamine [121-44-8] (0.7 mL, 5.036 mmol), followed by diphenylphosphoryl azide [26386-88-9] (800 mg, 2.9 mmol). The reaction mixture was stirred at 80° C. for 3h. The reaction mixture was then cooled to room temperature and benzyl alcohol [100-51-6] (251 mg, 2.3 mmol) was added. The resulting solution was heated to reflux 10 h. The crude was cooled and evapoarated in vacuo and treated with a saturated solution of NaHCO₃ and extracted with AcOEt (2×5 ml), the combined organic layers were evaporated to afford an oil. The crude was purified by columm chromatograpy (silica, AcOEt in heptane 0/100 to 20/80), the correspoinding fractions were evaporated in vacuo to yield ((1r,3 s)-3-((tert-butyldimethylsilyl)oxy)-3-ethylcyclobutyl)carb amate (400 mg, yield 57%) as oil wich solidified upon standing.

1H NMR (400 MHz, CHLOROFORM-d) d ppm 0.06 (s, 6 H) 0.88 (s, 9 H) 0.88-0.93 (m, 3 H) 1.48-1.61 (m, 2 H) 1.81-1.95 (m, 2 H) 2.42-2.61 (m, 2 H) 3.65-3.80 (m, 1 H) 4.83 (br d, J=5.1 Hz, 1 H) 5.08 (s, 2 H) 7.28-7.43 (m, 5 H)

Synthesis of 5-amino-1-(cyclopropylmethyl)pyridin-2(1H)-one

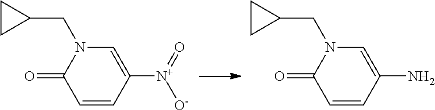 [SMILES: O=c1ccc([N+](=O)[O-])cn1CC1CC1 ; Nc1ccc(=O)n(CC2CC2)c1]

A solution of 1-(cyclopropylmethyl)-5-nitropyridin-2(1H)-one (500 mg, 2.6 mmol) in EtOH (50 mL) was hydrogenated in a H-cube reactor (1 mL/min, 30 mm Pd/C 10% cartridge, full H₂ mode, 50° C., 1 cycle). The solvent was evaporated in vacuo. The residue was filtered over SCX column, washed with DCM and the product was rinse with NH_{3/}MeOH 3.5 N to yield 5-amino-1-(cyclopropylmethyl)pyridin-2(1H)-one (390 mg, yield 92%) as a brown-black syrup.

1H NMR (500 MHz, DMSO-d₆) d ppm 0.27-0.36 (m, 3 H) 0.40-0.52 (m, 2 H) 3.62 (d, J=7.02 Hz, 2 H) 6.24 (d, J=9.46 Hz, 1 H) 6.89 (d, J=2.90 Hz, 1 H) 7.05 (dd, J=9.46, 3.05 Hz, 1 H) +2 exchange protons

Synthesis of (1r,3s)-3-((tert-butyldimethylsilyl)oxy)-3-ethylcyclobutan-1-amine

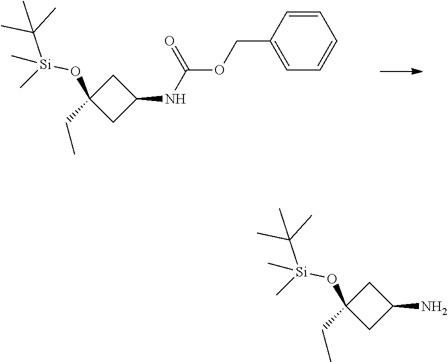 [SMILES: CC[C@]1(O[Si](C)(C)C(C)(C)C)C[C@@H](NC(=O)OCc2ccccc2)C1 ; CC[C@]1(O[Si](C)(C)C(C)(C)C)C[C@@H](N)C1]

To a mixture of benzyl ((1r,3s)-3-((tert-butyldimethylsilyl)oxy)-3-ethylcyclobutyl)carbamate (400 mg, 1.1 mmol) in THF (40 mL), Pd/C (10%) (120mg, 0.112 mmol) was added under N2 atmophere, the mixture was hidrogenated with baloon at rt for 16h. The crude was filtered over celite and evaporated in vacuo. The crude product was purified by flash column chromatography (silica, MeOH in DCM 0/100 to 10/90), the correspoinding fractions were evaporated in vacuo to yield (1r,3s)-3-((tert-butyldimethylsilyl)oxy)-3-ethylcyclobutan-1-amine (200 mg, yield 79%) as transparent oil.

1H NMR (400 MHz, CHLOROFORM-d) d 2.86-2.99 (m, 1H), 2.39-2.51 (m, 2H), 1.69-1.82 (m, 2H), 1.50 (q, J=7.24 Hz, 4H), 0.81-0.93 (m, 12H), 0.05-0.12 (m, 6H)

Structure analogs were synthesized using the same procedure.

| Starting material | Intermediate |
|---|---|
| 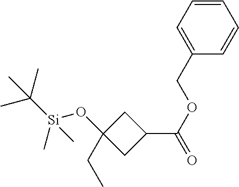 [SMILES: CCC1(O[Si](C)(C)C(C)(C)C)CC(C(=O)OCc2ccccc2)C1] | 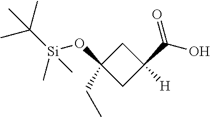 [SMILES: CC[C@]1(O[Si](C)(C)C(C)(C)C)C[C@@H](C(=O)O)C1] |

Synthesis of 3-bromo-2-hydrazinyl-5-nitropyridine

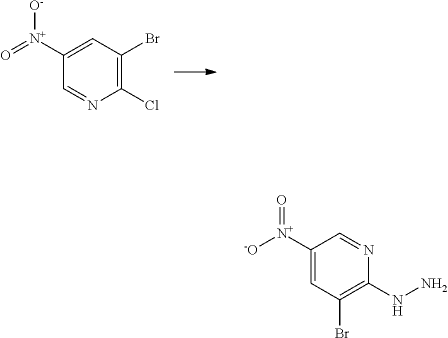

3-Bromo-2-chloro-5-nitropyridine [5470-17-7] (4 g, 16.85 mmol, 1 equiv) was dissolved in 1,4-dioxane (216.2 mL), the solution cooled to 0° C. and hydrazine hydrate [7803-57-8] (24.5 mL, 1.03 g/mL, 0.51 mol, 30 equiv) was added quickly (<15 seconds) at 0° C. After addition, the mixture was stirred vigorously at 0° C. for 10 minutes then allowed to warm to r.t. and stirred for a further hour. The mixture was concentrated on a rotary evaporator to about 150 mL of dark red mixture. It was then cooled to 0° C. and DI water (400 mL) was added, then this suspension was poured on additional 400 mL of DI water at 0° C. The mixture was kept at 0° C. for 15-20 min and the suspended solid was filtered off on a sinter funnel, washing the flask and solid with ca. 5+5 mL of DI water. The product was dried in the oven at 50° C. under vacuum for 16 hours to obtain 3-bromo-2-hydrazinyl-5-nitropyridine (3.32 g, yield 85%) as fluffy grey/greenish solid.

1H NMR (400 MHz, DMSO-d₆) ppm 5.07 (br s, 2 H), 8.39 (d, J=1.8 Hz, 1 H), 8.95 (d, J=2.2 Hz, 1 H), 9.22 (br s, 1 H).

Synthesis of 8-bromo-6-nitro-[1,2,4]triazolo[4,3-a]pyridine

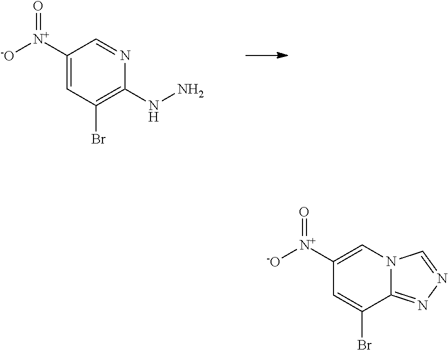

3-Bromo-2-hydrazinyl-5-nitropyridine (4 g, 17.2 mmol, 1 equiv) was suspended in trimethyl orthoformate [149-73-5] (28.2 mL, 0.97 g/mL, 0.26 mol, 10 equiv) in a pressure tube. The tube was sealed with a screw-cap and the mixture heated at 100° C. for 2.5 hours. The reaction was allowed to cool to r.t., then cooled to 0° C. for ca. 30 min and the suspension filtered off washing the reaction vial and filtered solid with a 1:1 mixture of Heptane/EtOAc (10 mL). The solid was dried on the filter under vacuum flow for 5 minutes then at 50° C. under vacuum for 2h to yield 8-bromo-6-nitro-[1,2,4]triazolo[4,3-a]pyridine (3.66 g, yield >98% purity, 88%) as pale brown solid.

1H NMR (400 MHz, DMSO-d₆) ppm 8.40 (d, J=1.6 Hz, 1 H), 9.56 (s, 1 H), 9.90 (d, J=1.6 Hz, 1 H).

Synthesis of 8-bromo-[1,2,4]triazolo[4,3-a]pyridin-6-amine

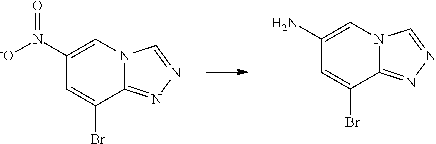

8-Bromo-6-nitro-[1,2,4]triazolo[4,3-a]pyridine (1 g, 4.11 mmol, 1 equiv) and iron powder [7439-89-6] (1.38 g, 24.7 mmol, 6 equiv) were placed in a screw-cap tube and

AcOH (18.8 mL) was added. The mixture was stirred vigorously at r.t. for 3 hours. The green thick suspension was diluted with DI water (30-40 mL). This dark mixture was concentrated in vacuo down to ca. 10 mL of volume left. The residue was neutralised by slow addition of 80 mL of a 1:1 mixture of aq. sat. NaHCO₃ and K₂CO3. It was then extracted with DCM/MeOH 95:5 (5×150 mL). The combined organic extracts were dried over Na₂SO₄, filtered and the filtrate concentrated in vacuo to afford 8-bromo-[1,2,4]triazolo[4,3-a]pyridin-6-amine (450 mg, yield 51%) as pale tan solid 1H NMR (400 MHz, DMSO-d₆) ppm 5.27 (s, 2 H), 7.37 (d, J=1.8 Hz, 1 H), 7.66 (d, J=1.8 Hz, 1 H), 9.13 (s, 1 H).

Synthesis of 6-aminoimidazo[1,2-a]pyridine-2-carboxamide

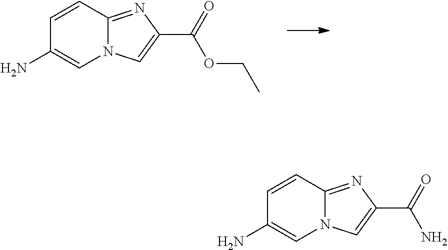

A mixture of ethyl 6-aminoimidazo[1,2-a]pyridine-2-carboxylate [158980-21-3] (1 g, 4.87 mmol) in NH₃ (28% in H₂O) [7664-41-7] (20 mL, 0.9 g/mL, 295.94 mmol) was stirred and heated in a PT at 90° C. for 3 h. The ammonia was evaporated on a rotavap and the crude was used without any purification in the next step.

Sythesis of N-(2-cyanoimidazo[1,2-a]pyridin-6-yl)-2,2,2-trifluoroacetamide

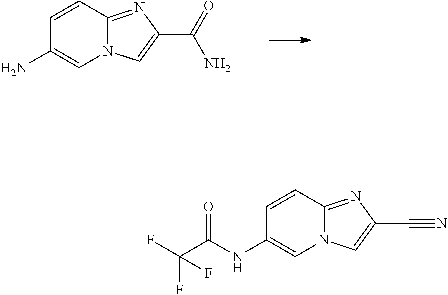 [SMILES: NC(=O)c1cn2cc(N)ccc2n1 ; N#Cc1cn2cc(NC(=O)C(F)(F)F)ccc2n1]

TFAA [407-25-0] (0.28 mL, 1.51 g/mL, 1.99 mmol) was added to a solution of 6-aminoimidazo[1,2-a]pyridine-2-carboxamide (100 mg, 0.57 mmol) and TEA [121-44-8] (0.39 mL, 0.73 g/mL, 2.84 mmol) in THF, dry (3 mL) under N2 at 0° C. The reaction was stirred at 0° C. for another 1 hr and then at rt for 2 h. The RM was decomposed with water and then extracted with DCM. The OL was dried on MgSO₄ and evaporated. The residue was used for the next step without any purification.

Synthesis of 6-aminoimidazo[1,2-a]pyridine-2-carbonitrile

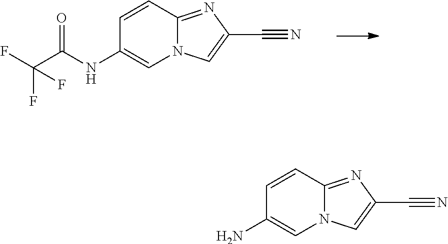 [SMILES: N#Cc1cn2cc(NC(=O)C(F)(F)F)ccc2n1 ; N#Cc1cn2cc(N)ccc2n1]

A solution of N-(2-cyanoimidazo[1,2-a]pyridin-6-yl)-2,2,2-trifluoroacetamide (150 mg, 0.59 mmol) and K₂CO_{3 [584}-08-7] (163.13 mg, 1.18 mmol) in water, distilled (3.11 ml) and MeOH [67-56-1] (3.11 mL) was stirred at rt overnight. The RM was diluted to 20 mL with water and then this fraction was extracted with 2-MeTHF, washed with brine, dried on MgSO₄ and evaporated to yield 6-aminoimidazo[1,2-a]pyridine-2-carbonitrile (93 mg, yield quantitative) as a brown/green solid.

Synthesis of ethyl 6-((tert-butoxycarbonyl)amino)imidazo[1,2-a]pyridine-2-carboxylate

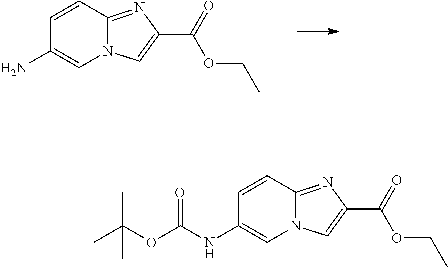 [SMILES: CCOC(=O)c1cn2cc(N)ccc2n1 ; CCOC(=O)c1cn2cc(NC(=O)OC(C)(C)C)ccc2n1]

BOC-anhydride [24424-99-5] (5.85 g, 1.02 g/mL, 26.8 mmol) was added portionwise to a solution of ethyl 6-aminoimidazo[1,2-a]pyridine-2-carboxylate [158980-21-3] (5 g, 24.36 mmol) and DMAP [1122-58-3] (2.98 g, 24.36 mmol) in DCM PA, (50 ml) while stirring at rt. The rm was stirred at rt for 48 h. The mixture was poured out in 300 ml water, extracted with EtOAc, then the 01 was washed with brine, dried on MgSO₄ and evaporated. The residue was purified on a column with silicagel, eluent : Me0H in DCM, from 0 to 5%. The pure fractions were evaporated, yielding 6-((tert-butoxycarbonyl)amino)imidazo[1,2-a]pyridine-2-carboxylate (1.1 g, yield 15%) as a brown foam.

Synthesis of tert-butyl (2-(hydroxymethyl)imidazo[1,2-a]pyridin-6-yl)carbamate

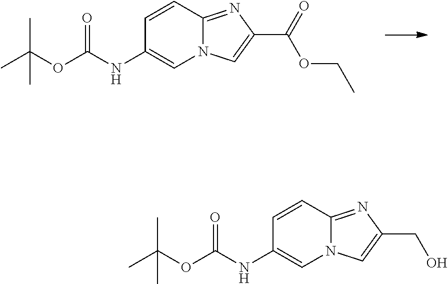

Lithium borohydride [16949-15-8] (1637.55 2 M, 3.28 mmol) was added dropwise to a solution of ethyl 6-((tert-butoxycarbonyl)amino)imidazo[1,2-a]pyridine-2-carboxylate (1000 mg, 3.28 mmol) in THF, PA, dry (20 ml) under a nitrogen atmosphere at rt. The rm was stirred at rt for 18 h. The RM was decomposed with water, extracted with DCM, dried on MgSo4 and evaporated. The residue was purified on a column with silica, eluent : MeOH in DCM from 0 to 8%. The pure fractions were evaporated, yielding tert-butyl (2-(hydroxymethyl)imidazo[1,2-a]pyridin-6-yl)carbamate (635 mg, yield 74%) as a white solid.

Synthesis of tert-butyl (2-formylimidazo[1,2-a]pyridin-6-yl)carbamate

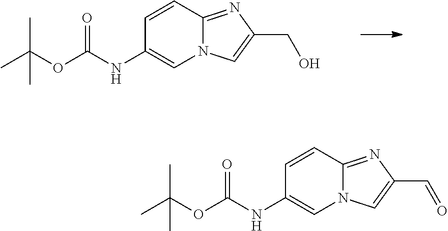 [SMILES: CC(C)(C)OC(=O)Nc1ccc2nc(CO)cn2c1 ; CC(C)(C)OC(=O)Nc1ccc2nc(C=O)cn2c1]

A mixture of tert-butyl (2-(hydroxymethyl)imidazo[1,2-a]pyridin-6-yl)carbamate (600 mg, 2.28 mmol) and manganese(IV) oxide activated [1313-13-9] (3000 mg, 34.51 mmol) in 1,4-dioxane (32 mL) was stirred at rt for 3 h. The solids were filtered and the filtrate was evaporated to yield (2-formylimidazo[1,2-a]pyridin-6-yl)carbamate (330 mg, yield 55%) as a yellow solid.

Synthesis of tert-butyl (2-(difluoromethyl)imidazo[1,2-a]pyridin-6-yl)carbamate

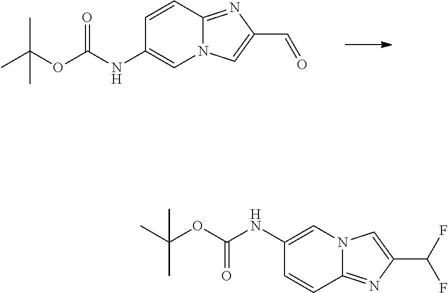 [SMILES: CC(C)(C)OC(=O)Nc1ccc2nc(C=O)cn2c1 ; CC(C)(C)OC(=O)Nc1ccc2nc(C(F)F)cn2c1]

To a mixture of tert-butyl (2-formylimidazo[1,2-a]pyridin-6-yl)carbamate (280 mg, 1.07 mmol) in DCM, PA, dry (11 mL), diethylaminosulfur trifluoride [38078-09-0] (424.77 1.22 g/mL, 3.21 mmol) was added at 0° C. The reaction was stired at 0° C. for lh then another 16 hours at rt. The mixture was poured in water and neutralized with NaHCO₃. The 01 was separated, dried on MgSO₄, filtered and evaporated. The residue was purified on a column with silicagel, eluent Me0H in DCM, from 0 to 5%. The pure fractions were evaporated, yielding tert-butyl (2-(difluoromethyl)imidazo[1,2-a]pyridin-6-yl)carbamate (124 mg, yield 401%) as a brown sticky solid.

Synthesis of 2-(difluoromethyl)imidazo[1,2-a]pyridin-6-amine

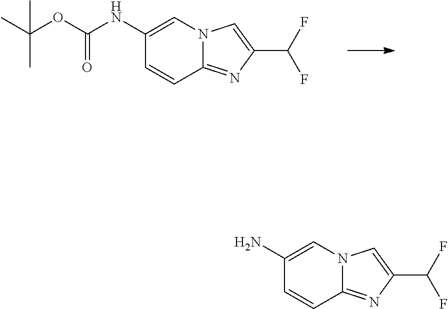 [SMILES: CC(C)(C)OC(=O)Nc1ccc2nc(C(F)F)cn2c1 ; Nc1ccc2nc(C(F)F)cn2c1]

A mixture of tert-butyl (2-(difluoromethyl)imidazo[1,2-a]pyridin-6-yl)carbamate (124 mg, 0.44 mmol) in HCl (4M in dioxane) [7647-01-0] (2 mL, 4 M, 8 mmol) was stirred at rt for 18 h. The solvent was evaporated to obtain 2-(difluoromethyl)imidazo[1,2-a]pyridin-6-amine (112 mg, yield quantitative).

Synthesis of 6-iodoimidazo[1,5-a]pyridine-1-carboxylate

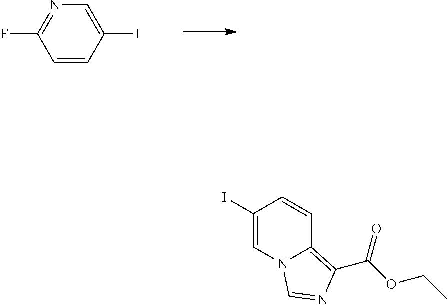

Anhydrous DMF (50 mL) was added to a vial charged with NaH (60% dispersion in mineral oil) [7646-69-7] (2.24 g, 56.06 mmol) under N2. The mixture was cooled down to 0° C. Ethyl isocyanoacetate [2999-46-4] (6.13 mL, 56.06 mmol) was added dropwise and then, after 30 min, 2-fluoro-5-iodo-pyridine [171197-80-1] (10.0 g, 44.85 mmol) was added in three portions. The reaction was allowed to warm up to rt and then heated to 60° C. for 16 h. The reaction was cooled to rt and diluted with EtOAc (500 mL) and water (300 mL). The organic layer was separated and washed with brine (2×100 mL). The combined aqueous layers were extracted with EtOAc (200 mL). The combined organic layers were dried over MgSO₄, filtered and evaporated. The crude product was purified by FCC on silica gel (330 g, gradient: from heptane 100% up to hept/EtOAc 3/7) to obtain 6-iodoimidazo[1,5-a]pyridine-1-carboxylate (2.94 g, yield 21%) as an off-white solid.

Synthesis of 6-iodoimidazo[1,5-a]pyridine-1-carboxylic acid

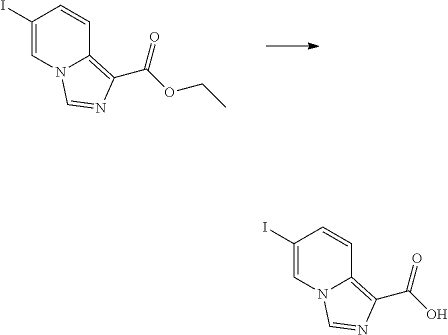

NaOH (1M in H₂O) (36 mL, 36 mmol) was added to a solution of ethyl 6-iodoimidazo[1,5-a]pyridine-1-carboxylate (3.75 g, 11.86 mmol) in THF (35 mL). The reaction was stirred at 60° C. for 2 h. The reaction was concentrated under reduced pressure to remove the THF and then treated with HCl until pH slightly acidic. The solid precipitate was filtered off, washed with water and then dried at 50° C. under vacuum to yield 6-iodoimidazo[1,5-a]pyridine-1-carboxylic acid (3.25 g, yield 95%) as an off-white solid.

Synthesis of 6-iodoimidazo[1,5-a]pyridine-1-carbonyl chloride

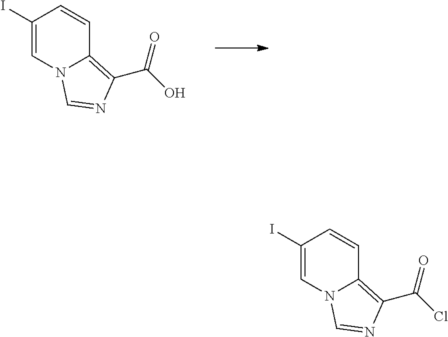 [SMILES: O=C(O)c1ncn2cc(I)ccc12 ; O=C(Cl)c1ncn2cc(I)ccc12]

SOCl[7719-09-7] (4.1 mL, 56.5 mmol) was added dropwise to a suspension of 6-iodoimidazo[1,5-a]pyridine-1-carboxylic acid (3.25 g, 11.28 mmol) in dry ACN (30 mL). The reaction was stirred at 60° C. for 1 h. Volatiles were removed under reduced pressure and the crude product was used as such in the following step.

Synthesis of 6-iodoimidazo[1,5-a]pyridine-1-carboxamide

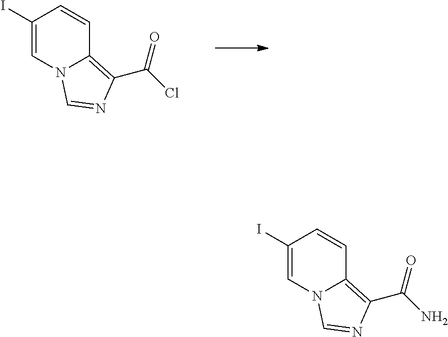 [SMILES: O=C(Cl)c1ncn2cc(I)ccc12 ; NC(=O)c1ncn2cc(I)ccc12]

A suspension of 6-iodoimidazo[1,5-a]pyridine-1-carbonyl chloride (3.45 g, 11.26 mmol) in DCM (50 mL) was cooled down to 0° C. NH₃ (28% in H₂O) (50 mL, 740 mmol) was added portionwise and the mixture was allowed to warm up to rt and stirred for 1 h. The reaction was filtered and the solid cake was washed with water and then dried in the vacuum oven at 50° C. for 16 h to obtain 6-iodoimidazo[1,5-a]pyridine carboxamide (2.29 g, yield 71%) as a brownish solid.

Synthesis of 6-iodoimidazo[1,5-a]pyridine-1-carbonitrile

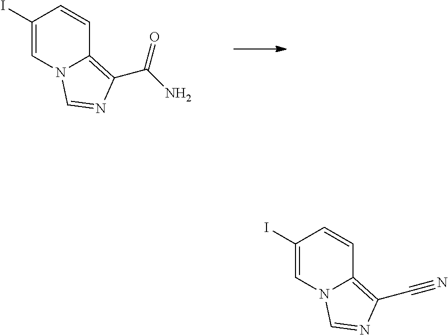 [SMILES: NC(=O)c1ncn2cc(I)ccc12 ; N#Cc1ncn2cc(I)ccc12]

POCl_{3 [10025}-87-3] (0.82 mL, 8.78 mmol) was added dropwise to a solution of 6-iodoimidazo[1,5-a]pyridine-1-carboxamide (2.29 g, 7.98 mmol) in anhydrous DMF (23 mL) stirring at 0° C. The reaction was allowed to warm up to rt and stirred for 30 min. The reaction was quenched with ice (50 mL approx) and diluted with EtOAc (400 mL) and water (150 mL). The organic layer was separated and the aqueous one was extracted with EtOAc (100 mL). The combined organic layers were dried over MgSO₄, filtered and concentrated under reduced pressure to yield 6-iodoimidazo[1,5-a]pyridine-1-carbonitrile (2.09 g, yield 97%) as a brown solid.

Synthesis of (E,Z)-N′45-bromopyridin-2-yl)methylene)-4-methylbenzene-sulfonohydrazide

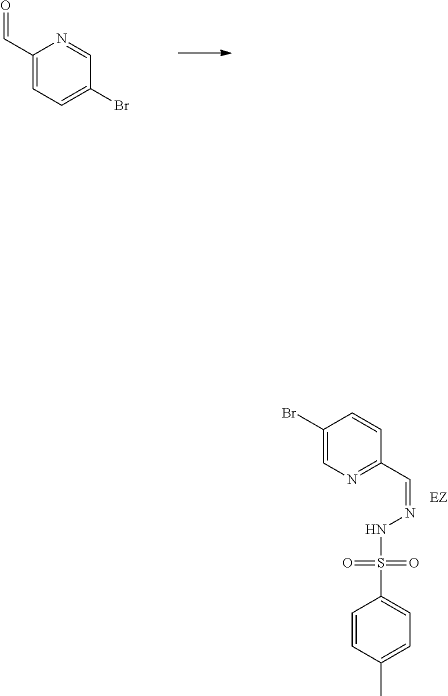 [SMILES: O=Cc1ccc(Br)cn1 ; Cc1ccc(S(=O)(=O)N/N=C\c2ccc(Br)cn2)cc1]

4-Methylbenzenesulfonhydrazide [1576-35-8] (1.0 g, 5.376 mmol) was added to a solution of 5-bromopyridine-2-carbaldehyde [31181-90-5] (1.0 g, 5.376 mmol) in DCM (10 mL) and MeOH (10 mL). The reaction was stirred at rt for 1 h. Volatiles were removed under reduced pressure and the solid obtained (E,Z)-N′-((5-bromopyridin-2-yl)methylene)-4-methylbenzenesulfonohydrazide was used as such in the following step.

Synthesis of 6-bromo-[1,2,3]triazolo[1,5-a]pyridine

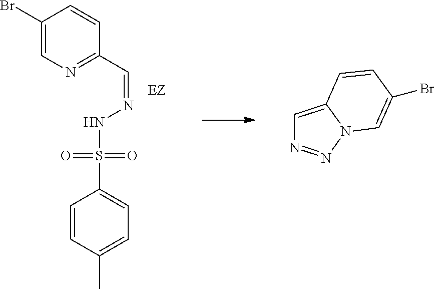 [SMILES: Cc1ccc(S(=O)(=O)N/N=C\c2ccc(Br)cn2)cc1 ; Brc1ccc2cnnn2c1]

A mixture of (E,Z)-N′-((5-bromopyridin-2-yl)methylene)-4-methylbenzene-sulfonohydrazide (1.90 g, 5.364 mmol) and [110-91-8] (10 mL, 115.9 mmol) were stirred at 90° C. for 1 h. The reaction was cooled down to rt and then cooled to 0° C. and treated with DIPE until a precipitate formed. The solid (morpholine toluenesulphinate) was discarded and the filtrate (containing the product) was evaporated under reduced pressure. The crude prodcut was purified by FCC on silica gel (40 g, gradient: from heptane 100% up to hept/EtOAc 4/6) to obtain 6-bromo-[1,2,3]triazolo[1,5-a]pyridine (970 mg, 91% yield) as a white solid.

Synthesis of 7-bromo-3-fluoroimidazo[1,2-a]pyridine

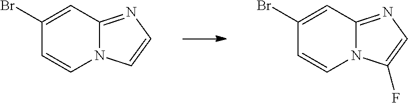 [SMILES: Brc1ccn2ccnc2c1 ; Fc1cnc2cc(Br)ccn12]

7-Bromoimidazo[1,2-a]pyridine [808744-34-5] (1.0 g, 5.08 mmol) was dissolved in dry THF (20 mL) and the solution was cooled down to 0° C. NaH (60% dispersion in mineral oil) [7646-69-7] (264 mg, 6.60 mmol) was added. After 5 min, Selectofluor

[55-6] (2.70 g, 7.61 mmol) was added and the reaction was allowed to warm up to rt and then heated to 60° C. for 16 h. The reaction was cooled down to rt, quenched with water (15 mL) and diluted with EtOAc (30 mL). The organic layer was separated and the aqueous one was extracted with EtOAc (2×20 mL). The combined organic layers were dried over MgSO₄, filtered and concnetrated in vacuo. The crude was purified by FCC on silica gel (40 g, gradient: from heptane 100% up to hept/EtOAc 1/1) to obtain 7-bromo-3-fluoroimidazo[1,2-a]pyridine (331 mg, yield 30%) as a white solid.

Synthesis of 6-((diphenylmethylene)amino)imidazo[1,5-a]pyridine-1-carbonitrile

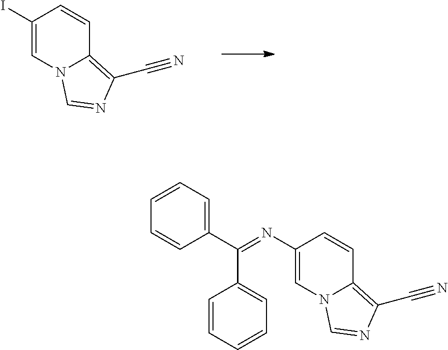 [SMILES: N#Cc1ncn2cc(I)ccc12 ; N#Cc1ncn2cc(N=C(c3ccccc3)c3ccccc3)ccc12]

A mixture of 6-iodoimidazo[1,5-a]pyridine-1-carbonitrile (300 mg, 1.115 mmol), benzophenone imine [1013-88-3] (0.281 mL, 1.673 mmol), BINAP [98327-87-8] (138.8 mg, 0.223 mmol) and sodium tert-butoxide [865-48-5] (171.5 mg, 1.784 mmol) in anhydrous 1,4-dioxane (12 mL) was degassed by bubbling N2 for a few minutes. Pd₂(dba)3 [51364-51-3] (102.1 mg, 0.112 mmol) was added and the reaction was heated to 50° C. After 2 h, the temperature was raised to 60° C. and the reaction was stirred for 2 additional h. The reaction was cooled down to rt and filtered through celite (washing wiht EtOAc). The filtrate was concentrated under reduced pressure to give a brown paste. The crude product was purified by FCC on silica gel (40 g, gradient: from heptane 100% up to hept/EtOAc 6/4) to obtain 6-((diphenylmethylene)amino)imidazo[1,5-a]pyridine-1-carbonitrile (160 mg, yield 45%) as a yellow solid.

Structure analogs were synthesized using the same procedure.

| Starting material | Intermediate |
|---|---|
| 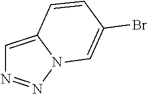 | 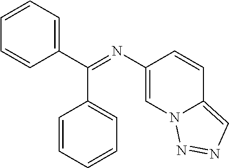 [SMILES: c1ccc(C(=Nc2ccc3cnnn3c2)c2ccccc2)cc1] |
| 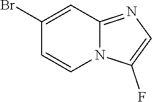 [SMILES: Fc1cnc2cc(Br)ccn12] | 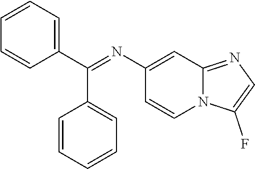 [SMILES: Fc1cnc2cc(N=C(c3ccccc3)c3ccccc3)ccn12] |

Synthesis of 6-aminoimidazo[1,5-a]pyridine-1-carbonitrile

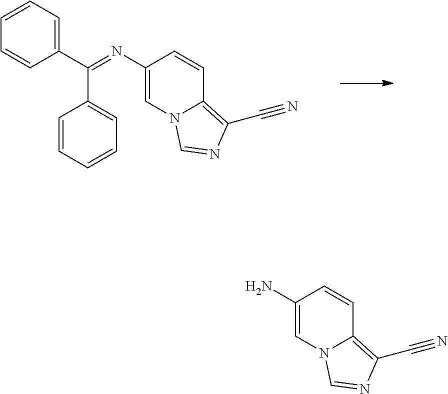 [SMILES: N#Cc1ncn2cc(N=C(c3ccccc3)c3ccccc3)ccc12 ; N#Cc1ncn2cc(N)ccc12]

HCl (1M in H₂O, (2.5 mL, 2.5 mmol) was added to a solution of 6-((diphenylmethylene)amino)imidazo[1,5-a]pyridine-1-carbonitrile (160 mg, 0.496 mmol) in THF (2 mL). The reaction was stirred at rt for 1 h. The reaction was diluted with DCM (10 ml) and transferred into a separating funnel. The organic layer was separated and the aqueous one was treated with sat aq NaHCO₃ until basic pH. The aqueous layer was then extracted with EtOAc (5×20 mL). The combined organic layers were dried over MgSO₄, filtered and evaporated to give 6-aminoimidazo[1,5-a]pyridine-1-carbonitrile (52 mg, yield 66%) as a light brown solid.

Structure analogs were synthesized using the same procedure.

| Starting material | Intermediate |
|---|---|
| 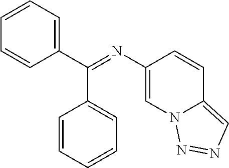 [SMILES: c1ccc(C(=Nc2ccc3cnnn3c2)c2ccccc2)cc1] | 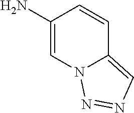 |
| 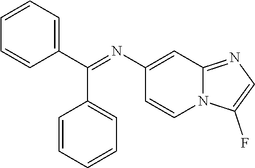 [SMILES: Fc1cnc2cc(N=C(c3ccccc3)c3ccccc3)ccn12] | 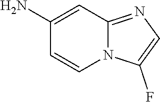 [SMILES: Nc1ccn2c(F)cnc2c1] |

Synthesis of (3-methoxypropyl)magnesium(II)

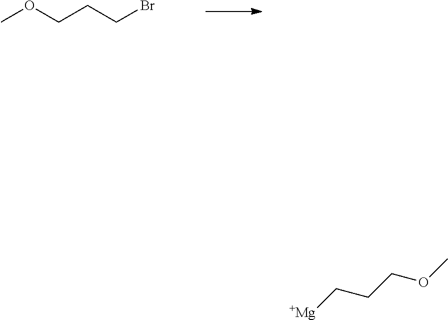

A solution of 1-bromo-3-methoxypropane [36865-41-5] (1071.13 mg, 7 mmol) in lithium chloride 0.5M in THF (7 mL, 0.5 M, 3.5 mmol) was pumped using R2/R4

Vapourtec through a column containing magnesium [7439-95-4] (4 g, 164.58 mmol) at 0.5 ml/min and at 40° C. The final solution was collected and titrated (0.45 M).

Synthesis of 2-(hex-5-yn-1-yloxy)tetrahydro-2H-pyran

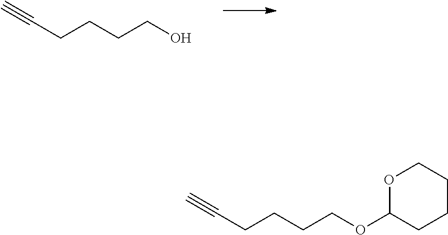

p-Toluenesulfonic acid monohydrate [6192-52-5] (0.17 g, 0.89 mmol) was added at 0° C. to a solution of 5-hexyn-1-ol [928-90-5] (8.77 g, 89.4 mmol) and 3,4-dihydro-2H-pyran [110-87-2] (9.78 g, 116.22 mmol) in DCM (100 mL). The resulting mixture was stirred overnight at rt. The mixture was washed with H₂O and brine. The organic layer was dried (MgSO₄), filtered and the solvents evaporated in vacuo. The crude product was purified by flash column chromatography (silica 330g; AcOEt in heptane 0/100 to 10/90). The desired fractions were collected and concentrated in vacuo to yield 2-(hex-5-yn-1-yloxy)tetrahydro-2H-pyran (12.09 g, yield 70%) as a colourless oil.

1H NMR (300 MHz, CDCl₃) d 4.60 (m, 1H), 3.88 (m, 1H), 3.77 (m, 1H), 3.54 (m, 1H), 3.45 (m, 1H), 2.25 (td, J=6.9, 2.6 Hz, 2H), 1.97 (t, J=2.6 Hz, 1H), 1.72 (m, 8H).

Synthesis of 2-(hept-5-yn-1-yloxy)tetrahydro-2H-pyran

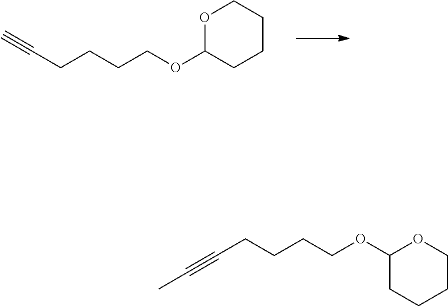

2.5M n-Butyl lithium [109-72-8] (42.8 mL, 2.5 M, 106.99 mmol) was added to a solution of 2-(hex-5-yn-1-yloxy)tetrahydro-2H-pyran (13 g, 71.33 mmol) in THF(300 mL) at −78° C. under nitrogen atmosphere. The reaction was stirred for 2 hours at 0° C. Iodomethane [74-88-4] (7.1 mL, 2.28 g/mL, 114.13 mmol) was added to the reaction at −78° C. The reaction was stirred at room temperature for 16 hours. The reaction was quenched with NH₄Cl and the aqueous phase was extracted with AcOEt. The organic phase was dried (MgSO₄), filtered and the solvent evaporated in vacuo. The crude product was purified by flash column chromatography (silica 330g; AcOEt in heptane 0/100 to 10/90) to yield 2-(hept-5-yn-1-yloxy)tetrahydro-2H-pyran (11.54 g, 76% pure, yield 63%) as a colourless oil.

1H NMR (400 MHz, CDCl₃) d 4.51 (m, 1H), 3.80 (m, 1H), 3.67 (m, 1H), 3.45 (m, 1H), 3.35 (m, 1H), 2.10 (m, 2H), 1.71 (t, J=2.5 Hz, 3H), 1.63 (m, 3H), 1.47 (m, 7H).

Synthesis of hept-5-yn-1-ol

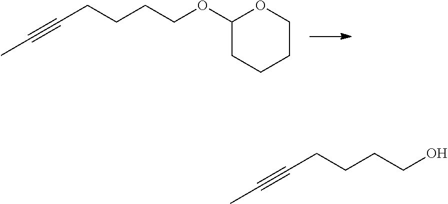

Pyridinium p-toluenesulfonate [24057-28-1] (233 mg, 0.93 mmol) was added to a solution of 2-(hept-5-yn-1-yloxy)tetrahydro-2H-pyran (11.95 g, 46.27 mmol) in methanol (210 mL). The mixture was stirred at room temperature overnight. The mixture was diluted with water and extracted with ether. The organic phase was dried (MgSO₄), filtered and the solvent evaporated in vacuo to dryness. The crude was purified by flash column chromatography (silica 120g; AcOEt/Heptane from 0/100 to 10/90). The desired fractions were collected and concentrated in vacuo toto yield hept-5-yn-1-ol (3.16 g, yield 58%) as a colourless oil.

1H NMR (400 MHz, CDCl₃) d 3.61 (t, J=6.4 Hz, 2H), 2.10 (s, 2H), 1.71 (t, J=2.6 Hz, 3H), 1.61 (s, 2H), 1.49 (s, 2H), 1.34 (s, 1H).

Synthesis of 3-methylenecyclobutane-1-carboxylate

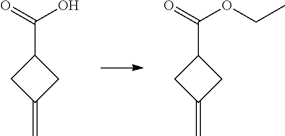

To a solution of 3-methylenecyclobutane-1-carboxylic acid [15760-36-8] (10 g, 89.18 mmol) and iodoethane [75-03-6] (14.34 mL, 178.37 mmol) in DMF dry (150 mL), Cs₂CO_{3 [534}-17-8] (72.65 g, 222.96 mmol) was added and the mixture was stirred at room temperature for 15 hr. The mixture was diluted with ethylacetate and washed with brine (5X). The organic layer was dried (MgSO₄), filtered and concentrated to yield 3-methylenecyclobutane-1-carboxylate (10600 mg, yield 85%) as a colourless oil.

1H NMR (300 MHz, CDCl₃) d 4.84 — 4.74 (m, 2H), 4.14 (q, J=7.1 Hz, 2H), 3.20 — 2.81 (m, 5H), 1.25 (t, J=7.1 Hz, 3H).

Synthesis of 3-(hydroxymethyl)cyclobutane-1-carboxylate

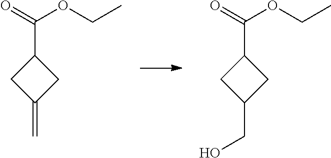

A solution of ethyl 3-methylenecyclobutane-1-carboxylate (2.5 g, 17.83 mmol) in tetrahydrofurane (25 mL) was treated with Borane-Dimethylsulfide complex 2M in THF [13292-87-0] (0.85 mL, 0.8 g/mL, 8.92 mmol) and the mixture was stirred at room temperature for 2.5 hr. A suspension of sodium perborate [10332-33-9] (2201 mg, 21.4 mmol) in water (15 mL) was added followed by dioxane (15 mL). The mixture was heated to 65° C. for 2 hr, then allowed to cool to room temperature for 15 hr. The mixture was diluted with water and extracted twice with ethylacetate. The combined organic layers were dried (MgSO₄), filtered and concentrad to yield 3-(hydroxymethyl)cyclobutane-1-carboxylate (1440 mg, yield 51%) as a colourless oil, mixture of isomers.

1H NMR (300 MHz, CDCl₃) d 4.22 — 4.05 (m, 2H), 3.70 — 3.57 (m, 2H), 3.20 — 2.96 (m, 1H), 2.66 — 2.22 (m, 3H), 2.13 — 1.98 (m, 2H), 1.32 — 1.19 (m, 3H).

Synthesis of ethyl 3-(iodomethyl)cyclobutane-1-carboxylate

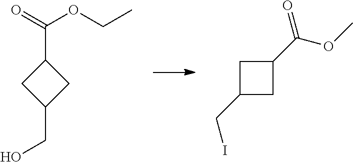

To a stirred soluiton of ethyl 3-(hydroxymethyl)cyclobutane-1-carboxylate (6036 mg, 38.16 mmol), imidazole [288-32-4] (3892 mg, 57.18 mmol) and triphenylphosphine [603-35-0] (10997 mg, 41.93 mmol) in tetrahydrofurane (200 mL) at 0° C. under nitrogen, iodine [7553-56-2] (10642 mg, 41.93 mmol) was added portionwise, and the mixture was stirred at 0° C. for 30 min, then at room temperature for 15 hr. The mixture was diluted with ethylacetate (200 ml) and washed with Na₂S2O_{3 0.05} M solution (200 ml) and with brine. The aqueous layers were re-extracted with ethylacetate. The combined organic layers were dried (MgSO₄) and concentrated. The crude was purified by flash chromatography (SiO2, 120 g, Ethylacetate-heptane gradient from 5% to 20%). The product fractions were combined and concentrated to yield 3-(iodomethyl)cyclobutane-1-carboxylate (7.8 g, yield 76.3%) as a colourless oil.

1H NMR (400 MHz, CDCl₃) d 4.18 — 4.09 (m, 2H), 3.30 — 3.14 (m, 2H), 3.07 — 2.50 (m, 2H), 2.43 — 2.28 (m, 2H), 2.00 — 1.82 (m, 2H), 1.30 — 1.19 (m, 3H).

Additional analogs were accessed using similar reaction conditions, using the appropriate reagent.

| Starting material | Intermediate |
|---|---|
| 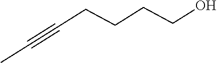 | 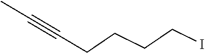 |

Synthesis of ((3-(ethoxycarbonyl)cyclobutyl)methyl)zinc(II) iodide

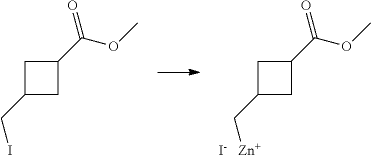

Dry THF (10 mL) was passed through a 10 mm internal diameter column containing

Zn (10 g) using a syringe pump at room temperature and 1mL/min flow rate. A solution of chlorotrimethylsilane [75-77-4] (1.2 mL, 9.45 mmol) and 1-bromo-2-chloroethane [107-04-0] (0.4 mL, 4.83 mmol) in dry THF (10 ml) was prepared under nitrogen (N2) atmosphere in a dried flask and passed through the Zn column using a syringe pump at room temperature and 1 mL/min flow rate. Then, dry THF (10 mL) and dry DMF (10 ml) were sequentially passed through the Zn column at room temperature and 1 mL/min, in order to stabilized the column. A solution of ethyl 3-(iodomethyl)cyclobutane-1-carboxylate (335 mg, 1.25 mmol) in dry DMF (4.4 ml) was passed through the column containing activated Zn using a syringe pump at 55° C. and flow rate of 0.4 mL/min. The outcoming solution of ((3-(ethoxycarbonyl)cyclobutyl)methyl)zinc(II) iodide was collected in a closed flask under nitrogen atmosphere.

Additional analogs were accessed using similar reaction conditions, using the appropriate reagent.

| Starting material | Intermediate |
|---|---|
| 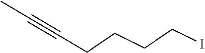 | 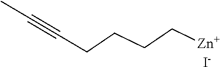 |

### Example A1

Synthesis of 2-(2-ethyl-7-isopropyl-4-oxo-pyrazolo[1,5-d][1,2,4]triazin-5-yl)-N-pyrimidin-4-yl-acetamide (Final compound 1)

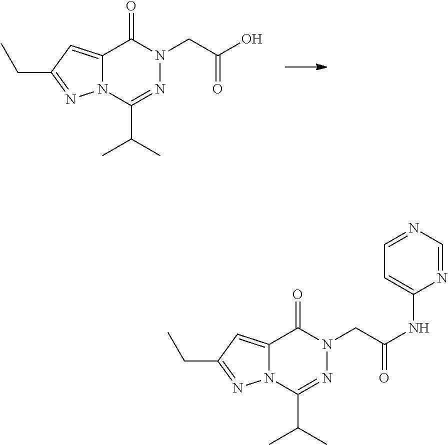 [SMILES: CCc1cc2c(=O)n(CC(=O)O)nc(C(C)C)n2n1 ; CCc1cc2c(=O)n(CC(=O)Nc3ccncn3)nc(C(C)C)n2n1]

4-Aminopyrimidine [591-54-8] (61 mg, 0.64 mmol), followed by HATU [148893-10-1] (0.26 g, 0.68 mmol) and Et₃N [121-44-8] (0.18 mL, 1.33 mmol) were added to a stirred solution of 2-(2-ethyl-7-isopropyl-4-oxo-pyrazolo [1,5-d] [1,2,4]triazin-5-yl)acetic acid (I-14) (117 mg, 0.44 mmol) in D1VIF (2.2 mL) under N2. The mixture was stirred at rt for 16 h. The mixture was treated with saturated aqueous solution of NaHCO₃ and EtOAc and stirred for 15 min. The mixture was diluted with more EtOAc and washed with saturated aqueous solution of NaHCO₃ and 20% aqueous solution of

NH₄C1. The organic layer was separated, dried (MgSO₄), filtered and the solvents evaporated in vacuo. The crude product was purified by RP flash chromatography (C18, ACN in NH₄HCO_{3 0.25}% solution in water 10/90 to 100/0). The desired fractions were collected and extracted with DCM. The organic layer was separated, dried (MgSO₄), filtered and the solvents evaporated in vacuo to yield 2-(2-ethyl-7-isopropyl-4-oxo-pyrazolo[1,5-d] [1,2,4]triazin-5-yl)-N-pyrimidin-4-yl-acetamide (Final compound 1) (80 mg, 53%) as a white solid.

Additional analogs were accessed using similar reaction conditions, using the appropriate reagent and conditions.

A=Et3N, DMF

B=DIPEA, DMF

C=Et3N, DCM

| | | | Num- |
|---|---|---|---|
| Reagent | Intermediate | Final compound | ber |
| 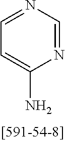 | 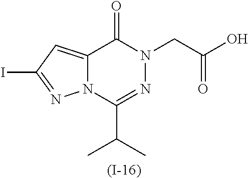 | 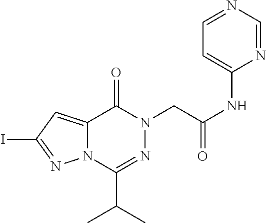 [SMILES: CC(C)c1nn(CC(=O)Nc2ccncn2)c(=O)c2cc(I)nn12] | 2 |
| 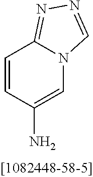 | 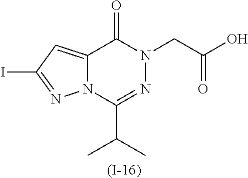 | 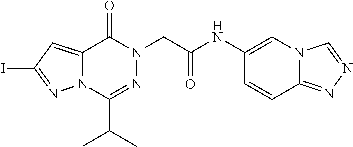 [SMILES: CC(C)c1nn(CC(=O)Nc2ccc3nncn3c2)c(=O)c2cc(I)nn12] | 23 |
| 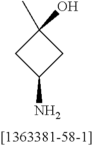 | 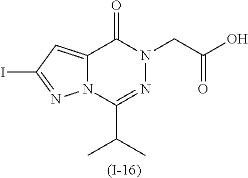 | 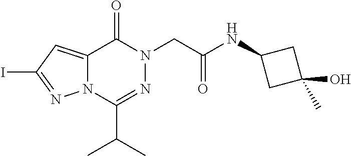 [SMILES: CC(C)c1nn(CC(=O)N[C@H]2C[C@@](C)(O)C2)c(=O)c2cc(I)nn12] | 24 |
| 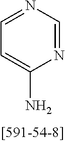 | 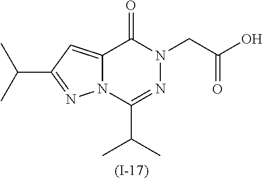 | 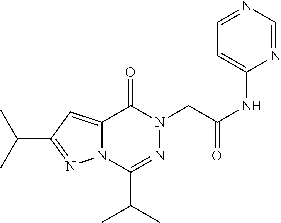 [SMILES: CC(C)c1cc2c(=O)n(CC(=O)Nc3ccncn3)nc(C(C)C)n2n1] | 3 |
| 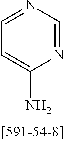 | 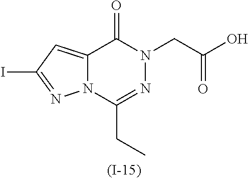 | 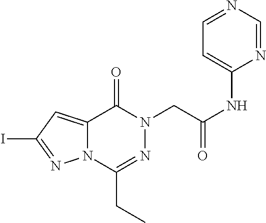 [SMILES: CCc1nn(CC(=O)Nc2ccncn2)c(=O)c2cc(I)nn12] | 4 |
| 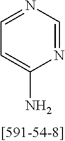 | 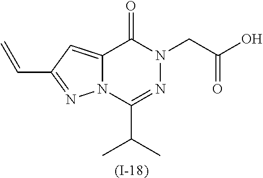 | 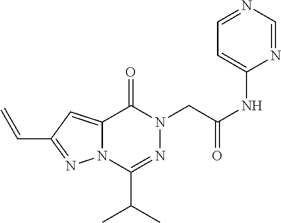 [SMILES: C=Cc1cc2c(=O)n(CC(=O)Nc3ccncn3)nc(C(C)C)n2n1] | 5 |
| 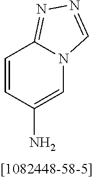 | 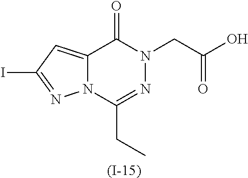 | 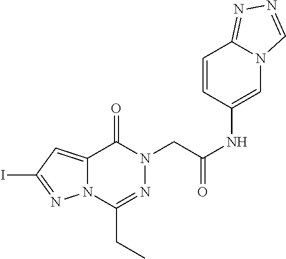 [SMILES: CCc1nn(CC(=O)Nc2ccc3nncn3c2)c(=O)c2cc(I)nn12] | 6 |
| 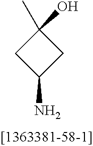 | 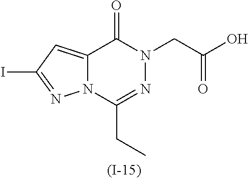 | 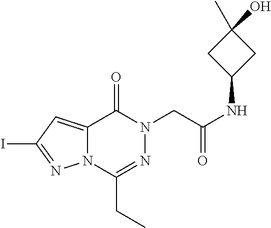 [SMILES: CCc1nn(CC(=O)N[C@H]2C[C@@](C)(O)C2)c(=O)c2cc(I)nn12] | 7 |
| 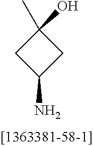 | 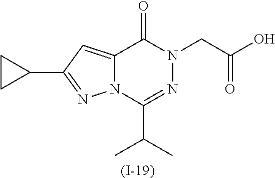 | 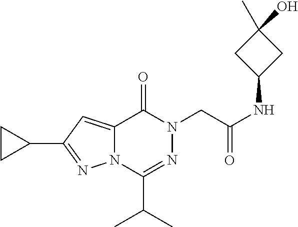 [SMILES: CC(C)c1nn(CC(=O)N[C@H]2C[C@@](C)(O)C2)c(=O)c2cc(C3CC3)nn12] | 8 |
| 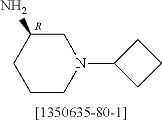 | 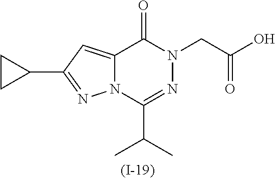 | 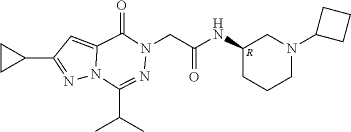 | 9 |
| 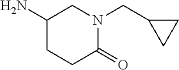 [SMILES: NC1CCC(=O)N(CC2CC2)C1] | 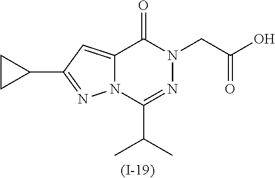 | 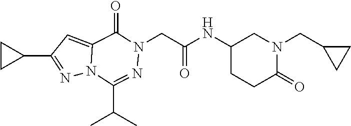 [SMILES: CC(C)c1nn(CC(=O)NC2CCC(=O)N(CC3CC3)C2)c(=O)c2cc(C3CC3)nn12] | 25 |
| 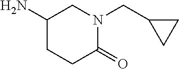 [SMILES: NC1CCC(=O)N(CC2CC2)C1] | 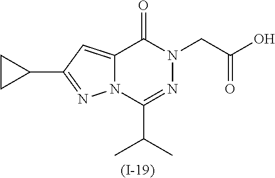 | 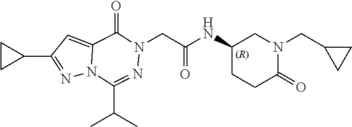 | 26 |
| 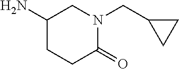 [SMILES: NC1CCC(=O)N(CC2CC2)C1] | 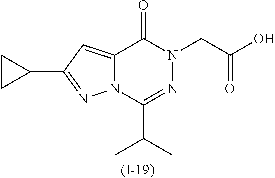 | 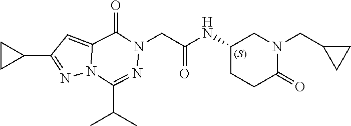 | 27 |
| 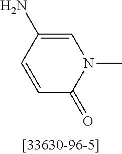 | 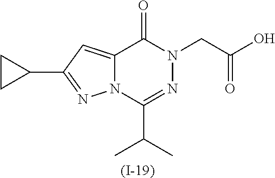 | 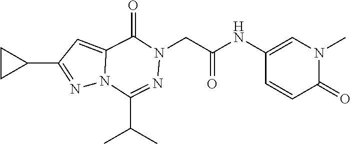 [SMILES: CC(C)c1nn(CC(=O)Nc2ccc(=O)n(C)c2)c(=O)c2cc(C3CC3)nn12] | 28 |
| 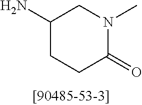 | 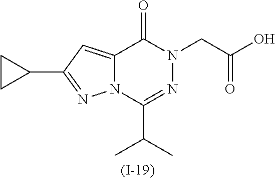 | 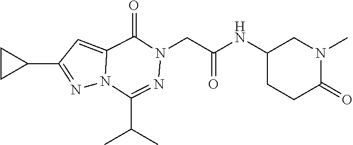 [SMILES: CC(C)c1nn(CC(=O)NC2CCC(=O)N(C)C2)c(=O)c2cc(C3CC3)nn12] | 29 |
| 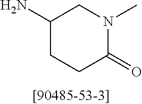 | 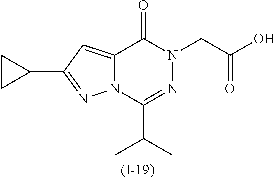 | 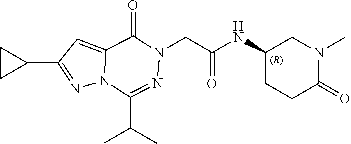 | 30 |
| 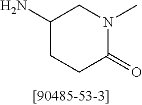 | 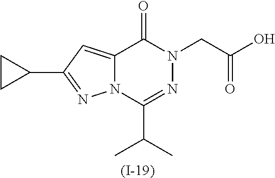 | 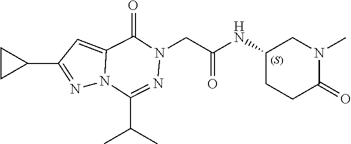 | 31 |
| 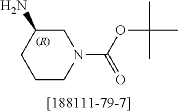 | 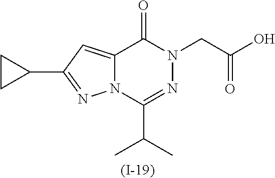 | 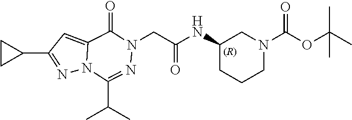 | |
| 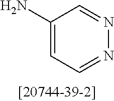 | 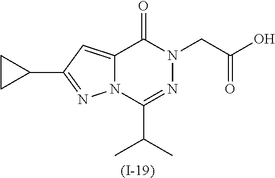 | 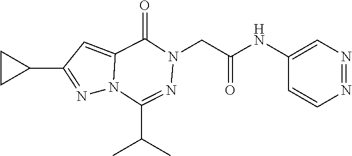 [SMILES: CC(C)c1nn(CC(=O)Nc2ccnnc2)c(=O)c2cc(C3CC3)nn12] | 32 |
| 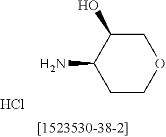 | 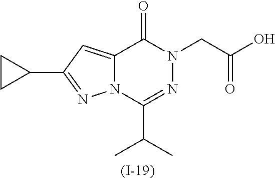 | 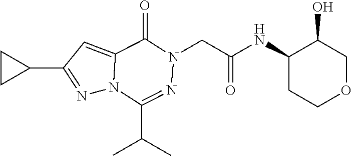 [SMILES: CC(C)c1nn(CC(=O)N[C@@H]2CCOC[C@@H]2O)c(=O)c2cc(C3CC3)nn12] | 33 |
| 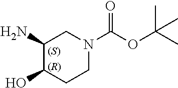 | 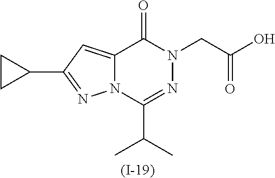 | 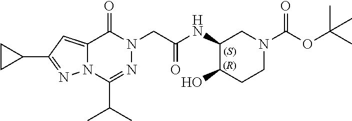 | 34 |
| 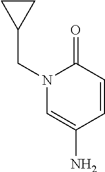 [SMILES: Nc1ccc(=O)n(CC2CC2)c1] | 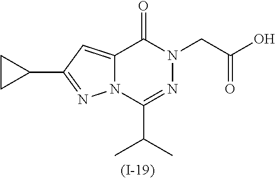 | 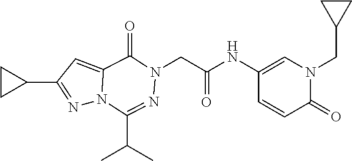 [SMILES: CC(C)c1nn(CC(=O)Nc2ccc(=O)n(CC3CC3)c2)c(=O)c2cc(C3CC3)nn12] | 35 |
| 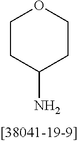 | 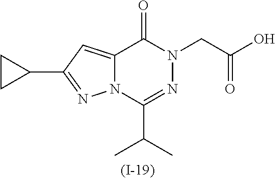 | 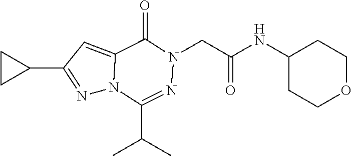 [SMILES: CC(C)c1nn(CC(=O)NC2CCOCC2)c(=O)c2cc(C3CC3)nn12] | 36 |
| 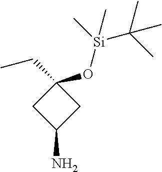 | 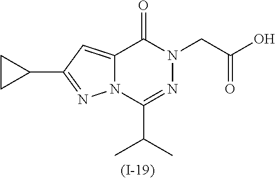 | 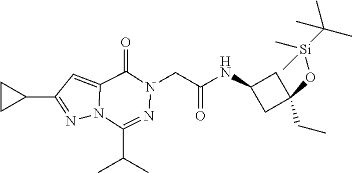 [SMILES: CC[C@]1(O[Si](C)(C)C(C)(C)C)C[C@@H](NC(=O)Cn2nc(C(C)C)n3nc(C4CC4)cc3c2=O)C1] | |
| 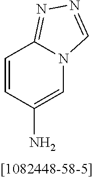 | 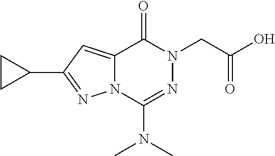 [SMILES: CN(C)c1nn(CC(=O)O)c(=O)c2cc(C3CC3)nn12] | 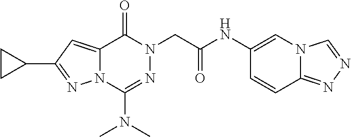 [SMILES: CN(C)c1nn(CC(=O)Nc2ccc3nncn3c2)c(=O)c2cc(C3CC3)nn12] | 37 |
| 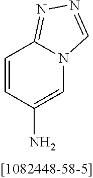 | 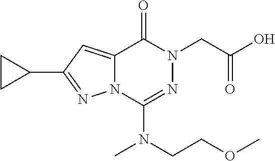 [SMILES: COCCN(C)c1nn(CC(=O)O)c(=O)c2cc(C3CC3)nn12] | 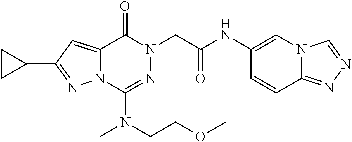 [SMILES: COCCN(C)c1nn(CC(=O)Nc2ccc3nncn3c2)c(=O)c2cc(C3CC3)nn12] | 38 |
| 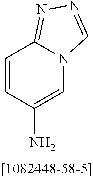 | 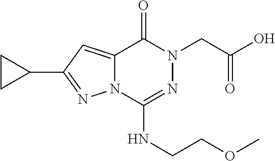 [SMILES: COCCNc1nn(CC(=O)O)c(=O)c2cc(C3CC3)nn12] | 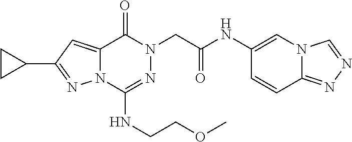 [SMILES: COCCNc1nn(CC(=O)Nc2ccc3nncn3c2)c(=O)c2cc(C3CC3)nn12] | 39 |
| 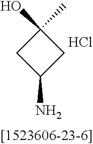 | 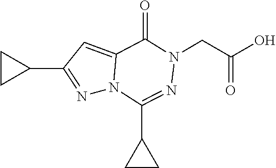 [SMILES: O=C(O)Cn1nc(C2CC2)n2nc(C3CC3)cc2c1=O] | 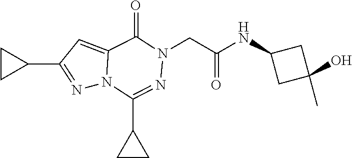 [SMILES: C[C@]1(O)C[C@@H](NC(=O)Cn2nc(C3CC3)n3nc(C4CC4)cc3c2=O)C1] | 40 |
| 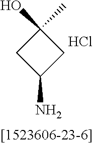 | 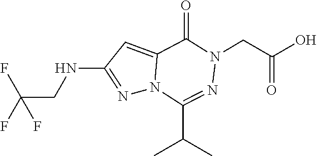 [SMILES: CC(C)c1nn(CC(=O)O)c(=O)c2cc(NCC(F)(F)F)nn12] | 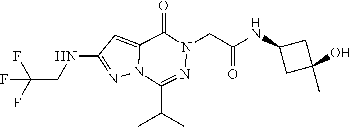 [SMILES: CC(C)c1nn(CC(=O)N[C@H]2C[C@@](C)(O)C2)c(=O)c2cc(NCC(F)(F)F)nn12] | 41 |
| 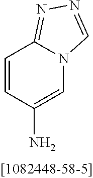 | 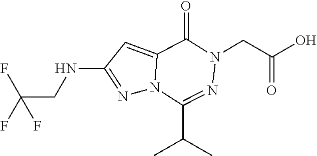 [SMILES: CC(C)c1nn(CC(=O)O)c(=O)c2cc(NCC(F)(F)F)nn12] | 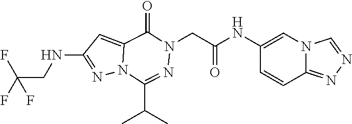 [SMILES: CC(C)c1nn(CC(=O)Nc2ccc3nncn3c2)c(=O)c2cc(NCC(F)(F)F)nn12] | 42 |
| 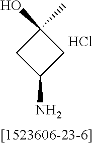 | 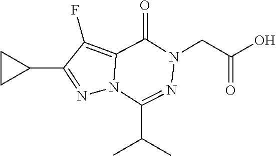 [SMILES: CC(C)c1nn(CC(=O)O)c(=O)c2c(F)c(C3CC3)nn12] | 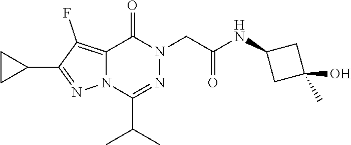 [SMILES: CC(C)c1nn(CC(=O)N[C@H]2C[C@@](C)(O)C2)c(=O)c2c(F)c(C3CC3)nn12] | 43 |
| 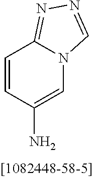 | 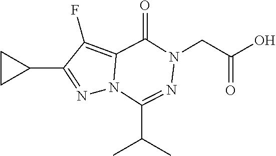 [SMILES: CC(C)c1nn(CC(=O)O)c(=O)c2c(F)c(C3CC3)nn12] | 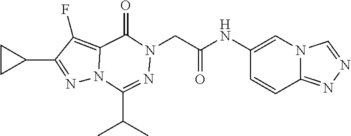 [SMILES: CC(C)c1nn(CC(=O)Nc2ccc3nncn3c2)c(=O)c2c(F)c(C3CC3)nn12] | 44 |
| 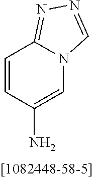 | 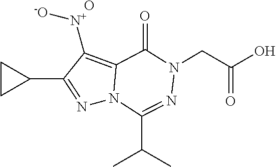 | 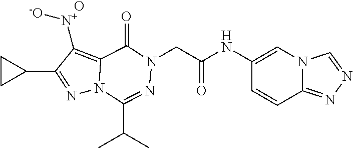 | 45 |
| 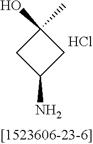 | 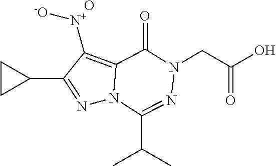 | 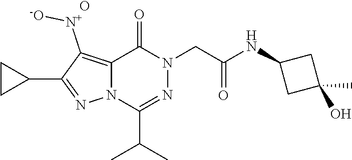 | 46 |
| 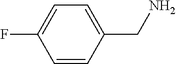 | 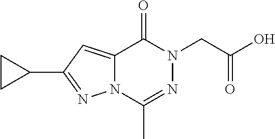 [SMILES: Cc1nn(CC(=O)O)c(=O)c2cc(C3CC3)nn12] | 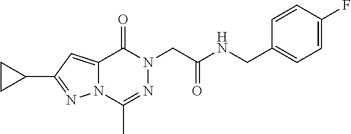 [SMILES: Cc1nn(CC(=O)NCc2ccc(F)cc2)c(=O)c2cc(C3CC3)nn12] | 47 |
| 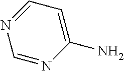 | 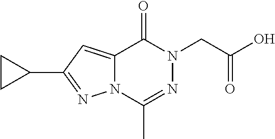 [SMILES: Cc1nn(CC(=O)O)c(=O)c2cc(C3CC3)nn12] | 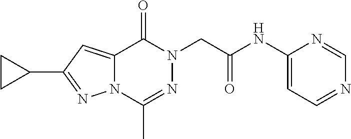 [SMILES: Cc1nn(CC(=O)Nc2ccncn2)c(=O)c2cc(C3CC3)nn12] | 48 |
| 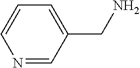 | 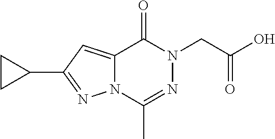 [SMILES: Cc1nn(CC(=O)O)c(=O)c2cc(C3CC3)nn12] | 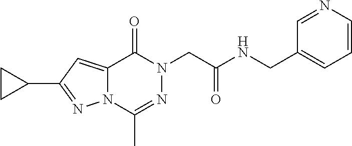 [SMILES: Cc1nn(CC(=O)NCc2cccnc2)c(=O)c2cc(C3CC3)nn12] | 49 |
| 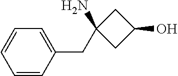 [SMILES: N[C@]1(Cc2ccccc2)C[C@H](O)C1] | 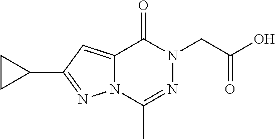 [SMILES: Cc1nn(CC(=O)O)c(=O)c2cc(C3CC3)nn12] | 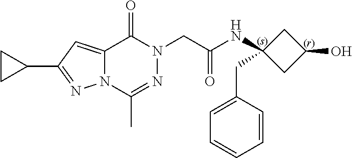 [SMILES: Cc1nn(CC(=O)N[C@]2(Cc3ccccc3)C[C@H](O)C2)c(=O)c2cc(C3CC3)nn12] | 50 |
| 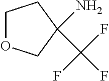 | 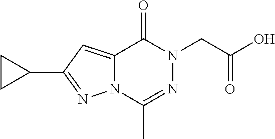 [SMILES: Cc1nn(CC(=O)O)c(=O)c2cc(C3CC3)nn12] | 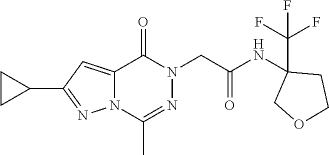 [SMILES: Cc1nn(CC(=O)NC2(C(F)(F)F)CCOC2)c(=O)c2cc(C3CC3)nn12] | 51 |
| 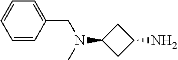 | 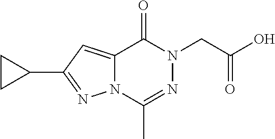 [SMILES: Cc1nn(CC(=O)O)c(=O)c2cc(C3CC3)nn12] | 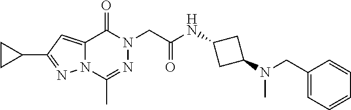 [SMILES: Cc1nn(CC(=O)N[C@H]2C[C@H](N(C)Cc3ccccc3)C2)c(=O)c2cc(C3CC3)nn12] | 52 |
| 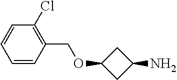 [SMILES: N[C@H]1C[C@@H](OCc2ccccc2Cl)C1] | 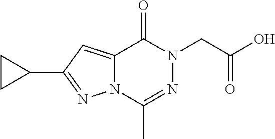 [SMILES: Cc1nn(CC(=O)O)c(=O)c2cc(C3CC3)nn12] | 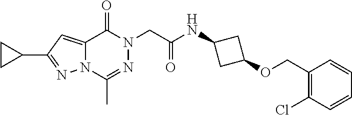 [SMILES: Cc1nn(CC(=O)N[C@H]2C[C@@H](OCc3ccccc3Cl)C2)c(=O)c2cc(C3CC3)nn12] | 53 |
| 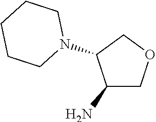 [SMILES: N[C@@H]1COC[C@H]1N1CCCCC1] | 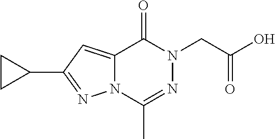 [SMILES: Cc1nn(CC(=O)O)c(=O)c2cc(C3CC3)nn12] | 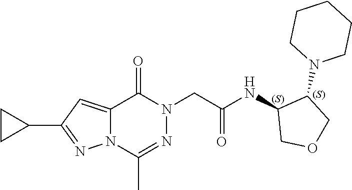 | 54 |
| 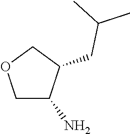 | 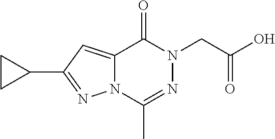 [SMILES: Cc1nn(CC(=O)O)c(=O)c2cc(C3CC3)nn12] | 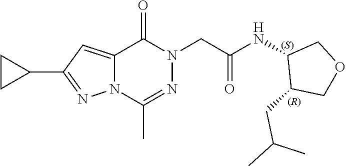 | 55 |
| 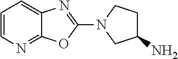 [SMILES: N[C@@H]1CCN(c2nc3cccnc3o2)C1] | 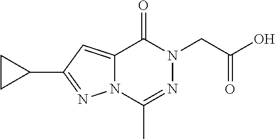 [SMILES: Cc1nn(CC(=O)O)c(=O)c2cc(C3CC3)nn12] | 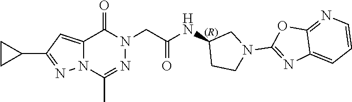 | 56 |
| 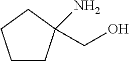 | 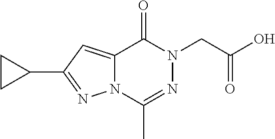 [SMILES: Cc1nn(CC(=O)O)c(=O)c2cc(C3CC3)nn12] | 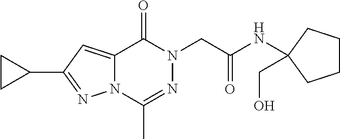 [SMILES: Cc1nn(CC(=O)NC2(CO)CCCC2)c(=O)c2cc(C3CC3)nn12] | 57 |
| 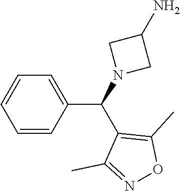 [SMILES: Cc1noc(C)c1[C@@H](c1ccccc1)N1CC(N)C1] | 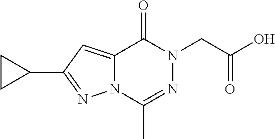 [SMILES: Cc1nn(CC(=O)O)c(=O)c2cc(C3CC3)nn12] | 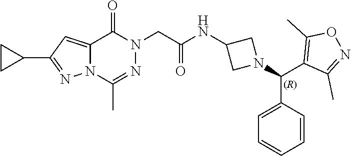 | 58 |
| 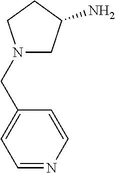 [SMILES: N[C@H]1CCN(Cc2ccncc2)C1] | 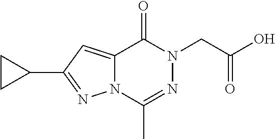 [SMILES: Cc1nn(CC(=O)O)c(=O)c2cc(C3CC3)nn12] | 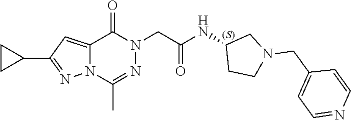 | 59 |
| 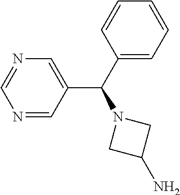 [SMILES: NC1CN([C@H](c2ccccc2)c2cncnc2)C1] | 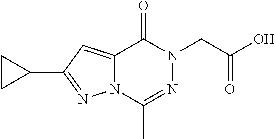 [SMILES: Cc1nn(CC(=O)O)c(=O)c2cc(C3CC3)nn12] | 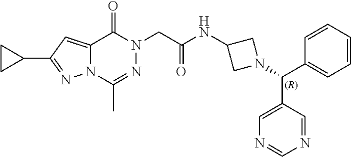 | 60 |
| 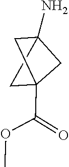 | 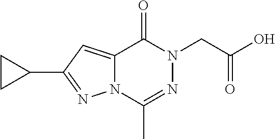 [SMILES: Cc1nn(CC(=O)O)c(=O)c2cc(C3CC3)nn12] | 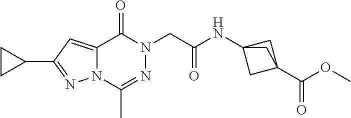 [SMILES: COC(=O)C12CC(NC(=O)Cn3nc(C)n4nc(C5CC5)cc4c3=O)(C1)C2] | 61 |
| 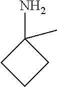 | 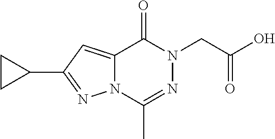 [SMILES: Cc1nn(CC(=O)O)c(=O)c2cc(C3CC3)nn12] | 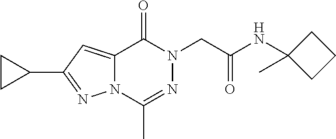 [SMILES: Cc1nn(CC(=O)NC2(C)CCC2)c(=O)c2cc(C3CC3)nn12] | 62 |
| 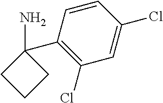 [SMILES: NC1(c2ccc(Cl)cc2Cl)CCC1] | 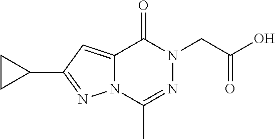 [SMILES: Cc1nn(CC(=O)O)c(=O)c2cc(C3CC3)nn12] | 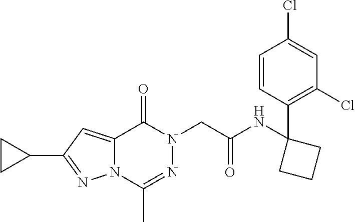 [SMILES: Cc1nn(CC(=O)NC2(c3ccc(Cl)cc3Cl)CCC2)c(=O)c2cc(C3CC3)nn12] | 63 |
| 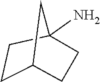 | 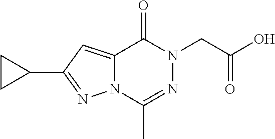 [SMILES: Cc1nn(CC(=O)O)c(=O)c2cc(C3CC3)nn12] | 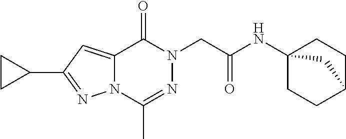 [SMILES: Cc1nn(CC(=O)N[C@]23CC[C@H](CC2)C3)c(=O)c2cc(C3CC3)nn12] | 64 |
| 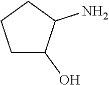 | 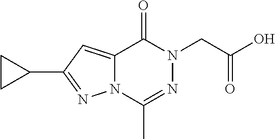 [SMILES: Cc1nn(CC(=O)O)c(=O)c2cc(C3CC3)nn12] | 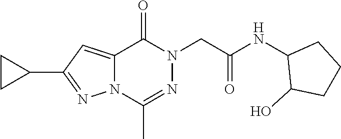 [SMILES: Cc1nn(CC(=O)NC2CCCC2O)c(=O)c2cc(C3CC3)nn12] | 65 |
| 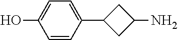 [SMILES: NC1CC(c2ccc(O)cc2)C1] | 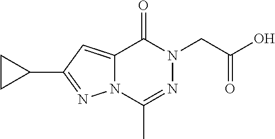 [SMILES: Cc1nn(CC(=O)O)c(=O)c2cc(C3CC3)nn12] | 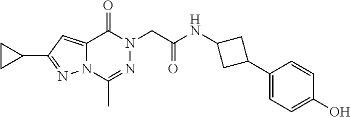 [SMILES: Cc1nn(CC(=O)NC2CC(c3ccc(O)cc3)C2)c(=O)c2cc(C3CC3)nn12] | 66 |
| 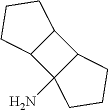 [SMILES: NC12CCCC1C1CCCC12] | 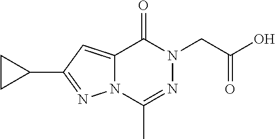 [SMILES: Cc1nn(CC(=O)O)c(=O)c2cc(C3CC3)nn12] | 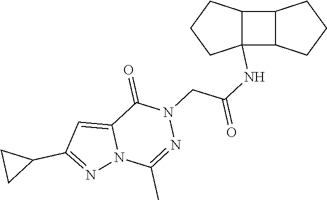 [SMILES: Cc1nn(CC(=O)NC23CCCC2C2CCCC23)c(=O)c2cc(C3CC3)nn12] | 67 |
| 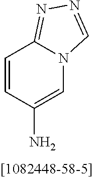 | 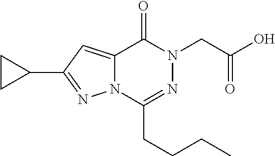 [SMILES: CCCCc1nn(CC(=O)O)c(=O)c2cc(C3CC3)nn12] | 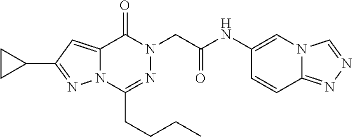 [SMILES: CCCCc1nn(CC(=O)Nc2ccc3nncn3c2)c(=O)c2cc(C3CC3)nn12] | 68 |
| 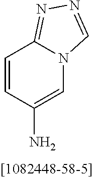 | 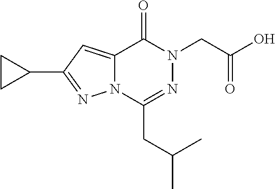 [SMILES: CC(C)Cc1nn(CC(=O)O)c(=O)c2cc(C3CC3)nn12] | 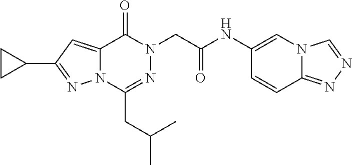 [SMILES: CC(C)Cc1nn(CC(=O)Nc2ccc3nncn3c2)c(=O)c2cc(C3CC3)nn12] | 69 |
| 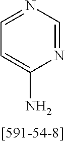 | 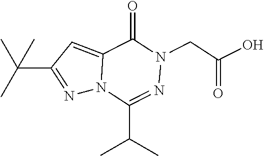 [SMILES: CC(C)c1nn(CC(=O)O)c(=O)c2cc(C(C)(C)C)nn12] | 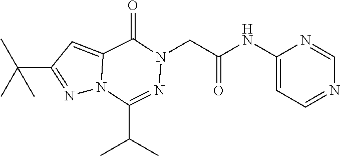 [SMILES: CC(C)c1nn(CC(=O)Nc2ccncn2)c(=O)c2cc(C(C)(C)C)nn12] | 70 |
| 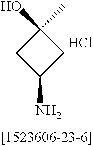 | 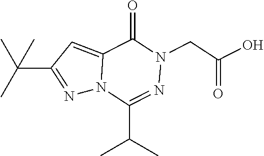 [SMILES: CC(C)c1nn(CC(=O)O)c(=O)c2cc(C(C)(C)C)nn12] | 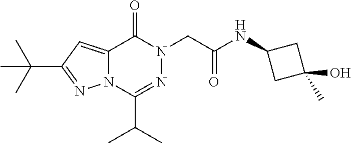 [SMILES: CC(C)c1nn(CC(=O)N[C@H]2C[C@@](C)(O)C2)c(=O)c2cc(C(C)(C)C)nn12] | 71 |
| 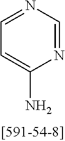 | 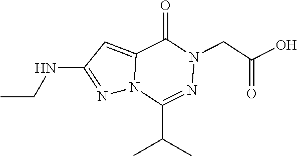 [SMILES: CCNc1cc2c(=O)n(CC(=O)O)nc(C(C)C)n2n1] | 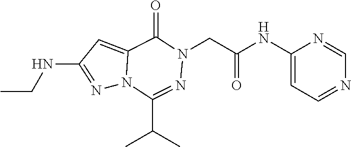 [SMILES: CCNc1cc2c(=O)n(CC(=O)Nc3ccncn3)nc(C(C)C)n2n1] | 72 |
| 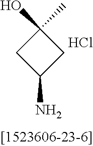 | 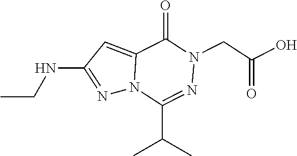 [SMILES: CCNc1cc2c(=O)n(CC(=O)O)nc(C(C)C)n2n1] | 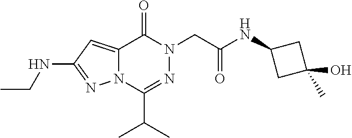 [SMILES: CCNc1cc2c(=O)n(CC(=O)N[C@H]3C[C@@](C)(O)C3)nc(C(C)C)n2n1] | 73 |
| 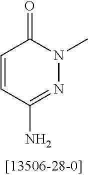 | 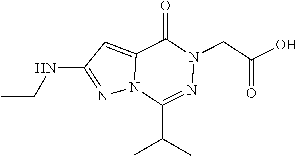 [SMILES: CCNc1cc2c(=O)n(CC(=O)O)nc(C(C)C)n2n1] | 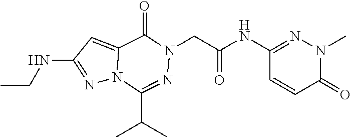 [SMILES: CCNc1cc2c(=O)n(CC(=O)Nc3ccc(=O)n(C)n3)nc(C(C)C)n2n1] | 74 |
| 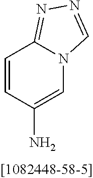 | 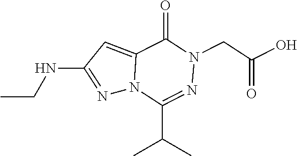 [SMILES: CCNc1cc2c(=O)n(CC(=O)O)nc(C(C)C)n2n1] | 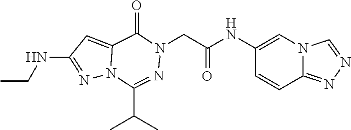 [SMILES: CCNc1cc2c(=O)n(CC(=O)Nc3ccc4nncn4c3)nc(C(C)C)n2n1] | 75 |

### Example A2

Synthesis of N-cyclopropyl-2-(7-ethyl-4-oxopyrazolo[1,5-d][1,2,4]triazin-5(4H)-yl)acetamide (final compound 76)

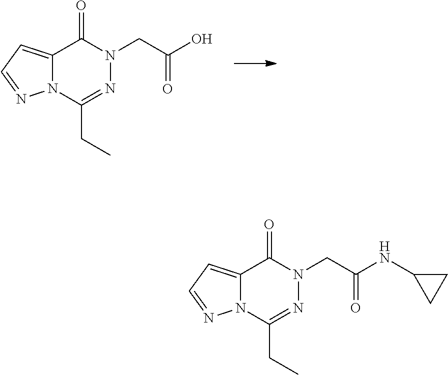 [SMILES: CCc1nn(CC(=O)O)c(=O)c2ccnn12 ; CCc1nn(CC(=O)NC2CC2)c(=O)c2ccnn12]

2-(7-ethyl-4-oxopyrazolo[1,5-d][1,2,4]triazin-5(4H)-yl)acetic acid (I-20) (120 mg, 0.54 mmol) was dissolved in DCM (1.38 mL). TEA [121-44-8] (149.72 0.73 g/mL, 1.08 mmol) and cyclopropylamine [765-30-0] (37 mg, 0.65 mmol) were added and finaly 1-propanephosponic anhydride [68957-94-8] (0.69 mL, 0.5 g/mL, 1.08 mmol) was added. The RM was stirred at rt for 2h.

The RM was poured out in 20 ml sat. NaHCO₃ solution and the organic layer was separated. The aquous phase was extracted once more with DCM and the combined organic layers were dried on MgSO₄. The solvent was evaporated and the residue was boiled in MeOH and the solids were filtered to yield N-cyclopropyl-2-(7-ethyl-4-oxopyrazolo[1,5-d][1,2,4]triazin-5(4H)-yl)acetamide (final compound 76) (60 mg, yield 43%) as a white solid.

Additional analogs were accessed using similar reaction conditions, using the appropriate reagent.

| | | | Num- |
|---|---|---|---|
| Reagent | Intermediate | Final compound | ber |
| 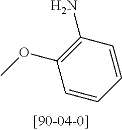 | 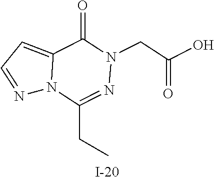 | 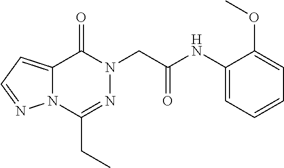 [SMILES: CCc1nn(CC(=O)Nc2ccccc2OC)c(=O)c2ccnn12] | 77 |
| 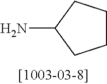 | 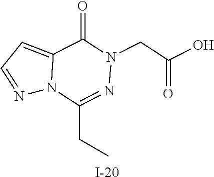 | 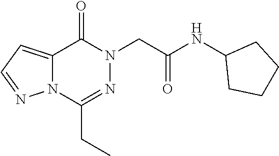 [SMILES: CCc1nn(CC(=O)NC2CCCC2)c(=O)c2ccnn12] | 78 |
| 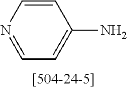 | 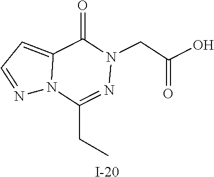 | 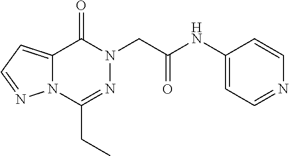 [SMILES: CCc1nn(CC(=O)Nc2ccncc2)c(=O)c2ccnn12] | 79 |
| 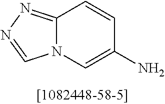 | 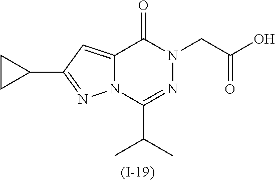 | 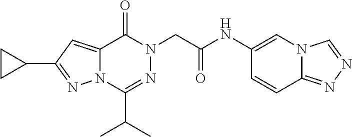 [SMILES: CC(C)c1nn(CC(=O)Nc2ccc3nncn3c2)c(=O)c2cc(C3CC3)nn12] | 80 |
| 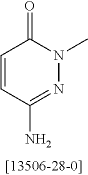 | 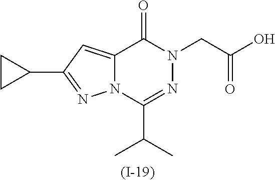 | 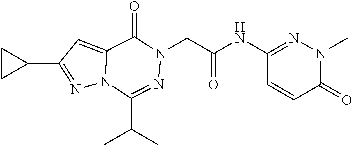 [SMILES: CC(C)c1nn(CC(=O)Nc2ccc(=O)n(C)n2)c(=O)c2cc(C3CC3)nn12] | 81 |
| 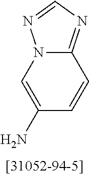 | 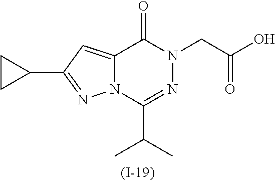 | 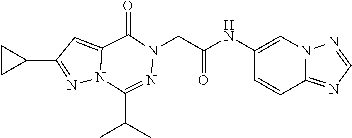 [SMILES: CC(C)c1nn(CC(=O)Nc2ccc3ncnn3c2)c(=O)c2cc(C3CC3)nn12] | 82 |
| 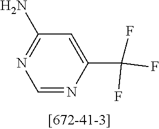 [SMILES: Nc1cc(C(F)(F)F)ncn1] | 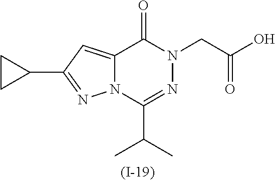 | 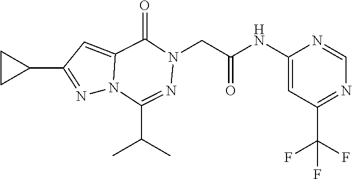 [SMILES: CC(C)c1nn(CC(=O)Nc2cc(C(F)(F)F)ncn2)c(=O)c2cc(C3CC3)nn12] | 83 |
| 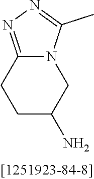 | 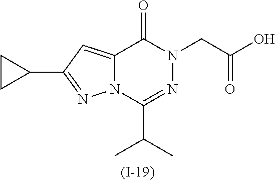 | 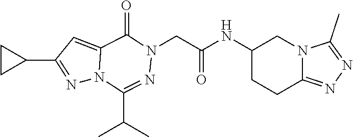 [SMILES: Cc1nnc2n1CC(NC(=O)Cn1nc(C(C)C)n3nc(C4CC4)cc3c1=O)CC2] | 84 |
| 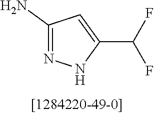 | 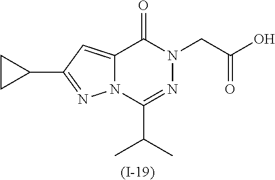 | 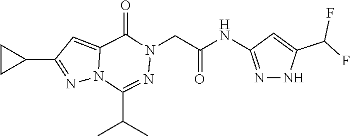 [SMILES: CC(C)c1nn(CC(=O)Nc2cc(C(F)F)[nH]n2)c(=O)c2cc(C3CC3)nn12] | 85 |
| 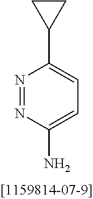 | 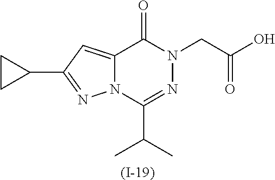 | 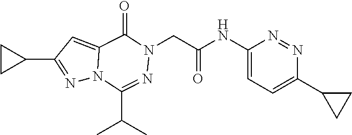 [SMILES: CC(C)c1nn(CC(=O)Nc2ccc(C3CC3)nn2)c(=O)c2cc(C3CC3)nn12] | 86 |
| 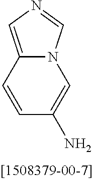 | 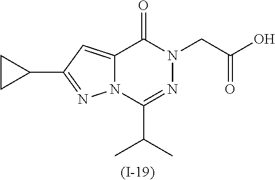 | 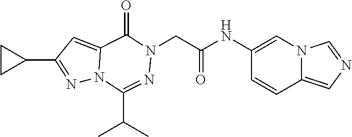 [SMILES: CC(C)c1nn(CC(=O)Nc2ccc3cncn3c2)c(=O)c2cc(C3CC3)nn12] | 87 |
| 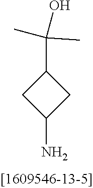 | 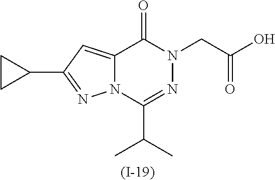 | 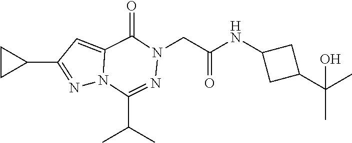 [SMILES: CC(C)c1nn(CC(=O)NC2CC(C(C)(C)O)C2)c(=O)c2cc(C3CC3)nn12] | 88 |
| 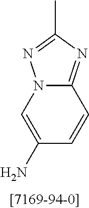 | 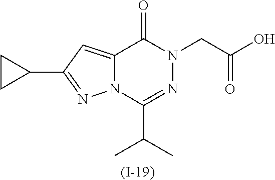 | 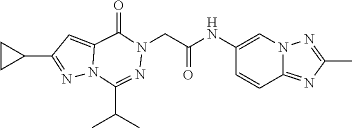 [SMILES: Cc1nc2ccc(NC(=O)Cn3nc(C(C)C)n4nc(C5CC5)cc4c3=O)cn2n1] | 89 |
| 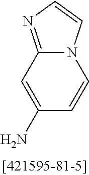 | 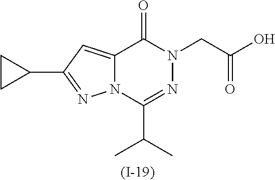 | 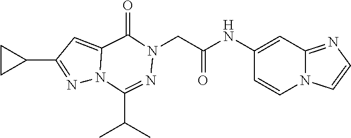 [SMILES: CC(C)c1nn(CC(=O)Nc2ccn3ccnc3c2)c(=O)c2cc(C3CC3)nn12] | 90 |
| 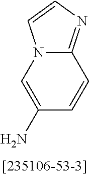 | 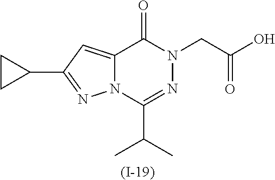 | 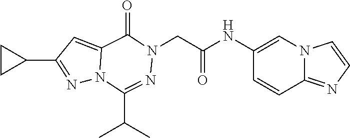 [SMILES: CC(C)c1nn(CC(=O)Nc2ccc3nccn3c2)c(=O)c2cc(C3CC3)nn12] | 91 |
| 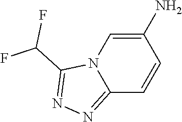 [SMILES: Nc1ccc2nnc(C(F)F)n2c1] | 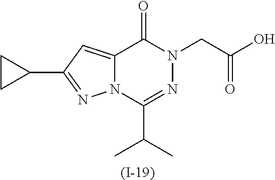 | 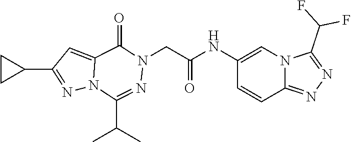 [SMILES: CC(C)c1nn(CC(=O)Nc2ccc3nnc(C(F)F)n3c2)c(=O)c2cc(C3CC3)nn12] | 92 |
| 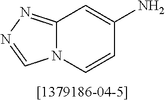 | 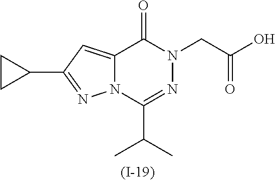 | 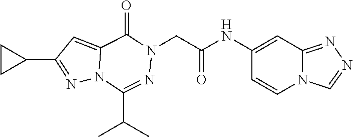 [SMILES: CC(C)c1nn(CC(=O)Nc2ccn3cnnc3c2)c(=O)c2cc(C3CC3)nn12] | 93 |
| 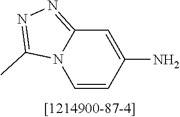 | 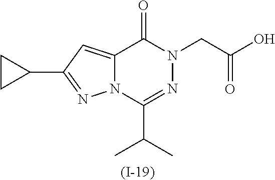 | 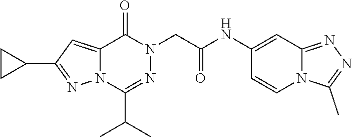 [SMILES: Cc1nnc2cc(NC(=O)Cn3nc(C(C)C)n4nc(C5CC5)cc4c3=O)ccn12] | 94 |
| 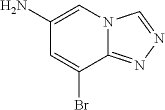 [SMILES: Nc1cc(Br)c2nncn2c1] | 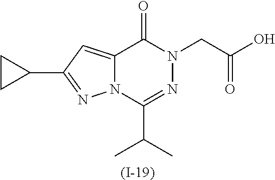 | 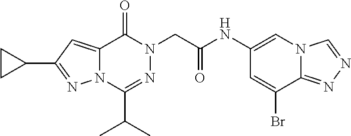 [SMILES: CC(C)c1nn(CC(=O)Nc2cc(Br)c3nncn3c2)c(=O)c2cc(C3CC3)nn12] | 95 |
| 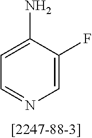 | 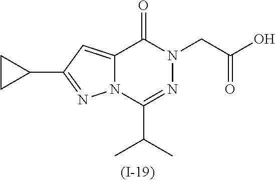 | 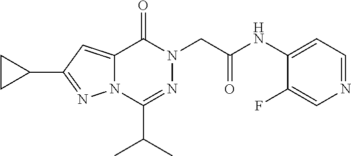 [SMILES: CC(C)c1nn(CC(=O)Nc2ccncc2F)c(=O)c2cc(C3CC3)nn12] | 96 |
| 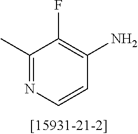 | 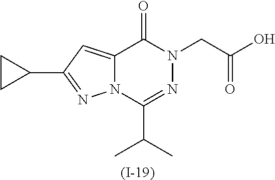 | 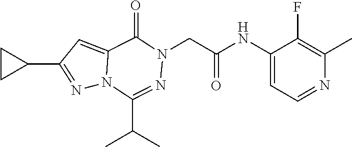 [SMILES: Cc1nccc(NC(=O)Cn2nc(C(C)C)n3nc(C4CC4)cc3c2=O)c1F] | 97 |
| 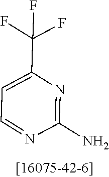 [SMILES: Nc1nccc(C(F)(F)F)n1] | 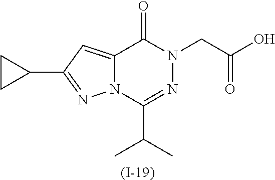 | 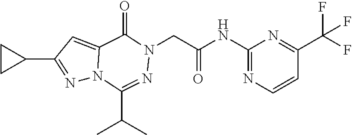 [SMILES: CC(C)c1nn(CC(=O)Nc2nccc(C(F)(F)F)n2)c(=O)c2cc(C3CC3)nn12] | 98 |
| 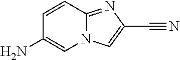 [SMILES: N#Cc1cn2cc(N)ccc2n1] | 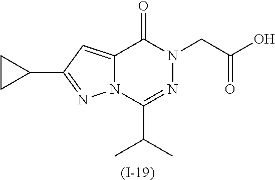 | 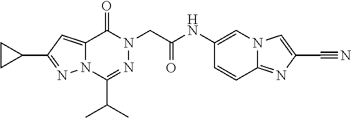 [SMILES: CC(C)c1nn(CC(=O)Nc2ccc3nc(C#N)cn3c2)c(=O)c2cc(C3CC3)nn12] | 99 |
| 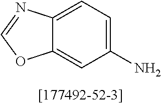 | 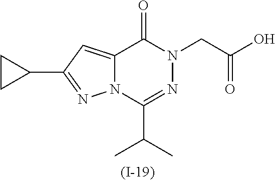 | 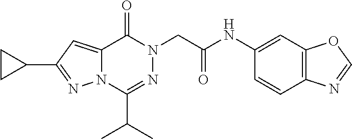 [SMILES: CC(C)c1nn(CC(=O)Nc2ccc3ncoc3c2)c(=O)c2cc(C3CC3)nn12] | 100 |
| 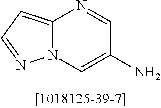 | 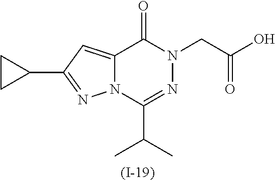 | 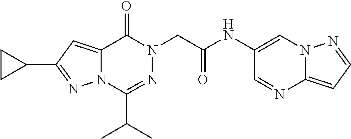 [SMILES: CC(C)c1nn(CC(=O)Nc2cnc3ccnn3c2)c(=O)c2cc(C3CC3)nn12] | 101 |
| 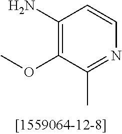 | 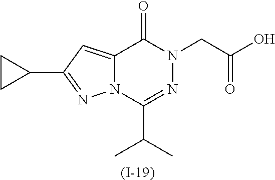 | 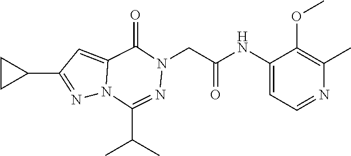 [SMILES: COc1c(NC(=O)Cn2nc(C(C)C)n3nc(C4CC4)cc3c2=O)ccnc1C] | 102 |
| 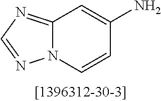 | 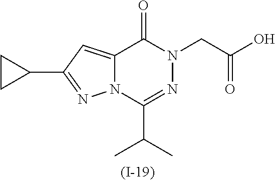 | 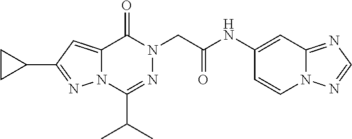 [SMILES: CC(C)c1nn(CC(=O)Nc2ccn3ncnc3c2)c(=O)c2cc(C3CC3)nn12] | 103 |
| 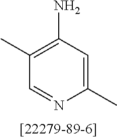 | 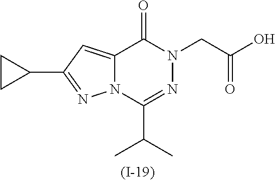 | 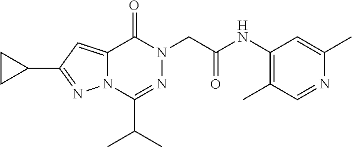 [SMILES: Cc1cc(NC(=O)Cn2nc(C(C)C)n3nc(C4CC4)cc3c2=O)c(C)cn1] | 104 |
| 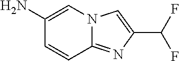 [SMILES: Nc1ccc2nc(C(F)F)cn2c1] | 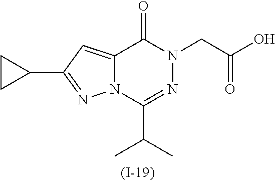 | 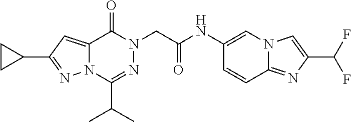 [SMILES: CC(C)c1nn(CC(=O)Nc2ccc3nc(C(F)F)cn3c2)c(=O)c2cc(C3CC3)nn12] | 105 |
| 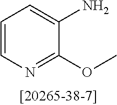 | 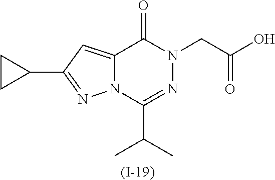 | 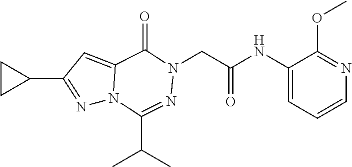 [SMILES: COc1ncccc1NC(=O)Cn1nc(C(C)C)n2nc(C3CC3)cc2c1=O] | 106 |
| 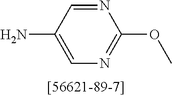 | 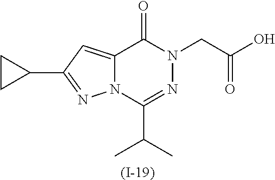 | 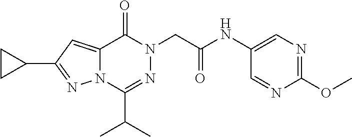 [SMILES: COc1ncc(NC(=O)Cn2nc(C(C)C)n3nc(C4CC4)cc3c2=O)cn1] | 107 |
| 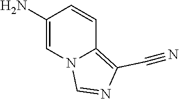 [SMILES: N#Cc1ncn2cc(N)ccc12] | 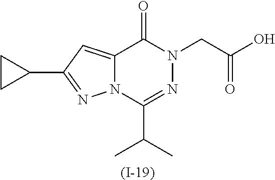 | 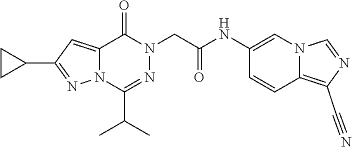 [SMILES: CC(C)c1nn(CC(=O)Nc2ccc3c(C#N)ncn3c2)c(=O)c2cc(C3CC3)nn12] | 108 |
| 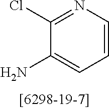 | 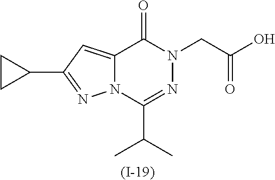 | 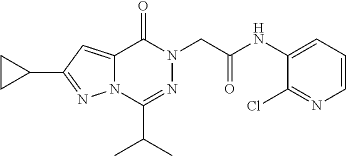 [SMILES: CC(C)c1nn(CC(=O)Nc2cccnc2Cl)c(=O)c2cc(C3CC3)nn12] | 109 |
| 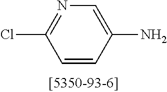 | 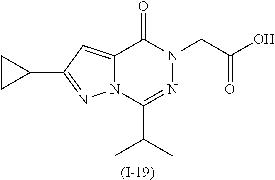 | 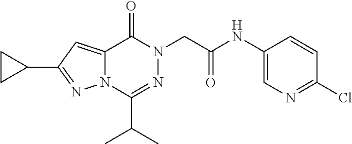 [SMILES: CC(C)c1nn(CC(=O)Nc2ccc(Cl)nc2)c(=O)c2cc(C3CC3)nn12] | 110 |
| 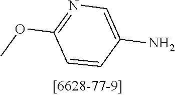 | 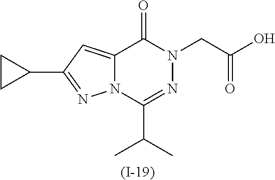 | 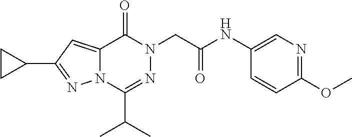 [SMILES: COc1ccc(NC(=O)Cn2nc(C(C)C)n3nc(C4CC4)cc3c2=O)cn1] | 111 |
| 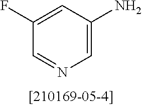 | 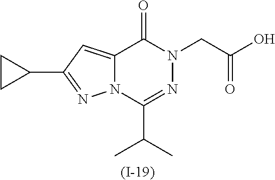 | 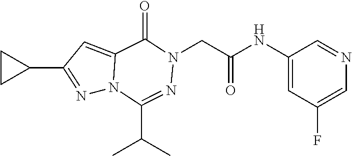 [SMILES: CC(C)c1nn(CC(=O)Nc2cncc(F)c2)c(=O)c2cc(C3CC3)nn12] | 112 |
| 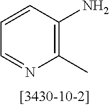 | 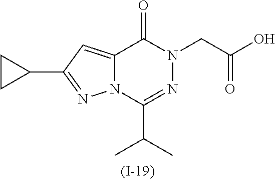 | 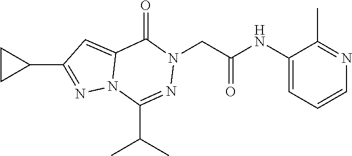 [SMILES: Cc1ncccc1NC(=O)Cn1nc(C(C)C)n2nc(C3CC3)cc2c1=O] | 113 |
| 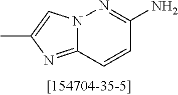 | 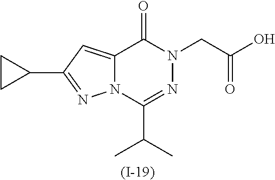 | 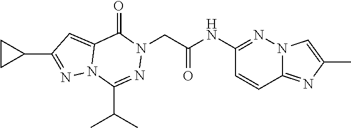 [SMILES: Cc1cn2nc(NC(=O)Cn3nc(C(C)C)n4nc(C5CC5)cc4c3=O)ccc2n1] | 114 |
| 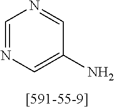 | 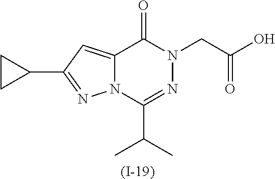 | 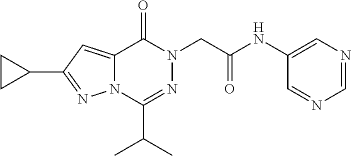 [SMILES: CC(C)c1nn(CC(=O)Nc2cncnc2)c(=O)c2cc(C3CC3)nn12] | 115 |
| 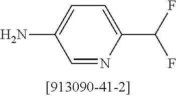 | 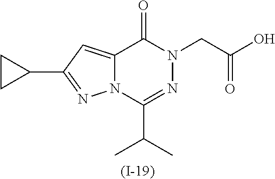 | 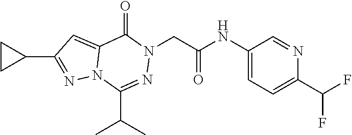 [SMILES: CC(C)c1nn(CC(=O)Nc2ccc(C(F)F)nc2)c(=O)c2cc(C3CC3)nn12] | 116 |
| 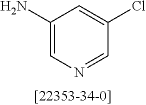 | 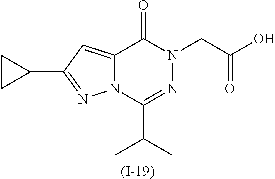 | 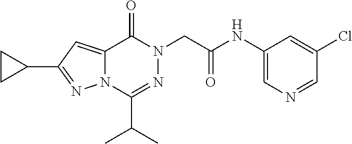 [SMILES: CC(C)c1nn(CC(=O)Nc2cncc(Cl)c2)c(=O)c2cc(C3CC3)nn12] | 117 |
| 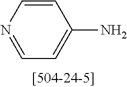 | 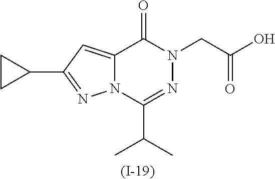 | 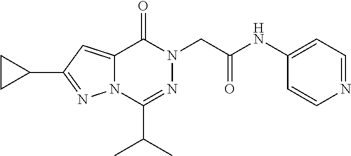 [SMILES: CC(C)c1nn(CC(=O)Nc2ccncc2)c(=O)c2cc(C3CC3)nn12] | 118 |
| 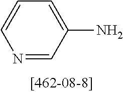 | 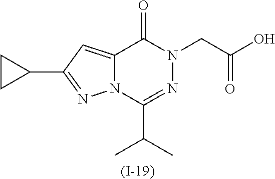 | 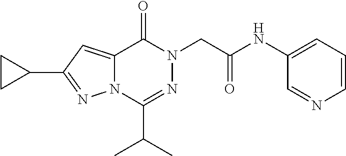 [SMILES: CC(C)c1nn(CC(=O)Nc2cccnc2)c(=O)c2cc(C3CC3)nn12] | 119 |
| 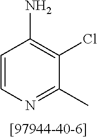 | 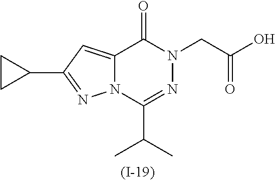 | 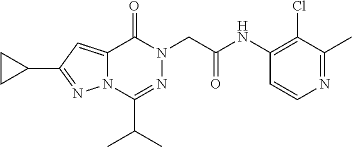 [SMILES: Cc1nccc(NC(=O)Cn2nc(C(C)C)n3nc(C4CC4)cc3c2=O)c1Cl] | 120 |
| 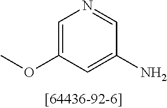 | 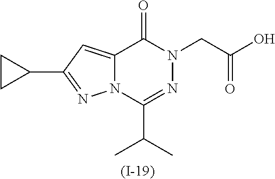 | 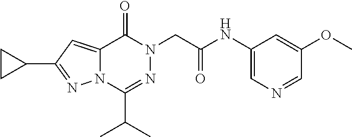 [SMILES: COc1cncc(NC(=O)Cn2nc(C(C)C)n3nc(C4CC4)cc3c2=O)c1] | 121 |
| 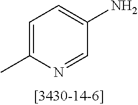 | 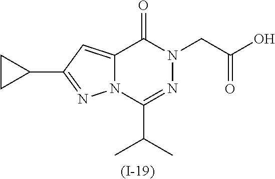 | 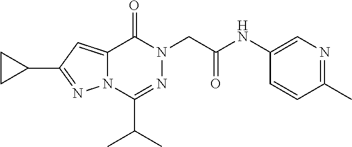 [SMILES: Cc1ccc(NC(=O)Cn2nc(C(C)C)n3nc(C4CC4)cc3c2=O)cn1] | 122 |
| 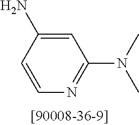 | 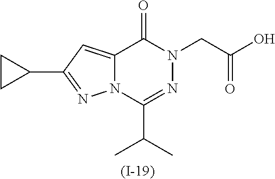 | 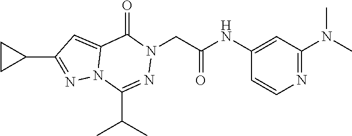 [SMILES: CC(C)c1nn(CC(=O)Nc2ccnc(N(C)C)c2)c(=O)c2cc(C3CC3)nn12] | 123 |
| 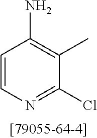 | 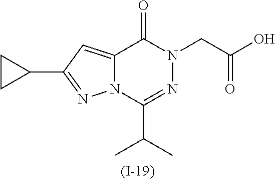 | 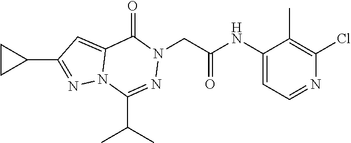 [SMILES: Cc1c(NC(=O)Cn2nc(C(C)C)n3nc(C4CC4)cc3c2=O)ccnc1Cl] | 124 |
| 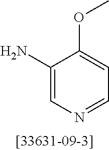 | 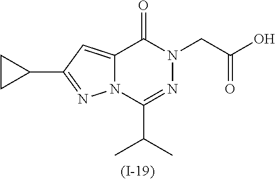 | 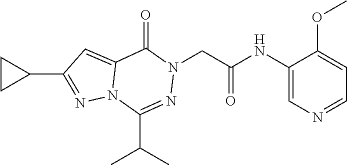 [SMILES: COc1ccncc1NC(=O)Cn1nc(C(C)C)n2nc(C3CC3)cc2c1=O] | 125 |
| 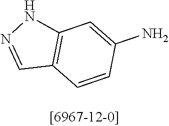 | 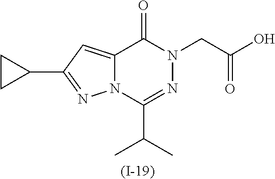 | 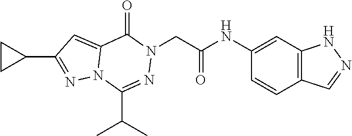 [SMILES: CC(C)c1nn(CC(=O)Nc2ccc3cn[nH]c3c2)c(=O)c2cc(C3CC3)nn12] | 126 |
| 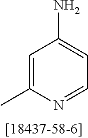 | 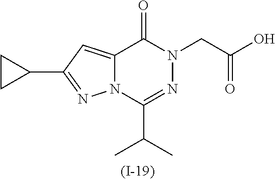 | 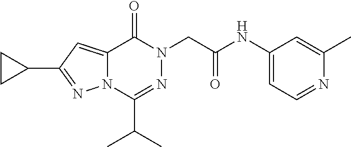 [SMILES: Cc1cc(NC(=O)Cn2nc(C(C)C)n3nc(C4CC4)cc3c2=O)ccn1] | 127 |
| 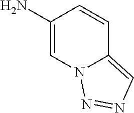 | 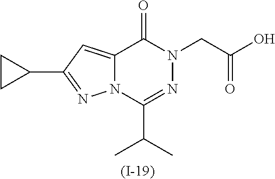 | 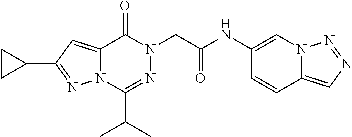 [SMILES: CC(C)c1nn(CC(=O)Nc2ccc3cnnn3c2)c(=O)c2cc(C3CC3)nn12] | 128 |
| 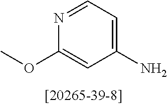 | 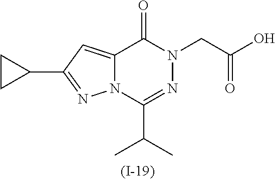 | 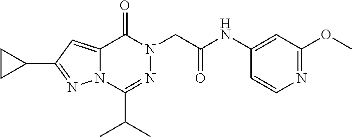 [SMILES: COc1cc(NC(=O)Cn2nc(C(C)C)n3nc(C4CC4)cc3c2=O)ccn1] | 129 |
| 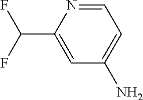 | 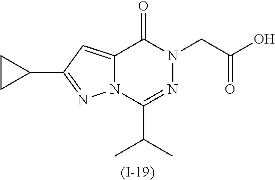 | 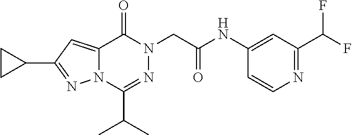 [SMILES: CC(C)c1nn(CC(=O)Nc2ccnc(C(F)F)c2)c(=O)c2cc(C3CC3)nn12] | 130 |
| 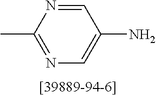 | 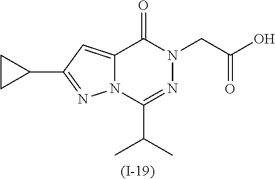 | 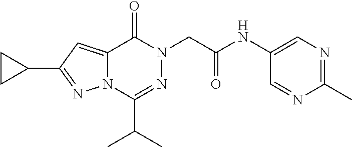 [SMILES: Cc1ncc(NC(=O)Cn2nc(C(C)C)n3nc(C4CC4)cc3c2=O)cn1] | 131 |
| 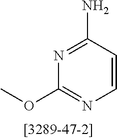 | 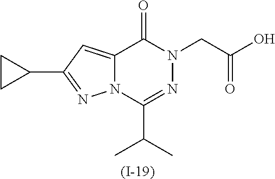 | 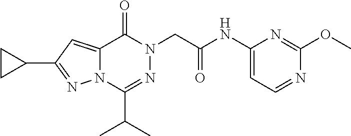 [SMILES: COc1nccc(NC(=O)Cn2nc(C(C)C)n3nc(C4CC4)cc3c2=O)n1] | 132 |
| 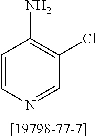 | 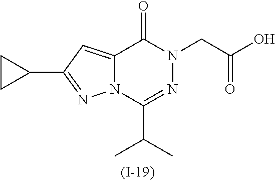 | 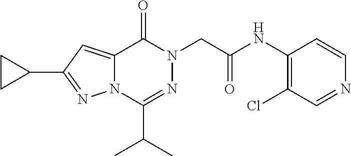 [SMILES: CC(C)c1nn(CC(=O)Nc2ccncc2Cl)c(=O)c2cc(C3CC3)nn12] | 133 |
| 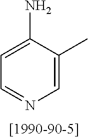 | 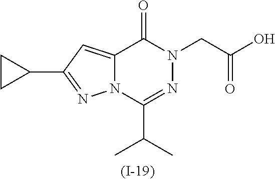 | 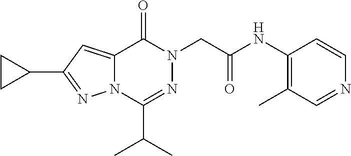 [SMILES: Cc1cnccc1NC(=O)Cn1nc(C(C)C)n2nc(C3CC3)cc2c1=O] | 134 |
| 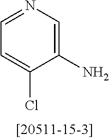 | 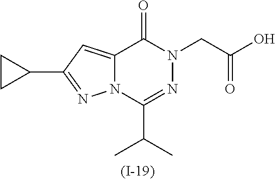 | 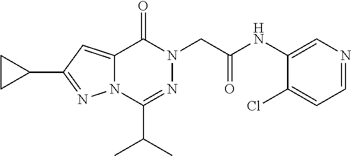 [SMILES: CC(C)c1nn(CC(=O)Nc2cnccc2Cl)c(=O)c2cc(C3CC3)nn12] | 135 |
| 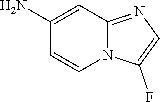 [SMILES: Nc1ccn2c(F)cnc2c1] | 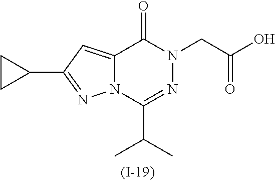 | 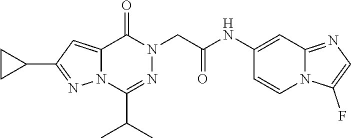 [SMILES: CC(C)c1nn(CC(=O)Nc2ccn3c(F)cnc3c2)c(=O)c2cc(C3CC3)nn12] | 136 |
| 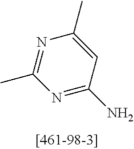 | 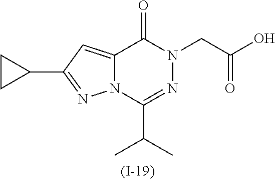 | 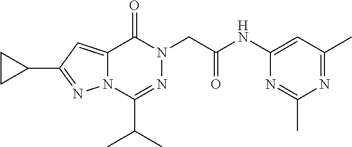 [SMILES: Cc1cc(NC(=O)Cn2nc(C(C)C)n3nc(C4CC4)cc3c2=O)nc(C)n1] | 137 |
| 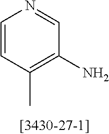 | 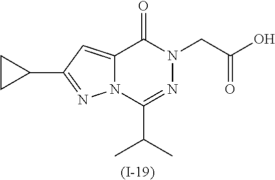 | 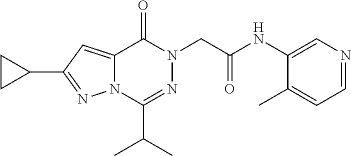 [SMILES: Cc1ccncc1NC(=O)Cn1nc(C(C)C)n2nc(C3CC3)cc2c1=O] | 138 |
| 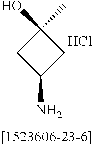 | 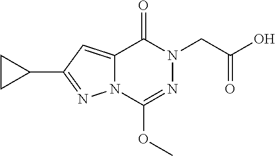 [SMILES: COc1nn(CC(=O)O)c(=O)c2cc(C3CC3)nn12] | 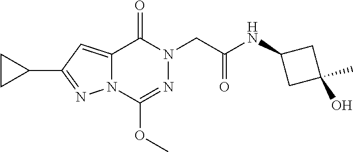 [SMILES: COc1nn(CC(=O)N[C@H]2C[C@@](C)(O)C2)c(=O)c2cc(C3CC3)nn12] | 139 |
| 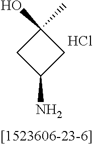 | 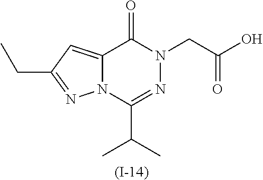 | 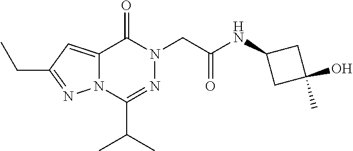 [SMILES: CCc1cc2c(=O)n(CC(=O)N[C@H]3C[C@@](C)(O)C3)nc(C(C)C)n2n1] | 140 |
| 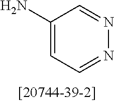 | 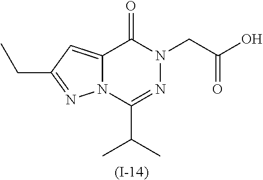 | 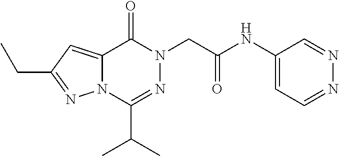 [SMILES: CCc1cc2c(=O)n(CC(=O)Nc3ccnnc3)nc(C(C)C)n2n1] | 141 |
| 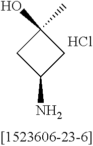 | 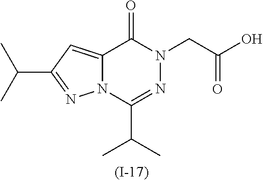 | 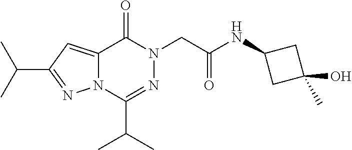 [SMILES: CC(C)c1cc2c(=O)n(CC(=O)N[C@H]3C[C@@](C)(O)C3)nc(C(C)C)n2n1] | 142 |
| 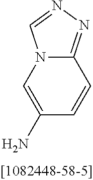 | 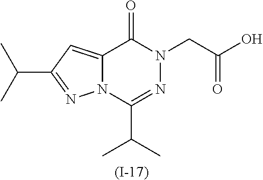 | 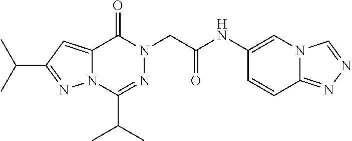 [SMILES: CC(C)c1cc2c(=O)n(CC(=O)Nc3ccc4nncn4c3)nc(C(C)C)n2n1] | 143 |
| 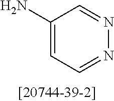 | 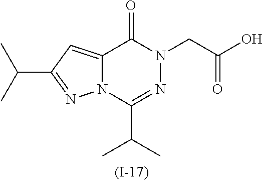 | 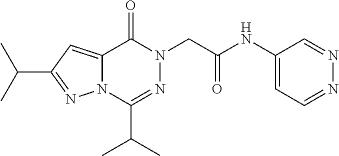 [SMILES: CC(C)c1cc2c(=O)n(CC(=O)Nc3ccnnc3)nc(C(C)C)n2n1] | 144 |
| 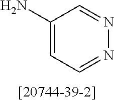 | 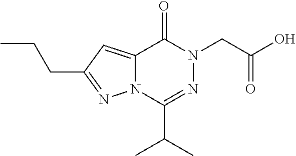 [SMILES: CCCc1cc2c(=O)n(CC(=O)O)nc(C(C)C)n2n1] | 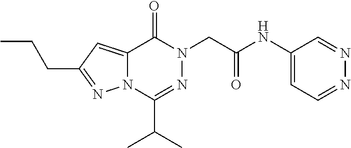 [SMILES: CCCc1cc2c(=O)n(CC(=O)Nc3ccnnc3)nc(C(C)C)n2n1] | 145 |
| 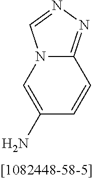 | 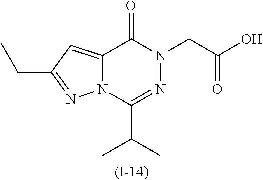 | 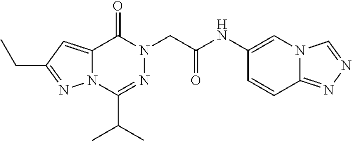 [SMILES: CCc1cc2c(=O)n(CC(=O)Nc3ccc4nncn4c3)nc(C(C)C)n2n1] | 146 |
| 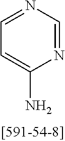 | 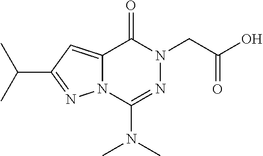 [SMILES: CC(C)c1cc2c(=O)n(CC(=O)O)nc(N(C)C)n2n1] | 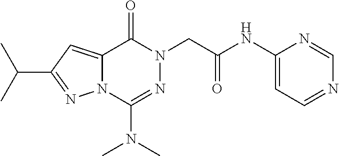 [SMILES: CC(C)c1cc2c(=O)n(CC(=O)Nc3ccncn3)nc(N(C)C)n2n1] | 147 |
| 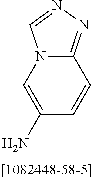 | 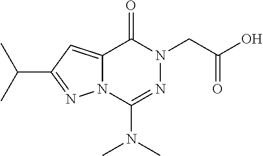 [SMILES: CC(C)c1cc2c(=O)n(CC(=O)O)nc(N(C)C)n2n1] | 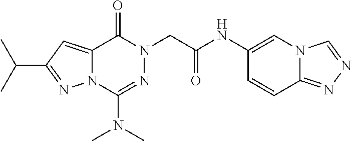 [SMILES: CC(C)c1cc2c(=O)n(CC(=O)Nc3ccc4nncn4c3)nc(N(C)C)n2n1] | 148 |
| 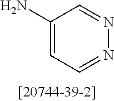 | 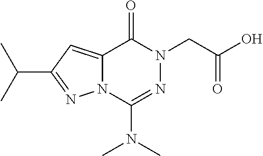 [SMILES: CC(C)c1cc2c(=O)n(CC(=O)O)nc(N(C)C)n2n1] | 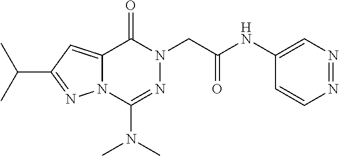 [SMILES: CC(C)c1cc2c(=O)n(CC(=O)Nc3ccnnc3)nc(N(C)C)n2n1] | 149 |
| 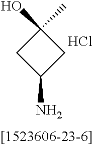 | 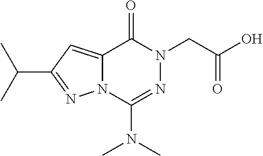 [SMILES: CC(C)c1cc2c(=O)n(CC(=O)O)nc(N(C)C)n2n1] | 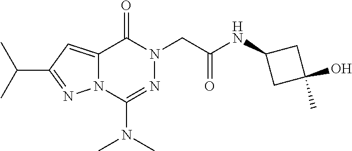 [SMILES: CC(C)c1cc2c(=O)n(CC(=O)N[C@H]3C[C@@](C)(O)C3)nc(N(C)C)n2n1] | 150 |
| 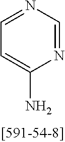 | 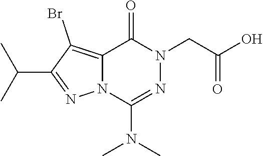 [SMILES: CC(C)c1nn2c(N(C)C)nn(CC(=O)O)c(=O)c2c1Br] | 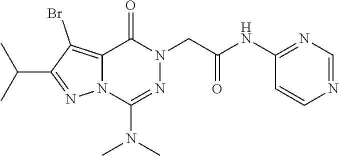 [SMILES: CC(C)c1nn2c(N(C)C)nn(CC(=O)Nc3ccncn3)c(=O)c2c1Br] | 151 |
| 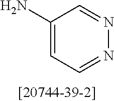 | 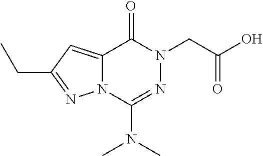 [SMILES: CCc1cc2c(=O)n(CC(=O)O)nc(N(C)C)n2n1] | 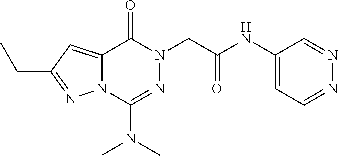 [SMILES: CCc1cc2c(=O)n(CC(=O)Nc3ccnnc3)nc(N(C)C)n2n1] | 152 |
| 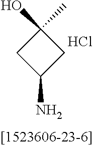 | 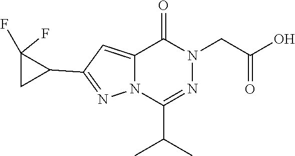 [SMILES: CC(C)c1nn(CC(=O)O)c(=O)c2cc(C3CC3(F)F)nn12] | 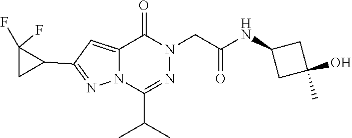 [SMILES: CC(C)c1nn(CC(=O)N[C@H]2C[C@@](C)(O)C2)c(=O)c2cc(C3CC3(F)F)nn12] | 153 |
| 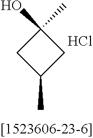 | 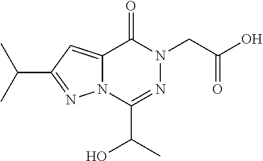 [SMILES: CC(C)c1cc2c(=O)n(CC(=O)O)nc(C(C)O)n2n1] | 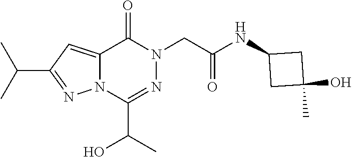 [SMILES: CC(C)c1cc2c(=O)n(CC(=O)N[C@H]3C[C@@](C)(O)C3)nc(C(C)O)n2n1] | 154 |
| 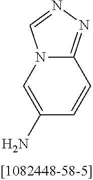 | 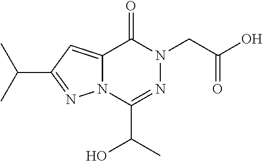 [SMILES: CC(C)c1cc2c(=O)n(CC(=O)O)nc(C(C)O)n2n1] | 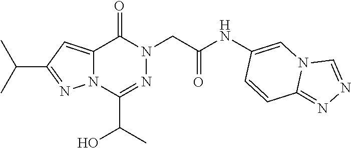 [SMILES: CC(C)c1cc2c(=O)n(CC(=O)Nc3ccc4nncn4c3)nc(C(C)O)n2n1] | 155 |
| 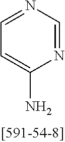 | 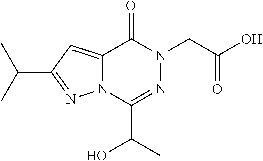 [SMILES: CC(C)c1cc2c(=O)n(CC(=O)O)nc(C(C)O)n2n1] | 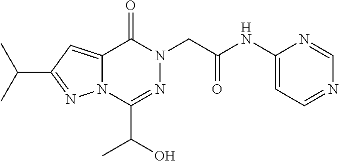 [SMILES: CC(C)c1cc2c(=O)n(CC(=O)Nc3ccncn3)nc(C(C)O)n2n1] | 156 |
| 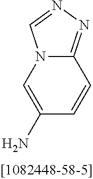 | 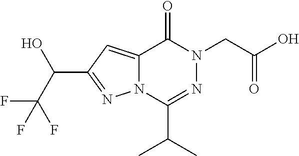 [SMILES: CC(C)c1nn(CC(=O)O)c(=O)c2cc(C(O)C(F)(F)F)nn12] | 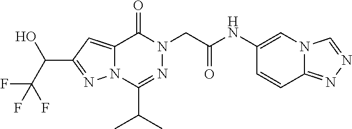 [SMILES: CC(C)c1nn(CC(=O)Nc2ccc3nncn3c2)c(=O)c2cc(C(O)C(F)(F)F)nn12] | 157 |
| 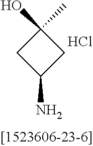 | 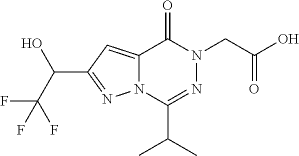 [SMILES: CC(C)c1nn(CC(=O)O)c(=O)c2cc(C(O)C(F)(F)F)nn12] | 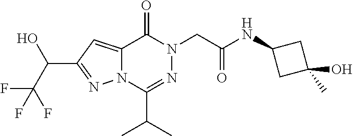 [SMILES: CC(C)c1nn(CC(=O)N[C@H]2C[C@@](C)(O)C2)c(=O)c2cc(C(O)C(F)(F)F)nn12] | 158 |
| 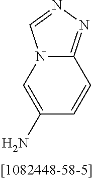 | 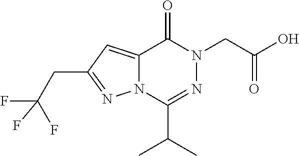 [SMILES: CC(C)c1nn(CC(=O)O)c(=O)c2cc(CC(F)(F)F)nn12] | 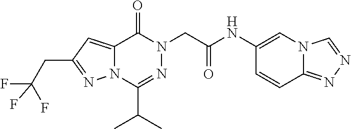 [SMILES: CC(C)c1nn(CC(=O)Nc2ccc3nncn3c2)c(=O)c2cc(CC(F)(F)F)nn12] | 159 |
| 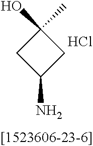 | 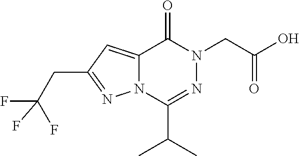 [SMILES: CC(C)c1nn(CC(=O)O)c(=O)c2cc(CC(F)(F)F)nn12] | 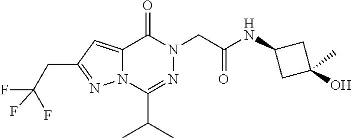 [SMILES: CC(C)c1nn(CC(=O)N[C@H]2C[C@@](C)(O)C2)c(=O)c2cc(CC(F)(F)F)nn12] | 160 |
| 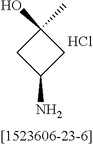 | 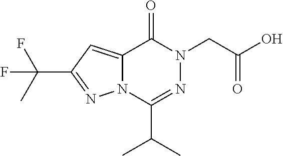 [SMILES: CC(C)c1nn(CC(=O)O)c(=O)c2cc(C(C)(F)F)nn12] | 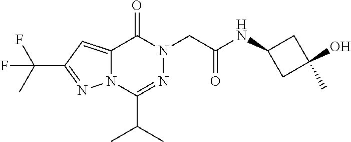 [SMILES: CC(C)c1nn(CC(=O)N[C@H]2C[C@@](C)(O)C2)c(=O)c2cc(C(C)(F)F)nn12] | 161 |
| 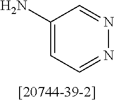 | 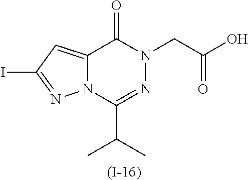 | 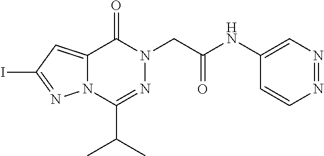 [SMILES: CC(C)c1nn(CC(=O)Nc2ccnnc2)c(=O)c2cc(I)nn12] | 162 |

### Example A3

Synthesis of N-([1,2,4]triazolo[4,3-b]pyridazin-6-yl)-2-(2,7-dicyclopropyl-4-oxopyrazolo[1,5-d][1,2,4]triazin-5(4H)-yl)acetamide—Final Cpd 163

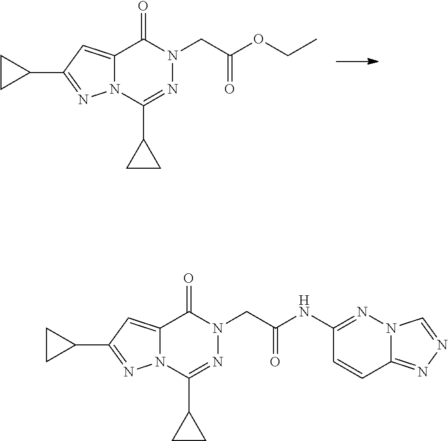 [SMILES: CCOC(=O)Cn1nc(C2CC2)n2nc(C3CC3)cc2c1=O ; O=C(Cn1nc(C2CC2)n2nc(C3CC3)cc2c1=O)Nc1ccc2nncn2n1]

Lithium bis(trimethylsilyl)amide [4039-32-1] (0.11 mL, 1 M, 0.11 mmol) was added to a stirred solution of [1,2,4]triazolo[4,3-b]pyridazin-6-amine (8 mg, 0.055 mmol) in

DMF (0.2 mL) at 0° C. under N2. The mixture was stirred at 0° C. for 10 min and then ethyl 2-(2,7-dicyclopropyl-4-oxopyrazolo[1,5-d][1,2,4]triazin-5(4H)-yl)acetate (15 mg, 0.05 mmol) in THF (0.3 mL) was added at 0° C. The resulting mixture was stirred at this temperature for 1.5h. Then, a new solution of additional mixture of lithium bis(trimethylsilyl)amide [4039-32-1] (0.11 mL, 1 M, 0.11 mmol) added to a stirred solution of [1,2,4]triazolo[4,3-b]pyridazin-6-amine VILL acandel12622 (8 mg, 0.055 mmol) in DMF (0.2 mL) was added to the reaction mixture at 0° C. and the resulting reaction mixture was left stirring from 0° C. to RT for 18h. The mixture was diluted with NH₄Cl (10% in water) and extracted with EtOAc (x3). The organic layer was separated, dried (Na₂SO₄), filtered and the solvents evaporated in vacuo. The crude material was triturated with CH₃CN to yield N-([1,2,4]triazolo[4,3-b]pyridazin-6-yl)-2-(2,7-dicyclopropyl-4-oxopyrazolo[1,5-d][1,2,4]triazin-5(4H)-yl)acetamide Final Cpd 163 (4 mg, yield 21%) as a beige solid. The filtrate was evaporated in vacuo to yield N-([1,2,4]triazolo[4,3-b]pyridazin-6-yl)-2-(2,7-dicyclopropyl oxopyrazolo[1,5-d][1,2,4]triazin-5(4H)-yl)acetamide Final Cpd 163 (10 mg, yield 52%) as a pale yellow solid.

Additional analogs were accessed using similar reaction conditions, using the appropriate reagent.

| Reagent | Intermediate | Final compound | Number |
|---|---|---|---|
| 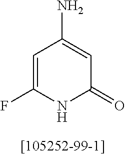 | 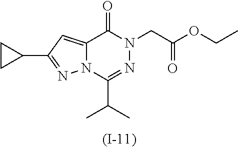 | 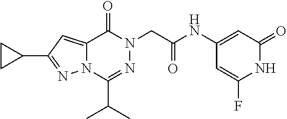 [SMILES: CC(C)c1nn(CC(=O)Nc2cc(F)[nH]c(=O)c2)c(=O)c2cc(C3CC3)nn12] | 164 |
| 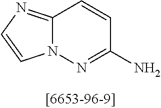 | 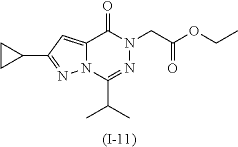 | 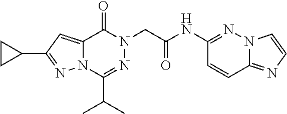 [SMILES: CC(C)c1nn(CC(=O)Nc2ccc3nccn3n2)c(=O)c2cc(C3CC3)nn12] | 165 |

### Example A4

Synthesis of N-([1,2,4]triazolo[4,3-b]pyridazin-6-yl)-2-(2-cyclopropyl-7-isopropyl-4-oxopyrazolo[1,5-d][1,2,4]triazin-5(4H)-yl)acetamide — Final Cpd 166

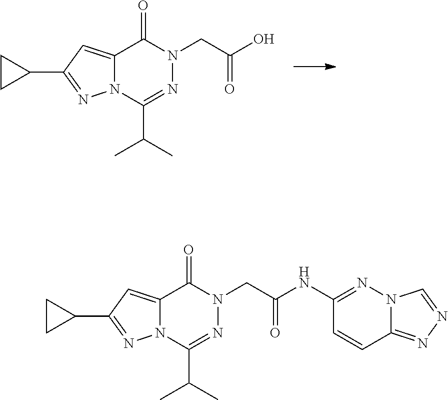 [SMILES: CC(C)c1nn(CC(=O)O)c(=O)c2cc(C3CC3)nn12 ; CC(C)c1nn(CC(=O)Nc2ccc3nncn3n2)c(=O)c2cc(C3CC3)nn12]

To a mixture of 2-(2-cyclopropyl-7-isopropyl-4-oxopyrazolo[1,5-d][1,2,4]triazin-5(4H)-yl)acetic acid (I 19) (80 mg, 0.2895 mmol) in dioxane (5 mL), 1-chloro-n,n,2-trimethyl-1-propenylamine [26189-59-3] (118 mg, 0.88 mmol) was added and the mixture was stirred for lh at rt. Then [1,2,4]triazolo[4,3-b]pyridazin-6-amine [19195-46-1] (54 mg, 0.4 mmol) was added followed by pyridine [110-86-1] (0.08 mL, 0.982 g/mL, 1 mmol). The mixture was stirred for 5h at rt. Water was added and the crude was extracted with AcOEt (3×5 ml); the combined organic layers were dried, filtered and evaporated in vacuo to afford an oil. The crude product was purified by flash column chromatography (silica; MeOH in DCM 0/100 to 5/95). The desired fractions were collected and the solvent evaporated in vacuo to yield N-([1,2,4]triazolo[4,3-b]pyridazin-6-yl)-2-(2-cyclopropyl-7-isopropyl-4-oxopyrazolo[1, 5-d] [1,2,4]triazin-5(4H)-yl)acetamide Final Cpd 166 (81 mg, yield 71%) as white solid.

Additional analogs were accessed using similar reaction conditions, using the appropriate reagent.

| Reagent | Intermediate | Final compound | Number |
|---|---|---|---|
| 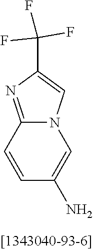 [SMILES: Nc1ccc2nc(C(F)(F)F)cn2c1] | 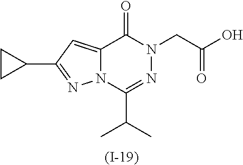 | 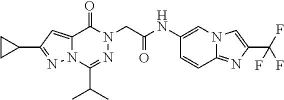 [SMILES: CC(C)c1nn(CC(=O)Nc2ccc3nc(C(F)(F)F)cn3c2)c(=O)c2cc(C3CC3)nn12] | 167 |
| 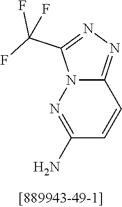 [SMILES: Nc1ccc2nnc(C(F)(F)F)n2n1] | 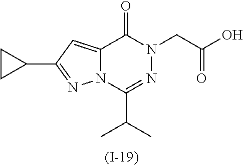 | 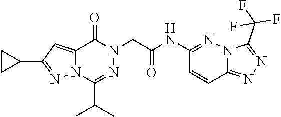 [SMILES: CC(C)c1nn(CC(=O)Nc2ccc3nnc(C(F)(F)F)n3n2)c(=O)c2cc(C3CC3)nn12] | 168 |
| 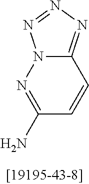 | 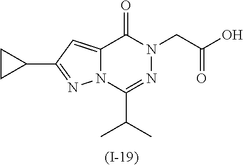 | 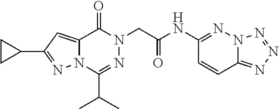 [SMILES: CC(C)c1nn(CC(=O)Nc2ccc3nnnn3n2)c(=O)c2cc(C3CC3)nn12] | 169 |
| 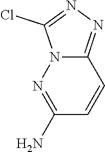 [SMILES: Nc1ccc2nnc(Cl)n2n1] | 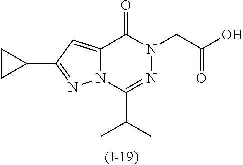 | 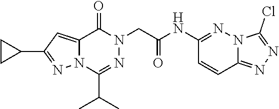 [SMILES: CC(C)c1nn(CC(=O)Nc2ccc3nnc(Cl)n3n2)c(=O)c2cc(C3CC3)nn12] | 170 |
| 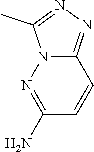 | 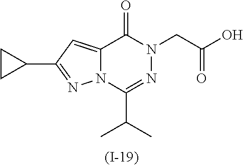 | 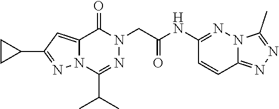 [SMILES: Cc1nnc2ccc(NC(=O)Cn3nc(C(C)C)n4nc(C5CC5)cc4c3=O)nn12] | 171 |

### Example A5

Synthesis of 2-(2-cyclopropyl-7-isopropyl-4-oxopyrazolo[1,5-d][1,2,4]triazin-5(4H)-yl)-N-(6-(trifluoromethylpyridazin-3-yl)acetamide Final Cpd 172

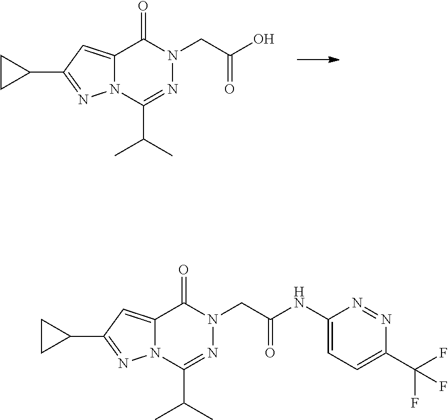 [SMILES: CC(C)c1nn(CC(=O)O)c(=O)c2cc(C3CC3)nn12 ; CC(C)c1nn(CC(=O)Nc2ccc(C(F)(F)F)nn2)c(=O)c2cc(C3CC3)nn12]

2-(2-cyclopropyl-7-isopropyl-4-oxopyrazolo[1,5-d][1,2,4]triazin-5(4H)-yl)acetic acid (I 19) (80 mg, 0.29 mmol, 1 equiv) and 6-(trifluoromethyl)pyridazin-3-amine (62.8 mg, 0.39 mmol, 1.33 equiv) were suspended in MeCN (2.3 mL) in a screw-cap vial. The suspension was stirred vigorously and 1-methylimidazole (115 1.03 g/mL, 1.45 mmol, 5 equiv) was added, followed by addition of solid TCFH (162.5 mg, 0.58 mmol, 2 equiv). The mixture was stirred vigorously at r.t. for 2.5 hours. The reaction mixture was concentrated in vacuo, the residue dissolved in DCM (ca. 5-6 mL) and purified by

FCC (Biotage, Sfar 25g, 80 mL/min, Hept/EtOAc 93:7 to 2:3 over 35 CV) to give 2-(2-cyclopropyl-7-isopropyl-4-oxopyrazolo[1,5-d][1,2,4]triazin-5(4H)-yl)-N-(6-(trifluoromethyl)pyridazin-3-yl)acetamide Final Cpd 172 (94 mg, 77%) as colorless solid.

Additional analogs were accessed using similar reaction conditions, using the appropriate reagent.

| Reagent | Intermediate | Final compound | Number |
|---|---|---|---|
| 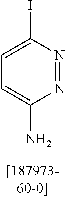 | 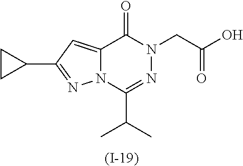 | 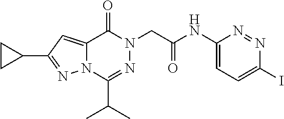 [SMILES: CC(C)c1nn(CC(=O)Nc2ccc(I)nn2)c(=O)c2cc(C3CC3)nn12] | 173 |
| 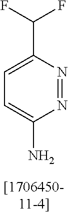 | 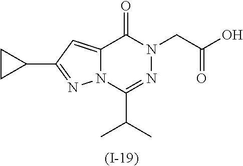 | 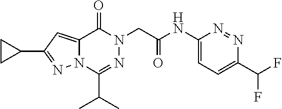 [SMILES: CC(C)c1nn(CC(=O)Nc2ccc(C(F)F)nn2)c(=O)c2cc(C3CC3)nn12] | 174 |

### Example A6

Synthesis of N-([1,2,4]triazolo[4,3-a]pyridin-6-yl)-2-(2-(1,1-difluoroethyl) isopropyl-4-oxopyrazolo[1,5-d][1,2,4]triazin-5(4H)-yl)acetamide — Final Cpd 175

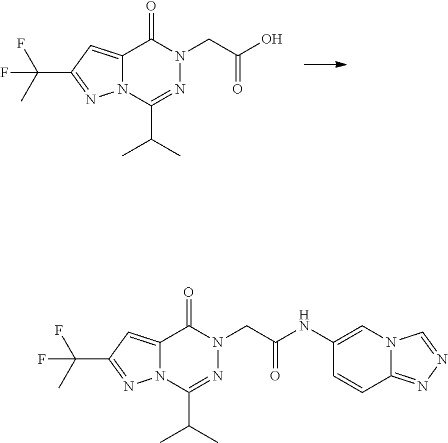 [SMILES: CC(C)c1nn(CC(=O)O)c(=O)c2cc(C(C)(F)F)nn12 ; CC(C)c1nn(CC(=O)Nc2ccc3nncn3c2)c(=O)c2cc(C(C)(F)F)nn12]

To a mixture of 2-(2-(1,1-difluoroethyl)-7-isopropyl-4-oxopyrazolo[1,5-d][1,2,4]triazin-5(4H)-yl)acetic acid (40 mg, 0.133 mmol) in dry pyridine (5 ml), [1,2,4]triazolo[4,3-a]pyridin-6-amine [1082448-58-5] (26.804 mg, 0.2 mmol) was added under N2. The mixture was sonicated for 10 min and then stirred for 40 min at rt. Titanium(IV) chloride [7550-45-0] (0.53 mL, 1 M, 0.53 mmol) was added dropwise at rt. The mixture was stirred for lh at rt and then heated at 80° C. for 24 h. The solvent was evaporated in vacuo and the crude was treated with HCl (2 N) till acid pH, the crude was extracted with AcOEt (3×5 ml) and the combined organic layer was evaporated in vacuo and purified by column chromatograpy (Silica; MeOH in CH₂Cl_{0/100} to 5/95). The desired fractions were collected and concentrated in vacuo to afford N-([1,2,4]triazolo[4,3-a]pyridin-6-yl)-2-(2-(1,1-difluoroethyl)-74 sopropyl-4-oxopyrazolo[1,5-d][1,2,4]triazin-5(4H)-yl)acetamide Final Cpd 175 (30 mg, yield 54%) as a solid.

Additional analogs were accessed using similar reaction conditions, using the appropriate reagent.

| Reagent | Intermediate | Final compound | Number |
|---|---|---|---|
| 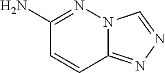 | 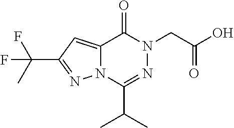 [SMILES: CC(C)c1nn(CC(=O)O)c(=O)c2cc(C(C)(F)F)nn12] | 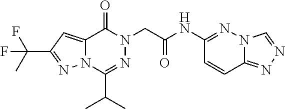 [SMILES: CC(C)c1nn(CC(=O)Nc2ccc3nncn3n2)c(=O)c2cc(C(C)(F)F)nn12] | 176 |

### Example A7

Synthesis of 2-(2-cyclopropyl-7-ethyl-4-oxo-pyrazolo[1,5-d][1,2,4]triazin-5-yl)-N-pyrimidin-4-yl-acetamide (Final compound 10)

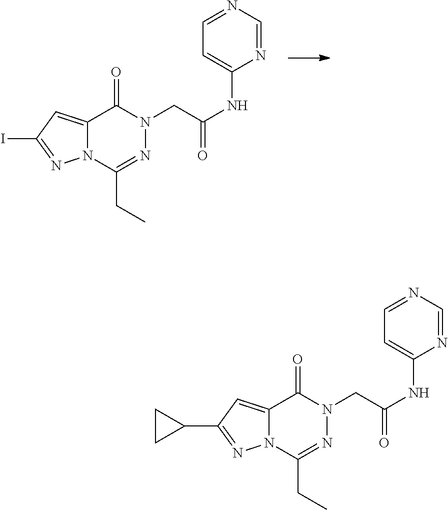 [SMILES: CCc1nn(CC(=O)Nc2ccncn2)c(=O)c2cc(I)nn12 ; CCc1nn(CC(=O)Nc2ccncn2)c(=O)c2cc(C3CC3)nn12]

Cyclopropylzinc bromide 0.5M in THF [126403-68-7] (1.2 mL, 0.5 M, 0.6 mmol) was added dropwise to a stirred suspension of 2-(7-ethyl-2-iodo-4-oxo-pyrazolo[1,5-d][1,2,4]triazin-5-yl)-N-pyrimidin-4-yl-acetamide (Final compound 4) (85 mg, 0.20 mmol) and bis(tri-tert-butylphosphine)palladium(O) [53199-31-8] (10 mg, 0.020 mmol) in THF (2 mL) at 0° C. in a sealed tube and under N2. The mixture was stirred at 0° C. for 2 min and at 70° C. for 1 h. The mixture was added dropwise to a 20% aqueous solution of NH₄Cl and extracted with DCM/iPrOH (9/1). The organic layer was separated, dried (MgSO₄), filtered and the solvents evaporated in vacuo. The crude product was purified by RP flash chromatography (C18, ACN in NH₄HCO_{3 0.25}% solution in water 15/85 to 100/0). The desired fractions were collected and concentrated in vacuo to yield 2-(2-cyclopropyl-7-ethyl-4-oxo-pyrazolo[1, 5-d] [1,2,4]triazin-5-yl)-N-pyrimidin-4-yl-acetamide (Final compound 10) (30 mg, 44%) as a white solid.

Additional analogs were accessed using similar reaction conditions, using the appropriate reagent.

| Reagent | Starting material | Final compound | Number |
|---|---|---|---|
| [126403-68-7] | 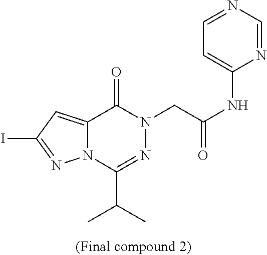 [SMILES: CC(C)c1nn(CC(=O)Nc2ccncn2)c(=O)c2cc(I)nn12] | 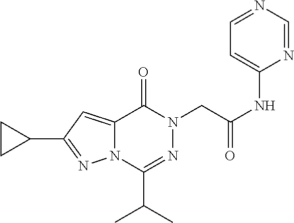 [SMILES: CC(C)c1nn(CC(=O)Nc2ccncn2)c(=O)c2cc(C3CC3)nn12] | 11 |
| [77047-87-1] | 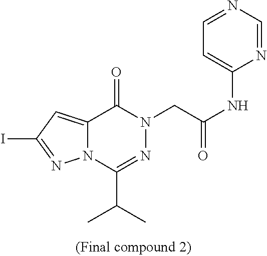 [SMILES: CC(C)c1nn(CC(=O)Nc2ccncn2)c(=O)c2cc(I)nn12] | 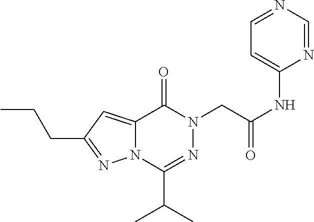 [SMILES: CCCc1cc2c(=O)n(CC(=O)Nc3ccncn3)nc(C(C)C)n2n1] | 12 |
| [557-20-0] | 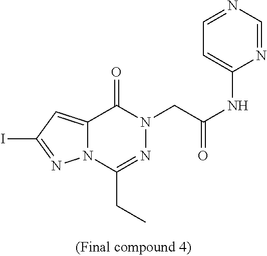 [SMILES: CCc1nn(CC(=O)Nc2ccncn2)c(=O)c2cc(I)nn12] | 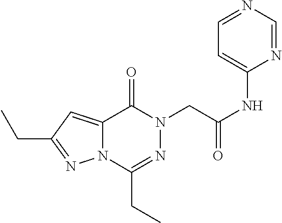 [SMILES: CCc1cc2c(=O)n(CC(=O)Nc3ccncn3)nc(CC)n2n1] | 13 |
| [544-97-8] | 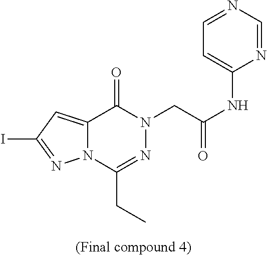 [SMILES: CCc1nn(CC(=O)Nc2ccncn2)c(=O)c2cc(I)nn12] | 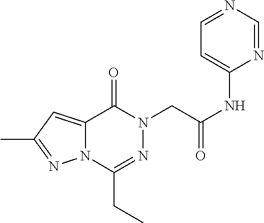 [SMILES: CCc1nn(CC(=O)Nc2ccncn2)c(=O)c2cc(C)nn12] | 14 |
| [77047-87-1] | 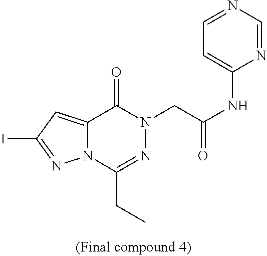 [SMILES: CCc1nn(CC(=O)Nc2ccncn2)c(=O)c2cc(I)nn12] | 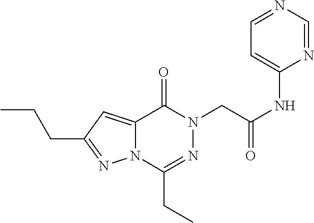 [SMILES: CCCc1cc2c(=O)n(CC(=O)Nc3ccncn3)nc(CC)n2n1] | 15 |
| [77047-87-1] | 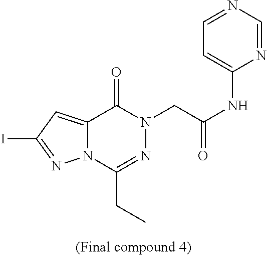 [SMILES: CCc1nn(CC(=O)Nc2ccncn2)c(=O)c2cc(I)nn12] | 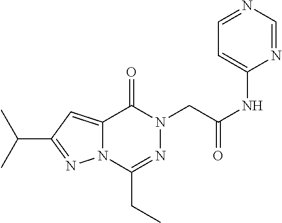 [SMILES: CCc1nn(CC(=O)Nc2ccncn2)c(=O)c2cc(C(C)C)nn12] | 16 |
| [544-97-8] | 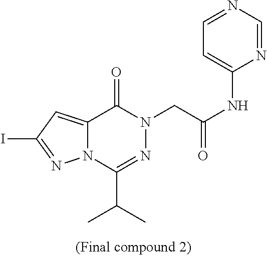 [SMILES: CC(C)c1nn(CC(=O)Nc2ccncn2)c(=O)c2cc(I)nn12] | 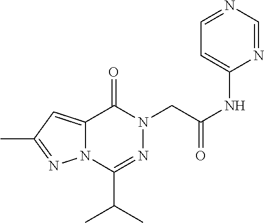 [SMILES: Cc1cc2c(=O)n(CC(=O)Nc3ccncn3)nc(C(C)C)n2n1] | 17 |

### Example A8

Synthesis of ethyl 3-((7-isopropyl-4-oxo-5-(2-oxo-2-(pyridazin-4-ylamino)ethyl)-4,5-dihydropyrazolo[1,5-d][1,2,4]triazin-2-yl)methyl)cyclobutane-1-carboxylate — Final Cpd 177

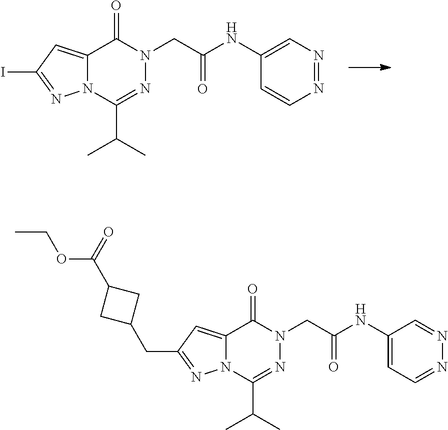 [SMILES: CC(C)c1nn(CC(=O)Nc2ccnnc2)c(=O)c2cc(I)nn12 ; CCOC(=O)C1CC(Cc2cc3c(=O)n(CC(=O)Nc4ccnnc4)nc(C(C)C)n3n2)C1]

Pd(dppf)Cl₂ CH2Cl2 [95464-05-4] (26 mg, 0.031 mmol) and copper(I) iodide [7681-65-4] (6 mg, 0.031 mmol) were added to a stirred solution of 2-(2-iodo-7-isopropyl-4-oxopyrazolo[1,5-d][1,2,4]triazin-5(4H)-yl)-N-(pyridazin-4-yl)acetamide — compound 162 — (137 mg, 0.31 mmol) in dry DMF at rt while nitrogen was bubbling. The mixture was stirred for 10 min at rt while nitrogen was bubbling. Then, ((3-(ethoxycarbonyl)cyclobutyl)methyl)zinc(II) iodide (0.25 M in DMF) (417 mg, 1.25 mmol) was added to the stirred suspension at rt while nitrogen was bubbling. The mixture was stirred in a sealed tube at 90° C. for 16h. The mixture was diluted with water and extracted with AcOEt. The organic layer was separated, dried (MgSO₄), filtered and the solvents evaporated in vacuo. The crude product was purified by flash column chromatography (silica 12 g; MeOH in DCM from 0:100 to 10:90). The desired fractions were collected and evaporated in vacuo. The residue was repurified by RPP-C18 using as column: Brand Phenomenex; Type Gemini; Product number 00D-4435-EO-AX; I.D. (mm) 100×30; Particle size 5 μm (C18) 110A; Installed Agilent 1. Method: MG3BIC. From 70:30 to 27:73 [25 mM NH₄HCO_{3/}ACN: MeOH 1:1]. Desired fractions were combined and concentrated under reduced pressure. The residue was repurified by RPP-C18 using as column: Brand Phenomenex; Type Gemini; Product number 00D-4435-EO-AX; I.D. (mm) 100×30; Particle size 5um (C18) 110A; Installed Agilent 1. Method: MG3AF. From 70:30 to 27:73 [0.1% HCOOH/[ACN:MeOH (1:1)]. Desired fractions were combined and diluted with DCM then the mixture was basified until pH 9 using an aqueous saturated Na₂CO₃ solution. The phases were separated and the aqueous phase was extracted three times with more DCM. The whole organic phase was dried over MgSO₄, filtered and evaporated in vacuo to yield ethyl 3-((7-isopropyl-4-oxo-5-(2-oxo-2-(pyridazin-4-ylamino)ethyl)-4,5-dihydropyrazolo[1,5-d] [1,2,4]triazin-2-yl)methyl)cyclobutane-1-carboxylate Final Cpd 177 (26.5 mg, yield 18%) as a white solid.

Additional analogs were accessed using similar reaction conditions, using the appropriate reagent.

| Reagent | Final compound | Number |
|---|---|---|
| 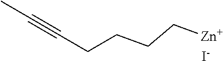 | 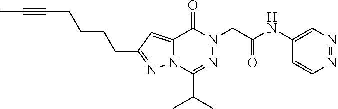 [SMILES: CC#CCCCCc1cc2c(=O)n(CC(=O)Nc3ccnnc3)nc(C(C)C)n2n1] | 178 |

### Example A9

Synthesis of 2-[7-isopropyl-2-(methylamino)-4-oxo-pyrazolo[1,5-d] [1,2,4]triazin-5-yl]—N-pyrimidin-4-yl-acetamide (Final compound 18) and 2-[7-isopropyl-2-(methylamino)-4-oxo-pyrazolo[1,5-d][1,2,4]triazin-5-yl]—N-methyl-acetamide (Compound 19)

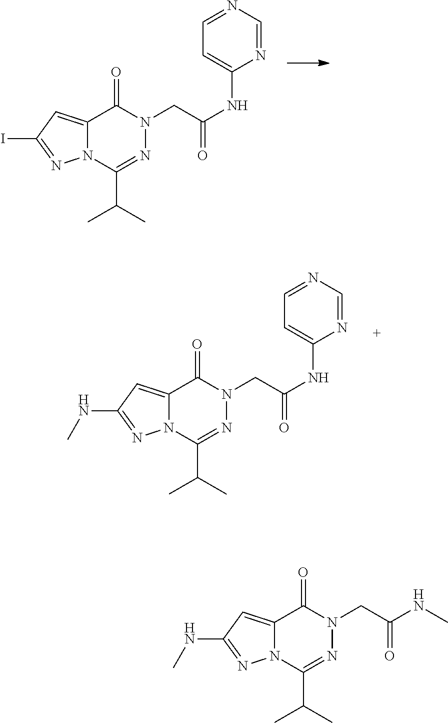

DMSO (2 mL) was added to a mixture of 2-(2-iodo-7-isopropyl-4-oxo-pyrazolo[1,5-d][1,2,4]triazin-5-yl)-N-pyrimidin-4-yl-acetamide (Final compound 2) (100 mg, 0.23 mmol), methylamine hydrochloride [593-51-1] (33 mg, 0.49 mmol), CuI [7681-65-4] (4 mg, 0.02 mmol), D/L-proline [609-36-9] (3 mg, 0.026 mmol) and K₂CO₃ [584-08-7] (100 mg, 0.73 mmol) in a sealed tube and under N2. The mixture was stirred at 100° C. for 2.5 days. The mixture was treated with 32% ammonia solution in water, diluted with brine and extracted with EtOAc. The organic layer was separated, dried (MgSO₄), filtered and the solvent was evaporated in vacuo. The crude product was purified by flash column chromatography (SiO2 amino functionalized, EtOAc in heptane 25/75 to 100/0). The desired fractions were collected and concentrated in vacuo to yield 2-[7-isopropyl-2-(methylamino)-4-oxo-pyrazolo[1,5-d] [1,2,4]triazin-5-yl]—N-pyrimidin-4-yl-acetamide (Final compound 18) (6 mg, 8%) as a white solid and 2-[7-isopropyl (methyl amino)-4-oxo-pyrazolo[1,5-d] [1,2,4]triazin-5-yl]—N-methyl-acetamide

(Compound 19) (13 mg, 21%) as a white solid. Additional analogs were accessed using similar reaction conditions, using the appropriate reagent.

| Reagent | Starting material | Final compound | Number |
|---|---|---|---|
| [593-51-1] | 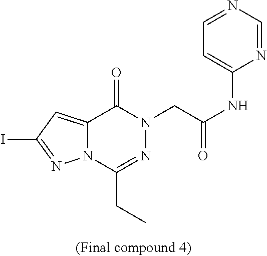 [SMILES: CCc1nn(CC(=O)Nc2ccncn2)c(=O)c2cc(I)nn12] | 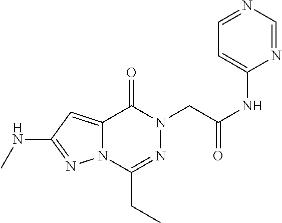 [SMILES: CCc1nn(CC(=O)Nc2ccncn2)c(=O)c2cc(NC)nn12] | Final Cpd 20 |
| [36520-39-5] | 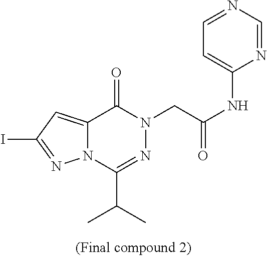 [SMILES: CC(C)c1nn(CC(=O)Nc2ccncn2)c(=O)c2cc(I)nn12] | 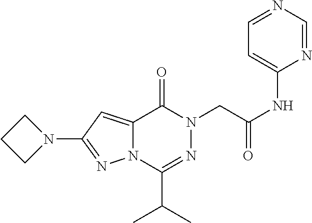 [SMILES: CC(C)c1nn(CC(=O)Nc2ccncn2)c(=O)c2cc(N3CCC3)nn12] | Final Cpd 21 |
| [506-59-2] | 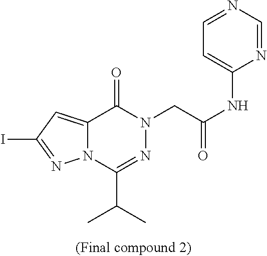 [SMILES: CC(C)c1nn(CC(=O)Nc2ccncn2)c(=O)c2cc(I)nn12] | 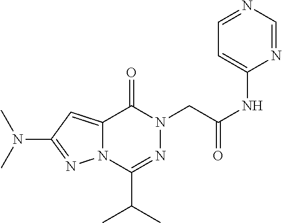 [SMILES: CC(C)c1nn(CC(=O)Nc2ccncn2)c(=O)c2cc(N(C)C)nn12] | Final Cpd 22 |
| [2393-23-9] | 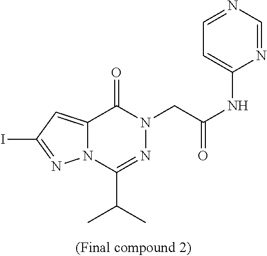 [SMILES: CC(C)c1nn(CC(=O)Nc2ccncn2)c(=O)c2cc(I)nn12] | 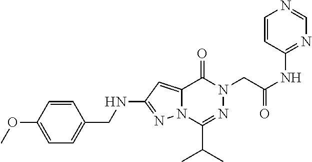 [SMILES: COc1ccc(CNc2cc3c(=O)n(CC(=O)Nc4ccncn4)nc(C(C)C)n3n2)cc1] | Final Cpd 178 |

Synthesis of 2-(2-(1-ethoxyvinyl)-7-isopropyl-4-oxopyrazolo[1,5-d][1,2,4]triazin-5(4H)-yl)-N-(pyrimidin-4-yl)acetamide Final Cpd 179

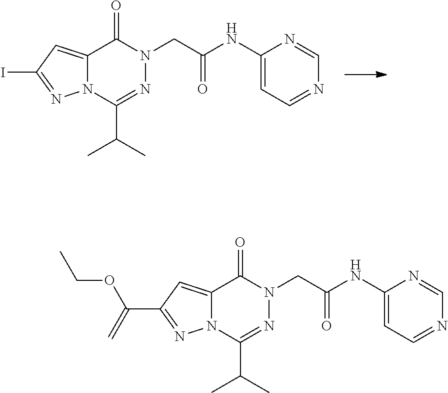 [SMILES: CC(C)c1nn(CC(=O)Nc2ccncn2)c(=O)c2cc(I)nn12 ; C=C(OCC)c1cc2c(=O)n(CC(=O)Nc3ccncn3)nc(C(C)C)n2n1]

1,4-dioxane (1.5 mL) followed by tributyl(1-ethoxyvinyl)stannane [97674-02-7] (0.13 mL, 1.07 g/mL, 0.38 mmol) were added to a stirred mixture of 2-(2-iodo-7-isopropyl oxopyrazolo[1,5-d] [1,2,4]triazin-5(4H)-yl)-N-(pyrimidin-4-yl)acetamide (Final compound 2) (113 mg, 0.26 mmol) and bis(tri-tert-butylphosphine)palladium(O) [53199-31-8] (12 mg, 0.023 mmol) in a sealed tube and under N2. The mixture was stirred at 100° C. for 4 h. The solvent was evaporated in vacuo and the crude product was purified by RP flash chromatography (C18, ACN in NH₄HCO_{3 0.25}% solution in

Water 10/90 to 100/0). The desired fractions were collected and concentrated in vacuo to yield 2-(2-(1-ethoxyvinyl)-7-isopropyl-4-oxopyrazolo[1,5-d] [1,2,4]triazin-5(4H)-yl)-N-(pyrimidin-4-yl)acetamide Final Cpd 179 (60 mg, yield 61%) as a white solid.

Synthesis of 2-(2-(1-ethoxyethyl)-74 sopropyl-4-oxopyrazolo[1,5-d][1,2,4]triazin-5(4H)-yl)-N-(pyrimidin-4-yl)acetamide Final Cpd 180

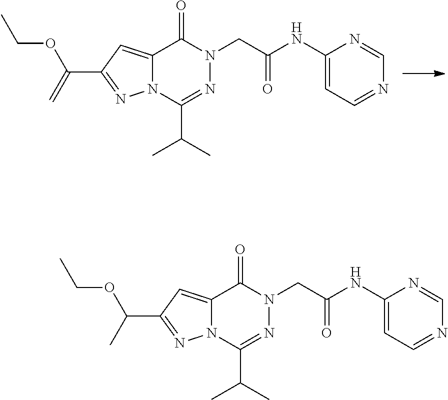 [SMILES: C=C(OCC)c1cc2c(=O)n(CC(=O)Nc3ccncn3)nc(C(C)C)n2n1 ; CCOC(C)c1cc2c(=O)n(CC(=O)Nc3ccncn3)nc(C(C)C)n2n1]

A solution of 2-(2-(1-ethoxyvinyl)-7-isopropyl-4-oxopyrazolo[1,5-d][1,2,4]triazin-5(4H)-yl)-N-(pyrimidin-4-yl)acetamide Final Cpd 179 (30 mg, 0.078 mmol) in EtOH (2.5 mL) and THF (2.5 mL) was hydrogenated in a H-cube reactor (1 mL/min, 30 mm Pd/C 10% cartridge, full H₂ mode, 25° C., 1 cycle). The solvent was evaporated in vacuo to yield 2-(2-(1-ethoxyethyl)-74 sopropyl-4-oxopyrazolo[1,5-d] [1,2,4]triazin-5(4H)-yl)-N-(pyrimidin-4-yl)acetamide Final Cpd 180 (30 mg, yield 73%) as a white solid.

Synthesis of 2-(3-amino-2-cyclopropyl-7-isopropyl-4-oxopyrazolo[1,5-d][1,2,4]triazin-5(4H)-yl)-N-((ls,3s)-3-hydroxy-3-methylcyclobutyl)acetamide Final Cpd 181

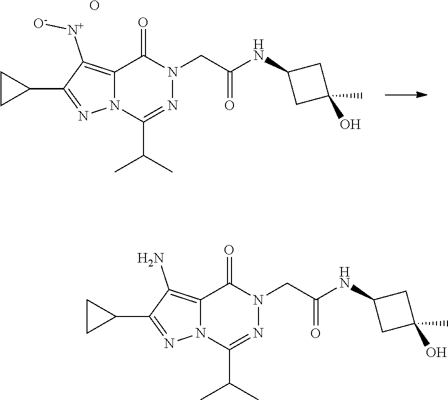

Pd/C (10%, degussa type) (23 mg, 0.022 mmol) was added to a stirred solution of 2-(2-cyclopropyl-7-isopropyl-3-nitro-4-oxopyrazolo [1,5-d] [1,2,4]triazin-5 (4H)-yl)-N-((1 s,3 s)-3-hydroxy-3-methylcyclobutyl)acetamide (84 mg, 0.21 mmol) in a mixture of MeOH (3 mL) and EtOAc (3 mL) under N2. The mixture was hydrogenated at atmospheric pressure (balloon) at RT for 20 h. The mixture was filtered through celite and the filtrated was evaporated in vacuo. The crude product was purified by RP HPLC

(Stationary phase: C18)(Bridge 30×100 mm 5 μm), Mobile phase: Gradient from 85% NH₄HCO_{3 0.25}% solution in Water, 15% CH₃CN to 55% NH₄HCO_{3 0.25}% solution in Water, 45% CH₃CN). Desired fractions were collected and evaporated in vacuo to yield 2-(3-amino-2-cyclopropyl-7-i sopropyl-4-oxopyrazolo[1,5-d] [1,2,4]triazin-5(4H)-yl)-N-((1 s,3 s)-3-hydroxy-3-methylcyclobutyl)acetamide Final Cpd 181 (42 mg, yield 54%) as a white solid.

Additional analogs were accessed using similar reaction conditions.

| Starting material | Final compound | Number |
|---|---|---|
| 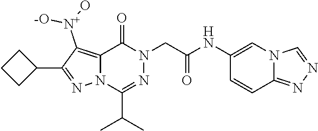 | 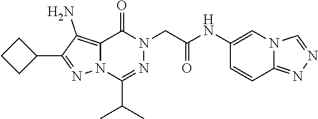 [SMILES: CC(C)c1nn(CC(=O)Nc2ccc3nncn3c2)c(=O)c2c(N)c(C3CCC3)nn12] | 182 |

Synthesis of 2-(2-acetyl-7-isopropyl-4-oxopyrazolo[1,5-d][1,2,4]triazin-5(4H)-yl)-N-(pyrimidin-4-yl)acetamide Final Cpd 183

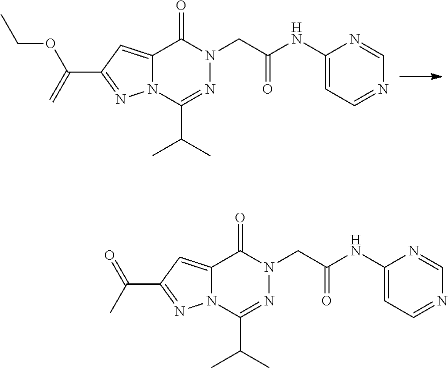 [SMILES: C=C(OCC)c1cc2c(=O)n(CC(=O)Nc3ccncn3)nc(C(C)C)n2n1 ; CC(=O)c1cc2c(=O)n(CC(=O)Nc3ccncn3)nc(C(C)C)n2n1]

HCl (2M in water) (0.4 mL, 2M, 0.8 mmol) was added to a stirred solution of 2-(2-(1-ethoxyvinyl)-7-isopropyl-4-oxopyrazolo[1,5-d][1,2,4]triazin-5(4H)-yl)-N-(pyrimidin-4-yl)acetamide Final Cpd 179 (154 mg, 0.4 mmol) in THF (4 mL) in a tube and under N2. The mixture was stirred at rt for 2 h. The mixture was carefully basified with 10% Na₂CO₃ and extracted with DCM/iPrOH (9/1). The organic layer was separated, dried (MgSO₄), filtered and the solvents were evaporated in vacuo. The crude product was purified by RP flash chromatography (C18, ACN in NH₄HCO_{3 0.25}% solution in Water 15/85 to 100/0). The desired fractions were collected and concentrated in vacuo to yield 2-(2-acetyl-7-isopropyl-4-oxopyrazolo[1,5-d][1,2,4]triazin-5(4H)-yl)-N-(pyrimidin-4-yl)acetamide Final Cpd 183 (89 mg, yield 62%) as a white solid.

Synthesis of 2-(2-acetamido-7-ethyl-4-oxopyrazolo[1,5-d][1,2,4]triazin-5(4H)-yl)-N-(pyrimidin-4-yl)acetamide Final Cpd 184

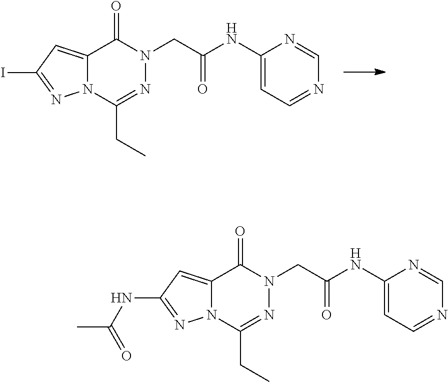 [SMILES: CCc1nn(CC(=O)Nc2ccncn2)c(=O)c2cc(I)nn12 ; CCc1nn(CC(=O)Nc2ccncn2)c(=O)c2cc(NC(C)=O)nn12]

Anhydrous 1,4-dioxane (1 mL) was added to a stirred mixture of 2-(7-ethyl-2-iodo-4-oxopyrazolo [1,5-d] [1,2,4]triazin-5(4H)-yl)-N-(pyrimidin-4-yl)acetamide (final compound 4) (45 mg, 0.11 mmol), acetamide [158734-79-3] (14 mg, 0.24 mmol), XantPhos Pd G3 [1445085-97-1] (9 mg, 0.0095 mmol), Cs₂CO_{3 [534}-17-8] (98 mg, 0.3 mmol) and 4,5-bis(diphenylphosphino)-9,9-dimethylxanthene [161265-03-8] (6 mg, 0.01 mmol) in a sealed tube and under N2. The mixture was stirred at 90° C. for 2.5 days. The mixture was treated with water and extracted with DCM. The organic layer was separated, filtered through a phase separator cartridge and the solvent concentrated in vacuo. The crude product was purified by flash column chromatography (SiO2, 7N solution of NH₃ in MeOH in DCM 0.5/99.5 to 10/90). The desired fractions were collected and concentrated in vacuo to yield 2-(2-acetamido-7-ethyl-4-oxopyrazolo[1,5-d][1,2,4]triazin-5(4H)-yl)-N-(pyrimidin-4-yl)acetamide Final Cpd 184 (38 mg, yield 32%) as a white solid.

Synthesis of 2-(2-(1-hydroxyethyl)-7-sopropyl-4-oxopyrazolo[1,5-d][1,2,4]triazin-5(4H)-yl)-N-(pyrimidin-4-yl)acetamide Final Cpd 185

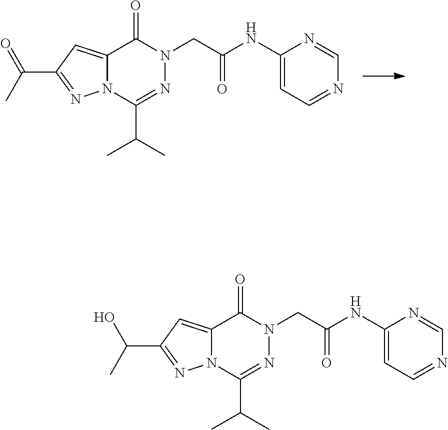 [SMILES: CC(=O)c1cc2c(=O)n(CC(=O)Nc3ccncn3)nc(C(C)C)n2n1 ; CC(C)c1nn(CC(=O)Nc2ccncn2)c(=O)c2cc(C(C)O)nn12]

Sodium borohydride [16940-66-2] (3 mg, 0.079 mmol) was added to a stirred suspension of 2-(2-acetyl-7-isopropyl-4-oxopyrazolo[1,5-d][1,2,4]triazin-5(4H)-yl)-N-(pyrimidin-4-yl)acetamide Final Cpd 183 (25 mg, 0.07 mmol) in iPrOH (0.7 mL) at 0° C. in a tube and under N2. The mixture was stirred at 0° C. for 2 h. The mixture was carefully treated with 20% NH₄Cl and extracted with DCM/iPrOH (9/1). The organic layer was separated, dried (MgSO₄), filtered and the solvents evaporated in vacuo. The crude product was purified by flash column chromatography (SiO2, 7N solution of NH₃ in MeOH in DCM 0.5/99.5 to 10/90). The desired fractions were collected and concentrated in vacuo to yield 2-(2-(1-hydroxyethyl)-74 sopropyl-4-oxopyrazolo[1,5-d][1,2,4]triazin-5(4H)-yl)-N-(pyrimidin-4-yl)acetamide Final Cpd 185 (14 mg, yield 56%) as a white solid.

Synthesis of 2-(2-amino-7-isopropyl-4-oxopyrazolo[1,5-d][1,2,4]triazin-5(4H)-yl)-N-(pyrimidin-4-yl)acetamide Final Cpd 186

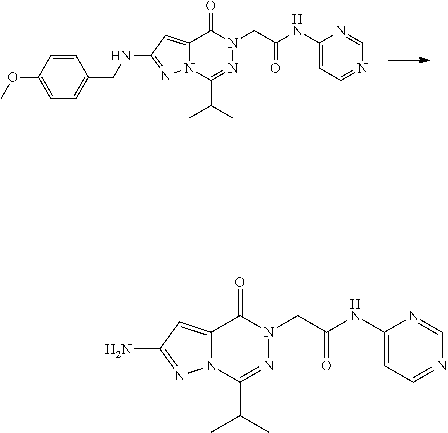 [SMILES: COc1ccc(CNc2cc3c(=O)n(CC(=O)Nc4ccncn4)nc(C(C)C)n3n2)cc1 ; CC(C)c1nn(CC(=O)Nc2ccncn2)c(=O)c2cc(N)nn12]

TFA (0.5 mL) was added to 2-(7-isopropyl-2-((4-methoxybenzyl)amino)-4-oxopyrazolo[1,5-d][1,2,4]triazin-5(4H)-yl)-N-(pyrimidin-4-yl)acetamide Final Cpd 178 (20 mg, 0.05 mmol) in a sealed tube and under N2. The mixture was stirred at rt for 2 h. The solvent was evaporated in vacuo. The crude was basified with 10% Na₂CO₃ and extracted with DCM/iPrOH (9/1). The organic layer was separated, dried (MgSO₄), filtered and the solvents evaporated in vacuo. The crude product was purified by RP flash chromatography (C18, ACN in NH₄HCO_{3 0.25}% solution in Water 15/85 to 100/0). The desired fractions were collected and concentrated in vacuo to yield 2-(2-amino-7-i sopropyl-4-oxopyrazol O[1,5-d] [1,2,4]triazin-5(4H)-yl)-N-(pyrimidin-4-yl)acetamide Final Cpd 186 (7 mg, yield 48%) as a white solid.

Synthesis of (R)-2-(2-cyclopropyl-7-isopropyl-4-oxopyrazolo[1,5-d][1,2,4]triazin-5(4H)-yl)-N-(piperidin-3-yl)acetamide Final Cpd 187

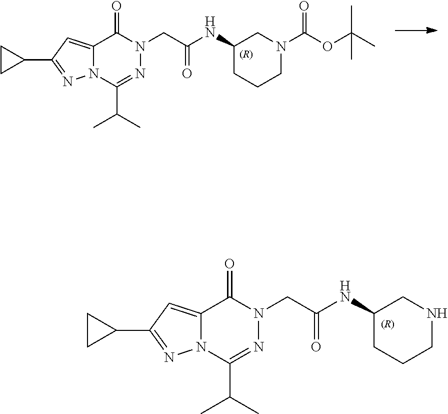

HCl (4M in dioxane) [7647-01-0] (1.28 mL, 4 M, 5.12 mmol) was added to a stirred solution of tert-butyl (R)-3-(2-(2-cyclopropyl-7-isopropyl-4-oxopyrazolo[1,5-d][1,2,4]triazin-5(4H)-yl)acetamido)piperidine-1-carboxylate (234 mg, 0.51 mmol) in 1,4-dioxane (5 mL). The mixture was stirred at RT for 16 h. The solvent was evaporated in vacuo and the product was triturated with Et2O to yield (R)-2-(2-cyclopropyl-7-isopropyl-4-oxopyrazolo[1,5-d][1,2,4]triazin-5(4H)-yl)-N-(piperidin yl)acetamide Final Cpd 187 (135 mg, yield 74%) as a white solid.

Additional analogs were accessed using similar reaction conditions.

| Starting material | Final compound | Number |
|---|---|---|
| 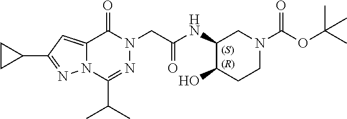 | 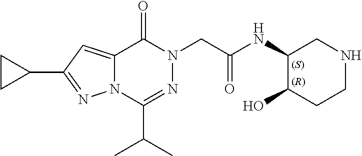 | 188 |

Syntesis of (R)-2-(2-cyclopropyl-7-isopropyl-4-oxopyrazolo[1,5-d][1,2,4]triazin-5(4H)-yl)-N-(1-(2,2,2-trifluoroethyl)piperidin-3-yl)acetamide Final Cpd 189

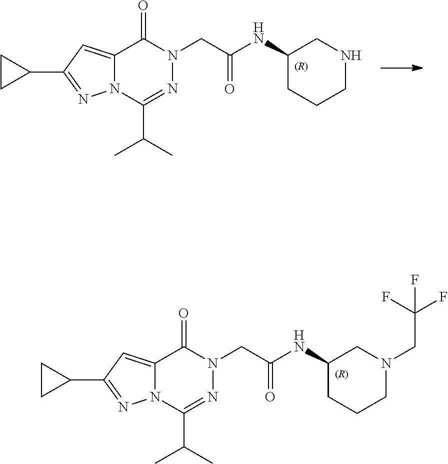

To stirred solution of (R)-2-(2-cyclopropyl-7-isopropyl-4-oxopyrazolo[1,5-d][1,2,4]triazin-5(4H)-yl)-N-(piperidin-3-yl)acetamide Final Cpd 187 (65 mg, 0.18 mmol) in AcOEt (10 mL) and water (6.5 ml) was added 2,2,2-trifluoroethyl trifluoromethanesulfonate [6226-25-1] (50 mg, 0.215 mmol) and NaHCO_{3 [144}-55-8 ] (105 mg, 1.25 mmol) at rt. The mixture was stirred at 50° C. for 16h. The crude was diluted with water and AcOEt and the layers were separated. The organic layer was dried over MgSO₄, filterd and aconcentrated in vacuo to afford a solid, the solid was washed with a few drops of cold MeCN and then with DIPE to yield (R)-2-(2-cyclopropyl-7-isopropyl-4-oxopyrazolo[1,5-d] [1,2,4]triazin-5(4H)-yl)-N-(1-(2,2,2-trifluoroethyl)piperidin-3-yl)acetamide Fianl Cpd 189 (61 mg, yield 76%).

Synthesis of 2-(2-cyclopropyl-7-isopropyl-4-oxopyrazolo[1,5-d][1,2,4]triazin-5(4H)-yl)-N-((3S,4R)-1-ethyl-4-hydroxypiperidin-3-yl)acetamide Final Cpd 190

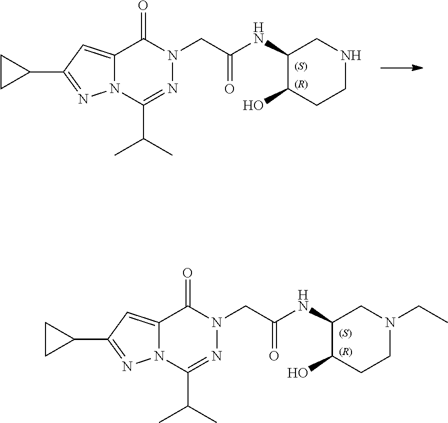

Et3N [121-44-8] (81 0.73 g/mL, 0.58 mmol) was added to a stirred suspension of 2-(2-cyclopropyl-7-isopropyl-4-oxopyrazolo[1,5-d][1,2,4]triazin-5(4H)-yl)-N-((3S,4R)-4-hydroxypiperidin-3-yl)acetamide (60 mg, 0.15 mmol) in ACN (1.56 mL) at rt , the mixture was stirred for 5 mins followed by the addition of iodoethane [75-03-6] (18 1.94 g/mL, 0.22 mmol). The mixture was stirred at rt for 2 h. Then, the mixture was diluted with water and aq NaHCO3 sat. soln. and extracted with DCM. A phase separator cartridge was used. The organic layer was evaporated in vacuo and the crude was purified by flash column chromatography (silica, MeOH in DCM 0/100 to 6/94).

The desired fractions were collected and the solvent evaporated in vacuo to yield a white solid. A purification was performed via chiral SFC (Stationary phase: Chiralpak IG 5 μm 250*20mm, Mobile phase: 60% CO2, 40% MeOH(0.3% iPrNH₂)) yielding 2-(2-cyclopropyl-7-isopropyl-4-oxopyrazolo[1, 5-d] [1,2,4]triazin-5(4H)-yl)-N-((3 S,4R)-1-ethyl-4-hydroxypiperidin-3-yl)acetamide Final Cpd 190 (34mg, yield 58%).

Synthesis of N-((3 S,4R)-1-cyclobutyl-4-hydroxypiperidin-3-yl)-2-(2-cyclopropyl-7-isopropyl-4-oxopyrazolo[1,5-d][1,2,4]triazin-5(4H)-yl)acetamide Final Cpd 191

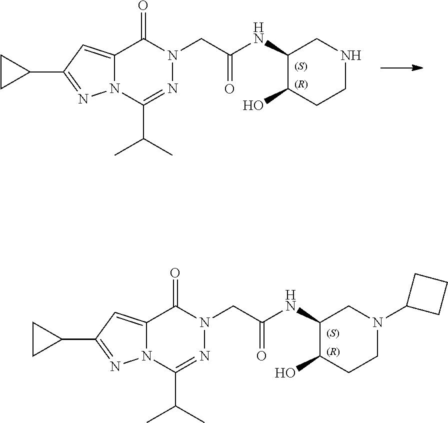

Sodium triacetoxyborohydride [56553-60-7] (42 mg, 0.2 mmol) was added portionwise to a stirred solution of 2-(2-cyclopropyl-7-isopropyl-4-oxopyrazolo[1,5-d][1,2,4]triazin-5(4H)-yl)-N-((3S,4R)-4-hydroxypiperidin-3-yl)acetamide (50 mg, 0.12 mmol) and cyclobutanone [1191-95-3] (13 0.94 g/mL, 0.17 mmol) in DCE (2 mL).

The resulting mixture was stirred at RT for 18h. The reaction mixture was diluted with water and 20% aq NaHCO₃ and extracted with DCM. The organic layer was separated and dried (Na₂SO₄), filtered and the solvent evaporated in vacuo. The crude product was purified by flash column chromatography (silica, MeOH in DCM 0/100 to 6/95). The desired fractions were collected and the solvent evaporated in vacuo to yield N-((3 S,4R)-1-cyclobutyl-4-hydroxypiperidin-3-yl)-2-(2-cyclopropyl-7-isopropyl oxopyrazolo[1,5-d][1,2,4]triazin-5(4H)-yl)acetamide Final Cpd 191 (45 mg, yield 90%) as a white solid.

Synthesis of 2-(2-cyclopropyl-7-isopropyl-4-oxopyrazolo[1,5-d][1,2,4]triazin-5(4H)-yl)-N-((1r,3s)-3-ethyl-3-hydroxycyclobutyl)acetamide Final Cpd 192

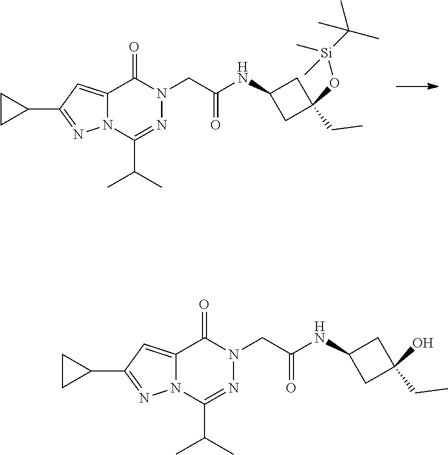 [SMILES: CC[C@]1(O[Si](C)(C)C(C)(C)C)C[C@@H](NC(=O)Cn2nc(C(C)C)n3nc(C4CC4)cc3c2=O)C1 ; CC[C@]1(O)C[C@@H](NC(=O)Cn2nc(C(C)C)n3nc(C4CC4)cc3c2=O)C1]

To a mixture of N-((1r,3s)-3-((tert-butyldimethylsilyl)oxy)-3-ethylcyclobutyl)-2-(2-cyclopropyl-7-isopropyl-4-oxopyrazolo[1,5-d][1,2,4]triazin-5(4H)-yl)acetamide (100 mg, 0.205 mmol) in THF (3mL), tetrabutylammonium fluoride 1M in THF [429-41-4] (0.82 mL, 1 M, 0.82 mmol) was added at rt. The mixture was stirred for 24 h at rt.

Water was added, the crude was extracted with DCM (2×3 ml) and the combined organic layers were evaporated in vacuo. The crude was purified by flash column chromatography (MeOH in DCM 0/100 to 5/95). The desired fractions were collected, the solvent evaporated in vacuo to yield 2-(2-cyclopropyl-7-isopropyl-4-oxopyrazolo[1,5-d][1,2,4]triazin-5(4H)-yl)-N-((1r,3 s)-3-ethyl-3-hydroxycyclobutyl)acetamide Final Cpd 192 (56 mg, yield 73%) as white solid.

Synthesis of N-(8-cyano-[1,2,4]triazolo[4,3-a]pyridin-6-yl)-2-(2-cyclopropyl-7-isopropyl-4-oxopyrazolo[1,5-d][1,2,4]triazin-5(4H)-yl)acetamide Final Cpd 193

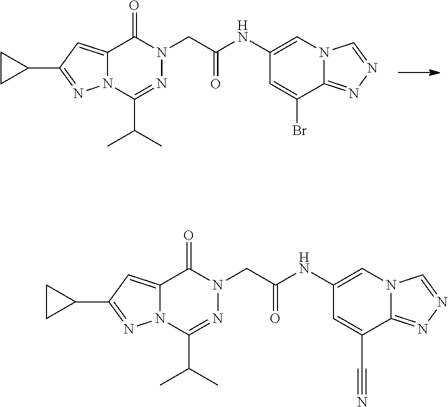 [SMILES: CC(C)c1nn(CC(=O)Nc2cc(Br)c3nncn3c2)c(=O)c2cc(C3CC3)nn12 ; CC(C)c1nn(CC(=O)Nc2cc(C#N)c3nncn3c2)c(=O)c2cc(C3CC3)nn12]

An 8 mL 1\4W vial was charged with: tBuXPhos Pd G3 (20.2 mg, 25.5 μmol, 15 mol%), Zn(CN)₂ (35.9 mg, 0.31 mmol, 1.8 equiv) and N-(8-bromo-[1,2,4]triazolo[4,3-a]pyridin-6-yl)-2-(2-cyclopropyl-7-isopropyl-4-oxopyrazolo[1, 5-d] [1,2,4]triazin-5(4H)-yl)acetamide (80 mg, 0.17 mmol, 1 equiv). The vial was sealed and placed under nitrogen (3 vacuum/nitrogen cycles) and 1.86 mL of degassed 1:2 mixture of THF/DI water was added. The vial was stirred vigorously at 60° C. for 22h. The mixture was then diluted with DI water (5 mL) and extracted with DCM/MeOH 95:5 (8×5 mL). The combined organic extracts were filtered over a short pad of Celite and the filtrate concentrated in vacuo to give an off-white solid. A purification was performed via Prep HPLC (Stationary phase: RP)(Bridge Prep C18 OBD-5 μm, 50×250mm, Mobile phase: 0.25% NH₄HCO₃ solution in water, CH₃CN) yielding N-(8-cyano-[1,2,4]triazolo[4,3-a]pyridin-6-yl)-2-(2-cyclopropyl-7-isopropyl-4-oxopyrazolo[1,5-d][1,2,4]triazin-5(4H)-yl)acetamide Final Cpd 193 as a colorless solid (15.6 mg, yield 22%, >98% purity).

Synthesis of 2-(2-cyclopropyl-7-(3-methoxypropyl)-4-oxopyrazolo[1,5-d][1,2,4]triazin-5(4H)-yl)-N-((ls,3s)-3-hydroxy-3-methylcyclobutyl)acetamide Final Cpd 194

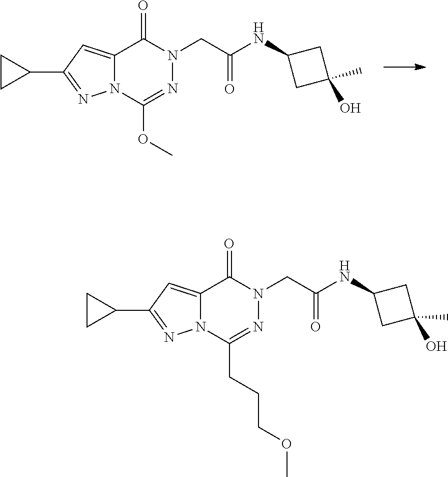 [SMILES: COc1nn(CC(=O)N[C@H]2C[C@@](C)(O)C2)c(=O)c2cc(C3CC3)nn12 ; COCCCc1nn(CC(=O)N[C@H]2C[C@@](C)(O)C2)c(=O)c2cc(C3CC3)nn12]

(3-Methoxypropyl)magnesium(II) (2.38 mL, 0.42 M, 1 mmol) was collected over a solution of 2-(2-cyclopropyl-7-methoxy-4-oxopyrazolo[1,5-d][1,2,4]triazin-5(4H)-yl)-N-((1s,3s)-3-hydroxy-3-methylcyclobutyl)acetamide (69.47 mg, 0.2 mmol) and (1,3-dimesityl-2,3-dihydro-1 h-imidazol-2-ylidene)nickel(O) bis(di-tert-butyl fumarate) [38-72-9] (16.39 mg, 0.02 mmol) in THF (0.5 ml) purged with a nitrogen atmosphere. The reaction was kept stirring for 1 hour at 80° C. and then quenched with citric acid and extracted with a mixture of DCM/MeOH 9/1. Ni Scavenger (0.49 g, 4.11 mmol/g, 2 mmol) was added to the organic layers and then they were left in the shaker for overnight and filtered to remove the scavenger. The filtrate was evaporated under vacuum and the residue was purified by RP HPLC (Stationary phase: C18 XBridge 30×100 mm 5 μm), Mobile phase: Gradient from 98% NH₄HCO3 0.25% solution in Water, 2% CH₃CN to 60% NH₄HCO_{3 0.25}% solution in Water, 40% CH₃CN), yielding 2-(2-cyclopropyl-7-(3-methoxypropyl)-4-oxopyrazolo[1,5-d][1,2,4]triazin-5(4H)-yl)-N-((1s,3s)-3-hydroxy-3-methylcyclobutyl)acetamide Final Cpd 194 (2.6 mg, yield 3%) as an off white solid.

Synthesis of 2-(2-cyclopropyl-4-oxo-7-(4,4,4-trifluorobutyl)pyrazolo[1,5-d][1,2,4]triazin-5(4H)-yl)-N-((ls,3s)-3-hydroxy-3-methylcyclobutyl)acetamide — Final Cpd 195

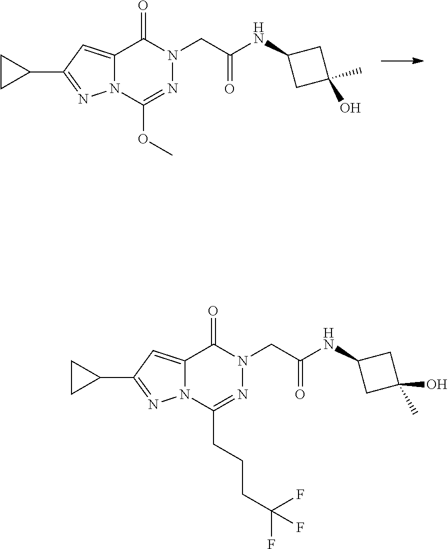 [SMILES: COc1nn(CC(=O)N[C@H]2C[C@@](C)(O)C2)c(=O)c2cc(C3CC3)nn12 ; C[C@]1(O)C[C@@H](NC(=O)Cn2nc(CCCC(F)(F)F)n3nc(C4CC4)cc3c2=O)C1]

2-(2—Cyclopropyl-7-methoxy-4-oxopyrazolo[1,5-d][1,2,4]triazin-5(4H)-yl)-N-((ls,3s)-3-hydroxy-3-methylcyclobutyl)acetamide (34.74 mg, 0.1 mmol) and (1,3-dimesityl-2,3-dihydro-1h-imidazol-2-ylidene)nickel(O) bis(di-tert-butyl fumarate) [2091838 9] (8.2 mg, 0.01 mmol) were placed in microwave vial and purged with a nitrogen atmosphere. Then, THF (1 mL) and 1-bromo-4,4,4-trifluorobutane [406-81-5] (458 mg, 2.4 mmol) were added in a sequential maner prepared in the R2-R4 Vapourtec. The reaction was kept stirring for 1 hour at 80° C. Then the crude was quenched with MeOH and Ni Scavenger (80.65 mg, 0.62 mmol/g, 0.05 mmol) was added and left stirring for 1 hour. The organic solvent was evaporated and the crude was submitted to purification and lyofilization to yield 2-(2-cyclopropyl-4-oxo-7-(4,4,4-trifluorobutyl)pyrazolo[1,5-d] [1,2,4]triazin-5(4H)-yl)-N-((1 s,3 s)-3-hydroxy-3-methylcyclobutyl)acetamide Final Cpd 195 (2.4 mg, yield 6%) as a white solid.

Additional analogs were accessed using similar reaction conditions, using the appropriate reagent.

| Reagent | Final compound | Number |
|---|---|---|
| 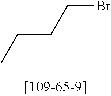 | 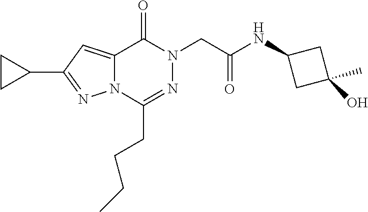 [SMILES: CCCCc1nn(CC(=O)N[C@H]2C[C@@](C)(O)C2)c(=O)c2cc(C3CC3)nn12] | 196 |
| 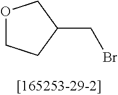 | 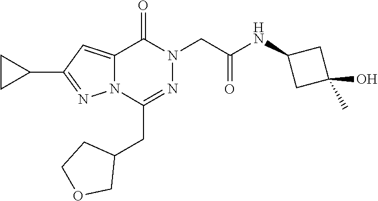 [SMILES: C[C@]1(O)C[C@@H](NC(=O)Cn2nc(CC3CCOC3)n3nc(C4CC4)cc3c2=O)C1] | 197 |
| 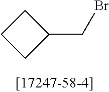 | 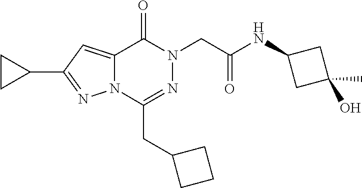 [SMILES: C[C@]1(O)C[C@@H](NC(=O)Cn2nc(CC3CCC3)n3nc(C4CC4)cc3c2=O)C1] | 198 |

Synthesis of 2-(2-cyclopropyl-7-(4-methoxyphenyl)-4-oxopyrazolo [1,5-d] [1,2,4]triazin-5(4H)-yl)-N-((1 s,3 s)-3-hydroxy-3-methylcyclobutyl)acetamide Final

Cpd 199

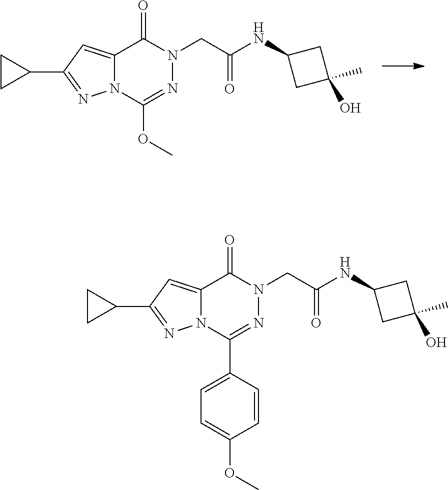 [SMILES: COc1nn(CC(=O)N[C@H]2C[C@@](C)(O)C2)c(=O)c2cc(C3CC3)nn12 ; COc1ccc(-c2nn(CC(=O)N[C@H]3C[C@@](C)(O)C3)c(=O)c3cc(C4CC4)nn23)cc1]

2-(2—Cyclopropyl-7-methoxy-4-oxopyrazolo[1,5-d] [1,2,4]triazin-5(4H)-yl)-N-((1s,3 s)-3-hydroxy-3-methylcyclobutyl)acetamide (60 mg, 0.155 mmol) and (1,3-dimesityl-2,3-dihydro-1 h-imidazol-2-ylidene)nickel(O) bis(di-tert-butyl fumarate) [2091838-72-9] (12.74 mg, 0.0155 mmol, were placed in microwave vials and purged with a nitrogen atmosphere. Then, THF (2.4 mL) and 4-methoxyphenylmagnesium bromide [13139-86-1] (1.55 mL, 0.5 M, 0.78 mmol) were added via syringe The reactions were kept stirring for 1 hour at 80° C. Then, AcOH in MeOH (0.1 M, 0.5 mL) was added, stirring at room temperature for 5 minutes. Water was added (5 mL), and the products were extracted in EtOAc (5 mL x 6). The combined organic layers were concentrated in vacuo and redissolved in DMSO (2 mL) and MeOH (0.5 mL), filtered and purified by RP HPLC (Stationary phase: C18)(Bridge, column with 100 mm length, 5 μm. Mobile phase: Gradient using NH₄HCO_{3 0.25}% solution in Water and CH₃CN as organic solvent), yielding 2-(2-cyclopropyl-7-(4-methoxyphenyl)-4-oxopyrazolo[1,5-d] [1,2,4]triazin-5(4H)-yl)-N-((1 s,3 s)-3-hydroxy-3-methylcyclobutyl)acetamide Final Cpd 199 (14 mg, yield 21%) as a white solid after lyophilization.

Additional analogs were accessed using similar reaction conditions, using the appropriate reagent.

| | Reagent | | |
|---|---|---|---|
| Catalyst | (0.78 mmol each) | Final compound | Number |
| [2230140-51-7] | [21969-32-4] | 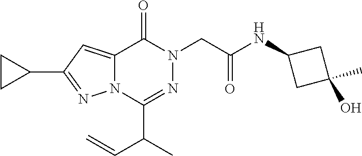 [SMILES: C=CC(C)c1nn(CC(=O)N[C@H]2C[C@@](C)(O)C2)c(=O)c2cc(C3CC3)nn12] | 200 |
| [2230140-51-7] | [15366-08-2] | 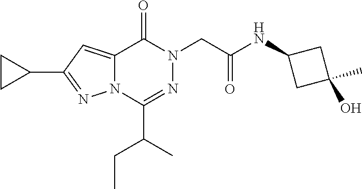 [SMILES: CCC(C)c1nn(CC(=O)N[C@H]2C[C@@](C)(O)C2)c(=O)c2cc(C3CC3)nn12] | 201 |
| [2091838-72-9] | [41251-37-0] | 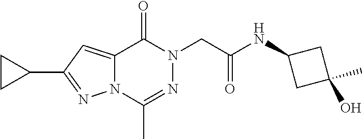 [SMILES: Cc1nn(CC(=O)N[C@H]2C[C@@](C)(O)C2)c(=O)c2cc(C3CC3)nn12] | 202 |

Characterising Data—LC-MS

| Compound | LC-MS (Rt, area %, MW, [M + H]⁺, Method) |
|---|---|
| Final Cpd 1 | Rt: 1.75, Area %: 95.86, MW: 341, [M + H]⁺: 342, Method: A |
| Final Cpd 2 | Rt: 1.79, Area %: 95.66, MW: 439, [M + H]+: 440, Method: A |
| Final Cpd 23 | RT: 1.53, Area %: 96.53, MW: 478.04, BPM1: 479.0, Method: A |
| Final Cpd 3 | Rt: 2.09, Area %: 100, MW: 355, [M + H]+: 356, Method: A |
| Final Cpd 4 | Rt: 1.48, Area %: 99.14, MW: 425, [M + H]+: 426, Method: A |
| Final Cpd 5 | Rt: 1.78, Area %: 97.95, MW: 339, [M + H]+: 340, Method: A |
| Final Cpd 6 | Rt: 1.28, Area %: 98.7, MW: 464, [M + H]+: 465, Method: A |
| Final Cpd 7 | Rt: 1.23, Area %: 99.11, MW: 431, [M + H]+: 432, Method: A |
| Final Cpd 8 | Rt: 1.59, Area %: 99.35, MW: 359, [M + H]⁺: 360, Method: A |
| Final Cpd 9 | Rt: 2.00, Area %: 96.84, MW: 412, [M + H]+: 413, Method: A |
| Final Cpd 25 | RT: 1.97, Area %: 94.35, MW: 426.24, BPM1: 427.33, BPM2: 425.24, Method: E |
| | SFC (Rt 4.86 min 48.47% isomer 1), (Rt 5.85 min 50.19% isomer 2) (RT: 4.86, |
| | Area %: 48.47, MW: 426.24, BPM1: 427, BPM2: 425, Method: 1H_EtOH_G2A1P1BSFC5) |
| Final Cpd 26 | RT: 1.76, Area %: 100.00, MW: 426.20, BPM1: 427, BPM2: 425, Method: N) |
| | SFC (Rt 4.86 min 100.00% isomer 1), (Rt 5.85 min 0.00% isomer 2) (RT: 4.86, |
| | Area %: 100.00, MW: 426.24, BPM1: 427, BPM2: 425, Method: 1H_EtOH_G2A1P1BSFC5) |
| Final Cpd 27 | RT: 1.76, Area %: 100.00, MW: 426.20, BPM1: 427, BPM2: 425, Method: N |
| | SFC (Rt 4.86 min 0.00% isomer 1), (Rt 5.85 min 100.00% isomer 2) (RT: 5.85, |
| | Area %: 100.00, MW: 426.24, BPM1: 427, BPM2: 425, Method: 1H_EtOH_G2A1P1BSFC5) |
| Final Cpd 28 | RT: 1.66, Area %: 100.00, MW: 382.18, BPM1: 383.29, BPM2: 381.18, Method: E |
| Final Cpd 10 | Rt: 1.54, Area %: 98.84, MW: 339, [M + H]+: 340, Method: A |
| Final Cpd 11 | Rt: 1.87, Area %: 100, MW: 353, [M + H]+: 354, Method: A |
| Final Cpd 12 | Rt: 2.05, Area %: 95.30, MW: 355, [M + H]+: 356, Method: A |
| Final Cpd 13 | Rt: 1.44, Area %: 95.98, MW: 327, [M + H]+: 328, Method: A |
| Final Cpd 14 | Rt: 1.11, Area %: 95.96, MW: 313, [M + H]⁺: 314, Method: A |
| Final Cpd 15 | Rt: 1.78, Area %: 100, MW: 341, [M + H]+: 342, Method: A |
| Final Cpd 16 | Rt: 1.82, Area %: 98.21, MW: 341, [M + H]+: 342, Method: A |
| Final Cpd 17 | Rt: 1.44, Area %: 95.67, MW: 327, [M + H]+: 328, Method: A |
| Final Cpd 18 | Rt: 1.38, Area %: 92.49, MW: 342, [M + H]⁺: 343, Method: A |
| Cpd 19 | Rt: 1.10, Area %: 95.51, MW: 278, [M + H]⁺: 279, Method: A |
| Final Cpd 20 | Rt: 1.09, Area %: 92.55, MW: 328, [M + H]⁺: 329, Method: A |
| Final Cpd 21 | Rt: 1.71, Area %: 95.57, MW: 368, [M + H]⁺: 369, Method: A |
| Final Cpd 22 | Rt: 1.82, Area %: 95.39, MW: 356, [M + H]⁺: 357, Method: A |
| Final Cpd 178 | RT: 2.09, Area %: 95.79, MW: 448.20, BPM2: 447.2, Method: A |
| Final cpd 76 | RT: 1.38, Area %: 100.00, MW: 261.00, BPM1: 262, BPM2: 260, Method: K |
| Final Cpd 77 | RT: 1.68, Area %: 100.00, MW: 327.00, BPM1: 205, BPM2: 326, Method: K |
| Final Cpd 78 | RT: 1.53, Area %: 100.00, MW: 289.10, BPM1: 290, BPM2: 288, Method: K |
| Final Cpd 79 | RT: 1.29, Area %: 100.00, MW: 298.00, BPM1: 299, BPM2: 297, Method: K |
| Final Cpd 80 | LCMS (MeCN, HCO3NH4) Rt = 1.61 min, 100% (UV), m/z (ES+) = 393.2; m/z (ES−) = 391.2. |
| Final Cpd 179 | RT: 2.08, Area %: 97.26, MW: 383.17, BPM2: 382.2, Method: A |
| Final Cpd 180 | RT: 1.90, Area %: 95.38, MW: 385.19, BPM2: 384.2, Method: A |
| Final Cpd 183 | RT: 1.42, Area %: 98.96, MW: 355.1, BPM1: 356.2, BPM2: 354.2, Method: C |
| Final Cpd 184 | RT: 0.76, Area %: 99.17, MW: 356.13, BPM1: 357.1, Method: A |
| Final Cpd 185 | RT: 1.17, Area %: 97.03, MW: 357.15, BPM1: 358.2, Method: A |
| Final Cpd 186 | RT: 1.02, Area %: 95.94, MW: 328.1, BPM1: 329.3, BPM2: 327.1, Method: E |
| Final Cpd 153 | RT: 2.45, Area %: 100, MH+: 396.2, MH−: 394.2, Method: O |
| Final Cpd 178 | RT: 3.32, Area %: 99, MH+: 408, Method: M |
| Final Cpd 157 | RT: 2.434, Area %: 99, MH+: 451.1, Method: M |
| Final Cpd 160 | RT: 2.778, Area %: 99, MH+: 402.1, Method: M |
| Final Cpd 159 | RT: 2.774, Area %: 99, MH+: 435.1, Method: M |
| Final Cpd 156 | RT: 2.327, Area %: 99, MH+: 358.1, Method: M |
| Fianl Cpd 158 | RT: 2.444, Area %: 99, MH+: 418.1, Method: M |
| Final Cpd 144 | RT: 2.958, Area %: 99, MH+: 356.1, Method: M |
| Final Cpd 145 | RT: 2.912, Area %: 99, MH+: 356.1, Method: M |
| Final Cpd 142 | RT: 2.901, Area %: 99, MH+: 362.1, Method: M |
| Final Cpd 143 | RT: 2.903, Area %: 99, MH+: 395.1, Method: M |
| Final Cpd 147 | RT: 2.838, Area %: 96, MH+: 357.1, MH−: M |
| Final Cpd 141 | RT: 3.447, Area %: 98, MH+: 342.2, Method: M |
| Final Cpd 140 | RT: 2.573, Area %: 99, MH+: 348.1, Method: M |
| Final Cpd 146 | RT: 2.592, Area %: 98, MH+: 381.1, Method: M |
| Final Cpd 152 | RT: 2.498, Area %: 97, MH+: 343.1, Method: M |
| Final Cpd 155 | RT: 2.136, Area %: 99, MH+: 397.1, Method: M |
| Final Cpd 154 | RT: 2.112, Area %: 99, MH+: 364.1, Method: M |
| Final Cpd 177 | RT: 3.104, Area %: 98, MH+: 454.2, Method: M |
| Final Cpd 148 | RT: 2.534, Area %: 99, MH+: 396.1, Method: M |
| Final Cpd 151 | RT: 3.304, Area %: 99, MH+: 435.0, Method: M |
| Final Cpd 149 | RT: 2.530, Area %: 98, MH+: 357.1, Method: M |
| Final Cpd 150 | RT: 2.509, Area %: 97, MH+: 363.1, Method: M |
| Final Cpd 34 | RT: 1.32, Area %: 100.00, MW: 474.26, BPM1: 475.4, BPM2: 473.2, Method: D |
| Final Cpd 188 | RT: 0.82, Area %: 96.00, MW: 374.00, BPM1: 375.3, BPM2: 373.2, Method: D |
| Fianl Cpd 188 | RT: 1.21, Area %: 96.99, MW: 374.21, BPM1: 375.2, Method: A |
| Final Cpd 191 | RT: 1.80, Area %: 97.56, MW: 428.25, BPM1: 429, BPM2: 427, Method: A |
| Final Cpd 191 | RT: 1.80, Area %: 97.57, MW: 428.25, BPM1: 429.3, Method: A |
| Final cpd 190 | RT: 1.55, Area %: 99.47, MW: 402.24, BPM1: 403.2, BPM2: 401, Method: A |
| Final Cpd 190 | RT: 2.26, Area %: 100.00, MW: 402.20, BPM1: 403.3, BPM2: 401.4, Method: O |
| Final Cpd 108 | RT: 1.83, Area %: 100.00, MW: 416.00, BPM1: 417, BPM2: 415, Method: N |
| Final Cpd 128 | RT: 0.93, Area %: 100.00, MW: 392.00, BPM1: 393, BPM2: 391, Method: R |
| Final Cpd 136 | RT: 0.95, Area %: 100.00, MW: 409.00, BPM1: 410, BPM2: 408, Method: R |
| Final Cpd 138 | RT: 0.92, Area %: 100.00, MW: 366.00, BPM1: 367, BPM2: 365, Method: R |
| Final Cpd 135 | RT: 0.99, Area %: 100.00, MW: 386.00, BPM1: 387, BPM2: 385, Method: R |
| Final Cpd 24 | RT: 1.50, Area %: 99.31, MW: 445.06, BPM2: 444.1, Method: A |
| Final Cpd 24 | RT: 1.50, Area %: 99.31, MW: 445.06, BPM2: 444.1, Method: A |
| Final Cpd 198 | RT: 1.14, Area %: 98.85, MW: 385.2, BPM1: 386.4, BPM2: 384.2, Method: C |
| Final Cpd 195 | RT: 1.21, Area %: 99.30, MW: 427.2, BPM1: 428.3, BPM2: 426.2, Method: C |
| Final Cpd 196 | RT: 1.12, Area %: 96.53, MW: 373.2, BPM1: 374.4, BPM2: 372.2, Method: C |
| Final Cpd 197 | RT: 0.80, Area %: 97.59, MW: 401.2, BPM1: 402.2, BPM2: 400.2, Method: C |
| Final Cpd 194 | RT: 0.99, Area %: 100.00, MW: 389.21, BPM2: 388.2, Method: A |
| Final Cpd 194 | RT: 0.99, Area %: 100.00, MW: 389.21, BPM1: 390.2, Method: A |
| Final Cpd 189 | RT: 1.48, Area %: 95.91, MW: 440.10, BPM1: 441.3, BPM2: 439.2, Method: D |
| Final Cpd 166 | RT: 0.94, Area %: 95.28, MW: 393.10, BPM1: 394.2, BPM2: 392.3, Method: S8015S8009 |
| Final Cpd 192 | RT: 1.13, Area %: 100.00, MW: 373.10, BPM1: 374.3, BPM2: 372.2, Method: D |
| Final Cpd 175 | RT: 1.06, Area %: 95.80, MW: 416.10, BPM1: 417.2, BPM2: 415.1, Method: D |
| Final Cpd 161 | RT: 1.07, Area %: 92, MW: 383.10, BPM1: 384.2, BPM2: 382.2, Method: D |
| Final Cpd 176 | RT: 1.08, Area %: 93.99, MW: 417.14, BPM1: 418.3, BPM2: 416.3, Method: D |
| Final Cpd 83 | RT: 2.15, Area %: 100.00, MW: 421.00, BPM1: 422, BPM2: 420, Method: G |
| Final Cpd 82 | RT: 1.70, Area %: 100.00, MW: 392.00, BPM1: 393, BPM2: 393, Method: L |
| Final Cpd 85 | RT: 1.80, Area %: 0.68, MW: 391.20, BPM1: 392, BPM2: 392, Method: L |
| Final Cpd 167 | RT: 1.99, Area %: 94.88, MW: 459.00, BPM1: 460, BPM2: 460, Method: L |
| Final Cpd 168 | RT: 1.93, Area %: 100.00, MW: 461.00, BPM1: 462, BPM2: 462, Method: L |
| Final Cpd 81 | RT: 1.71, Area %: 100.00, MW: 383.20, BPM1: 768, BPM2: 768, Method: L |
| Final Cpd 84 | RT: 1.53, Area %: 98.84, MW: 410.00, BPM1: 411, BPM2: 411, Method: L |
| Final Cpd 86 | RT: 1.93, Area %: 96.02, MW: 393.20, BPM1: 394, BPM2: 394, Method: L |
| Final Cpd 87 | RT: 1.75, Area %: 96.70, MW: 391.00, BPM1: 392, BPM2: 392, Method: L |
| Final Cpd 169 | RT: 1.78, Area %: 100.00, MW: 394.20, BPM1: 789, BPM2: 789, Method: L |
| Final Cpd 88 | RT: 1.77, Area %: 100.00, MW: 387.00, BPM1: 388, BPM2: 388, Method: L |
| Final Cpd 89 | Method: G |
| Fianl Cpd 170 | RT: 0.96, Area %: 98.89, MW: 427.10, BPM1: 428, BPM2: 584, Method: R |
| Final Cpd 90 | RT: 1.71, Area %: 98.60, MW: 391.00, BPM1: 392, BPM2: 392, Method: L |
| Final Cpd 91 | RT: 1.74, Area %: 100.00, MW: 391.00, BPM1: 392, BPM2: 392, Method: L |
| Final Cpd 171 | RT: 1.71, Area %: 100.00, MW: 407.00, BPM1: 408, BPM2: 408, Method: L |
| Final Cpd 92 | RT: 1.80, Area %: 100.00, MW: 442.00, BPM1: 443, BPM2: 441, Method: L |
| Final Cpd 93 | RT: 1.63, Area %: 0.71, MW: 392.00, BPM1: 393, BPM2: 393, Method: L |
| Final Cpd 98 | RT: 2.04, Area %: 100.00, MW: 421.00, BPM1: 422, BPM2: 420, Method: N |
| Final Cpd 99 | RT: 1.83, Area %: 95.70, MW: 416.00, BPM1: 417, BPM2: 415, Method: N |
| Final Cpd 100 | RT: 1.93, Area %: 100.00, MW: 392.00, BPM1: 393, BPM2: 393, Method: L |
| Final Cpd 101 | RT: 1.78, Area %: 100.00, MW: 392.00, BPM1: 393, BPM2: 391, Method: G |
| Final Cpd 104 | RT: 1.86, Area %: 100.00, MW: 380.00, BPM1: 381, BPM2: 379, Method: G |
| Final Cpd 105 | Method: L |
| Final Cpd 116 | RT: 1.97, Area %: 100.00, MW: 402.00, BPM1: 403, BPM2: 403, Method: L |
| Final Cpd 134 | RT: 1.82, Area %: 100.00, MW: 366.00, BPM1: 367, BPM2: 365, Method: G |
| Final Cpd 70 | RT: 2.06, Area %: 96.83, MW: 369.2, BPM1: 370.2, BPM2: 368.0, Method: C |
| Final Cpd 70 | RT: 2.43, Area %: 99.29, MW: 369.19, BPM2: 368.2, Method: A |
| Final Cpd 70 | confirms the MW (RT: 2.40, Area %: 99.29, MW: 369.19, BPM1: 370.2, Method: A) |
| Final Cpd 70 | RT: 2.06, Area %: 98.54, MW: 369.2, BPM1: 370.3, BPM2: 368.2, Method: C |
| Final Cpd 71 | RT: 2.14, Area %: 99.09, MW: 375.23, BPM1: 376.2, Method: A |
| Final Cpd 71 | RT: 2.14, Area %: 98.94, MW: 375.23, BPM2: 374.2, Method: A |
| Final Cpd 72 | RT: 1.65, Area %: 96.59, MW: 356.1709, BPM2: 357.1789, Method: A |
| Final Cpd 72 | RT: 1.63, Area %: 96.51, MW: 356.1709, BPM1: 355.1629, Method: A |
| Final Cpd 72 | RT: 1.51, Area %: 96.11, MW: 356.2, BPM1: 357.2, Method: C |
| Final Cpd 73 | RT: 1.30, Area %: 99.15, MW: 362.2, BPM1: 363.3, BPM2: 361.2, Method: C |
| Final Cpd 73 | RT: 1.42, Area %: 100.00, MW: 362.2066, BPM2: 361.1987, Method: A |
| Final Cpd 73 | RT: 1.40, Area %: 100.00, MW: 362.2066, BPM1: 363.2144, Method: A |
| Final Cpd 74 | RT: 1.52, Area %: 95.39, MW: 386.1814, BPM1: 387.1895, Method: A |
| Final Cpd 74 | RT: 1.39, Area %: 98.88, MW: 386.2, BPM1: 387.2, BPM2: 385.0, Method: C |
| Final Cpd 74 | RT: 1.52, Area %: 95.22, MW: 386.1814, BPM2: 385.2, Method: A |
| Final Cpd 75 | RT: 1.35, Area %: 98.5, MW: 395.2, BPM1: 396.2, BPM2: 394.2 |
| Final Cpd 75 | RT: 1.57, Area %: 98.41, MW: 395.1818, BPM1: 394.1740, BPM2: 394.2, Method: A |
| Final Cpd 75 | RT: 1.57, Area %: 98.51, MW: 395.1818, BPM1: 396.1903, Method: A |
| Final Cpd 94 | RT: 1.61, Area %: 99.51, MW: 406.20, BPM1: 407, BPM2: 405, Method: N |
| Final Cpd 109 | RT: 1.95, Area %: 100.00, MW: 386.00, BPM1: 387, BPM2: 385, Method: N |
| Final Cpd 115 | RT: 1.73, Area %: 100.00, MW: 353.00, BPM1: 354, BPM2: 354, Method: L |
| Final Cpd 110 | RT: 1.99, Area %: 100.00, MW: 386.00, BPM1: 387, BPM2: 145, Method: N |
| Final Cpd 111 | RT: 1.89, Area %: 100.00, MW: 382.00, BPM1: 383, BPM2: 381, Method: N |
| Final Cpd 117 | RT: 1.97, Area %: 100.00, MW: 386.00, BPM1: 387, BPM2: 385, Method: N |
| Final Cpd 118 | RT: 1.78, Area %: 100.00, MW: 352.00, BPM1: 353, BPM2: 353, Method: L |
| Final Cpd 119 | RT: 1.81, Area %: 100.00, MW: 352.00, BPM1: 353, BPM2: 353, Method: L |
| Final Cpd 127 | RT: 1.81, Area %: 100.00, MW: 366.00, BPM1: 367, BPM2: 365, Method: N |
| Final Cpd 133 | RT: 1.95, Area %: 100.00, MW: 386.00, BPM1: 387, BPM2: 385, Method: N |
| Final Cpd 26 | RT: 1.76, Area %: 100.00, MW: 426.20, BPM1: 427, BPM2: 425, Method: N |
| Final Cpd 27 | RT: 1.76, Area %: 100.00, MW: 426.20, BPM1: 427, BPM2: 425, Method: N |
| Final Cpd 29 | RT: 1.53, Area %: 100.00, MW: 386.21, BPM2: 385.19, Method: A |
| Final Cpd 29 | RT: 1.53, Area %: 100.00, MW: 386.21, BPM1: 387.21, Method: A |
| Final Cpd 30 | RT: 1.57, Area %: 100.00, MW: 386.20, BPM1: 387, BPM2: 385, Method: N |
| Final Cpd 31 | RT: 1.57, Area %: 100.00, MW: 386.20, BPM1: 387, BPM2: 385, Method: N |
| Final Cpd 187 | RT: 1.33, Area %: 100.00, MW: 358.21, BPM1: 359.21, Method: A |
| Final Cpd 187 | RT: 1.32, Area %: 100.00, MW: 358.21, BPM2: 357.20, Method: A |
| Final Cpd 35 | RT: 1.95, Area %: 96.97, MW: 422.21, BPM1: 423.21, Method: A |
| Final Cpd 35 | RT: 1.95, Area %: 97.68, MW: 422.21, BPM2: 421.19, Method: A) |
| Final Cpd 32 | RT: 1.68, Area %: 100.00, MW: 353.16, BPM1: 354.16, Method: A |
| Final Cpd 32 | RT: 1.68, Area %: 100.00, MW: 353.16, BPM2: 352.15, Method: A |
| Final Cpd 33 | RT: 1.47, Area %: 100.00, MW: 375.19, BPM1: 376.19, Method: A |
| Final Cpd 33 | RT: 1.47, Area %: 100.00, MW: 375.19, BPM2: 374.18, Method: A |
| Final Cpd 36 | RT: 1.72, Area %: 100.00, MW: 359.20, BPM2: 358.19, Method: A |
| Final Cpd 36 | RT: 1.72, Area %: 98.69, MW: 359.20, BPM1: 360.20, Method: A |
| Final Cpd 81 | RT: 1.67, Area %: 97.91, MW: 383.17, BPM2: 382.16, Method: A |
| Final Cpd 81 | RT: 1.70, Area %: 97.93, MW: 383.17, BPM1: 384.17, Method: A |
| Final Cpd 44 | RT: 1.86, Area %: 100.00, MW: 410.16, BPM1: 411.2, Method: A |
| Final Cpd 44 | RT: 1.86, Area %: 100.00, MW: 410.16, BPM2: 409.2, Method: A |
| Final Cpd 45 | RT: 1.10, Area %: 100.00, MW: 437.16, BPM1: 438.16, Method: P |
| Final Cpd 43 | RT: 1.81, Area %: 100.00, MW: 377.20, BPM1: 378.3, BPM2: 376.4, Method: Q |
| Final Cpd 182 | RT: 1.60, Area %: 98.62, MW: 407.18, BPM2: 406.2, Method: A |
| Final Cpd 182 | RT: 1.60, Area %: 98.96, MW: 407.18, BPM1: 408.2, Method: A |
| Final Cpd 46 | RT: 1.88, Area %: 100.00, MW: 404.18, BPM2: 403.17, Method: A |
| Final Cpd 46 | RT: 1.90, Area %: 100.00, MW: 404.18, BPM1: 405.18, Method: A |
| Final Cpd 181 | RT: 1.44, Area %: 100.00, MW: 374.21, BPM1: 375.33, Method: Q |
| Final Cpd 181 | RT: 1.50, Area %: 62.68, MW: 374.21, BPM1: 375.21, Method: A |
| Final Cpd 181 | degradation of the product RT: 1.50, Area %: 63.13, MW: 374.21, BPM2: 373.19, Method: A) |
| Final Cpd 41 | RT: 1.63, Area %: 93.91, MW: 416.18, BPM2: 415.2, Method: A |
| Final Cpd 41 | RT: 1.63, Area %: 94.11, MW: 416.18, BPM1: 417.2, Method: A |
| Final Cpd 42 | RT: 1.63, Area %: 100.00, MW: 449.15, BPM1: 450.2, Method: A |
| Final Cpd 42 | RT: 1.63, Area %: 100.00, MW: 449.15, BPM2: 448.1, Method: A |
| Final Cpd 163 | RT: 1.50, Area %: 5.39, MW: 391.15, BPM1: 392.24, BPM2: 390.22, Method: E |
| Final Cpd 40 | RT: 1.51, Area %: 99.31, MW: 357.18, BPM1: 358.24, BPM2: 356.26, Method: E |
| Final Cpd 37 | RT: 1.26, Area %: 98.93, MW: 393.17, BPM1: 394.2, Method: A |
| Final Cpd 39 | RT: 1.11, Area %: 100.00, MW: 423.17, BPM1: 424.2, Method: A |
| Final Cpd 38 | RT: 1.33, Area %: 94.60, MW: 437.19, BPM1: 438.2, Method: A |
| Final Cpd 68 | RT: 1.82, Area %: 95.32, MW: 406.19, BPM1: 407.2, Method: A |
| Final Cpd 69 | RT: 1.75, Area %: 93.15, MW: 406.19, BPM1: 407.2, Method: A |
| Final Cpd 164 | RT: 1.56, Area %: 98.77, MW: 386.00, BPM1: 387.2, BPM2: 385.2, Method: L |
| Final Cpd 172 | confirms the MW, Pure compound (RT: 2.05, Area %: 100.00, MW: 421.00, |
| | BPM1: 422.3, BPM2: 420.3, Method: L) |
| Final Cpd 173 | confirms the MW, Pure compound (RT: 1.98, Area %: 87.26, MW: 479.00, |
| | BPM1: 480.2, BPM2: 478.2, Method: L) |
| Final Cpd 95 | confirms the MW, Pure compound (>98%) (RT: 1.70, Area %: 100.00, MW: 470.00, |
| | BPM1: 471.1/473.1, BPM2: 469.1/471.1, Method: G) |
| Final Cpd 96 | confirms the MW, Pure compound (>95%) (RT: 1.85, Area %: 100.00, MW: 370.00, |
| | BPM1: 371.2, BPM2: 369.2, Method: G) |
| Final Cpd 97 | confirms the MW, Pure compound (>98%) (RT: 1.90, Area %: 100.00, MW: 384.00, |
| | BPM1: 385.2, BPM2: 383.3, Method: G) |
| Final Cpd 102 | confirms the MW, Pure compound (>98%) (RT: 1.93, Area %: 100.00, MW: 396.00, |
| | BPM1: 397.2, BPM2: 395.2, Method: G) |
| Final Cpd 193 | confirms the MW, After RP purification and evaporation of collection solvent |
| | (RT: 1.67, Area %: 100.00, MW: 417.00, BPM1: 418, BPM2: 416, Method: N) |
| Final Cpd 103 | confirms the MW, Pure compound (>98%) (RT: 1.69, Area %: 100.00, MW: 392.00, |
| | BPM1: 393.2, BPM2: 391.2, Method: G) |
| Final Cpd 114 | confirms the MW, >98% purity (RT: 1.82, Area %: 100.00, MW: 406.00, |
| | BPM1: 407.2, BPM2: 405.2, Method: G) |
| Final Cpd 165 | confirms the MW, >98% purity (RT: 1.76, Area %: 100.00, MW: 392.00, |
| | BPM1: 393.2, BPM2: 391.2, Method: G) |
| Final Cpd 106 | confirms the MW, >98% purity (RT: 2.05, Area %: 100.00, MW: 382.00, |
| | BPM1: 383.3, BPM2: 381.1, Method: G) |
| Final Cpd 112 | confirms the MW, >98% purity (RT: 1.90, Area %: 98.74, MW: 370.00, |
| | BPM1: 371.2, BPM2: 369.2, Method: G) |
| Final Cpd 113 | confirms the MW, >98% purity (RT: 1.74, Area %: 100.00, MW: 366.00, |
| | BPM1: 367.2, BPM2: 365.2, Method: G) |
| Final Cpd 125 | confirms the MW, Looks like >98% purity (RT: 1.75, Area %: 100.00, MW: 382.00, |
| | BPM1: 383.2, BPM2: 381.1, Method: G) |
| Final Cpd 124 | confirms the MW, ca. 95% purity (RT: 2.00, Area %: 94.71, MW: 400.00, |
| | BPM1: 401.2, BPM2: 399.2, Method: G) |
| Final Cpd 123 | confirms the MW, >97% purity (RT: 1.97, Area %: 100.00, MW: 395.00, |
| | BPM1: 396.3, BPM2: 394.3, Method: G) |
| Fianl Cpd 126 | confirms the MW, >98% purity (RT: 1.80, Area %: 98.50, MW: 391.00, |
| | BPM1: 392.2, BPM2: 390.1, Method: G) |
| Final Cpd 174 | confirms the MW (RT: 1.93, Area %: 98, MW: 403.10, BPM1: 404, BPM2: 402, Method: G) |
| Final Cpd 107 | confirms the MW (RT: 1.80, Area %: 100, MW: 383.1, BPM1: 384, BPM2: 382, Method: L) |
| Final Cpd 122 | confirms the MW (RT: 1.87, Area %: 100, MW: 366.1, BPM1: 367, BPM2: 365, Method: L) |
| Final Cpd 121 | confirms the MW (RT: 1.84, Area %: 100, MW: 382.1, BPM1: 383, BPM2: 381, Method: L) |
| Final Cpd 120 | confirms the MW (RT: 2.06, Area %: 100, MW: 400.1, BPM1: 401, BPM2: 399, Method: L) |
| Final Cpd 130 | confirms the MW (RT: 1.96, Area %: 100, MW: 402.1, BPM1: 403, BPM2: 401, Method: G) |
| Final Cpd 131 | confirms the MW (RT: 1.73, Area %: 100, MW: 367.1, BPM1: 368, BPM2: 366, Method: L) |
| Final Cpd 129 | confirms the MW (RT: 1.96, Area %: 100, MW: 382.1, BPM1: 383, BPM2: 381, Method: L) |
| Fianl Cpd 132 | confirms the MW (RT: 1.91, Area %: 100, MW: 383.1, BPM1: 384, BPM2: 382, Method: G) |
| Final Cpd 137 | confirms the MW (RT: 1.91, Area %: 100, MW: 381.1, BPM1: 382, BPM2: 380, Method: G) |
| Final Cpd 139 | confirms the MW (RT: 0.89, Area %: 90, MW: 347.2, BPM1: 348.2, BPM2: 346.2, Method: E) |
| Final Cpd 139 | confirms the MW (RT: 0.89, Area %: 100.00, MW: 347.20, BPM1: 348.2, BPM2: 346.3, |
| | Method: E) |
| Final Cpd 199 | confirms the MW, sample in ACN/DMSO (RT: 1.19, Area %: 99.58, MW: 423.2, |
| | BPM1: 424.4, BPM2: 422.2, Method: C) |
| Final Cpd 200 | confirms the MW (RT: 0.57, Area %: 96, MW: 371.20, BPM1: 372.2, Method: F) |
| Final Cpd 201 | confirms the MW, sample in ACN/DMSO (RT: 1.07, Area %: 97.33, MW: 373.2, |
| | BPM1: 374.4, BPM2: 372.2, Method: C) |
| Final Cpd 202 | confirms the MW (RT: 0.36, Area %: 97.88, MW: 331.20, BPM1: 332.1, Method: F) |
| Final Cpd 199 | confirms the MW (RT: 1.26, Area %: 100.00, MW: 423.20, BPM1: 424.3, BPM2: 422.2, |
| | Method: E) |
| Final Cpd 40 | confirms the MW, sample in ACN/DMSO (RT: 0.87, Area %: 96.32, MW: 357.2, |
| | BPM1: 358.2, BPM2: 356.2, Method: C) |
| Final Cpd 40 | confirms the MW (RT: 0.88, Area %: 98.79, MW: 357.2, BPM1: 358.2, BPM2: 356.0, |
| | Method: C) |
| Final Cpd 40 | confirms the MW (RT: 0.79, Area %: 98, MW: 357.1801, BPM1: 358.1884, BPM2: 356.1726, |
| | Method: A) |
| Final Cpd 202 | confirms the MW (RT: 0.96, Area %: 100.00, MW: 331.2, BPM1: 332.2, Method: A) |
| Final Cpd 47 | confirms the MW (RT: 1.72, Area %: 96.11, MW: 355.14, BPM1: 356.2, Method: A) |
| Final Cpd 55 | confirms the structure (RT: 0.89, Area %: 98.51, MW: 373.21, BPM1: 374.3, BPM2: 372.4, |
| | Method: F) |
| Final Cpd 56 | confirms the MW (RT: 0.64, Area %: 100.00, MW: 434.18, BPM1: 435.3, BPM2: 433.3, |
| | Method: F) |
| Final Cpd 57 | confirms the MW (RT: 0.63, Area %: 96.45, MW: 345.18, BPM1: 346.2, BPM2: 344.3, |
| | Method: F) |
| Final Cpd 58 | confirms the MW (RT: 1.02, Area %: 98.00, MW: 487.23, BPM1: 488.3, BPM2: 486.3, |
| | Method: F) |
| Final Cpd 59 | confirms the MW (RT: 0.56, Area %: 97.44, MW: 407.21, BPM1: 408.3, BPM2: 406.3, |
| | Method: F) |
| Final Cpd 60 | confirms the MW (RT: 0.75, Area %: 99.34, MW: 470.22, BPM1: 471.3, BPM2: 469.3, |
| | Method: F) |
| Final Cpd 61 | confirms the MW (RT: 0.72, Area %: 97.26, MW: 371.16, BPM1: 372.2, BPM2: 370.3, |
| | Method: F) |
| Final Cpd 62 | confirms the MW (RT: 0.78, Area %: 96.98, MW: 315.17, BPM1: 316.2, BPM2: 314.2, |
| | Method: F) |
| Final Cpd 63 | confirms the MW (RT: 1.30, Area %: 100.00, MW: 445.11, BPM1: 446.2, BPM2: 444.2, |
| | Method: F) |
| Final Cpd 48 | confirms the MW (RT: 0.59, Area %: 92.90, MW: 325.13, BPM1: 326.1, BPM2: 324.2, |
| | Method: F) |
| Final Cpd 64 | confirms the MW (RT: 0.94, Area %: 100.00, MW: 341.19, BPM1: 342.3, BPM2: 340.3, |
| | Method: F) |
| Final Cpd 65 | confirms the MW (RT: 0.51, Area %: 100.00, MW: 331.16, BPM1: 332.2, BPM2: 330.2, |
| | Method: F) |
| Final Cpd 66 | confirms the MW (RT: 0.77, Area %: 89.89, MW: 393.18, BPM1: 394.2, BPM2: 392.3, |
| | Method: F) |
| Final Cpd 67 | confirms the MW (RT: 1.15, Area %: 100.00, MW: 381.22, BPM1: 382.3, BPM2: 380.3, |
| | Method: F) |
| Final Cpd 49 | confirms the MW (RT: 0.53, Area %: 98.77, MW: 338.15, BPM1: 339.2, BPM2: 337.2, |
| | Method: F) |
| Final Cpd 50 | confirms the MW (RT: 0.78, Area %: 100.00, MW: 407.20, BPM1: 408.3, BPM2: 406.3, |
| | Method: F) |
| Final Cpd 51 | confirms the MW (RT: 0.76, Area %: 93.95, MW: 385.14, BPM1: 386.2, BPM2: 384.3, |
| | Method: F) |
| Final Cpd 52 | confirms the MW (RT: 0.90, Area %: 94.94, MW: 420.23, BPM1: 421.3, BPM2: 419.4, |
| | Method: F) |
| Final Cpd 53 | confirms the MW (RT: 1.10, Area %: 100.00, MW: 441.16, BPM1: 442.2, BPM2: 440.3, |
| | Method: F) |
| Final Cpd 54 | confirms the MW (RT: 0.63, Area %: 100.00, MW: 400.22, BPM1: 401.3, BPM2: 399.3, |
| | Method: F) |

Characterising Data—NMR

| Compound | NMR |
|---|---|
| Final Cpd 1 | 1H NMR (400 MHz, DMSO-d6) δ ppm 1.26-1.33 (m, 9 H) 2.80 (q, |
| | J = 7.63 Hz, 2 H) 3.52-3.72 (m, 1 H) 4.93 (s, 2 H) 7.12 (s, 1 H) 7.98 |
| | (dd, J = 5.78, 1.16 Hz, 1 H) 8.66 (d, J = 6.01 Hz, 1 H) 8.91 (d, J = 1.16 Hz, |
| | 1H) 11.26 (s, 1H) |
| Final Cpd 23 | 1H NMR (500 MHz, DMSO-d6) d ppm 1.31 (d, J = 6.87 Hz, 6 H) 3.59 |
| | (quin, J = 6.83 Hz, 1 H) 4.88 (s, 2 H) 7.29 (dd, J = 9.77, 1.83 Hz, 1 H) |
| | 7.52 (s, 1 H) 7.79 (d, J = 9.77 Hz, 1 H) 9.19 (s, 1 H) 9.24 (s, 1 H) 10.25- |
| | 10.78 (m, 1 H) |
| Final Cpd 3 | 1H NMR (500 MHz, DMSO-d6) δ ppm 1.31 (d, J = 6.9 Hz, 6 H), 1.31 |
| | (br d, J = 6.9 Hz, 6 H), 3.14 (spt, J = 6.9 Hz, 1 H), 3.59 (spt, J = 6.9 Hz, 1 |
| | H), 4.92 (s, 2 H), 7.16 (s, 1 H), 7.98 (dd, J = 5.8, 1.3 Hz, 1 H), 8.67 (d, |
| | J = 5.8 Hz, 1 H), 8.91 (d, J = 1.3 Hz, 1 H), 11.25 (br s, 1 H) |
| Final Cpd 5 | 1H NMR (400 MHz, DMSO-d6) δ ppm 1.32 (d, J = 6.9 Hz, 6 H), 3.60 |
| | (spt, J = 6.9 Hz, 1 H), 4.94 (s, 2 H), 5.64 (dd, J = 11.0, 1.1 Hz, 1 H), 6.22 |
| | (dd, J = 17.8, 1.2 Hz, 1 H), 6.87 (dd, J = 17.8, 11.1 Hz, 1 H), 7.56 (s, 1 H), |
| | 7.99 (dd, J = 5.8, 1.3 Hz, 1 H), 8.67 (d, J = 5.8 Hz, 1 H), 8.91 (d, J = 1.3 |
| | Hz, 1 H), 11.26 (s, 1 H) |
| Final Cpd 6 | 1H NMR (400 MHz, DMSO-d6) δ ppm 1.28 (t, J = 7.4 Hz, 3 H), 3.03 (q, |
| | J = 7.4 Hz, 2 H), 4.88 (s, 2 H), 7.29 (dd, J = 9.8, 1.9 Hz, 1 H), 7.52 (s, 1 |
| | H), 7.79 (dt, J = 9.7, 1.0 Hz, 1 H), 9.18 (dd, J = 2.1, 1.0 Hz, 1 H), 9.24 (d, |
| | J = 0.8 Hz, 1 H), 10.49 (br s, 1 H) |
| Final Cpd 7 | 1H NMR (400 MHz, DMSO-d6) δ ppm 1.21 (s, 3 H), 1.25 (t, J = 7.4 Hz, |
| | 3 H), 1.88-1.96 (m, 2 H), 2.17-2.25 (m, 2 H), 2.99 (q, J = 7.4 Hz, 2 |
| | H), 3.77 (sxt, J = 8.0 Hz, 1 H), 4.53 (s, 2 H), 4.96 (s, 1 H), 7.46 (s, 1 H), |
| | 8.17 (d, J = 7.1 Hz, 1 H) |
| Final Cpd 8 | 1H NMR (400 MHz, DMSO-d6) δ ppm 0.83-0.89 (m, 2 H), 1.01- |
| | 1.08 (m, 2 H), 1.21 (s, 3 H), 1.29 (d, J = 6.9 Hz, 6 H), 1.89-1.96 (m, 2 |
| | H), 2.14 (tt, J = 8.4, 5.0 Hz, 1 H), 2.17-2.25 (m, 2 H), 3.54 (spt, J = 6.9 |
| | Hz, 1 H), 3.78 (sxt, J = 8.0 Hz, 1 H), 4.51 (s, 2 H), 4.95 (s, 1 H), 6.95 (s, |
| | 1 H), 8.17 (d, J = 7.2 Hz, 1 H) |
| Final Cpd 9 | 1H NMR (400 MHz, CHLOROFORM-d) δ ppm 0.86-0.93 (m, 2 H) |
| | 1.05-1.12 (m, 2 H) 1.39 (d, J = 6.9 Hz, 3 H) 1.40 (d, J = 6.9 Hz, 3 H) |
| | 1.44-1.72 (m, 8 H) 1.83-1.96 (m, 3 H) 2.11 (tt, J = 8.4, 5.0 Hz, 2 H) |
| | 2.23-2.36 (m, 1 H) 2.37-2.50 (m, 1 H) 2.55-2.67 (m, 1 H) 3.67 (spt, |
| | J = 6.9 Hz, 1 H) 4.01-4.13 (m, 1 H) 4.61-4.86 (m, 2 H) 6.55 (br s, 1 H) |
| | 6.81 (s, 1 H) |
| Final Cpd 25 | 1H NMR (500 MHz, DMSO-d6) d ppm 0.13-0.21 (m, 2 H) 0.40-0.46 |
| | (m, 2 H) 0.84-0.93 (m, 3 H) 1.02-1.08 (m, 2 H) 1.29 (d, J = 6.87 Hz, 6 |
| | H) 1.69-1.79 (m, 1 H) 1.81-1.91 (m, 1 H) 2.14 (tt, J = 8.37, 4.98 Hz, 1 |
| | H) 2.27-2.40 (m, 2 H) 3.06-3.14 (m, 1 H) 3.15-3.23 (m, 2 H) 3.47 |
| | (dd, J = 11.83, 4.65 Hz, 1 H) 3.50-3.60 (m, 1 H) 4.02-4.13 (m, 1 H) |
| | 4.51-4.65 (m, 2 H) 6.96 (s, 1 H) 8.24 (d, J = 7.32 Hz, 1 H) |
| Final Cpd 28 | 1H NMR (500 MHz, DMSO-d6) d ppm 0.85-0.90 (m, 2 H) 1.03-1.08 |
| | (m, 2 H) 1.31 (d, J = 6.87 Hz, 6 H) 2.14 (tt, J = 8.39, 4.88 Hz, 1 H) 3.39 |
| | (s, 3 H) 3.52-3.62 (m, 1 H) 4.73 (s, 2 H) 6.39 (d, J = 9.61 Hz, 1 H) 6.98 |
| | (s, 1 H) 7.37 (dd, J = 9.61, 2.90 Hz, 1 H) 8.03 (d, J = 2.75 Hz, 1 H) 9.87 |
| | (s, 1 H) |
| Final Cpd 10 | 1H NMR (500 MHz, DMSO-d6) δ ppm 0.82-0.91 (m, 2 H) 0.97-1.11 |
| | (m, 2H) 1.25-1.31 (m, 3 H) 2.14 (tt, J = 8.37, 4.98 Hz, 1 H) 2.99 (q, |
| | J = 7.43 Hz, 2 H) 4.92 (s, 2 H) 6.99 (s, 1 H) 7.97 (dd, J = 5.80, 1.07 Hz, 1 |
| | H) 8.66 (d, J = 5.80 Hz, 1 H) 8.91 (d, J = 0.92 Hz, 1 H) 10.99-11.37 (m, 1H) |
| Final Cpd 11 | 1H NMR (400 MHz, CHLOROFORM-d) δ ppm 0.83-0.97 (m, 2 H), |
| | 1.03-1.16 (m, 2 H), 1.39 (d, J = 6.9 Hz, 6 H), 2.11 (tt, J = 8.4, 4.9 Hz, 1 |
| | H), 3.68 (spt, J = 6.9 Hz, 1 H), 4.93 (s, 2 H), 6.84 (s, 1 H), 8.12 (dd, |
| | J = 5.8, 1.3 Hz, 1 H), 8.63 (d, J = 5.8 Hz, 1 H), 8.84 (br s, 1 H), 8.86 (d, |
| | J = 1.3 Hz, 1 H) |
| Final Cpd 12 | 1H NMR (400 MHz, CHLOROFORM-d) δ ppm 1.00 (t, J = 7.4 Hz, 3 |
| | H), 1.41 (d, J = 6.9 Hz, 6 H), 1.76 (sxt, J = 7.4 Hz, 2 H), 2.80 (t, J = 7.6 |
| | Hz, 2 H), 3.70 (spt, J = 6.9 Hz, 1 H), 4.95 (s, 2 H), 7.00 (s, 1 H), 8.13 |
| | (dd, J = 5.8, 1.4 Hz, 1 H), 8.63 (d, J = 5.8 Hz, 1 H), 8.82-8.90 (m, 2 H) |
| Final Cpd 16 | 1HNMR (400 MHz, CHLOROFORM-d) δ ppm 1.35 (d, J = 6.9 Hz, 6 |
| | H), 1.40 (t, J = 7.5 Hz, 3 H), 3.11 (q, J = 7.4 Hz, 2 H), 3.17 (spt, J = 7.0 Hz, |
| | 1 H), 4.94 (s, 2 H), 7.03 (s, 1 H), 8.13 (dd, J = 5.8, 1.3 Hz, 1 H), 8.63 (d, |
| | J = 5.8 Hz, 1 H), 8.68 (br s, 1 H), 8.86 (d, J = 1.3 Hz, 1 H) |
| Final Cpd 18 | 1H NMR (400 MHz, DMSO-d6) δ ppm 1.29 (d, J = 6.94 Hz, 6 H) 2.80 |
| | (d, J = 5.09 Hz, 3 H) 3.48 (quin, J = 6.88 Hz, 1 H) 4.87 (s, 2 H) 6.29- |
| | 6.43 (m, 2 H) 7.98 (dd, J = 5.78, 1.39 Hz, 1 H) 8.66 (d, J = 6.01 Hz, 1 H) |
| | 8.91 (d, J = 0.92 Hz, 1 H) 11.23 (br s, 1 H) |
| Cpd 19 | 1H NMR (400 MHz, DMSO-d6) δ ppm 1.28 (d, J = 6.94 Hz, 6 H) 2.59 |
| | (d, J = 4.39 Hz, 3 H) 2.79 (d, J = 5.09 Hz, 3 H) 3.39-3.52 (m, 1 H) 4.49 |
| | (s, 2 H) 6.22-6.41 (m, 2 H) 7.88 (br d, J = 4.39 Hz, 1 H) |
| Final Cpd 20 | 1H NMR (400 MHz, DMSO-d6) δ ppm 1.29 (d, J = 6.94 Hz, 6 H) 2.80 |
| | (d, J = 5.09 Hz, 3 H) 3.48 (quin, J = 6.88 Hz, 1 H) 4.87 (s, 2 H) 6.29- |
| | 6.43 (m, 2 H) 7.98 (dd, J = 5.78, 1.39 Hz, 1 H) 8.66 (d, J = 6.01 Hz, 1 H) |
| | 8.91 (d, J = 0.92 Hz, 1 H) 11.23 (br s, 1 H) |
| Final Cpd 21 | 1H NMR (400 MHz, CHLOROFORM-d) δ ppm 1.37 (d, J = 6.94 Hz, 6 |
| | H) 2.45 (quin, J = 7.46 Hz, 2 H) 3.60 (spt, J = 6.88 Hz, 1 H) 4.03 (t, |
| | J = 7.40 Hz, 4 H) 4.93 (s, 2 H) 6.37 (s, 1 H) 8.13 (dd, J = 5.78, 1.16 Hz, 1 |
| | H) 8.61 (d, J = 5.78 Hz, 1 H) 8.84 (d, J = 0.92 Hz, 1 H) 9.17 (s, 1 H) |
| Final Cpd 22 | 1H NMR (400 MHz, CHLOROFORM-d) δ ppm 1.39 (d, J = 6.70 Hz, 6 |
| | H) 3.01 (s, 6 H) 3.55 (s, 1 H) 4.93 (s, 2 H) 6.49 (s, 1 H) 8.13 (dd, |
| | J = 5.78, 1.39 Hz, 1 H) 8.62 (d, J = 5.55 Hz, 1 H) 8.85 (d, J = 1.16 Hz, 1 |
| | H) 9.06 (br s, 1 H) |
| Final Cpd 80 | 1H NMR (400 MHz, DMSO-d6) ppm 0.85-0.92 (m, 2 H), 1.03-1.11 |
| | (m, 2 H), 1.32 (d, J = 6.9 Hz, 6 H), 2.15 (tt, J = 8.4, 4.9 Hz, 1 H), 3.52- |
| | 3.65 (m, 1 H), 4.86 (s, 2 H), 7.00 (s, 1 H), 7.30 (dd, J = 9.8, 1.9 Hz, 1 H), |
| | 7.79 (d, J = 9.7 Hz, 1 H), 9.18 (dd, J = 1.7, 0.9 Hz, 1 H), 9.24 (d, J = 0.7 |
| | Hz, 1 H), 10.46 (s, 1 H). |
| Final Cpd 186 | 1H NMR (400 MHz, DMSO-d6) d ppm 1.27 (d, J = 6.70 Hz, 7 H) 4.87 |
| | (s, 2 H) 5.92 (s, 2 H) 6.26 (s, 1 H) 7.98 (dd, J = 5.78, 1.16 Hz, 1 H) 8.66 |
| | (d, J = 5.78 Hz, 1 H) 8.91 (d, J = 0.92 Hz, 1 H) 10.84-11.46 (m, 1 H) |
| Final Cpd 153 | 1H NMR (400 MHz, DMSO): d ppm 8.20 (d, J = 7.2 Hz, 1H), 7.19 (s, |
| | 1H), 4.97 (s, 1H), 4.53 (s, 2H), 3.83-3.73 (m, 1H), 3.57 (sept, J = 6.8 Hz, |
| | 1H), 3.28-3.20 (m, 1H), 2.19 (m, 4H), 1.92 (t, J = 8.8 Hz, 2H), 1.30 (d, |
| | 6.8 Hz, 6H), 1.21 (s, 3H) |
| Fianl Cpd 178 | 1H NMR (400 MHz, CHLOROFORM-d, 25° C.): Shift = 9.79 (br s, |
| | 1H), 9.16 (br s, 1H), 8.95 (br d, J = 5.8 Hz, 1H), 8.07 (br d, J = 3.7 Hz, |
| | 1H), 6.94 (s, 1H), 4.94 (s, 2H), 3.57-3.67 (m, 1H), 2.77 (t, J = 7.5 Hz, |
| | 2H), 2.07-2.16 (m, 2H), 1.72-1.84 (m, 7H), 1.44-1.55 (m, 2H), 1.33 |
| | ppm (d, J = 6.9 Hz, 6H) |
| Final Cpd 157 | 1H NMR (400 MHz, DMSO) d 10.46 (s, 1H), 9.24 (s, 1H), 9.17 (s, |
| | 1H), 7.79 (d, J = 9.8 Hz, 1H), 7.30 (dd, J = 9.8, 1.8 Hz, 1H), 7.26 (d, J = |
| | 5.0 Hz, 1H), 7.25 (s, 1H), 5.46 (p, J = 7.1 Hz, 1H), 4.89 (d, J = 1.7 |
| | Hz, 2H), 3.66-3.55 (m, 1H), 1.33 (dd, J = 6.8, 0.8 Hz, 6H). |
| Final Cpd 160 | 1H NMR (400 MHz, DMSO) d 8.19 (d, J = 7.1 Hz, 1H), 7.19 (s, 1H), |
| | 4.95 (s, 1H), 4.54 (s, 2H), 3.99 (q, J = 11.3 Hz, 2H), 3.78 (dd, J = 15.8, |
| | 8.1 Hz, 1H), 3.57 (dt, J = 13.8, 6.9 Hz, 1H), 2.26-2.16 (m, 2H), 1.93 |
| | (t, J = 10.0 Hz, 2H), 1.31 (d, J = 6.9 Hz, 6H), 1.21 (s, 3H). |
| Final Cpd 159 | 1H NMR (400 MHz, DMSO) d 10.45 (s, 1H), 9.24 (s, 1H), 9.17 (s, |
| | 1H), 7.79 (d, J = 9.8 Hz, 1H), 7.30 (dd, J = 9.8, 1.6 Hz, 1H), 7.24 (s, |
| | 1H), 4.89 (s, 2H), 4.01 (q, J = 11.2 Hz, 2H), 3.68-3.55 (m, 1H), 1.34 |
| | (s, 3H), 1.33 (s, 3H). |
| Final Cpd 156 | 1H NMR (400 MHz, DMSO) d 11.29 (br s, 1H), 8.91 (s, 1H), 8.67 (d, J = |
| | 5.8 Hz, 1H), 7.98 (d, J = 5.3 Hz, 1H), 7.18 (s, 1H), 5.64 (br d, J = 6.0 |
| | Hz, 1H), 5.27 (p, J = 6.4 Hz, 1H), 4.95 (s, 2H), 3.14 (hept, J = 6.9 Hz, |
| | 1H), 1.50 (d, J = 6.6 Hz, 3H), 1.31 (d, J = 6.9 Hz, 6H). |
| Final Cpd 158 | 1H NMR (400 MHz, DMSO) d 8.20 (br d, J = 7.2 Hz, 1H), 7.26 (br s, |
| | 1H), 7.20 (s, 1H), 5.44 (q, J = 7.2 Hz, 1H), 4.96 (s, 1H), 4.57 (d, J = |
| | 16.2 Hz, 1H), 4.51 (d, J = 16.2 Hz, 1H), 3.85-3.73 (m, 1H), 3.57 |
| | (hept, J = 6.9 Hz, 1H), 2.25-2.17 (m, 2H), 1.97-1.88 (m, 2H), 1.31 |
| | (d, J = 6.9 Hz, 6H), 1.21 (s, 3H). |
| Final Cpd 144 | 1H NMR (400 MHz, DMSO) d 10.90 (br s, 1H), 9.28 (d, J = 2.5 Hz, |
| | 1H), 9.04 (d, J = 5.9 Hz, 1H), 7.87 (dd, J = 5.9, 2.5 Hz, 1H), 7.17 (s, |
| | 1H), 4.90 (s, 2H), 3.60 (hept, J = 6.9 Hz, 1H), 3.14 (hept, J = 6.9 Hz, |
| | 1H), 1.32 (d, J = 6.9 Hz, 6H), 1.31 (d, J = 6.9 Hz, 6H). |
| Final Cpd 145 | 1H NMR (400 MHz, DMSO) d 10.92 (br s, 1H), 9.28 (d, J = 2.7, 0.8 |
| | Hz, 1H), 9.05 (dd, J = 5.9, 0.8 Hz, 1H), 7.87 (dd, J = 5.9, 2.7 Hz, 1H), |
| | 7.12 (s, 1H), 4.90 (s, 2H), 3.60 (hept, J = 6.9 Hz, 1H), 2.76 (t, J = 7.5 |
| | Hz, 3H). |
| Final Cpd 142 | 1H NMR (400 MHz, DMSO) d 8.18 (d, J = 7.2 Hz, 1H), 7.12 (s, 1H), |
| | 4.95 (s, 1H), 4.52 (s, 2H), 3.84-3.73 (m, 1H), 3.56 (hept, J = 6.9 Hz, |
| | 1H), 3.13 (hept, J = 6.9 Hz, 1H), 2.25-2.16 (m, 2H), 1.98-1.88 (m, |
| | 2H), 1.30 (d, J = 6.9 Hz, 6H), 1.30 (d, J = 6.9 Hz, 6H), 1.21 (s, 3H). |
| Final Cpd 143 | 1H NMR (400 MHz, DMSO) d 10.46 (br s, 1H), 9.25 (d, J = 0.5 Hz, |
| | 1H), 9.18 (s, 1H), 7.79 (d, J = 9.8 Hz, 1H), 7.30 (dd, J = 9.8, 1.9 Hz, |
| | 1H), 7.17 (s, 1H), 4.87 (s, 2H), 3.61 (hept, J = 6.9 Hz, 1H), 3.13 (hept, J = |
| | 6.9 Hz, 1H), 1.31 (d, J = 6.9 Hz, 6H), 1.31 (d, J = 6.9 Hz, 6H). |
| Final Cpd 147 | 1H NMR (400 MHz, DMSO) d 11.23 (br s, 1H), 8.91 (d, J = 1.0 Hz, |
| | 1H), 8.67 (d, J = 5.8 Hz, 1H), 7.99 (dd, J = 5.8, 1.0 Hz, 1H), 7.10 (s, |
| | 1H), 4.84 (s, 2H), 3.12 (hept, J = 6.9 Hz, 1H), 3.01 (s, 5H), 1.30 (d, J = |
| | 6.9 Hz, 6H). |
| Final Cpd 141 | 1H NMR (400 MHz, CDCl3) d 9.57 (s, 1H), 9.12 (d, J = 2.5 Hz, 1H), |
| | 9.00 (d, J = 5.7 Hz, 1H), 8.03 (dd, J = 5.9, 2.7 Hz, 1H), 7.00 (d, J = 6.6 |
| | Hz, 1H), 4.97 (d, J = 7.6 Hz, 2H), 3.78-3.65 (m, 1H), 2.86 (q, J = 7.6 |
| | Hz, 2H), 1.40 (d, J = 6.9 Hz, 6H), 1.34 (t, J = 7.6 Hz, 3H). |
| Final Cpd 140 | 1H NMR (400 MHz, CDCl3) d 6.98 (s, 1H), 6.31 (s, 1H), 4.71 (s, 2H), |
| | 4.01 (dt, J = 15.7, 7.9 Hz, 1H), 3.69 (hept, J = 6.8 Hz, 1H), 2.86 (q, J = |
| | 7.6 Hz, 2H), 2.59-2.46 (m, 2H), 2.08-2.00 (m, 2H), 1.57 (s, 1H), |
| | 1.43-1.33 (m, 12H). |
| Final Cpd 146 | 1H NMR (400 MHz, CDCl3) d 9.19 (s, 1H), 8.74 (s, 1H), 7.70 (d, J = |
| | 9.5 Hz, 1H), 7.00 (dd, J = 7.0, 2.4 Hz, 2H), 4.95 (d, J = 1.9 Hz, 2H), |
| | 3.78-3.64 (m, 1H), 2.92-2.79 (m, 2H), 1.41 (d, J = 6.9 Hz, 6H), 1.34 |
| | (t, J = 7.6 Hz, 3H). |
| Final Cpd 152 | 1H NMR (400 MHz, CDCl3) d 8.85 (d, J = 4.8 Hz, 2H), 8.63 (d, J = |
| | 5.6 Hz, 1H), 8.14 (d, J = 5.8 Hz, 1H), 7.01 (s, 1H), 4.86 (s, 2H), 3.14 (s, |
| | 6H), 2.86 (q, J = 7.6 Hz, 2H), 1.34 (t, J = 7.6 Hz, 3H). |
| Final Cpd 155 | 1H NMR (400 MHz, CDCl3) d 9.18 (s, 1H), 8.76 (d, J = 7.3 Hz, 2H), |
| | 7.72 (d, J = 9.7 Hz, 1H), 7.05 (s, 1H), 6.99 (dd, J = 9.7, 1.7 Hz, 1H), |
| | 5.26 (p, J = 6.6 Hz, 1H), 4.94 (d, J = 1.9 Hz, 2H), 4.05 (d, J = 6.3 Hz, |
| | 1H), 3.19 (dt, J = 13.8, 6.9 Hz, 1H), 1.71 (d, J = 6.6 Hz, 3H), 1.36 (d, J = |
| | 6.9 Hz, 6H). |
| Final Cpd 154 | 1H NMR (400 MHz, CDCl3) d 7.00 (s, 1H), 6.19 (d, J = 7.4 Hz, 1H), |
| | 5.22 (p, J = 6.6 Hz, 1H), 4.69 (d, J = 4.4 Hz, 2H), 4.11 (d, J = 6.3 Hz, |
| | 1H), 4.01 (dd, J = 15.6, 7.8 Hz, 1H), 3.16 (dq, J = 13.6, 6.8 Hz, 1H), |
| | 2.56-2.47 (m, 2H), 2.03 (dd, J = 11.8, 8.9 Hz, 2H), 1.68 (d, J = 6.6 |
| | Hz, 3H), 1.39-1.33 (m, 9H). |
| Final Cpd 177 | 1H NMR (400 MHz, DMSO) d ppm 10.92 (s, 1H), 9.28 (d, J = 2.2 Hz, |
| | 1H), 9.05 (d, J = 5.8 Hz, 1H), 7.87 (dd, J = 5.9, 2.7 Hz, 1H), 7.09 (d, J = |
| | 7.1 Hz, 1H), 4.90 (s, 2H), 4.05 (dq, J = 9.2, 7.1 Hz, 2H), 3.59 (dt, J = |
| | 13.7, 6.9 Hz, 1H), 3.23-2.98 (m, 1H), 2.91 (dd, J = 37.2, 7.6 Hz, 2H), |
| | 2.75-2.56 (m, 1H), 2.35-2.23 (m, 2H), 2.09-1.88 (m, 2H), 1.32 (s, |
| | 3H), 1.31 (s, 3H), 1.17 (td, J = 7.1, 3.7 Hz, 3H). |
| Final Cpd 148 | 1H NMR (400 MHz, DMSO) d 10.39 (s, 1H), 9.25 (s, 1H), 9.18 (s, |
| | 1H), 7.78 (d, J = 9.8 Hz, 1H), 7.30 (dd, J = 9.8, 1.8 Hz, 1H), 7.12 (s, |
| | 1H), 4.78 (s, 2H), 3.18-3.07 (m, 1H), 3.02 (s, 6H), 1.31 (d, J = 6.9 Hz, 6H). |
| Final Cpd 151 | 1H NMR (400 MHz, CDCl3) d 8.87 (s, 1H), 8.70 (s, 1H), 8.65 (s, 1H), |
| | 8.14 (d, J = 4.1 Hz, 1H), 4.83 (s, 2H), 3.30-3.18 (m, 1H), 3.12 (s, 6H), |
| | 1.37 (d, J = 6.9 Hz, 6H). |
| Final Cpd 149 | 1H NMR (400 MHz, CDCl3) d 9.57 (s, 1H), 9.12-9.10 (m, 1H), 8.99 |
| | (d, J = 5.9 Hz, 1H), 7.99 (dd, J = 5.9, 2.7 Hz, 1H), 7.02 (s, 1H), 4.88 (s, |
| | 2H), 3.23-3.12 (m, 7H), 1.35 (d, J = 6.9 Hz, 6H). |
| Final Cpd 150 | 1H NMR (400 MHz, CDCl3) d 6.96 (s, 1H), 6.48 (d, J = 7.4 Hz, 1H), |
| | 4.63 (s, 2H), 3.99 (h, J = 7.9 Hz, 1H), 3.22-3.10 (m, 7H), 2.54-2.46 |
| | (m, 2H), 2.19 (s, 1H), 2.06-1.96 (m, 2H), 1.37-1.32 (m, 9H). |
| Final Cpd 188 | 1H NMR (500 MHz, DMSO-d6) d ppm 0.80-0.91 (m, 2 H) 0.98-1.10 |
| | (m, 2 H) 1.28 (d, J = 6.9 Hz, 6 H) 1.47-1.60 (m, 2 H) 2.13 (tt, J = 8.4, 5.0 |
| | Hz, 1 H) 2.50 (s, 2 H) 2.67 (br dd, J = 12.1, 8.5 Hz, 1 H) 2.74 (ddd, |
| | J = 12.4, 8.8, 3.8 Hz, 1 H) 3.53 (spt, J = 6.9 Hz, 1 H) 3.68-3.77 (m, 2 H) |
| | 4.57-4.67 (m, 2 H) 4.72 (d, J = 3.7 Hz, 1 H) 6.93 (s, 1 H) 7.70 (d, J = 8.1 |
| | Hz, 1 H); 1 H exchanged |
| Final Cpd 191 | 1H NMR (500 MHz, DMSO-d6) d ppm 0.80-0.91 (m, 2 H) 0.99-1.10 |
| | (m, 2 H) 1.28 (d, J = 6.9 Hz, 6 H) 1.50-1.64 (m, 4 H) 1.64-1.75 (m, 2 |
| | H) 1.86-1.95 (m, 2 H) 1.95-2.34 (m, 4 H) 2.13 (tt, J = 8.4, 4.8 Hz, 1 |
| | H) 2.60-2.69 (m, 1 H) 3.53 (spt, J = 6.9 Hz, 1 H) 3.63-3.71 (m, 1 H) |
| | 3.74-3.82 (m, 1 H) 4.55-4.67 (m, 2 H) 4.75 (br d, J = 3.4 Hz, 1 H) |
| | 6.94 (s, 1 H) 7.70 (br d, J = 8.2 Hz, 1 H) |
| Final Cpd 24 | 1H NMR (500 MHz, DMSO-d6) d ppm 1.21 (s, 3 H) 1.29 (d, J = 6.87 |
| | Hz, 6 H) 1.92 (td, J = 9.00, 2.14 Hz, 2 H) 2.17-2.25 (m, 2 H) 3.55 |
| | (quin, J = 6.87 Hz, 1 H) 3.71-3.86 (m, 1 H) 4.53 (s, 2 H) 4.96 (s, 1 H) |
| | 7.46 (s, 1 H) 8.16 (d, J = 7.17 Hz, 1 H) |
| Final Cpd 195 | 1H NMR (400 MHz, DMSO-d6) d ppm 0.79-0.92 (m, 2 H) 0.98-1.13 |
| | (m, 2 H) 1.22 (s, 3 H) 1.77-1.90 (m, 2 H) 1.97 (td, J = 9.0, 2.1 Hz, 2 H) |
| | 2.11-2.19 (m, 1 H) 2.20-2.30 (m, 2 H) 2.35-2.51 (m, 2 H) 3.03- |
| | 3.15 (m, 2 H) 3.79 (sxt, J = 7.9 Hz, 1 H) 4.94 (s, 2 H) 5.04 (s, 1 H) 7.18 |
| | (s, 1 H) 8.65 (d, J = 7.2 Hz, 1 H) |
| Final Cpd 196 | 1H NMR (400 MHz, DMSO-d6) d ppm 0.82-0.89 (m, 2 H) 0.89 (t, |
| | J = 7.3 Hz, 3 H) 0.99-1.10 (m, 2 H) 1.22 (s, 3 H) 1.39 (dq, J = 15.0, 7.4 |
| | Hz, 2 H) 1.52-1.66 (m, 2 H) 1.97 (td, J = 9.0, 2.4 Hz, 2 H) 2.14 (tt, |
| | J = 8.4, 4.9 Hz, 1 H) 2.21-2.30 (m, 2 H) 2.92-3.03 (m, 2 H) 3.79 (dq, |
| | J = 15.6, 8.0 Hz, 1 H) 4.91 (s, 2 H) 5.03 (s, 1 H) 7.13 (s, 1 H) 8.63 (d, |
| | J = 7.2 Hz, 1 H) |
| Final Cpd 194 | 1H NMR (400 MHz, DMSO-d6) d ppm 0.82-0.92 (m, 2 H) 0.99-1.10 |
| | (m, 2 H) 1.22 (s, 3 H) 1.85-2.02 (m, 4 H) 2.10-2.20 (m, 1 H) 2.22- |
| | 2.30 (m, 2 H) 2.99-3.08 (m, 2 H) 3.15-3.23 (m, 3 H) 3.35-3.41 (m, |
| | 2 H) 3.67-3.88 (m, 1H) 4.87-4.97 (m, 2 H) 5.00-5.11 (m, 1 H) 7.04- |
| | 7.13 (m, 1 H) 8.60-8.75 (m, 1 H) |
| Final Cpd 189 | 1H NMR (400 MHz, DMSO-d6) d ppm 0.80-0.93 (m, 2 H) 0.97-1.10 |
| | (m, 2 H) 1.13-1.24 (m, 1 H) 1.29 (d, J = 6.9 Hz, 6 H) 1.39-1.53 (m, 1 |
| | H) 1.56-1.76 (m, 2 H) 2.09-2.20 (m, 2 H) 2.23-2.35 (m, 1 H) 2.72- |
| | 2.81 (m, 1 H) 2.89 (dd, J = 10.6, 3.7 Hz, 1 H) 3.17 (q, J = 10.3 Hz, 2 H) |
| | 3.54 (spt, J = 6.9 Hz, 1 H) 3.65-3.77 (m, 1 H) 4.48-4.59 (m, 2 H) 6.95 |
| | (s, 1 H) 7.88 (d, J = 8.1 Hz, 1 H) |
| Final Cpd 166 | 1H NMR (400 MHz, DMSO-d6) d 11.39 (s, 1H), 9.51 (d, J = 0.69 Hz, |
| | 1H), 8.35 (dd, J = 0.69, 10.17 Hz, 1H), 7.90 (br d, J = 9.94 Hz, 1H), 7.00 |
| | (s, 1H), 4.93 (s, 2H), 3.57 (quin, J = 6.88 Hz, 1H), 2.15 (tt, J = 4.91, 8.38 |
| | Hz, 1H), 1.31 (d, J = 6.94 Hz, 6H), 0.99-1.10 (m, 2H), 0.78-0.95 (m, 2H) |
| Final Cpd 192 | 1H NMR (400 MHz, DMSO-d6) d 8.20 (d, J = 7.17 Hz, 1H), 6.95 (s, |
| | 1H), 4.76 (s, 1H), 4.51 (s, 2H), 3.64-3.79 (m, 1H), 3.54 (quin, J = 6.88 |
| | Hz, 1H), 2.26 (ddd, J = 2.77, 7.69, 9.19 Hz, 2H), 2.07-2.17 (m, 1H), |
| | 1.80-1.90 (m, 2H), 1.44 (q, J = 7.24 Hz, 2H), 1.29 (d, J = 6.70 Hz, 6H), |
| | 1.05 (dd, J = 2.43, 8.44 Hz, 2H), 0.85-0.89 (m, 2H), 0.80-0.85 (m, 3H) |
| Final Cpd 175 | 1H NMR (500 MHz, DMSO-d6) d ppm 1.34 (d, J = 6.9 Hz, 6 H) 2.12 (t, |
| | J = 19.1 Hz, 3 H) 3.63 (quin, J = 6.9 Hz, 1 H) 4.91 (s, 2 H) 7.29 (dd, |
| | J = 9.8, 1.8 Hz, 1 H) 7.55 (s, 1 H) 7.79 (s, 1H) 9.18 (dd, J = 1.8, 1.0 Hz, 1 |
| | H) 9.22-9.33 (m, 1 H) 10.47 (s, 1 H). |
| Final Cpd 161 | 1H NMR (400 MHz, DMSO-d6) d ppm 1.21 (s, 3 H) 1.32 (d, J = 6.9 Hz, |
| | 6 H) 1.92 (br d, J = 2.5 Hz, 2 H) 2.11 (t, J = 19.1 Hz, 3 H) 2.17-2.26 (m, |
| | 2 H) 3.59 (quin, J = 6.9 Hz, 1 H) 3.72-3.87 (m, 1 H) 4.56 (s, 2 H) 4.91- |
| | 4.99 (m, 1 H) 7.48 (s, 1 H) 8.16 (d, J = 7.2 Hz, 1 H). |
| Final Cpd 176 | 1H NMR (400 MHz, DMSO-d6) d ppm 1.34 (d, J = 6.9 Hz, 6 H) 2.12 (t, |
| | J = 19.1 Hz, 3 H) 3.62 (quin, J = 6.9 Hz, 1 H) 4.98 (s, 2 H) 7.53 (s, 1 H) |
| | 7.84-8.02 (m, 1 H) 8.36 (dd, J = 9.9, 0.7 Hz, 1 H) 9.52 (d, J = 0.7 Hz, 1 |
| | H) 11.40 (s, 1 H). |
| Final Cpd 70 | 1H NMR (500 MHz, DMSO-d6) d ppm 1.32 (d, J = 6.87 Hz, 6 H) 1.36 |
| | (s, 9 H) 3.58 (quin, J = 6.87 Hz, 1 H) 4.92 (s, 2 H) 7.20 (s, 1 H) 7.97 (dd, |
| | J = 5.72, 0.99 Hz, 1 H) 8.66 (d, J = 5.95 Hz, 1 H) 8.90 (d, J = 0.92 Hz, 1 H) |
| | 10.53-12.11 (m, 1 H) |
| Final Cpd 70 | 1H NMR (400 MHz, DMSO-d6) d ppm 1.32 (d, J = 6.94 Hz, 6 H) 1.36 |
| | (s, 9 H) 3.51-3.70 (m, 1 H) 4.91 (s, 2 H) 7.20 (s, 1 H) 7.97 (dd, |
| | J = 5.78, 1.16 Hz, 1 H) 8.65 (d, J = 5.78 Hz, 1 H) 8.90 (d, J = 0.93 Hz, 1 H) |
| | 10.44-12.03 (m, 1 H) |
| Final Cpd 71 | 1H NMR (400 MHz, DMSO-d6) d ppm 1.21 (s, 3 H) 1.31 (d, J = 6.94 |
| | Hz, 6 H) 1.35 (s, 9 H) 1.86-2.01 (m, 2 H) 2.16-2.25 (m, 2 H) 3.56 |
| | (quin, J = 6.88 Hz, 1 H) 3.71-3.91 (m, 1 H) 4.52 (s, 2 H) 4.83-5.28 (m, |
| | 1H) 7.15 (s, 1H) 8.18 (d, J = 7.17 Hz, 1 H) |
| Final Cpd 72 | 1H NMR (500 MHz, DMSO-d6) d ppm 1.16 (t, J = 7.2 Hz, 3 H) 1.28 (d, |
| | J = 6.9 Hz, 6 H) 3.13-3.25 (m, 2 H) 3.46 (spt, J = 6.9 Hz, 1 H) 4.87 (s, 2 |
| | H) 6.35 (s, 1 H) 6.40 (t, J = 5.6 Hz, 1 H) 7.98 (dd, J = 5.8, 1.1 Hz, 1 H) |
| | 8.66 (d, J = 5.8 Hz, 1 H) 8.90 (d, J = 1.1 Hz, 1 H) 10.95-11.48 (m, 1 H) |
| Final Cpd 73 | 1H NMR (500 MHz, DMSO-d6) d ppm 1.16 (t, J = 7.1 Hz, 3 H) 1.21 (s, |
| | 3 H) 1.27 (d, J = 6.9 Hz, 6 H) 1.84-2.01 (m, 2 H) 2.15-2.27 (m, 2 H) |
| | 3.12-3.26 (m, 2 H) 3.43 (quin, J = 6.9 Hz, 1 H) 3.77 (sxt, J = 8.0 Hz, 1 |
| | H) 4.47 (s, 2 H) 4.96 (br s, 1 H) 6.31 (s, 1 H) 6.36 (t, J = 5.6 Hz, 1 H) |
| | 8.18 (d, J = 7.2 Hz, 1 H) |
| Final Cpd 74 | 1H NMR (500 MHz, DMSO-d6) d ppm 1.16 (t, J = 7.2 Hz, 3 H) 1.28 (d, |
| | J = 6.9 Hz, 6 H) 3.14-3.24 (m, 2 H) 3.45 (quin, J = 6.9 Hz, 1 H) 3.58 (s, |
| | 3 H) 4.76 (s, 2 H) 6.34 (s, 1 H) 6.39 (t, J = 5.6 Hz, 1 H) 6.97 (d, J = 9.9 |
| | Hz, 1 H) 7.91 (br d, J = 9.5 Hz, 1 H) 10.49-11.15 (m, 1 H) |
| Final Cpd 75 | 1H NMR (500 MHz, DMSO-d6) d ppm 1.17 (t, J = 7.2 Hz, 3 H) 1.29 (d, |
| | J = 6.9 Hz, 6 H) 3.12-3.26 (m, 2 H) 3.47 (quin, J = 6.9 Hz, 1 H) 4.81 (s, |
| | 2 H) 6.36 (s, 1 H) 6.38-6.47 (m, 1 H) 7.30 (dd, J = 9.8, 2.0 Hz, 1 H) |
| | 7.79 (d, J = 9.8 Hz, 1 H) 9.19 (dd, J = 1.7, 0.9 Hz, 1 H) 9.24 (d, J = 0.8 Hz, |
| | 1 H) 10.37-10.63 (m, 1 H) |
| Final Cpd 94 | 1H NMR (400 MHz, DMSO-d6, 27° C.): Shift = 11.21-11.30 (m, 1H), |
| | 8.62 (d, J = 7.0 Hz, 1H), 8.19-8.23 (m, 1H), 7.30 (d, J = 7.0 Hz, 1H), |
| | 7.01 (s, 1H), 4.90-4.97 (m, 2H), 3.47-3.75 (m, 1H), 2.68-2.80 (m, 3H), |
| | 2.12-2.20 (m, 1H), 1.91 (s, 1H), 1.23-1.36 (m, 6H), 1.03-1.12 (m, 2H), |
| | 0.85-0.93 ppm (m, 2H) |
| Final Cpd 109 | 1H NMR (400 MHz, DMSO-d6, 27° C.): Shift = 10.00 (br s, 1H), 8.23 |
| | (dd, J = 4.7, 1.7 Hz, 1H), 8.14 (dd, J = 8.0, 1.7 Hz, 1H), 7.44 (dd, J = |
| | 8.0, 4.7 Hz, 1H), 6.99 (s, 1H), 4.92 (s, 2H), 3.57 (spt, J = 6.9 Hz, 1H), |
| | 2.08-2.18 (m, 1H), 1.32 (d, J = 6.9 Hz, 6H), 1.00-1.10 (m, 2H), 0.82- |
| | 0.93 ppm (m, 2H) |
| Final Cpd 115 | 1H NMR (400 MHz, DMSO-d6, 27° C.): Shift = 10.63 (br s, 1H), 8.94- |
| | 8.99 (m, 2H), 8.91 (d, J = 0.2 Hz, 1H), 7.00 (s, 1H), 4.84-4.89 (m, 2H), |
| | 3.58 (dt, J = 13.8, 6.8 Hz, 1H), 2.12-2.19 (m, 1H), 1.29-1.34 (m, 6H), |
| | 1.03-1.09 (m, 2H), 0.85-0.91 ppm (m, 2H) |
| Final Cpd 110 | 1H NMR (400 MHz, DMSO-d6, 27° C.): Shift = 10.60 (br s, 1H), 8.58 |
| | (d, J = 2.7 Hz, 1H), 8.04 (dd, J = 8.7, 2.8 Hz, 1H), 7.49 (d, J = 8.7 Hz, |
| | 1H), 7.00 (s, 1H), 4.84 (s, 2H), 3.57 (quin, J = 6.9 Hz, 1H), 2.11-2.19 |
| | (m, 1H), 1.31 (d, J = 6.9 Hz, 6H), 1.02-1.10 (m, 2H), 0.82-0.94 ppm |
| | (m, 2H) |
| Final Cpd 111 | 1H NMR (400 MHz, DMSO-d6, 27° C.): Shift = 10.21 (s, 1H), 8.32 (d, J = |
| | 2.6 Hz, 1H), 7.85 (dd, J = 8.9, 2.7 Hz, 1H), 6.99 (s, 1H), 6.81 (d, J = |
| | 8.9 Hz, 1H), 4.76-4.82 (m, 2H), 3.81 (s, 3H), 3.57 (quin, J = 6.8 Hz, |
| | 1H), 2.11-2.19 (m, 1H), 1.26-1.36 (m, 6H), 1.02-1.10 (m, 2H), 0.84- |
| | 0.92 ppm (m, 2H) |
| Final Cpd 117 | 1H NMR (400 MHz, DMSO-d6, 27° C.): Shift = 10.61-10.72 (m, 1H), |
| | 8.62 (d, J = 2.2 Hz, 1H), 8.33 (d, J = 2.1 Hz, 1H), 8.19 (t, J = 2.0 Hz, |
| | 1H), 7.00 (s, 1H), 4.86 (s, 2H), 3.57 (spt, J = 6.9 Hz, 1H), 2.10-2.19 (m, |
| | 1H), 1.31 (d, J = 6.9 Hz, 6H), 1.00-1.09 (m, 2H), 0.82-0.94 ppm (m, 2H) |
| Final Cpd 118 | 1H NMR (400 MHz, DMSO-d6, 27° C.): Shift = 10.62 (s, 1H), 8.44 (d, J = |
| | 5.2 Hz, 2H), 7.53 (d, J = 5.2 Hz, 2H), 7.00 (s, 1H), 4.84 (s, 2H), 3.57 |
| | (spt, J = 6.8 Hz, 1H), 2.12-2.19 (m, 1H), 1.31 (d, J = 6.9 Hz, 6H), 1.00- |
| | 1.16 (m, 2H), 0.84-0.94 ppm (m, 2H) |
| Final Cpd 119 | 1H NMR (400 MHz, DMSO-d6, 27° C.): Shift = 10.39-10.44 (m, 1H), |
| | 8.71 (s, 1H), 8.29 (dd, J = 4.7, 1.5 Hz, 1H), 7.99 (ddd, J = 8.3, 2.6, 1.5 |
| | Hz, 1H), 7.34-7.39 (m, 1H), 7.00 (s, 1H), 4.81-4.86 (m, 2H), 3.51-3.63 |
| | (m, 1H), 2.12-2.19 (m, 1H), 1.31 (dt, J = 6.8, 0.7 Hz, 6H), 1.03-1.09 |
| | (m, 2H), 0.85-0.91 ppm (m, 2H) |
| Final Cpd 127 | 1H NMR (400 MHz, DMSO-d6, 27° C.): Shift = 10.52 (s, 1H), 8.30 (d, J = |
| | 5.6 Hz, 1H), 7.41 (d, J = 1.8 Hz, 1H), 7.33 (dd, J = 5.6, 1.9 Hz, 1H), |
| | 6.99 (s, 1H), 4.83 (s, 2H), 3.57 (spt, J = 6.8 Hz, 1H), 2.40 (s, 3H), 2.12- |
| | 2.24 (m, 1H), 1.31 (d, J = 6.9 Hz, 6H), 1.00-1.15 (m, 2H), 0.84-0.94 |
| | ppm (m, 2H) |
| Final Cpd 133 | 1H NMR (DMSO-d6, 400 MHz) Shift 10.0-10.1 (m, 1H), 8.62 (s, 1H), |
| | 8.42 (d, 1H, J = 5.5 Hz), 8.01 (d, 1H, J = 5.5 Hz), 6.99 (s, 1H), 4.99 (s, |
| | 2H), 3.57 (quin, 1H, J = 6.9 Hz), 2.1-2.2 (m, 1H), 1.31 (d, 6H, J = 6.9 |
| | Hz), 1.0-1.1 (m, 2H), 0.8-0.9 (m, 2H) |
| Final Cpd 29 | 1H NMR (400 MHz, DMSO-d6) d ppm 0.84-0.90 (m, 2 H) 1.02-1.08 |
| | (m, 2H) 1.29 (d, J = 6.70 Hz, 6 H) 1.71-1.80 (m, 1 H) 1.80-1.90 (m, 1 |
| | H) 2.14 (tt, J = 8.38, 5.03 Hz, 1 H) 2.21-2.38 (m, 2 H) 2.78 (s, 3 H) |
| | 3.08 (dd, J = 12.14, 7.05 Hz, 1 H) 3.37-3.45 (m, 1 H) 3.55 (spt, J = 6.86 |
| | Hz, 1 H) 4.00-4.15 (m, 1 H) 4.59 (d, J = 2.08 Hz, 2 H) 6.96 (s, 1 H) |
| | 8.28 (d, J = 7.40 Hz, 1 H) |
| Final Cpd 30 | 1H NMR (400 MHz, DMSO-d6) d ppm 0.84-0.89 (m, 2 H) 1.02-1.09 |
| | (m, 2H) 1.29 (d, J = 6.82 Hz, 6 H) 1.69-1.81 (m, 1 H) 1.81-1.89 (m, 1 |
| | H) 2.14 (tt, J = 8.39, 4.92 Hz, 1 H) 2.22-2.39 (m, 2 H) 2.78 (s, 3 H) |
| | 3.08 (dd, J = 12.10, 7.04 Hz, 1H) 3.41 (dd, J = 11.88, 4.84 Hz, 1H) 3.48- |
| | 3.62 (m, 1 H) 4.00-4.15 (m, 1 H) 4.58 (d, J = 2.20 Hz, 2 H) 6.96 (s, 1 |
| | H) 8.27 (d, J = 7.26 Hz, 1 H) |
| Final Cpd 31 | 1H NMR (400 MHz, DMSO-d6) d ppm 0.83-0.90 (m, 2 H) 1.01-1.09 |
| | (m, 2H) 1.29 (d, J = 6.82 Hz, 6 H) 1.70-1.80 (m, 1 H) 1.81-1.91 (m, 1 |
| | H) 2.08-2.19 (m, 1 H) 2.22-2.38 (m, 2 H) 2.78 (s, 3 H) 3.08 (dd, |
| | J = 12.10, 7.26 Hz, 1 H) 3.36-3.46 (m, 1 H) 3.55 (quin, J = 6.88 Hz, 1 H) |
| | 4.00-4.13 (m, 1 H) 4.58 (d, J = 1.98 Hz, 2 H) 6.96 (s, 1 H) 8.27 (d, |
| | J = 7.26 Hz, 1 H) |
| Final Cpd 187 | 1H NMR (400 MHz, DMSO-d6) d ppm 0.80-0.93 (m, 2 H) 0.99-1.12 |
| | (m, 2 H) 1.20-1.41 (m, 9 H) 1.53-1.63 (m, 1 H) 1.69-1.78 (m, 1 H) |
| | 2.14 (tt, J = 8.38, 4.91 Hz, 1 H) 2.26 (dd, J = 11.67, 9.13 Hz, 1 H) 2.31- |
| | 2.42 (m, 1 H) 2.68-2.77 (m, 1 H) 2.84 (dd, J = 11.67, 3.35 Hz, 1 H) |
| | 3.47-3.65 (m, 2 H) 4.48-4.60 (m, 2 H) 6.95 (s, 1 H) 7.84 (d, J = 8.09 |
| | Hz, 1 H) |
| Final Cpd 35 | 1H NMR (400 MHz, DMSO-d6) d ppm 0.30-0.37 (m, 2 H) 0.42-0.51 |
| | (m, 2 H) 0.84-0.91 (m, 2 H) 1.02-1.10 (m, 2 H) 1.10-1.20 (m, 1 H) |
| | 1.31 (d, J = 6.94 Hz, 6 H) 2.09-2.21 (m, 1 H) 3.49-3.64 (m, 1 H) 3.72 |
| | (d, J = 6.94 Hz, 2 H) 4.74 (s, 2 H) 6.40 (d, J = 9.48 Hz, 1 H) 6.98 (s, 1 H) |
| | 7.37 (dd, J = 9.71, 2.77 Hz, 1 H) 8.07 (d, J = 2.77 Hz, 1 H) 9.90 (s, 1 H) |
| Final Cpd 32 | 1H NMR (400 MHz, DMSO-d6) d ppm 0.84-0.92 (m, 2 H) 1.03-1.11 |
| | (m, 2 H) 1.31 (d, J = 6.94 Hz, 6 H) 2.15 (tt, J = 8.44, 4.85 Hz, 1 H) 3.52- |
| | 3.65 (m, 1 H) 4.89 (s, 2 H) 7.01 (s, 1 H) 7.87 (dd, J = 5.90, 2.66 Hz, 1 H) |
| | 9.05 (dd, J = 5.90, 1.04 Hz, 1 H) 9.28 (dd, J = 2.77, 0.92 Hz, 1 H) 10.72- |
| | 11.06 (m, 1 H) |
| Final Cpd 33 | 1H NMR (500 MHz, DMSO-d6) d ppm 0.83-0.90 (m, 2 H) 1.02-1.08 |
| | (m, 2 H) 1.29 (d, J = 6.87 Hz, 6 H) 1.39-1.46 (m, 1 H) 1.76-1.87 (m, 1 |
| | H) 2.13 (tt, J = 8.39, 4.88 Hz, 1 H) 3.33-3.38 (m, 1 H) 3.41 (dd, |
| | J = 11.98, 1.60 Hz, 1 H) 3.49-3.60 (m, 2 H) 3.68 (dd, J = 11.98, 2.98 Hz, |
| | 1 H) 3.75 (dt, J = 11.25, 3.45 Hz, 1 H) 3.85 (ddt, J = 11.22, 7.40, 3.97, |
| | 3.97 Hz, 1 H) 4.54-4.70 (m, 2 H) 4.92 (d, J = 4.73 Hz, 1 H) 6.95 (s, 1 |
| | H) 7.90 (d, J = 7.93 Hz, 1 H) |
| Final Cpd 36 | 1H NMR (400 MHz, DMSO-d6) d ppm 0.83-0.90 (m, 2 H) 1.02-1.09 |
| | (m, 2 H) 1.29 (d, J = 6.94 Hz, 6 H) 1.34-1.48 (m, 2 H) 1.67 (br dd, |
| | J = 12.48, 2.31 Hz, 2 H) 2.14 (tt, J = 8.44, 4.85 Hz, 1 H) 3.32-3.43 (m, 2 |
| | H) 3.54 (quin, J = 6.88 Hz, 1 H) 3.73-3.88 (m, 3 H) 4.54 (s, 2 H) 6.95 |
| | (s, 1 H) 7.99 (d, J = 7.63 Hz, 1 H) |
| Final Cpd 81 | 1H NMR (500 MHz, DMSO-d6) d ppm 0.84-0.91 (m, 2 H) 1.03-1.09 |
| | (m, 2 H) 1.30 (d, J = 6.87 Hz, 6 H) 2.14 (tt, J = 8.37, 4.98 Hz, 1 H) 3.49- |
| | 3.57 (m, 1 H) 3.58 (s, 3 H) 4.67-4.91 (m, 2 H) 6.90-7.05 (m, 1 H) |
| | 6.94-7.02 (m, 1 H) 7.90 (br d, J = 9.77 Hz, 1 H) 10.86 (s, 1 H) |
| Final Cpd 45 | 1H NMR (400 MHz, DMSO-d6) d ppm 1.03-1.11 (m, 2 H) 1.15-1.22 |
| | (m, 2 H) 1.23-1.38 (m, 6 H) 2.43-2.50 (m, 1 H) 3.56 (dt, J = 13.70, |
| | 6.91 Hz, 1 H) 4.91 (s, 2 H) 7.29 (br dd, J = 9.71, 1.85 Hz, 1 H) 7.80 (d, |
| | J = 9.71 Hz, 1 H) 9.18 (br d, J = 0.93 Hz, 1 H) 9.25 (d, J = 0.69 Hz, 1 H) |
| | 10.46 (s, 1 H) |
| Final Cpd 43 | 1H NMR (400 MHz, DMSO-d6) d ppm 0.93-1.02 (m, 2 H) 1.04-1.15 |
| | (m, 2 H) 1.21 (s, 3 H) 1.26 (d, J = 6.94 Hz, 6 H) 1.84-1.99 (m, 2 H) |
| | 2.12 (tt, J = 8.38, 5.03 Hz, 1 H) 2.17-2.27 (m, 2 H) 3.42-3.58 (m, 1 H) |
| | 3.70-3.86 (m, 1 H) 4.48 (s, 2 H) 4.96 (s, 1 H) 8.15 (d, J = 7.17 Hz, 1 H) |
| Final Cpd 182 | 1H NMR (400 MHz, DMSO-d6) d ppm 0.84-0.91 (m, 2 H) 0.96-1.04 |
| | (m, 2 H) 1.23 (d, J = 6.94 Hz, 6 H) 2.08 (tt, J = 8.24, 5.06 Hz, 1 H) 3.34- |
| | 3.46 (m, 1 H) 4.71 (s, 2 H) 5.09 (s, 2 H) 7.30 (dd, J = 9.83, 1.97 Hz, 1 H) |
| | 7.73-7.83 (m, 1 H) 9.19 (dd, J = 1.62, 0.92 Hz, 1 H) 9.25 (d, J = 0.69 Hz, |
| | 1 H) 10.23-10.59 (m, 1 H) |
| Final Cpd 46 | 1H NMR (500 MHz, DMSO-d6) d ppm 1.02-1.08 (m, 2 H) 1.15-1.20 |
| | (m, 2 H) 1.21 (s, 3 H) 1.27 (d, J = 6.87 Hz, 6 H) 1.92 (td, J = 8.96, 2.37 |
| | Hz, 2 H) 2.15-2.28 (m, 2 H) 2.44-2.49 (m, 1 H) 3.52 (spt, J = 6.84 Hz, |
| | 1 H) 3.79 (sxt, J = 8.00 Hz, 1 H) 4.56 (s, 2 H) 4.98 (s, 1 H) 8.14 (d, |
| | J = 7.17 Hz, 1 H) |
| Final Cpd 181 | 1H NMR (500 MHz, DMSO-d6) d ppm 0.82-0.89 (m, 2 H) 0.94-1.03 |
| | (m, 2H) 1.16-1.26 (m, 9 H) 1.89-1.98 (m, 2 H) 2.06 (tt, J = 8.32, 4.96 |
| | Hz, 1 H) 2.16-2.25 (m, 2 H) 3.33-3.39 (m, 1 H) 3.70-3.83 (m, 1 H) |
| | 4.37 (s, 2 H) 4.95 (s, 1 H) 5.04 (s, 2 H) 8.15 (d, J = 7.32 Hz, 1 H) |
| Final Cpd 41 | 1H NMR (500 MHz, DMSO-d6) d ppm 1.21 (s, 3 H) 1.27 (d, J = 6.87 |
| | Hz, 6 H) 1.87-1.99 (m, 2 H) 2.16-2.26 (m, 2 H) 3.38-3.48 (m, 1 H) |
| | 3.70-3.85 (m, 1 H) 3.97-4.12 (m, 2 H) 4.48 (s, 2 H) 4.96 (br s, 1 H) |
| | 6.50 (s, 1 H) 7.15 (br t, J = 6.71 Hz, 1 H) 8.18 (d, J = 7.17 Hz, 1 H) |
| Final Cpd 42 | 1H NMR (500 MHz, DMSO-d6) d ppm 1.30 (d, J = 6.87 Hz, 6 H) 3.47 |
| | (spt, J = 6.89 Hz, 1 H) 3.98-4.14 (m, 2 H) 4.83 (s, 2 H) 6.56 (s, 1 H) |
| | 7.20 (t, J = 6.79 Hz, 1 H) 7.30 (dd, J = 9.77, 1.83 Hz, 1 H) 7.79 (d, J = 9.77 |
| | Hz, 1 H) 9.19 (dd, J = 1.60, 0.99 Hz, 1 H) 9.25 (d, J = 0.76 Hz, 1 H) 10.29- |
| | 10.61 (m, 1 H) |
| Final Cpd 163 | 1H NMR (500 MHz, DMSO-d6) d ppm 0.87-0.93 (m, 2 H) 0.98-1.04 |
| | (m, 2 H) 1.05-1.13 (m, 4 H) 2.16 (tt, J = 8.41, 4.94 Hz, 1 H) 2.56-2.63 |
| | (m, 1 H) 4.87 (s, 2 H) 7.01 (s, 1 H) 7.89 (br d, J = 10.07 Hz, 1 H) 8.35 |
| | (dd, J = 10.07, 0.61 Hz, 1 H) 9.51 (s, 1 H) 11.23-11.50 (m, 1 H) |
| Final Cpd 37 | 1H NMR (500 MHz, DMSO-d6) d ppm 0.84-0.91 (m, 2 H) 1.01-1.09 |
| | (m, 2 H) 2.13 (tt, J = 8.39, 4.96 Hz, 1 H) 3.00 (s, 6 H) 4.77 (s, 2 H) 6.97 |
| | (s, 1 H) 7.30 (dd, J = 9.77, 1.83 Hz, 1 H) 7.78 (d, J = 9.77 Hz, 1 H) 9.18 |
| | (d, J = 0.61 Hz, 1 H) 9.25 (d, J = 0.61 Hz, 1 H) 10.40 (s, 1 H) |
| Final Cpd 39 | 1H NMR (400 MHz, DMSO-d6) d ppm 0.85-0.93 (m, 2 H) 1.01-1.09 |
| | (m, 2 H) 2.13 (tt, J = 8.44, 4.85 Hz, 1 H) 3.22 (s, 3 H) 3.36-3.45 (m, 2 |
| | H) 3.47-3.56 (m, 2 H) 4.74 (s, 2 H) 6.94 (s, 1 H) 7.02 (t, J = 5.78 Hz, 1 |
| | H) 7.31 (dd, J = 9.71, 1.85 Hz, 1 H) 7.78 (d, J = 9.94 Hz, 1 H) 9.18 (d, |
| | J = 0.69 Hz, 1 H) 9.25 (d, J = 0.69 Hz, 1 H) 9.87-11.02 (m, 1 H) |
| Final Cpd 38 | 1H NMR (400 MHz, DMSO-d6) d ppm 0.82-0.92 (m, 2 H) 1.00-1.11 |
| | (m, 2 H) 2.12 (tt, J = 8.38, 4.91 Hz, 1 H) 2.97 (s, 3 H) 3.17 (s, 3 H) 3.53- |
| | 3.63 (m, 2 H) 3.69-3.80 (m, 2 H) 4.77 (s, 2 H) 6.97 (s, 1 H) 7.30 (dd, |
| | J = 9.71, 1.85 Hz, 1 H) 7.78 (d, J = 9.71 Hz, 1 H) 9.18 (s, 1 H) 9.25 (d, |
| | J = 0.69 Hz, 1 H) 10.39 (s, 1 H) |
| Final Cpd 68 | 1H NMR (500 MHz, DMSO-d6) d ppm 0.83-0.94 (m, 5 H) 1.02-1.09 |
| | (m, 2 H) 1.39 (sxt, J = 7.45 Hz, 2 H) 1.73 (quin, J = 7.51 Hz, 2 H) 2.14 (tt, |
| | J = 8.39, 4.96 Hz, 1 H) 2.92-3.01 (m, 2 H) 4.86 (s, 2 H) 7.00 (s, 1 H) |
| | 7.29 (dd, J = 9.84, 1.91 Hz, 1 H) 7.79 (d, J = 9.77 Hz, 1 H) 9.16-9.21 (m, |
| | 1 H) 9.25 (d, J = 0.76 Hz, 1 H) 10.48 (s, 1 H) |
| Final Cpd 69 | 1H NMR (500 MHz, DMSO-d6) d ppm 0.84-0.90 (m, 2 H) 0.96 (d, |
| | J = 6.56 Hz, 6 H) 1.02-1.10 (m, 2 H) 2.11-2.19 (m, 1 H) 2.26 (dquin, |
| | J = 13.59, 6.83, 6.83, 6.83, 6.83 Hz, 1 H) 2.84 (d, J = 7.17 Hz, 2 H) 4.87 |
| | (s, 2 H) 7.00 (s, 1 H) 7.29 (dd, J = 9.84, 1.91 Hz, 1 H) 7.75-7.83 (m, 1 |
| | H) 9.19 (dd, J = 1.75, 0.99 Hz, 1 H) 9.25 (d, J = 0.76 Hz, 1 H) 10.48 (s, 1 |
| | H) |
| Final Cpd 164 | 1H NMR (400 MHz, DMSO-d6) ppm 0.84-0.92 (m, 2 H), 1.02-1.10 |
| | (m, 2H), 1.31 (d, J = 6.8 Hz, 6 H), 2.11-2.19 (m, 1 H), 3.30 (s, 8 H), |
| | 3.57 (spt, J = 6.8 Hz, 1 H), 4.82 (s, 2 H), 6.68-6.72 (m, 1 H), 6.73 (s, 1 |
| | H), 7.00 (s, 1 H), 10.69 (s, 1 H), 11.22 (br s, 1 H). |
| | 19F NMR (377 MHz, DMSO-d6) ppm −70.34 (s, 1 F). |
| | — |
| | 1H NMR (400 MHz, DMSO-d6) ppm 11.22 (br s, 1 H), 10.69 (s, 1 H), |
| | 7.00 (s, 1 H), 6.73 (s, 1 H), 6.68-6.72 (m, 1 H), 4.82 (s, 2 H), 3.57 (spt, |
| | J = 6.8 Hz, 1 H), 3.30 (s, 8 H), 2.19-2.11 (m, 1 H), 1.31 (d, J = 6.8 Hz, 6 |
| | H), 1.10-1.02 (m, 2 H), 0.92-0.84 (m, 2 H). |
| Final Cpd 172 | 1H NMR (400 MHz, CHLOROFORM-d) ppm 0.87-0.94 (m, 2 H), |
| | 1.06-1.14 (m, 2 H), 1.40 (d, J = 6.9 Hz, 6 H), 2.12 (tt, J = 8.4, 5.0 Hz, 1 |
| | H), 3.69 (spt, J = 6.9 Hz, 1 H), 5.10 (s, 2 H), 6.84 (s, 1 H), 7.83 (d, J = 9.2 |
| | Hz, 1 H), 8.69 (d, J = 9.3 Hz, 1 H), 10.09 (br s, 1 H). |
| | 19F NMR (377 MHz, CHLOROFORM-d) ppm −66.62 (s, 3 F). |
| | — |
| | 1H NMR (400 MHz, CHLOROFORM-d) ppm 10.09 (br s, 1 H), 8.69 |
| | (d, J = 9.3 Hz, 1 H), 7.83 (d, J = 9.2 Hz, 1 H), 6.84 (s, 1 H), 5.10 (s, 2 H), |
| | 3.69 (spt, J = 6.9 Hz, 1 H), 2.12 (tt, J = 8.4, 5.0 Hz, 1 H), 1.40 (d, J = 6.9 |
| | Hz, 6 H), 1.06-1.14 (m, 2 H), 0.87-0.94 (m, 2 H). |
| Final Cpd 173 | 1H NMR (400 MHz, CHLOROFORM-d) ppm 0.86-0.94 (m, 2 H), |
| | 1.06-1.13 (m, 2 H), 1.39 (d, J = 6.9 Hz, 6 H), 2.11 (tt, J = 8.4, 4.9 Hz, 1 |
| | H), 3.67 (spt, J = 6.8 Hz, 1 H), 5.08 (s, 2 H), 6.83 (s, 1 H), 7.81 (d, J = 9.3 |
| | Hz, 1 H), 8.24 (d, J = 9.2 Hz, 1 H), 10.09 (br s, 1 H). |
| | — |
| | 1H NMR (400 MHz, CHLOROFORM-d) ppm 10.09 (br s, 1 H), 8.24 |
| | (d, J = 9.2 Hz, 1 H), 7.81 (d, J = 9.3 Hz, 1 H), 6.83 (s, 1 H), 5.08 (s, 2 H), |
| | 3.67 (spt, J = 6.8 Hz, 1 H), 2.11 (tt, J = 8.4, 4.9 Hz, 1 H), 1.39 (d, J = 6.9 |
| | Hz, 6H), 1.06-1.13 (m, 2 H), 0.86-0.94 (m, 2 H). |
| Final Cpd 95 | 1H NMR (400 MHz, DMSO-d6) ppm 0.83-0.94 (m, 2 H), 1.01-1.12 |
| | (m, 2 H), 1.32 (d, J = 6.9 Hz, 6 H), 2.10-2.22 (m, 1 H), 3.58 (spt, J = 6.9 |
| | Hz, 1 H), 4.85 (s, 2 H), 7.01 (s, 1 H), 7.66 (d, J = 1.6 Hz, 1 H), 9.19 (d, |
| | J = 1.4 Hz, 1 H), 9.38 (s, 1 H), 10.45 (br s, 1 H). |
| | — |
| | 1H NMR (400 MHz, DMSO-d6) ppm 10.45 (br s, 1 H), 9.38 (s, 1 H), |
| | 9.19 (d, J = 1.4 Hz, 1 H), 7.66 (d, J = 1.6 Hz, 1 H), 7.01 (s, 1 H), 4.85 (s, 2 |
| | H), 3.58 (spt, J = 6.9 Hz, 1 H), 2.22-2.10 (m, 1 H), 1.32 (d, J = 6.9 Hz, 6 |
| | H), 1.12-1.01 (m, 2 H), 0.94-0.83 (m, 2 H). |
| Final Cpd 96 | 1H NMR (400 MHz, CHLOROFORM-d) ppm 0.88-0.94 (m, 2 H), |
| | 1.07-1.14 (m, 2 H), 1.40 (d, J = 6.9 Hz, 6 H), 2.11 (tt, J = 8.4, 4.9 Hz, 1 |
| | H), 3.68 (spt, J = 6.9 Hz, 1 H), 4.92 (s, 2 H), 6.86 (s, 1 H), 8.28-8.36 |
| | (m, 2 H), 8.41 (d, J = 2.3 Hz, 1 H), 8.77 (br s, 1 H). |
| | 19F NMR (376 MHz, CHLOROFORM-d) ppm −146.03 (s, 1 F). |
| | — |
| | 1H NMR (400 MHz, CHLOROFORM-d) 8.77 (br s, 1 H), 8.41 (d, |
| | J = 2.3 Hz, 1 H), 8.36-8.28 (m, 2 H), 6.86 (s, 1 H), 4.92 (s, 2 H), 3.68 |
| | (spt, J = 6.9 Hz, 1 H), 2.11 (tt, J = 8.4, 4.9 Hz, 1 H), 1.40 (d, J = 6.9 Hz, 6 |
| | H), 1.14-1.07 (m, 2 H), 0.94-0.88 (m, 2 H). |
| Final Cpd 97 | 1H NMR (400 MHz, CHLOROFORM-d) ppm 0.87-0.94 (m, 2 H), |
| | 1.06-1.14 (m, 2 H), 1.40 (d, J = 7.0 Hz, 6 H), 2.11 (tt, J = 8.4, 5.0 Hz, 1 |
| | H), 2.49 (d, J = 3.3 Hz, 3 H), 3.68 (spt, J = 6.9 Hz, 1 H), 4.91 (s, 2 H), |
| | 6.86 (s, 1 H), 8.15 (t, J = 5.7 Hz, 1 H), 8.20 (d, J = 5.7 Hz, 1 H), 8.71 (br |
| | s, 1 H). |
| | 19F NMR (376 MHz, DMSO-d6) ppm −144.55 (spt, J = 3.0 Hz, 1 F). |
| | — |
| | 1H NMR (400 MHz, CHLOROFORM-d) ppm 8.71 (br s, 1 H), 8.20 (d, |
| | J = 5.7 Hz, 1 H), 8.15 (t, J = 5.7 Hz, 1 H), 6.86 (s, 1 H), 4.91 (s, 2 H), 3.68 |
| | (spt, J = 6.9 Hz, 1 H), 2.49 (d, J = 3.3 Hz, 3 H), 2.11 (tt, J = 8.4, 5.0 Hz, 1 |
| | H), 1.40 (d, J = 7.0 Hz, 6 H), 1.14-1.06 (m, 2 H), 0.94-0.87 (m, 2 H). |
| Final Cpd 102 | 1H NMR (400 MHz, CHLOROFORM-d) ppm 0.87-0.94 (m, 2 H), |
| | 1.06-1.14 (m, 2 H), 1.39 (d, J = 6.9 Hz, 6 H), 2.08-2.15 (m, 1 H), 2.49 |
| | (s, 3 H), 3.66 (spt, J = 6.9 Hz, 1 H), 3.73 (s, 3 H), 4.91 (s, 2 H), 6.85 (s, 1 |
| | H), 8.11 (d, J = 5.4 Hz, 1 H), 8.19 (d, J = 5.5 Hz, 1 H), 8.83 (s, 1 H). |
| | — |
| | 1H NMR (400 MHz, CHLOROFORM-d) ppm 8.83 (s, 1 H), 8.19 (d, |
| | J = 5.5 Hz, 1 H), 8.11 (d, J = 5.4 Hz, 1 H), 6.85 (s, 1 H), 4.91 (s, 2 H), |
| | 3.73 (s, 3 H), 3.66 (spt, J = 6.9 Hz, 1 H), 2.49 (s, 3 H), 2.15-2.08 (m, 1 |
| | H), 1.39 (d, J = 6.9 Hz, 6 H), 1.14-1.06 (m, 2 H), 0.94-0.87 (m, 2 H). |
| Final Cpd 193 | 1H NMR (400 MHz, DMSO-d6) ppm 0.85-0.93 (m, 2 H), 1.02-1.11 |
| | (m, 2 H), 1.32 (d, J = 6.9 Hz, 6 H), 2.15 (tt, J = 8.4, 5.0 Hz, 1 H), 3.58 |
| | (spt, J = 6.9 Hz, 1 H), 4.87 (s, 2 H), 7.01 (s, 1 H), 7.99 (d, J = 1.8 Hz, 1 |
| | H), 9.42 (d, J = 1.7 Hz, 1 H), 9.42 (s, 1 H), 10.62 (br s, 1 H). |
| | — |
| | 1H NMR (400 MHz, DMSO-d6) ppm 10.62 (br s, 1 H), 9.42 (s, 1 H), |
| | 9.42 (d, J = 1.7 Hz, 1 H), 7.99 (d, J = 1.8 Hz, 1 H), 7.01 (s, 1 H), 4.87 (s, 2 |
| | H), 3.58 (spt, J = 6.9 Hz, 1 H), 2.15 (tt, J = 8.4, 5.0 Hz, 1 H), 1.32 (d, |
| | J = 6.9 Hz, 6 H), 1.11-1.02 (m, 2 H), 0.93-0.85 (m, 2 H). |
| Final Cpd 103 | 1H NMR (400 MHz, DMSO-d6) ppm 0.85-0.93 (m, 2 H), 1.02-1.11 |
| | (m, 2 H), 1.32 (d, J = 6.9 Hz, 6 H), 2.10-2.21 (m, 1 H), 3.58 (spt, J = 6.8 |
| | Hz, 1 H), 4.89 (s, 2 H), 7.01 (s, 1 H), 7.22 (dd, J = 7.4, 2.1 Hz, 1 H), 8.11 |
| | (d, J = 1.7 Hz, 1 H), 8.38 (s, 1 H), 8.86 (d, J = 7.3 Hz, 1 H), 10.80 (br s, 1 H). |
| | — |
| | 1H NMR (400 MHz, DMSO-d6) ppm 10.80 (br s, 1 H), 8.86 (d, J = 7.3 |
| | Hz, 1 H), 8.38 (s, 1 H), 8.11 (d, J = 1.7 Hz, 1 H), 7.22 (dd, J = 7.4, 2.1 Hz, |
| | 1 H), 7.01 (s, 1 H), 4.89 (s, 2 H), 3.58 (spt, J = 6.8 Hz, 1 H), 2.21-2.10 |
| | (m, 1 H), 1.32 (d, J = 6.9 Hz, 6 H), 1.11-1.02 (m, 2 H), 0.93-0.85 (m, 2H). |
| Final Cpd 165 | 1H NMR (400 MHz, DMSO-d6) ppm 0.85-0.92 (m, 2 H), 1.02-1.10 |
| | (m, 2 H), 1.31 (d, J = 6.9 Hz, 6 H), 2.15 (tt, J = 8.4, 4.9 Hz, 1 H), 3.57 |
| | (spt, J = 6.9 Hz, 1 H), 4.91 (s, 2 H), 7.00 (s, 1 H), 7.73 (d, J = 1.1 Hz, 1 |
| | H), 7.81 (br d, J = 9.7 Hz, 1 H), 8.10 (d, J = 0.6 Hz, 1 H), 8.15 (d, J = 0.9 |
| | Hz, 1 H), 11.16 (br s, 1 H). |
| | — |
| | 1H NMR (400 MHz, DMSO-d6) ppm 11.16 (br s, 1 H), 8.15 (d, J = 0.9 |
| | Hz, 1 H), 8.10 (d, J = 0.6 Hz, 1 H), 7.81 (br d, J = 9.7 Hz, 1 H), 7.73 (d, |
| | J = 1.1 Hz, 1 H), 7.00 (s, 1 H), 4.91 (s, 2 H), 3.57 (spt, J = 6.9 Hz, 1 H), |
| | 2.15 (tt, J = 8.4, 4.9 Hz, 1 H), 1.31 (d, J = 6.9 Hz, 6 H), 1.10-1.02 (m, 2 |
| | H), 0.92-0.85 (m, 2 H). |
| Final Cpd 106 | 1H NMR (400 MHz, DMSO-d6) ppm 0.83-0.92 (m, 2 H), 1.01-1.10 |
| | (m, 2 H), 1.31 (d, J = 6.8 Hz, 6 H), 2.15 (tt, J = 8.4, 4.9 Hz, 1 H), 3.57 |
| | (spt, J = 6.8 Hz, 1 H), 3.96 (s, 3 H), 4.90 (s, 2 H), 6.93-7.00 (m, 2 H), |
| | 7.89 (dd, J = 5.0, 1.7 Hz, 1 H), 8.24 (dd, J = 7.8, 1.5 Hz, 1 H), 9.75 (s, 1 H). |
| | — |
| | 1H NMR (400 MHz, DMSO-d6) ppm 9.75 (s, 1 H), 8.24 (dd, J = 7.8, 1.5 |
| | Hz, 1 H), 7.89 (dd, J = 5.0, 1.7 Hz, 1 H), 7.00-6.93 (m, 2 H), 4.90 (s, 2 |
| | H), 3.96 (s, 3 H), 3.57 (spt, J = 6.8 Hz, 1 H), 2.15 (tt, J = 8.4, 4.9 Hz, 1 |
| | H), 1.31 (d, J = 6.8 Hz, 6 H), 1.10-1.01 (m, 2 H), 0.92-0.83 (m, 2 H). |
| Final Cpd 112 | 1H NMR (400 MHz, DMSO-d6) ppm 0.85-0.92 (m, 2 H), 1.03-1.10 |
| | (m, 2H), 1.31 (d, J = 6.8 Hz, 6 H), 2.12-2.19 (m, 1 H), 3.58 (spt, J = 6.8 |
| | Hz, 1 H), 4.86 (s, 2 H), 7.00 (s, 1 H), 8.00 (dt, J = 11.2, 2.3 Hz, 1 H), |
| | 8.31 (d, J = 2.4 Hz, 1 H), 8.53 (s, 1 H), 10.69 (br s, 1 H). |
| | 19F NMR (377 MHz, DMSO-d6) ppm −126.33 (s, 1 F) |
| | — |
| | 1H NMR (400 MHz, DMSO-d6) 10.69 (br s, 1 H), 8.53 (s, 1 H), 8.31 |
| | (d, J = 2.4 Hz, 1 H), 8.00 (dt, J = 11.2, 2.3 Hz, 1 H), 7.00 (s, 1 H), 4.86 (s, |
| | 2 H), 3.58 (spt, J = 6.8 Hz, 1 H), 2.19-2.12 (m, 1 H), 1.31 (d, J = 6.8 Hz, |
| | 6 H), 1.10-1.03 (m, 2 H), 0.92-0.85 (m, 2 H). |
| Final Cpd 113 | 1H NMR (400 MHz, DMSO-d6) ppm 0.83-0.91 (m, 2 H), 1.02-1.10 |
| | (m, 2 H), 1.32 (d, J = 6.9 Hz, 6 H), 2.15 (tt, J = 8.4, 4.9 Hz, 1 H), 2.41 (s, |
| | 3 H), 3.57 (spt, J = 6.9 Hz, 1 H), 4.84 (s, 2 H), 6.99 (s, 1 H), 7.22 (dd, |
| | J = 7.9, 4.9 Hz, 1 H), 7.72 (dd, J = 8.0, 1.6 Hz, 1 H), 8.28 (dd, J = 4.8, 1.5 |
| | Hz, 1 H), 9.70 (s, 1 H). |
| | — |
| | 1H NMR (400 MHz, DMSO-d6) ppm 9.70 (s, 1 H), 8.28 (dd, J = 4.8, 1.5 |
| | Hz, 1 H), 7.72 (dd, J = 8.0, 1.6 Hz, 1 H), 7.22 (dd, J = 7.9, 4.9 Hz, 1 H), |
| | 6.99 (s, 1 H), 4.84 (s, 2 H), 3.57 (spt, J = 6.9 Hz, 1 H), 2.41 (s, 3 H), 2.15 |
| | (tt, J = 8.4, 4.9 Hz, 1 H), 1.32 (d, J = 6.9 Hz, 6 H), 1.10-1.02 (m, 2 H), |
| | 0.91-0.83 (m, 2H). |
| Final Cpd 125 | 1H NMR (400 MHz, CHLOROFORM-d) ppm 0.86-0.93 (m, 2 H), |
| | 1.05-1.13 (m, 2H), 1.40 (d, J = 6.8 Hz, 6 H), 2.11 (tt, J = 8.4, 4.9 Hz, 1 |
| | H), 3.67 (spt, J = 6.9 Hz, 1 H), 3.90 (s, 3 H), 4.91 (s, 2 H), 6.79 (d, J = 5.5 |
| | Hz, 1 H), 6.82 (s, 1 H), 8.26 (br s, 1 H), 8.28 (d, J = 5.5 Hz, 1 H), 9.43 |
| | (s, 1 H). |
| | — |
| | 1H NMR (400 MHz, CHLOROFORM-d) ppm 9.43 (s, 1 H), 8.28 (d, |
| | J = 5.5 Hz, 1 H), 8.26 (br s, 1 H), 6.82 (s, 1 H), 6.79 (d, J = 5.5 Hz, 1 H), |
| | 4.91 (s, 2 H), 3.90 (s, 3 H), 3.67 (spt, J = 6.9 Hz, 1 H), 2.11 (tt, J = 8.4, 4.9 |
| | Hz, 1 H), 1.40 (d, J = 6.8 Hz, 6 H), 1.13-1.05 (m, 2 H), 0.93-0.86 (m, 2H). |
| Final Cpd 124 | 1H NMR (400 MHz, CHLOROFORM-d) ppm 0.88-0.95 (m, 2 H), |
| | 1.06-1.15 (m, 2 H), 1.39 (d, J = 6.9 Hz, 6 H), 2.12 (tt, J = 8.4, 4.9 Hz, 1 |
| | H), 2.34 (s, 3 H), 3.67 (spt, J = 6.8 Hz, 1 H), 4.92 (s, 2 H), 6.86 (s, 1 H), |
| | 8.11-8.18 (m, 2 H), 8.92 (s, 1 H). |
| | — |
| | 1H NMR (400 MHz, CHLOROFORM-d) ppm 8.92 (s, 1 H), 8.18- |
| | 8.11 (m, 2 H), 6.86 (s, 1 H), 4.92 (s, 2 H), 3.67 (spt, J = 6.8 Hz, 1 H), |
| | 2.34 (s, 3 H), 2.12 (tt, J = 8.4, 4.9 Hz, 1 H), 1.39 (d, J = 6.9 Hz, 6 H), 1.15- |
| | 1.06 (m, 2 H), 0.95-0.88 (m, 2 H). |
| Final Cpd 123 | 1H NMR (400 MHz, CHLOROFORM-d) ppm 0.86-0.94 (m, 2 H), |
| | 1.05-1.13 (m, 2 H), 1.39 (d, J = 6.9 Hz, 6 H), 2.11 (tt, J = 8.4, 5.0 Hz, 1 |
| | H), 3.06 (s, 6 H), 3.67 (spt, J = 6.8 Hz, 1 H), 4.85 (s, 2 H), 6.46 (dd, |
| | J = 5.5, 1.8 Hz, 1 H), 6.83 (s, 1 H), 6.96 (d, J = 1.6 Hz, 1 H), 8.02 (d, |
| | J = 5.6 Hz, 1 H), 8.30 (s, 1 H). |
| | — |
| | 1H NMR (400 MHz, CHLOROFORM-d) ppm 8.30 (s, 1 H), 8.02 (d, |
| | J = 5.6 Hz, 1 H), 6.96 (d, J = 1.6 Hz, 1 H), 6.83 (s, 1 H), 6.46 (dd, J = 5.5, |
| | 1.8 Hz, 1 H), 4.85 (s, 2 H), 3.67 (spt, J = 6.8 Hz, 1 H), 3.06 (s, 6 H), 2.11 |
| | (tt, J = 8.4, 5.0 Hz, 1 H), 1.39 (d, J = 6.9 Hz, 6 H), 1.13-1.05 (m, 2 H), |
| | 0.94-0.86 (m, 2H). |
| Final Cpd 126 | 1H NMR (400 MHz, DMSO-d6) ppm 0.85-0.93 (m, 2 H), 1.03-1.10 |
| | (m, 2 H), 1.32 (d, J = 6.8 Hz, 6 H), 2.16 (tt, J = 8.4, 4.9 Hz, 1 H), 3.58 |
| | (spt, J = 6.8 Hz, 1 H), 4.84 (s, 2 H), 7.00 (s, 1 H), 7.10 (dd, J = 8.7, 1.8 |
| | Hz, 1 H), 7.68 (d, J = 8.7 Hz, 1 H), 7.97 (s, 1 H), 8.04 (s, 1 H), 10.31 (s, |
| | 1 H), 12.90 (s, 1 H). |
| | — |
| | 1H NMR (400 MHz, DMSO-d6) ppm 12.90 (s, 1 H), 10.31 (s, 1 H), |
| | 8.04 (s, 1 H), 7.97 (s, 1 H), 7.68 (d, J = 8.7 Hz, 1 H), 7.10 (dd, J = 8.7, 1.8 |
| | Hz, 1 H), 7.00 (s, 1 H), 4.84 (s, 2 H), 3.58 (spt, J = 6.8 Hz, 1 H), 2.16 (tt, |
| | J = 8.4, 4.9 Hz, 1 H), 1.32 (d, J = 6.8 Hz, 6 H), 1.10-1.03 (m, 2 H), 0.93- |
| | 0.85 (m, 2H). |
| Final Cpd 174 | 1H NMR (400 MHz, DMSO-d6, 27° C.) d ppm 0.82-0.93 (m, 2 H) 1.02- |
| | 1.10 (m, 2 H) 1.31 (d, J = 6.82 Hz, 7 H) 2.10-2.20 (m, 1H) 3.51- |
| | 3.64 (m, 1 H) 4.96 (s, 2 H) 6.99 (s, 1 H) 7.06-7.10 (m, 1 H) 7.21 (t, |
| | J = 54.36 Hz, 1 H) 8.02 (d, J = 9.24 Hz, 1 H) 8.45 (s, 1 H) 11.77 (s, 1 H) |
| | 19F NMR (377 MHz, DMSO-d6, 27° C.) d ppm −115.00 (d, J = 54.21 Hz, 1 F) |
| | 13C NMR (101 MHz, DMSO-d6, 27° C.) d ppm 9.67 (s, 1 C) 9.72 (s, 2 |
| | C) 19.87 (s, 2 C) 28.85 (s, 1 C) 53.47 (s, 1 C) 103.07 (s, 1 C) 113.86 (t, |
| | J = 237.36 Hz, 1 C) 119.66 (s, 1 C) 126.71 (s, 1 C) 134.47 (s, 1 C) |
| | 144.17 (s, 1 C) 152.31 (t, J = 26.20 Hz, 1 C) 153.39 (s, 1 C) 156.98 (s, 1 |
| | C) 160.43 (s, 1 C) 167.78 (s, 1 C) |
| Final Cpd 107 | 1H NMR (400 MHz, DMSO-d6, 27° C.) d ppm 0.86-0.91 (m, 2 H) 0.99- |
| | 1.12 (m, 2 H) 1.31 (d, J = 6.90 Hz, 6 H) 2.11-2.19 (m, 1H) 3.57 (spt, |
| | J = 6.85 Hz, 1 H) 3.88 (s, 3 H) 4.82 (s, 2 H) 7.00 (s, 1 H) 8.73 (s, 2 H) |
| | 10.39 (s, 1 H) |
| | 13C NMR (101 MHz, DMSO-d6, 27° C.) d ppm 9.24 (s, 1 C) 9.30 (s, 2 |
| | C) 19.43 (s, 2 C) 28.40 (s, 1 C) 52.53 (s, 1 C) 54.69 (s, 1 C) 102.59 (s, |
| | 1 C) 128.43 (s, 1 C) 134.05 (s, 1 C) 143.70 (s, 1 C) 150.80 (s, 2 C) |
| | 152.93 (s, 1 C) 159.98 (s, 1 C) 161.39 (s, 1 C) 165.74 (s, 1 C) |
| Final Cpd 122 | 1H NMR (400 MHz, DMSO-d6, 27° C.) d ppm 0.86-0.90 (m, 2 H) 1.03- |
| | 1.09 (m, 2H) 1.31 (d, J = 6.82 Hz, 6 H) 2.12-2.18 (m, 1H) 2.41 (s, 3 |
| | H) 3.57 (spt, J = 6.82 Hz, 1 H) 4.81 (s, 2 H) 6.99 (s, 1 H) 7.20 (d, J = 8.36 |
| | Hz, 1 H) 7.86 (dd, J = 8.36, 2.42 Hz, 1 H) 8.58 (d, J = 2.42 Hz, 1 H) 10.27 |
| | (s, 1 H) |
| | 13C NMR (101 MHz, DMSO-d6, 27° C.) d ppm 9.68 (s, 1 C) 9.71 (s, 2 |
| | C) 19.89 (s, 2 C) 23.81 (s, 1 C) 28.85 (s, 1 C) 53.12 (s, 1 C) 103.02 (s, |
| | 1 C) 123.29 (s, 1 C) 127.56 (s, 1 C) 133.19 (s, 1 C) 134.53 (s, 1 C) |
| | 140.72 (s, 1 C) 144.08 (s, 1 C) 153.23 (s, 1 C) 153.39 (s, 1 C) 160.39 |
| | (s, 1 C) 165.99 (s, 1 C) |
| Final Cpd 121 | 1H NMR (400 MHz, DMSO-d6, 27° C.) d ppm 0.85-0.91 (m, 2 H) 1.03- |
| | 1.08 (m, 2H) 1.31 (d, J = 6.82 Hz, 6 H) 2.11-2.18 (m, 1H) 3.57 |
| | (dquin, J = 13.69, 6.76, 6.76, 6.76, 6.76 Hz, 1 H) 3.79 (s, 3 H) 4.83 (s, 2 |
| | H) 6.99 (s, 1 H) 7.69 (br s, 1 H) 8.02 (d, J = 1.98 Hz, 1 H) 8.29 (s, 1 H) |
| | 10.44 (s, 1 H) |
| | 13C NMR (101 MHz, DMSO-d6, 27° C.) d ppm 9.68 (s, 1 C) 9.72 (s, 2 |
| | C) 19.88 (s, 2 C) 28.85 (s, 1 C) 53.26 (s, 1 C) 55.93 (s, 1 C) 103.03 (s, |
| | 1 C) 111.44 (s, 1 C) 132.68 (s, 1 C) 133.41 (s, 1 C) 134.49 (s, 1 C) |
| | 136.35 (s, 1 C) 144.08 (s, 1 C) 153.38 (s, 1 C) 155.77 (s, 1 C) 160.42 |
| | (s, 1 C) 166.37 (s, 1 C) |
| Final Cpd 120 | 1H NMR (400 MHz, DMSO-d6, 27° C.) d ppm 0.85-0.92 (m, 2 H) 1.02- |
| | 1.11 (m, 2H) 1.32 (d, J = 6.79 Hz, 6 H) 2.10-2.21 (m, 1H) 2.57 (s, 3 |
| | H) 3.58 (dt, J = 13.69, 6.85 Hz, 1 H) 5.00 (s, 2 H) 7.00 (s, 1 H) 7.86 (d, |
| | J = 5.43 Hz, 1 H) 8.29 (d, J = 5.43 Hz, 1 H) 9.97 (s, 1 H) |
| | 13C NMR (101 MHz, DMSO-d6, 27° C.) d ppm 9.69 (s, 1 C) 9.75 (s, 2 |
| | C) 19.88 (s, 2 C) 23.57 (s, 1 C) 28.87 (s, 1 C) 53.40 (s, 1 C) 103.09 (s, |
| | 1 C) 115.52 (s, 1 C) 120.71 (s, 1 C) 134.44 (s, 1 C) 142.15 (s, 1 C) |
| | 144.18 (s, 1 C) 147.69 (s, 1 C) 153.38 (s, 1 C) 156.78 (s, 1 C) 160.46 |
| | (s, 1 C) 167.05 (s, 1 C) |
| Final Cpd 130 | 1H NMR (400 MHz, DMSO-d6, 27° C.) d ppm 0.89 (br s, 2 H) 1.07 (br |
| | d, J = 4.84 Hz, 2 H) 1.31 (br d, J = 5.72 Hz, 6 H) 2.16 (br s, 1 H) 3.50- |
| | 3.71 (m, 1 H) 4.88 (br s, 2 H) 6.69-7.15 (m, 1 H) 6.71-7.08 (m, 1 H) |
| | 7.65 (br s, 1 H) 7.92 (br s, 1 H) 8.55 (br d, J = 3.74 Hz, 1 H) 10.85 (br s, 1H) |
| | 13C NMR (101 MHz, DMSO-d6, 27° C.) d ppm 9.68 (s, 1 C) 9.73 (s, 2 |
| | C) 19.87 (s, 2 C) 28.85 (s, 1 C) 53.50 (s, 1 C) 103.11 (s, 1 C) 110.07 (t, |
| | J = 3.85 Hz, 1 C) 114.17 (t, J = 238.90 Hz, 1 C) 115.24 (s, 1 C) 134.41 (s, |
| | 1 C) 144.18 (s, 1 C) 146.90 (s, 1 C) 151.13 (s, 1 C) 153.51 (t, J = 23.89 |
| | Hz, 1 C) 153.38 (s, 1 C) 160.47 (s, 1 C) 167.21 (s, 1 C) |
| | 19F NMR (377 MHz, DMSO-d6, 27° C.) d ppm −115.92 (d, J = 55.21 Hz, 2F) |
| Final Cpd 131 | 1H NMR (400 MHz, DMSO-d6, 27° C.) d ppm 0.83-0.91 (m, 2 H) 1.02- |
| | 1.10 (m, 2 H) 1.31 (d, J = 6.90 Hz, 6 H) 2.10-2.20 (m, 1H) 2.57 (s, 3 |
| | H) 3.57 (spt, J = 6.88 Hz, 1 H) 4.84 (s, 2 H) 7.00 (s, 1 H) 8.84 (s, 2 H) |
| | 10.49 (s, 1 H) |
| | 13C NMR (101 MHz, DMSO-d6, 27° C.) d ppm 9.69 (s, 1 C) 9.75 (s, 2 |
| | C) 19.90 (s, 2C) 25.40 (s, 1 C) 28.86 (s, 1 C) 53.11 (s, 1 C) 103.09 (s, |
| | 1 C) 131.72 (s, 1 C) 134.49 (s, 1 C) 144.18 (s, 1 C) 148.13 (s, 2 C) |
| | 153.40 (s, 1 C) 160.45 (s, 1 C) 162.61 (s, 1 C) 166.49 (s, 1 C) |
| Final Cpd 129 | 1H NMR (400 MHz, DMSO-d6, 27° C.) d ppm 0.85-0.92 (m, 2 H) 1.01- |
| | 1.12 (m, 2 H) 1.31 (d, J = 6.79 Hz, 6 H) 2.10-2.20 (m, 1H) 3.58 (dt, |
| | J = 13.69, 6.85 Hz, 1 H) 3.82 (s, 3 H) 4.83 (s, 2 H) 7.00 (s, 1 H) 7.05- |
| | 7.11 (m, 2 H) 8.05 (d, J = 6.27 Hz, 1 H) 10.56 (s, 1 H) |
| | 13C NMR (101 MHz, DMSO-d6, 27° C.) d ppm 9.69 (s, 1 C) 9.74 (s, 2 |
| | C) 19.88 (s, 2 C) 28.85 (s, 1 C) 53.42 (s, 1 C) 53.60 (s, 1 C) 99.00 (s, 1 |
| | C) 103.07 (s, 1 C) 108.56 (s, 1 C) 134.43 (s, 1 C) 144.10 (s, 1 C) |
| | 147.96 (s, 1 C) 147.99 (s, 1 C) 153.35 (s, 1 C) 160.46 (s, 1 C) 165.07 |
| | (s, 1 C) 166.76 (s, 1 C) |
| Final Cpd 132 | 1H NMR (400 MHz, DMSO-d6, 27° C.) d ppm 0.88 (br s, 2 H) 1.05 (m, |
| | J = 6.10 Hz, 2 H) 1.29 (br d, J = 6.69 Hz, 7 H) 2.03-2.24 (m, 1 H) 3.56 |
| | (dt, J = 13.27, 6.53 Hz, 1 H) 3.90 (s, 3 H) 4.90 (s, 2 H) 6.98 (s, 1 H) 7.60 |
| | (br d, J = 5.12 Hz, 1 H) 8.46 (br d, J = 5.33 Hz, 1 H) 11.20 (br s, 1 H) |
| | 13C NMR (101 MHz, DMSO-d6, 27° C.) d ppm 9.70 (s, 1 C) 9.76 (s, 2 |
| | C) 19.87 (s, 2 C) 28.84 (s, 1 C) 53.57 (s, 1 C) 54.91 (s, 1 C) 103.04 (s, |
| | 1 C) 104.12 (s, 1 C) 134.41 (s, 1 C) 144.11 (s, 1 C) 153.33 (s, 1 C) |
| | 159.75 (s, 1 C) 160.46 (s, 1 C) 161.08 (s, 1 C) 165.25 (s, 1 C) 167.72 |
| | (s, 1 C) |
| Final Cpd 137 | 1H NMR (400 MHz, DMSO-d6, 27° C.) d ppm 0.84-0.92 (m, 2 H) 1.02- |
| | 1.11 (m, 2 H) 1.30 (d, J = 6.90 Hz, 6 H) 2.09-2.22 (m, 1 H) 2.36 (s, 3 |
| | H) 2.49 (s, 3 H) 3.57 (dt, J = 13.69, 6.85 Hz, 1 H) 4.87 (s, 2 H) 6.99 (s, 1 |
| | H) 7.68 (s, 1H) 11.10 (s, 1 H) |
| | 13C NMR (101 MHz, DMSO-d6, 27° C.) d ppm 9.69 (s, 1 C) 9.74 (s, 2 |
| | C) 19.88 (s, 2 C) 24.41 (s, 1 C) 25.65 (s, 1 C) 28.83 (s, 1 C) 53.47 (s, 1 |
| | C) 103.02 (s, 1 C) 106.02 (s, 1 C) 134.46 (s, 1 C) 144.08 (s, 1 C) |
| | 153.37 (s, 1 C) 157.96 (s, 1 C) 160.42 (s, 1 C) 167.05 (s, 1 C) 167.59 |
| | (s, 1 C) 168.65 (s, 1 C) |
| Final Cpd 40 | 1H NMR (500 MHz, DMSO-d6) d ppm 0.84-0.94 (m, 2 H) 1.01-1.11 |
| | (m, 4H) 1.16-1.28 (m, 5 H) 1.96 (td, J = 9.0, 2.3 Hz, 2 H) 2.08-2.19 |
| | (m, 2 H) 2.22-2.30 (m, 2 H) 3.79 (sxt, J = 8.0 Hz, 1 H) 5.05 (br s, 1 H) |
| | 5.06 (s, 2 H) 7.03 (s, 1 H) 8.62 (d, J = 7.0 Hz, 1 H) |
| Final Cpd 202 | 1H NMR (500 MHz, DMSO-d6) d ppm 0.81-0.92 (m, 2 H) 0.98-1.10 |
| | (m, 2 H) 1.20 (s, 3 H) 1.88-1.96 (m, 2 H) 2.13 (tt, J = 8.4, 4.9 Hz, 1 H) |
| | 2.17-2.25 (m, 2 H) 2.54 (s, 3 H) 3.69-3.82 (m, 1 H) 4.50 (s, 2 H) 4.96 |
| | (s, 1 H) 6.96 (s, 1 H) 8.20 (d, J = 7.2 Hz, 1 H) |
| Final Cpd 47 | 1H NMR (500 MHz, DMSO-d6) d ppm 0.79-0.92 (m, 2 H) 0.97-1.11 |
| | (m, 2 H) 2.13 (tt, J = 8.4, 4.9 Hz, 1 H) 2.55 (s, 3 H) 4.27 (d, J = 6.0 Hz, 2 |
| | H) 4.61 (s, 2 H) 6.98 (s, 1 H) 7.10-7.20 (m, 2 H) 7.22-7.33 (m, 2 H) |
| | 8.53 (t, J = 5.9 Hz, 1 H) |
| Final Cpd 59 | 1H NMR (500 MHz, DMSO-d6) d ppm 0.79-0.93 (m, 2 H) 0.97-1.12 |
| | (m, 2 H) 1.54-1.66 (m, 1 H) 2.05-2.18 (m, 2 H) 2.33-2.44 (m, 2 H) |
| | 2.53 (s, 3 H) 2.60-2.71 (m, 2 H) 3.54-3.67 (m, 2 H) 4.14-4.24 (m, 1 |
| | H) 4.43-4.61 (m, 2 H) 6.96 (s, 1 H) 7.29-7.36 (m, 2 H) 8.25 (d, J = 7.2 |
| | Hz, 1 H) 8.44-8.54 (m, 2 H) |
| Final Cpd 65 | 1H NMR (500 MHz, DMSO-d6) d ppm 0.81-0.92 (m, 2 H) 0.98-1.10 |
| | (m, 2H) 1.28-1.37 (m, 1 H) 1.39-1.48 (m, 1 H) 1.53-1.69 (m, 2 H) |
| | 1.78 (ddt, J = 12.8, 8.8, 6.4, 6.4 Hz, 1 H) 1.92 (dtd, J = 13.4, 7.8, 7.8, 5.9 |
| | Hz, 1 H) 2.13 (tt, J = 8.4, 4.9 Hz, 1 H) 2.54 (s, 3 H) 3.73-3.85 (m, 2 H) |
| | 4.54 (s, 2 H) 4.72 (d, J = 4.3 Hz, 1 H) 6.96 (s, 1 H) 7.96 (d, J = 7.3 Hz, 1 H) |
| Final Cpd 53 | 1H NMR (500 MHz, DMSO-d6) d ppm 0.80-0.91 (m, 2 H) 0.98-1.10 |
| | (m, 2H) 1.80-1.90 (m, 2 H) 2.13 (tt, J = 8.4, 4.9 Hz, 1H) 2.51-2.59 |
| | (m, 5 H) 3.74-3.90 (m, 2 H) 4.43 (s, 2 H) 4.50 (s, 2 H) 6.96 (s, 1 H) |
| | 7.30-7.37 (m, 2 H) 7.40-7.51 (m, 2 H) 8.25 (d, J = 7.8 Hz, 1 H) |

### Example B

### Pharmaceutical Compositions

A compound of the invention (without the provisos) (for instance, a compound of the examples) is brought into association with a pharmaceutically acceptable carrier, thereby providing a pharmaceutical composition comprising such active compound. A therapeutically effective amount of a compound of the invention (without the provisos) (e.g. a compound of the examples) is intimately mixed with a pharmaceutically acceptable carrier, in a process for preparing a pharmaceutical composition.

### Example C

### Biological Examples

The activity of a compound according to the present invention can be assessed by in vitro methods. A compound the invention exhibits valuable pharmacological properties, e.g. properties susceptible to inhibit NLRP3 activity, for instance as indicated the following test, and are therefore indicated for therapy related to NLRP3 inflammasome activity.

### PBMC Assay

Peripheral venous blood was collected from healthy individuals and human peripheral blood mononuclear cells (PBMCs) were isolated from blood by Ficoll-Histopaque (Sigma-Aldrich, A0561) density gradient centrifugation. After isolation, PBMCs were stored in liquid nitrogen for later use. Upon thawing, PBMC cell viability was determined in growth medium (RPMI media supplemented with 10% fetal bovine serum, 1% Pen-Strep and 1% L-glutamine). Compounds were spotted in a 1:3 serial dilution in DMSO and diluted to the final concentration in 30 μl medium in 96 well plates (Falcon, 353072). PBMCs were added at a density of 7.5×10⁴ cells per well and incubated for 30 min in a 5% CO₂ incubator at 37° C. LPS stimulation was performed by addition of 100 ng/ml LPS (final concentration, Invivogen, tlrl-smlps) for 6 hrs followed by collection of cellular supernatant and the analysis of IL-1β (μM) and TNF cytokines levels (μM) via MSD technology according to manufacturers' guidelines (MSD, K₁₅₁A0H).

The IC₅₀ values (for IL-1β) and EC50 values (TNF) were obtained on compounds of the invention/examples, and are depicted in the following table:

| | | IL1β | TNF |
|---|---|---|---|
| | | IC₅₀ | EC₅₀ |
| Number | Compound | (μM) | (μM) |
| Final Cpd 2 | 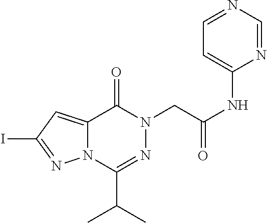 [SMILES: CC(C)c1nn(CC(=O)Nc2ccncn2)c(=O)c2cc(I)nn12] | <0.12 | >10 |
| Final Cpd 11 | 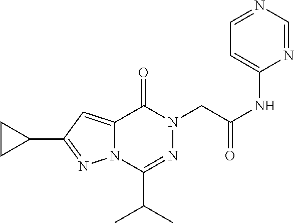 [SMILES: CC(C)c1nn(CC(=O)Nc2ccncn2)c(=O)c2cc(C3CC3)nn12] | 0.17 | >10 |
| Final Cpd 12 | 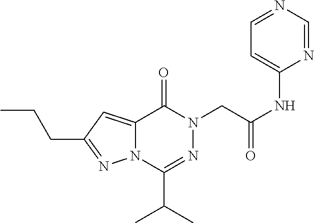 [SMILES: CCCc1cc2c(=O)n(CC(=O)Nc3ccncn3)nc(C(C)C)n2n1] | 0.60 | >10 |
| Final Cpd 1 | 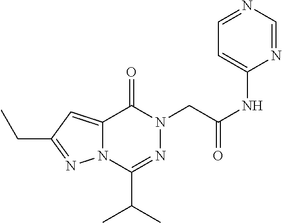 [SMILES: CCc1cc2c(=O)n(CC(=O)Nc3ccncn3)nc(C(C)C)n2n1] | 0.34 | >10 |
| Final Cpd 3 | 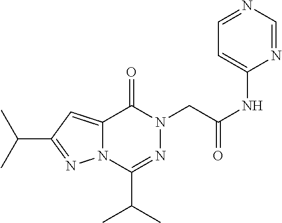 [SMILES: CC(C)c1cc2c(=O)n(CC(=O)Nc3ccncn3)nc(C(C)C)n2n1] | 0.28 | >10 |
| Final Cpd 4 | 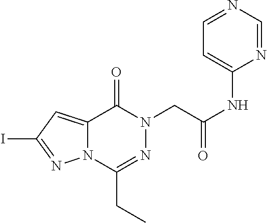 [SMILES: CCc1nn(CC(=O)Nc2ccncn2)c(=O)c2cc(I)nn12] | 0.29 | >10 |
| Final Cpd 10 | 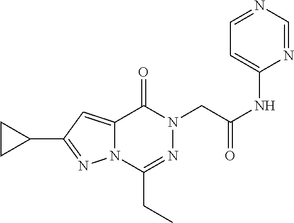 [SMILES: CCc1nn(CC(=O)Nc2ccncn2)c(=O)c2cc(C3CC3)nn12] | 0.64 | >10 |
| Final Cpd 13 | 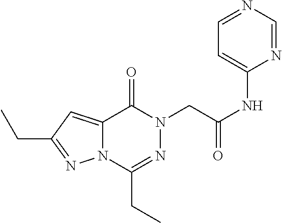 [SMILES: CCc1cc2c(=O)n(CC(=O)Nc3ccncn3)nc(CC)n2n1] | 1.71 | >10 |
| Final Cpd 5 | 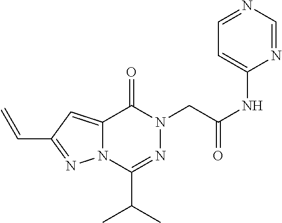 [SMILES: C=Cc1cc2c(=O)n(CC(=O)Nc3ccncn3)nc(C(C)C)n2n1] | | |
| Final Cpd 14 | 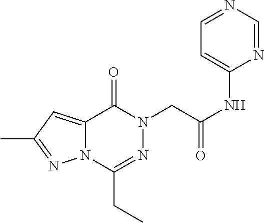 [SMILES: CCc1nn(CC(=O)Nc2ccncn2)c(=O)c2cc(C)nn12] | 8.11 | >10 |
| Final Cpd 15 | 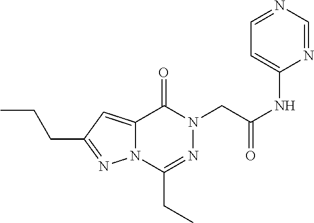 [SMILES: CCCc1cc2c(=O)n(CC(=O)Nc3ccncn3)nc(CC)n2n1] | 2.4 | >10 |
| Final Cpd 16 | 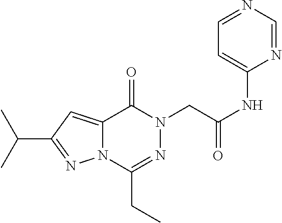 [SMILES: CCc1nn(CC(=O)Nc2ccncn2)c(=O)c2cc(C(C)C)nn12] | 0.84 | >10 |
| Final Cpd 17 | 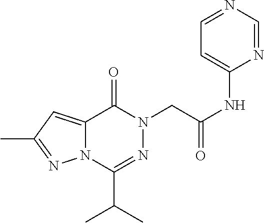 [SMILES: Cc1cc2c(=O)n(CC(=O)Nc3ccncn3)nc(C(C)C)n2n1] | 1.1 | >10 |
| Final Cpd 6 | 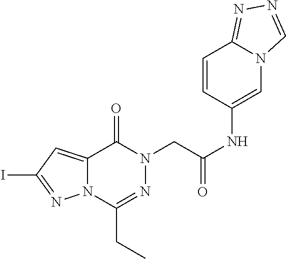 [SMILES: CCc1nn(CC(=O)Nc2ccc3nncn3c2)c(=O)c2cc(I)nn12] | 0.23 | >10 |
| Final Cpd 7 | 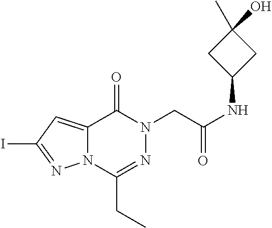 [SMILES: CCc1nn(CC(=O)N[C@H]2C[C@@](C)(O)C2)c(=O)c2cc(I)nn12] | 0.6 | >10 |
| Final Cpd 8 | 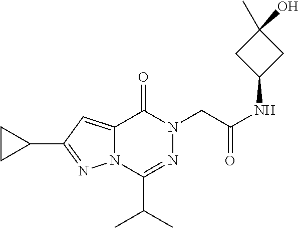 [SMILES: CC(C)c1nn(CC(=O)N[C@H]2C[C@@](C)(O)C2)c(=O)c2cc(C3CC3)nn12] | 0.24 | >10 |
| Final Cpd 9 | 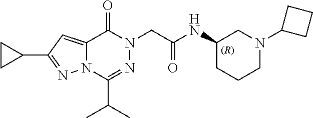 | <0.12 | >10 |
| Final Cpd 18 | 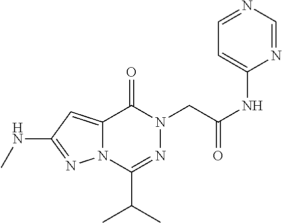 [SMILES: CNc1cc2c(=O)n(CC(=O)Nc3ccncn3)nc(C(C)C)n2n1] | 0.16 | >10 |
| Final Cpd 20 | 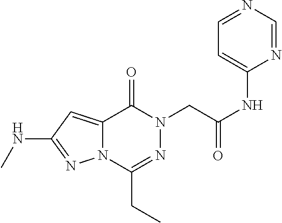 [SMILES: CCc1nn(CC(=O)Nc2ccncn2)c(=O)c2cc(NC)nn12] | 1.14 | >10 |
| Final Cpd 21 | 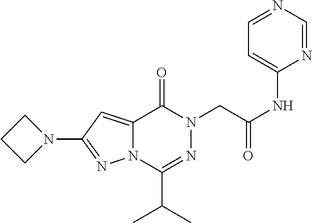 [SMILES: CC(C)c1nn(CC(=O)Nc2ccncn2)c(=O)c2cc(N3CCC3)nn12] | 2.75 | >10 |
| Final Cpd 22 | 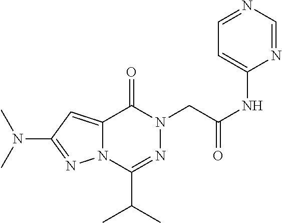 [SMILES: CC(C)c1nn(CC(=O)Nc2ccncn2)c(=O)c2cc(N(C)C)nn12] | 1.35 | >10 |
| Final Cpd 23 | | 0.055 | >10 |
| Final Cpd 25 | | 2.34 | >10 |
| Final Cpd 26 | | >10 | >10 |
| Final Cpd 27 | | 0.99 | >10 |
| Final Cpd 28 | | 0.38 | >10 |
| Final Cpd 178 | | 3.90 | >10 |
| Final Cpd 76 | | >10 | >10 |
| Final Cpd 77 | | >10 | >10 |
| Final Cpd 78 | | >10 | >10 |
| Final Cpd 79 | | >10 | >10 |
| Final Cpd 80 | | 0.08 | >10 |
| Final Cpd 179 | | >10 | >10 |
| Final Cpd 180 | | >10 | >10 |
| Final Cpd 183 | | 5.04 | >10 |
| Final Cpd 184 | | >10 | >10 |
| Final Cpd 185 | | 4.11 | >10 |
| Final Cpd 186 | | >10 | >10 |
| Final Cpd 153 | | 0.090 | >10 |
| Final Cpd 178 | | 6.36 | >10 |
| Final Cpd 157 | | >10 | >10 |
| Final Cpd 160 | | >10 | >10 |
| Final Cpd 159 | | 2.24 | >10 |
| Final Cpd 156 | | 1.96 | >10 |
| Final Cpd 158 | | >10 | >10 |
| Final Cpd 144 | | 0.32 | >10 |
| Final Cpd 145 | | 0.51 | >10 |
| Final Cpd 142 | | 0.14 | >10 |
| Final Cpd 143 | | 0.10 | >10 |
| Final Cpd 147 | | 0.40 | >10 |
| Final Cpd 141 | | 0.42 | >10 |
| Final Cpd 140 | | 0.21 | >10 |
| Final Cpd 146 | | 0.21 | >10 |
| Final Cpd 152 | | 0.86 | >10 |
| Final Cpd 155 | | 1.14 | >10 |
| Final Cpd 154 | | 3.00 | >10 |
| Final Cpd 177 | | 2.06 | |
| Final Cpd 148 | | 0.32 | >10 |
| Final Cpd 151 | | >10 | >10 |
| Final Cpd 149 | | 0.78 | >10 |
| Final Cpd 150 | | 0.36 | >10 |
| Final Cpd 34 | | 5.66 | >10 |
| Final Cpd 188 | | 0.74 | >10 |
| Final Cpd 191 | | 0.021 | >10 |
| Final Cpd 190 | | 0.054 | >10 |
| Final Cpd 108 | | 0.54 | >10 |
| Final Cpd 128 | | 2.55 | >10 |
| Final Cpd 136 | | 1.41 | >10 |
| Final Cpd 138 | | >10 | >10 |
| Final Cpd 135 | | 6.93 | >10 |
| Final Cpd 24 | | 0.11 | >10 |
| Final Cpd 198 | | >10 | >10 |
| Final Cpd 195 | | >10 | >10 |
| Final Cpd 196 | | >10 | >10 |
| Final Cpd 197 | | >10 | >10 |
| Final Cpd 194 | | >10 | >10 |
| Final Cpd 189 | | 1.16 | >10 |
| Final Cpd 166 | | 0.044 | >10 |
| Final Cpd 192 | | 0.61 | >10 |
| Final Cpd 175 | | 0.22 | >10 |
| Final Cpd 161 | | 0.11 | >10 |
| Final Cpd 176 | | 0.082 | >10 |
| Final Cpd 83 | | >10 | >10 |
| Final Cpd 82 | | 0.28 | >10 |
| Final Cpd 85 | | 0.34 | >10 |
| Final Cpd 167 | | 0.38 | >10 |
| Final Cpd 168 | | 0.84 | >10 |
| Final Cpd 81 | | 0.16 | >10 |
| Final Cpd 84 | | 0.63 | >10 |
| Final Cpd 86 | | >10 | >10 |
| Final Cpd 87 | | 0.063 | 6.17 |
| Final Cpd 169 | | 0.16 | >10 |
| Final Cpd 88 | | 1.29 | >10 |
| Final Cpd 89 | | 0.23 | >10 |
| Final Cpd 170 | | 0.55 | >10 |
| Final Cpd 90 | | 0.39 | >10 |
| Final Cpd 91 | | 0.31 | >10 |
| Final Cpd 171 | | 0.22 | >10 |
| Final Cpd 92 | | 0.92 | >10 |
| Final Cpd 93 | | 1.51 | >10 |
| Final Cpd 98 | | >10 | >10 |
| Final Cpd 99 | | 0.49 | >10 |
| Final Cpd 100 | | 0.31 | >10 |
| Final Cpd 101 | | 7.75 | >10 |
| Final Cpd 104 | | 1.62 | >10 |
| Final Cpd 105 | | 0.18 | >10 |
| Final Cpd 116 | | 1.23 | >10 |
| Final Cpd 134 | | 0.23 | >10 |
| Final Cpd 70 | | 0.62 | >10 |
| Final Cpd 71 | | 0.54 | >10 |
| Final Cpd 72 | | 0.054 | >10 |
| Final Cpd 73 | | 0.11 | >10 |
| Final Cpd 74 | | 0.22 | >10 |
| Final Cpd 75 | | 0.024 | 7.04 |
| Final Cpd 94 | | 2.34 | >10 |
| Final Cpd 109 | | 1.08 | >10 |
| Final Cpd 115 | | 1.72 | >10 |
| Final Cpd 110 | | 0.59 | >10 |
| Final Cpd 111 | | 0.83 | >10 |
| Final Cpd 117 | | 1.41 | >10 |
| Final Cpd 118 | | 0.11 | >10 |
| Final Cpd 119 | | 0.52 | >10 |
| Final Cpd 127 | | 0.18 | >10 |
| Final Cpd 133 | | 0.44 | 10 |
| Final Cpd 29 | | 1.79 | >10 |
| Final Cpd 30 | | 1.43 | >10 |
| Final Cpd 31 | | 0.69 | >10 |
| Final Cpd 187 | | 0.80 | >10 |
| Final Cpd 35 | | 0.38 | >10 |
| Final Cpd 32 | | 0.15 | >10 |
| Final Cpd 33 | | 1.56 | >10 |
| Final Cpd 36 | | 0.97 | >10 |
| Final Cpd 44 | | 0.62 | >10 |
| Final Cpd 45 | | 4.48 | >10 |
| Final Cpd 43 | | 1.06 | >10 |
| Final Cpd 182 | | 0.10 | >10 |
| Final Cpd 46 | | >10 | >10 |
| Final Cpd 181 | | 0.48 | >10 |
| Final Cpd 41 | | 0.88 | >10 |
| Final Cpd 42 | | 0.22 | >10 |
| Final Cpd 163 | | 0.62 | >10 |
| Final Cpd 37 | | 0.56 | >10 |
| Final Cpd 39 | | 2.49 | >10 |
| Final Cpd 38 | | >10 | >10 |
| Final Cpd 68 | | 0.78 | >10 |
| Final Cpd 69 | | ~2.45 | >10 |
| Final Cpd 164 | | 0.54 | >10 |
| Final Cpd 172 | | >10 | >10 |
| Final Cpd 173 | | >10 | >10 |
| Final Cpd 95 | | 0.37 | >10 |
| Final Cpd 96 | | 0.24 | >10 |
| Final Cpd 97 | | 0.20 | >10 |
| Final Cpd 102 | | 0.52 | >10 |
| Final Cpd 193 | | 0.98 | >10 |
| Final Cpd 103 | | 0.56 | >10 |
| Final Cpd 114 | | 0.35 | >10 |
| Final Cpd 165 | | 0.10 | >10 |
| Final Cpd 106 | | 0.71 | >10 |
| Final Cpd 112 | | 0.88 | >10 |
| Final Cpd 113 | | 0.85 | >10 |
| Final Cpd 125 | | 9.37 | >10 |
| Final Cpd 124 | | 0.61 | >10 |
| Final Cpd 123 | | 1.04 | >10 |
| Final Cpd 126 | | 0.07 | >10 |
| Final Cpd 174 | | 6.75 | >10 |
| Final Cpd 107 | | 4.23 | >10 |
| Final Cpd 122 | | 0.82 | >10 |
| Final Cpd 121 | | 3.06 | >10 |
| Final Cpd 120 | | 0.55 | >10 |
| Final Cpd 130 | | 0.53 | >10 |
| Final Cpd 131 | | 3.57 | >10 |
| Final Cpd 129 | | 0.67 | >10 |
| Final Cpd 132 | | 0.72 | >10 |
| Final Cpd 137 | | 1.23 | >10 |
| Final Cpd 139 | | 6.56 | >10 |
| Final Cpd 199 | | >10 | >10 |
| Final Cpd 200 | | >10 | >10 |
| Final Cpd 201 | | >10 | >10 |
| Final Cpd 47 | | >10 | >10 |
| Final Cpd 55 | | >10 | >10 |
| Final Cpd 56 | | 6.97 | >10 |
| Final Cpd 57 | | >10 | >10 |
| Final Cpd 58 | | 9.41 | >10 |
| Final Cpd 59 | | >10 | >10 |
| Final Cpd 60 | | >10 | >10 |
| Final Cpd 61 | | 0.064 | >10 |
| Final Cpd 62 | | 8.53 | >10 |
| Final Cpd 63 | | 7.89 | >10 |
| Final Cpd 48 | | 0.89 | >10 |
| Final Cpd 64 | | 5.50 | >10 |
| Final Cpd 65 | | 4.19 | >10 |
| Final Cpd 66 | | 3.55 | >10 |
| Final Cpd 67 | | >10 | >10 |
| Final Cpd 49 | | >10 | >10 |
| Final Cpd 50 | | >10 | >10 |
| Final Cpd 51 | | >10 | >10 |
| Final Cpd 52 | | 2.31 | >10 |
| Final Cpd 53 | | >10 | >10 |
| Final Cpd 54 | | >10 | >10 |

### Example D

### Further Testing

One or more compound(s) of the invention (including compounds of the final examples) is/are tested in a number of other methods to evaluate, amongst other properties, permeability, stability (including metabolic stability and blood stability) and solubility.

### Permeability Test

The in vitro passive permeability and the ability to be a transported substrate of P-glycoprotein (P-gp) is tested using MDCKcells stably transduced with MDR1 (this may be performed at a commercial organisaiton offering ADME, PK services, e.g. Cyprotex). Permeability experiments are conducted in duplicate at a single concentration (5 μM) in a transwell system with an incubation of 120 min. The apical to basolateral (AtoB) transport in the presence and absence of the P-gp inhibitor GF120918 and the basolateral to apical (BtoA) transport in the absence of the P-gp inhibitor is measured and permeation rates (Apparent Permeability) of the test compounds (Pₐₚₑ 10⁻⁶ cm/sec) are calculated.

### Metabolic Stability Test in Liver Microsomes

The metabolic stability of a test compound is tested (this may be performed at a commercial organisaiton offering ADME, PK services, e.g. Cyprotex) by using liver microsomes (0.5 mg/ml protein) from human and preclinical species incubated up to 60 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (ml/min/mg microsomal protein) is calculated using the following formula:

$${Cl}_{int} = \frac{0.693}{{t}_{1 / 2}} \times \frac{{V}_{inc}}{{W}_{mic prot , inc}}$$

Where: V_{inc}=incubation volume,

W_{mic prot,inc}=weight of microsomal protein in the incubation.

### Metabolic Stability Test in Liver Hepatocytes

The metabolic stability of a test compound is tested using liver hepatocytes (1 milj cells) from human and preclinical species incubated up to 120 minutes at 37° C. with 1 μM test compound.

The in vitro metabolic half-life (tv2) is calculated using the slope of the log-linear regression from the percentage parent compound remaining versus time relationship (κ), t_{1/2}=−ln(2)/κ.

The in vitro intrinsic clearance (Clᵢₙₜ) (μl/min/million cells) is calculated using the following formula:

$${Cl}_{int} = \frac{0.693}{{t}_{1 / 2}} \times \frac{{V}_{inc}}{\# {cells}_{inc}} \times 1000$$

Where: V_{inc}=incubation volume,

# cells_{inc}=number of cells (×10⁶) in the incubation

Solubility test

The test/assay is run in triplicate and is semi-automated using the Tecan Fluent for all liquid handling with the following general steps:
20 μl of 10 mM stock solution is dispensed in a 500 μl 96 well plate
DMSO is evaporated (Genevac)
a stir bar and 400 μl of buffer/biorelevant media is added
the solution is stirred for 72 h (pH₂ and pH₇) or 24 h (FaSSIF and FeSSIF)
the solution is filtered
the filtrate is quantified by UPLC/UV using a three-points calibration curve

The LC conditions are:
Waters Acquity UPLC
Mobile phase A: 0.1% formic acid in H₂O, B: 0.1% formic acid in CH3CN
Column: Waters HSS T3 1.8 μm 2.1×50mm
Column temp.: 55° C.
Inj.vo.: 2 μl
Flow: 0.6ml/min
Wavelength UV: 250_350 nm
Gradient : 0 min: 0%B, 0.3min: 5%B, 1.8 min: 95%B, 2.6 min: 95%B

### Blood Stability Assay

The compound of the invention/examples is spiked at a certain concentration in plasma or blood from the agreed preclinical species; then after incubating to predetermined times and conditions (37° C., 0° C. (ice) or room temperature) the concentration of the test compound in the blood or plasma matrix with LCMS/MS can then be determined.

1. A compound of Formula (I) 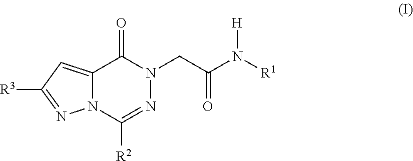
or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
R² represents:
(i) C₁₋₃ alkyl optionally substituted with one or more substituents independently selected from halo, —OH, —OC₁₋₃alkyl and oxo;
(ii) C3-6cycloalkyl;
(iii) C₂₋₄alkenyl optionally substituted with —O—C₁₋₃alkyl;
(iv) —O—C₁₋₃alkyl;
(v) —N(H)alkyl or —N-(C₁₋₃alkyl)₂, where each alkyl may be optionally substituted with —OC₁₋₃ alkyl; or
(vi) heterocyclyl;
R³ represents:
(i) hydrogen;
(ii) halo;
(iii) C₁₋₄ alkyl optionally substituted with one or more substituents independently selected from halo, —OH, oxo, —O—C₁₋₃alkyl, —C(O)OH, —C(O)N(H)heteroaryl, —C(O)N(H)aryl, —C(O)N(H)C₁₋₃ alkyl and —C(O)N(C₁₋₃alkyl)₂;
(iv) C₂₋₄ alkenyl;
(V) C₃₋₆ cycloalkyl;
(vi) —OC₁₋₄ alkyl;
(vii) —N(H)C₁₋₃alkyl or —N(C₁₋₃alkyl)₂;
(viii) —C(O)N(H)C₁₋₃alkyl or —C(O)N(C₁₋₃alkyl)₂;
(ix) aryl or heteroaryl; or
(x) heterocyclyl,
provided that:
(i) when R² represents methyl, and R³ represents 4-methoxyphenyl, then R¹ does not represent 3-ethylphenyl, 2,4-dimethylphenyl,3-fluorophenyl, 2,4-dimethoxyphenyl, 2-methoxy-5-methylphenyl, 3,5-dimethylphenyl, 3-chloro-4-methylphenyl, 2-ethylphenyl, 4-fluorophenyl, 3,5-dimethoxyphenyl, cyclopropyl, 2-methylphenyl, 2,3-dimethylphenyl, 3-(methylthio)phenyl, 4-methoxyphenyl, cyclohexyl, 3-chlorophenyl, cyclopentyl, cycloheptyl, 4-chlorophenyl, 1,2,3,4-tetrahydro-1-naphthalenyl or 1,3-benzodioxol-5-yl;
(ii) when R² represents methyl, and R³ represents 4-ethylphenyl, then R¹ does not represent 2,3-dimethylcyclohexyl, 4-chlorophenyl, 1,2,3,4-tetrahydro-1-naphthalenyl, 3-chlorophenyl, cyclohexyl, 2-methylcyclohexyl, 3,5-dimethoxyphenyl, 2,4-difluorophenyl, cyclopentyl, cycloheptyl or 4-acetylphenyl;
(iii) when R² represents methyl, R³ represents phenyl, then R¹ does not represent cycloheptyl, 4-chlorophenyl, 3-chlorophenyl, 4-methoxyphenyl, cyclohexyl, cyclopentyl or 4-acetylphenyl.

2. The compound of claim 1, wherein R¹ represents C₃₋₆ cycloalkyl optionally substituted by one or two substituents selected from C₁₋₃ alkyl and —OH.

3. The compound of claim 2, wherein:
R¹ represents: 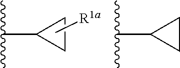
where R^{1a} represents an optional substituent selected from —OH and C₁₋₃ alkyl, or, is not present; or, R¹ represents: 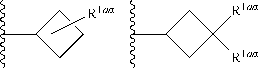
where each R^{1aa} represents one or two optional substituents selected from —OH and C₁₋₃ alkyl.

4. The compound of claim 1, wherein R¹ represents a mono-cyclic 5- or 6-membered heterocyclyl group containing at least one nitrogen heteroatom, and which is optionally substituted by one substituent selected from C₁₋₃ alkyl and C₃₋₆ cycloalkyl.

5. The compound of claim 1, wherein R¹ represents: (i) phenyl; (ii) a 5- or 6-membered mono-cyclic heteroaryl group; or (iii) a 9- or 10-membered bicyclic heteroaryl group, all of which are optionally substituted with one or two substituent(s) selected from halo, —OH and —OC₁₋₃ alkyl.

6. The compound of claim 5, wherein R¹ represents phenyl or a mono-cyclic 6-membered heteroaryl group: 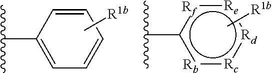
wherein R¹′ represents one or two optional substituents selected from halo, —OH and —OCH₃, and, either one or two of Rb, Rc, Rd, Re and Rf represent(s) a nitrogen heteroatom (and the others represent a CH).

7. The compound of claim 6, wherein R¹ represents: 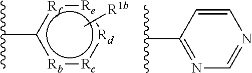
in which Rb and Rd represent a nitrogen atom, and, in an embodiment, there is no R^{1b} substituent present.

8. The compound of claim 5, wherein R¹ represents a 9- or 10-membered bicyclic heteroaryl group, for instance: 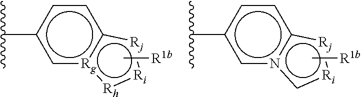
wherein R^{ib} represents one or two optional substituent selected from halo, —OH and —OCH₃, each ring of the bicyclic system is aromatic, R_{g} represents a N or C atom and any one or two of Rₕ, Rᵢ and Rⱼ represents N and the other(s) represent(s) C.

9. The compound of claim 8, wherein R¹ represents: 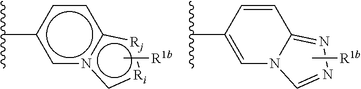
in which one of Rᵢ and Rⱼ represents N and the other represents C, or, both Rᵢ and Rⱼ represent N, and there is no R^{1b} substituent present.

10. The compound of claim 1, wherein R¹ represents cyclopropyl, as defined in claim 2 or claim 3, or a phenyl or mono-cyclic heteraryl group, as defined in claim 6 or claim 7.

11. The compound of claim 1, wherein R² represents (i) C₁₋₃ alkyl optionally substituted by one or two substituent(s) selected from —OH, methoxy, ethoxy and oxo; (ii) C₂₋₄alkenyl optionally substituted with methoxy or ethoxy; (iii)
—N—(C₁₋₃alkyl)₂, where the alkyl moiety is unsubstituted; (iv) a 6-membered heterocyclyl group in which there is at least one nitrogen heteroatom and optionally an oxygen heteroatom.

12. The compound of claim 11, wherein R² represents unsubstituted C₁₋₃ alkyl.

13. The compound of claim 1, wherein R³ represents: (i) hydrogen; (ii) halo; (iii) C14 alkyl optionally substituted by one or more substituent selected from fluoro, —OH and —O—C₁₋₂ alkyl; (iv) C₂₋₄ alkenyl; or (v) C₃₋₄ cycloalkyl.

14. A pharmaceutical composition comprising a therapeutically effective amount of a compound as defined in claim 1 but without the provisos and a pharmaceutically acceptable carrier.

15. A process for preparing a pharmaceutical composition comprising a therapeutically effective amount of a compound as defined in claim 1 but without the provisos and a pharmaceutically acceptable carrier, characterized in that a pharmaceutically acceptable carrier is intimately mixed with a therapeutically effective amount of a compound as defined in claim 1 but without the provisos.

16. (canceled)

17. A combination comprising: (a) a compound according to claim 1 but without the provisos; and (b) one or more other therapeutic agents.

18. (canceled)

19. A method of treating a disease or disorder associated with inhibition of NLRP3 inflammasome activity in a subject in need thereof, the method comprising administering to said subject a therapeutically effective amount of a compound according to claim 1.

20. The compound method of treating according to claim 19 wherein the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

21. A process for the preparation of a compound of formula (I) as claimed in claim 1, which comprises:
(i) reaction of a compound of formula (II), 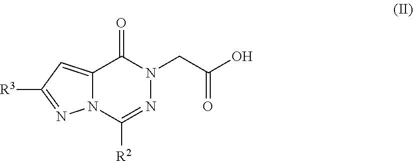
or a derivative thereof, wherein R² and R³ are as defined in claim 1, with a compound of formula (III),
H₂N—R¹ (III)
or a derivative thereof, wherein R¹ is as defined in claim 1, under amide-forming reaction conditions;
(ii) reaction of a compound of formula (IV), 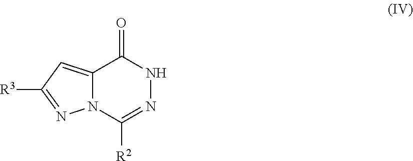
wherein R² and R³ are as defined in claim 1, with a compound of formula (V),
LG^{a}CH₂—C(O)—N(H)R¹ (V)
wherein LG^{a} represents a suitable leaving group and R¹ is as defined in claim 1;
(iii) by transformation of a certain compound of formula (I) into another.

22. A compound of formula (II) or a compound of formula (IV), as depicted in claim 21: 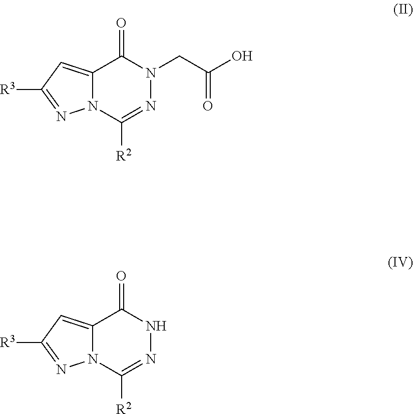
wherein R² and R³ are as defined in claim 1.
